SEQUENCE LISTING (142 sequences)

SEQ ID NO: 1 - protein, 109 aa, unidentified
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 31 aa, synthetic construct
HAEGTFTSDVSSYLEGQAAKEFIAWLVKGRG

SEQ ID NO: 3 - protein, 31 aa, synthetic construct
  SITE 2: note = 2-Aminoisobutyric acid
HXEGTFTSDYSSYLEEQAAKEFIAWLVKGGG

SEQ ID NO: 4 - protein, 120 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 5 - protein, 107 aa, synthetic construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 6 - protein, 8 aa, synthetic construct
GFTFSAYA

SEQ ID NO: 7 - protein, 8 aa, synthetic construct
ISGSGGSA

SEQ ID NO: 8 - protein, 13 aa, synthetic construct
AKDGAWKMSGLDV

SEQ ID NO: 9 - protein, 6 aa, synthetic construct
QDISDY

SEQ ID NO: 10 - skipped

SEQ ID NO: 11 - protein, 9 aa, synthetic construct
QKYDSAPLT

SEQ ID NO: 12 - protein, 31 aa, synthetic construct
  SITE 2: note = 2-Aminoisobutyric acid
  SITE 3: note = 3-(tetrazol-5-yl)Alanine
HXXGTFTSDYSSYLEEQAAKEFIAWLVKGGG

SEQ ID NO: 13 - protein, 47 aa, synthetic construct
  SITE 2: note = 2-Aminoisobutyric acid
  SITE 47: note = Amidated 2-amino-6-(2-(cyclooct-2-yn-1-yloxy)acetamido)hexanamide
HXEGTFTSDYSSYLEEQAAKEFIAWLVKGGGGGGGSGGGGSGGGGSK

SEQ ID NO: 14 - protein, 42 aa, synthetic construct
  SITE 2: note = 2-Aminoisobutyric acid
  SITE 42: note = Amidated 2-amino-6-(2-(cyclooct-2-yn-1-yloxy)acetamido)hexanamide
HXEGTFTSDYSSYLEEQAAKEFIAWLVKGGGGGGGSGGGGSK

SEQ ID NO: 15 - protein, 37 aa, synthetic construct
  SITE 2: note = 2-Aminoisobutyric acid
  SITE 37: note = Amidated 2-amino-6-(2-(cyclooct-2-yn-1-yloxy)acetamido)hexanamide
HXEGTFTSDYSSYLEEQAAKEFIAWLVKGGGGGGGSK

SEQ ID NO: 16 - protein, 47 aa, synthetic construct; identical to SEQ ID NO: 13
  SITE 2: note = 2-Aminoisobutyric acid
  SITE 47: note = Amidated azido lysine modified residue

SEQ ID NO: 17 - protein, 47 aa, synthetic construct; identical to SEQ ID NO: 13
  SITE 2: note = 2-Aminoisobutyric acid
  SITE 47: note = Amidated acetyl-2'-maleimido modified residue

SEQ ID NO: 18 - protein, 32 aa, synthetic construct
  SITE 2: note = 2-Aminoisobutyric acid
  SITE 32: note = Amidated residue linked to another sequence via the side chain
HXEGTFTSDYSSYLEEQAAKEFIAWLVKGGGK

SEQ ID NO: 19 - protein, 32 aa, synthetic construct; identical to SEQ ID NO: 18
  SITE 2: note = 2-Aminoisobutyric acid
  SITE 32: note = Amidated azide modified residue

SEQ ID NO: 20 - protein, 47 aa, synthetic construct; identical to SEQ ID NO: 13
  SITE 2: note = 2-Aminoisobutyric acid
  SITE 47: note = Amidated propanoyl-3'-maleimido modified residue

SEQ ID NO: 21 - protein, 352 aa, synthetic construct
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRRRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 22 - protein, 451 aa, synthetic construct
QVQLVQSGAEVKKPGASVKVSCKVSGYTLTELSIHWVRQVPGKGLEWMGGFDPEEGETIFAQEFRDRVTLTEDTSPDTAYMELSSLKSEDAAVYYCTTPRYCNNGICYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 215 aa, synthetic construct
DIQMTQSPSSLSASVGDRVTITCRASQSISNYLNWYQQKPGKAPKLLIYTASSLQSGVPSRFSGSGSGTDFTLTISSLQTEDFATYYCQQSYSNFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 451 aa, synthetic construct
EVQLVESGGGLVQPGGSLRLSCAASGFTFSDHYMDWVRQAPGKGLEWVGRTRNKANSHTTEYTASVTGRFTISRDDSRNSLYLQMNSLKTEDTAVYYCVRAGIIGTLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 215 aa, synthetic construct
DIQMTQFPSSLSASVGDRVTITCRASQSISSFLNWFQQKPGKAPKFLIYAASSLQSGVPSRFSGSGSGTDFTLTISSLQPEDFATYYCQQSYSSPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 449 aa, synthetic construct
QVQLQESGPGLVKPSETLSLTCTVTGGSISRNYWSWIRQPPGKGLEWIGYIYYSGSIDYNPSLKSRVTISVDTSKNQFSLKLSSMTAADTAVYYCARDRWNWKYGMDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 215 aa, synthetic construct
EIVLTQSPGTLSLSPGERATLSCRASQTVRNNYLAWYHQKPGQAPRLLIYGASSRATGIPDRFSGSGSGTDFTLTISRLEPEDFTVYYCHQYGNSPWTFGQGTKMEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 447 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 214 aa, synthetic construct
  SITE 55: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 452 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDLLVRFLEWSHYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 215 aa, synthetic construct
QSVLTQPPSASGTPGQRVTISCSGSSSNIGSNTVHWYQQLPGTAPKLLIYSDNQRPSGVPDRFSGSKSGTSASLAISGLQSEDEADYYCAAWDDSLNGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 445 aa, synthetic construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 217 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 445 aa, synthetic construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 214 aa, synthetic construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 439 aa, synthetic construct
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 215 aa, synthetic construct
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSGYHFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 31 aa, synthetic construct
  SITE 2: note = 2-Aminoisobutyric acid
  SITE 6: note = Alpha-methyl tyrosine
  SITE 10: note = 2-amino-3-(4'-(4-aminobutoxy)-[1,1'-biphenyl]-4-yl) propanoic acid
HXEGTYTSDXSSYLEEQAAKEFIAWLVKGGG

SEQ ID NO: 39 - protein, 31 aa, synthetic construct; identical to SEQ ID NO: 38
  SITE 2: note = 2-Aminoisobutyric acid
  SITE 6: note = Alpha-methyl tyrosine
  SITE 10: note = 2-amino-3-(4'-(4-aminobutoxy)-[1,1'-biphenyl]-4-yl) propanoic acid
  SITE 20: note = Alpha-methyl lysine
  SITE 22: note = Alpha-methyl phenylalanine
  SITE 28: note = Alpha-methyl lysine

SEQ ID NO: 40 - protein, 31 aa, synthetic construct; identical to SEQ ID NO: 3
  SITE 2: note = 2-Aminoisobutyric acid
  SITE 6: note = Alpha-methyl 2-fluorophenylalanine

SEQ ID NO: 41 - protein, 31 aa, synthetic construct; identical to SEQ ID NO: 12
  SITE 2: note = 2-Aminoisobutyric acid
  SITE 3: note = 3-(tetrazol-5-yl)Alanine

SEQ ID NO: 42 - protein, 30 aa, synthetic construct
  SITE 2: note = 2-Aminoisobutyric acid
  SITE 6: note = Alpha-methyl 2-fluorophenylalanine
HXEGTFTSDYSSYLEEQAAKEFIWLVKGGG

SEQ ID NO: 43 - protein, 31 aa, synthetic construct; identical to SEQ ID NO: 38
  SITE 2: note = 2-Aminoisobutyric acid
  SITE 6: note = Alpha-methyl tyrosine
  SITE 10: note = 2-amino-3-(4'-(4-aminobutoxy)-[1,1'-biphenyl]-4-yl) propanoic acid
  SITE 20: note = Alpha-methyl lysine
  SITE 22: note = Alpha-methyl phenylalanine
  SITE 28: note = Alpha-methyl lysine

SEQ ID NO: 44 - protein, 31 aa, synthetic construct; identical to SEQ ID NO: 3
  SITE 2: note = 2-Aminoisobutyric acid
  SITE 6: note = Alpha-methyl 2-fluorophenylalanine

SEQ ID NO: 45 - protein, 31 aa, synthetic construct; identical to SEQ ID NO: 3
  SITE 2: note = 2-Aminoisobutyric acid
  SITE 6: note = 2-amino-3-(4-aminophenyl)propanoic acid

SEQ ID NO: 46 - protein, 31 aa, synthetic construct
  SITE 2: note = 2-Aminoisobutyric acid
  SITE 11: note = 2-amino-5-(3,5-dimethylphenyl)pentanoic acid
HXEGTFTSDYXSYLEEQAAKEFIAWLVKGGG

SEQ ID NO: 47 - protein, 32 aa, synthetic construct
  SITE 2: note = 2-Aminoisobutyric acid
  SITE 6: note = Alpha-methyl tyrosine
  SITE 10: note = 2-amino-3-(4'-(4-aminobutoxy)-[1,1'-biphenyl]-4-yl) propanoic acid
  SITE 32: note = Amidated azide modified residue
HXEGTYTSDXSSYLEEQAAKEFIAWLVKGGGK

SEQ ID NO: 48 - protein, 32 aa, synthetic construct; identical to SEQ ID NO: 47
  SITE 2: note = 2-Aminoisobutyric acid
  SITE 6: note = Alpha-methyl tyrosine
  SITE 10: note = 2-amino-3-(4'-(4-aminobutoxy)-[1,1'-biphenyl]-4-yl) propanoic acid
  SITE 20: note = Alpha-methyl lysine
  SITE 22: note = Alpha-methyl phenylalanine
  SITE 28: note = Alpha-methyl lysine
  SITE 32: note = Amidated azide modified residue

SEQ ID NO: 49 - protein, 32 aa, synthetic construct; identical to SEQ ID NO: 18
  SITE 2: note = 2-Aminoisobutyric acid
  SITE 6: note = Alpha-methyl 2-fluorophenylalanine
  SITE 32: note = Amidated azide modified residue

SEQ ID NO: 50 - protein, 32 aa, synthetic construct
  SITE 2: note = 2-Aminoisobutyric acid
  SITE 3: note = 3-(tetrazol-5-yl)Alanine
  SITE 32: note = Amidated azido lysine modified residue
HXXGTFTSDYSSYLEEQAAKEFIAWLVKGGGK

SEQ ID NO: 51 - protein, 32 aa, synthetic construct; identical to SEQ ID NO: 47
  SITE 2: note = 2-Aminoisobutyric acid
  SITE 6: note = Alpha-methyl tyrosine
  SITE 10: note = 2-amino-3-(4'-(4-aminobutoxy)-[1,1'-biphenyl]-4-yl) propanoic acid
  SITE 20: note = Alpha-methyl lysine
  SITE 22: note = Alpha-methyl phenylalanine
  SITE 28: note = Alpha-methyl lysine
  SITE 32: note = Amidated residue linked to another sequence via the side chain

SEQ ID NO: 52 - protein, 47 aa, synthetic construct; identical to SEQ ID NO: 13
  SITE 2: note = 2-Aminoisobutyric acid
  SITE 6: note = Alpha-methyl 2-fluorophenylalanine
  SITE 47: note = Amidated residue

SEQ ID NO: 53 - protein, 47 aa, synthetic construct; identical to SEQ ID NO: 13
  SITE 2: note = 2-Aminoisobutyric acid
  SITE 6: note = 2-amino-3-(4-aminophenyl)propanoic acid
  SITE 47: note = Amidated residue

SEQ ID NO: 54 - protein, 47 aa, synthetic construct
  SITE 2: note = 2-Aminoisobutyric acid
  SITE 11: note = 2-amino-5-(3,5-dimethylphenyl)pentanoic acid
HXEGTFTSDYXSYLEEQAAKEFIAWLVKGGGGGGGSGGGGSGGGGSK

SEQ ID NO: 55 - protein, 47 aa, synthetic construct; identical to SEQ ID NO: 13
  SITE 2: note = 2-Aminoisobutyric acid
  SITE 6: note = Alpha-methyl 2-fluorophenylalanine
  SITE 47: note = Amidated azido lysine modified residue

SEQ ID NO: 56 - protein, 47 aa, synthetic construct
  SITE 2: note = 2-Aminoisobutyric acid
  SITE 3: note = 3-(tetrazol-5-yl)Alanine
  SITE 47: note = Amidated azido lysine modified residue
HXXGTFTSDYSSYLEEQAAKEFIAWLVKGGGGGGGSGGGGSGGGGSK

SEQ ID NO: 57 - protein, 32 aa, synthetic construct; identical to SEQ ID NO: 18
  SITE 2: note = 2-Aminoisobutyric acid
  SITE 32: note = Amidated azido lysine modified residue

SEQ ID NO: 58 - protein, 32 aa, synthetic construct; identical to SEQ ID NO: 18
  SITE 2: note = 2-Aminoisobutyric acid
  SITE 32: note = Amidated 2-amino-6-(4-((2-(cyclooct-2-yn-1-yloxy)ac etamido)methyl)-1H-1,2,3-triazol-1-yl)hexanamide

SEQ ID NO: 59 - protein, 47 aa, synthetic construct; identical to SEQ ID NO: 13
  SITE 2: note = 2-Aminoisobutyric acid
  SITE 47: note = Amidated azide modified residue

SEQ ID NO: 60 - protein, 47 aa, synthetic construct; identical to SEQ ID NO: 13
  SITE 2: note = 2-Aminoisobutyric acid
  SITE 47: note = Amidated 2-amino-6-(4-((2-(cyclooct-2-yn-1-yloxy)ac etamido)methyl)-1H-1,2,3-triazol-1-yl)hexanamide

SEQ ID NO: 61 - protein, 125 aa, synthetic construct
QVQLVESGPGLVKPSETLSLTCTVYGGSISSGNYYWGWIRQPPGKGLEWIGTIYYSGSAYYNPSLKSRVTMSVDTSKNQFSLKLSSVTAADTAVYYCVRDYYDSSGHYYNWFDPWGQGTTVTVSS

SEQ ID NO: 62 - protein, 108 aa, synthetic construct
AIQMTQSPSSLSASVGDRVTITCRASQDIRHDLGWYQQKPGKAPKRLIYAASSLQSGVPSRFSGSGSGTEFTLTISSLQPEDFATYYCLQHNTYPWTFGQGTKVEIKR

SEQ ID NO: 63 - protein, 118 aa, synthetic construct
QVQLVESGGGVVQPGRSLRLSCAASGFTFSRYGIHWVRQAPGKGLEWVAVISYDGSDEYYVDSVKGRFSISRDNSKNTLYLQMNSLRPADSAVYYCVKGDLELGFDYWGQGTLVTVSS

SEQ ID NO: 64 - protein, 112 aa, synthetic construct
DIVMTQAAPSIPVIPGESVSMSCRSSKSLLYSNGHTYVYWFVQRPGQSPQLLIYRMSNLASGVPDRFSGSGSGTAFTLRISRVEAEDVGVYYCMQNLEFPLTFGAGTKLELK

SEQ ID NO: 65 - protein, 122 aa, synthetic construct
EVQLLESGGGLVQPGGSLRLSCAASGFTSITYAMSWVRQAPGKGLEWVSAISVSGTNTYYADSVKGRFTISRDKSKNMLYLQMNSLRAEDTAVYYCAKDLLHNWKYGTFDIWGQGTMVTVSS

SEQ ID NO: 66 - protein, 107 aa, synthetic construct
EIVMTQSPATLSVSPGERATLSCRASQSVDSNLVWYQQKPGQVPRLLIYGASTRATGIPARFSGSGSGTEFTLTISSLQSEDFAVYYCQQYNKWPLTFGGGTKVEIK

SEQ ID NO: 67 - protein, 120 aa, synthetic construct; identical to SEQ ID NO: 4

SEQ ID NO: 68 - protein, 107 aa, synthetic construct; identical to SEQ ID NO: 5

SEQ ID NO: 69 - protein, 121 aa, synthetic construct
QVQLVESGGGVVQPGKSLRLSCAASGFTFSSFGMHWVRQAPGKGLEWVAVIGYDGGNEYYADSVKGRFTISRDNSKNTLNLQMSSLRAEDTAVYYCSTISHYDILSGMDVWGRGTTVTVSS

SEQ ID NO: 70 - protein, 108 aa, synthetic construct
DIQMTQSPSSVSASVGDRVTITCRASQGISNWLAWYQQKPGKAPKLLIFAASSLQSGVPSRFSGSASGTDFTLTINSLQPEDFATYYCQQANSFPLTFGGGTKVEIKR

SEQ ID NO: 71 - protein, 31 aa, synthetic construct
  SITE 2: note = 2-Aminoisobutyric acid
  VARIANT 3: note = E or 3-(tetrazol-5-yl)Alanine
  VARIANT 6: note = Alpha-methyl tyrosine, F, alpha-methyl 2-fluorophenylalanine, or 2-amino-3-(4-aminophenyl)propanoic acid
  VARIANT 10: note = 2-amino-3-(4'-(4-aminobutoxy)-[1,1'-biphenyl]-4-yl) propanoic acid or Y
  VARIANT 11: note = S or 2-amino-5-(3,5-dimethylphenyl)pentanoic acid
  VARIANT 20: note = K or alpha-methyl lysine
  VARIANT 22: note = F or alpha-methyl phenylalanine
  VARIANT 28: note = K or alpha-methyl lysine
HXXGTXTSDXXSYLEEQAAXEXIAWLVXGGG

SEQ ID NO: 72 - protein, 4 aa, synthetic construct
GGGS

SEQ ID NO: 73 - protein, 5 aa, synthetic construct
GGGGS

SEQ ID NO: 74 - protein, 8 aa, synthetic construct
GGGSGGGS

SEQ ID NO: 75 - protein, 10 aa, synthetic construct
GGGGSGGGGS

SEQ ID NO: 76 - protein, 12 aa, synthetic construct
GGGSGGGSGGGS

SEQ ID NO: 77 - protein, 15 aa, synthetic construct
GGGGSGGGGSGGGGS

SEQ ID NO: 78 - protein, 16 aa, synthetic construct
GGGSGGGSGGGSGGGS

SEQ ID NO: 79 - protein, 20 aa, synthetic construct
GGGGSGGGGSGGGGSGGGGS

SEQ ID NO: 80 - protein, 20 aa, synthetic construct
GGGSGGGSGGGSGGGSGGGS

SEQ ID NO: 81 - protein, 25 aa, synthetic construct
GGGGSGGGGSGGGGSGGGGSGGGGS

SEQ ID NO: 82 - protein, 24 aa, synthetic construct
GGGSGGGSGGGSGGGSGGGSGGGS

SEQ ID NO: 83 - protein, 30 aa, synthetic construct
GGGGSGGGGSGGGGSGGGGSGGGGSGGGGS

SEQ ID NO: 84 - protein, 24 aa, synthetic construct
  VARIANT 1..24: note = This sequence may encompass 1-6 GGGS repeating units
GGGSGGGSGGGSGGGSGGGSGGGS

SEQ ID NO: 85 - protein, 30 aa, synthetic construct; identical to SEQ ID NO: 83
  VARIANT 1..30: note = This sequence may encompass 1-6 GGGGS repeating units

SEQ ID NO: 86 - protein, 25 aa, synthetic construct
  VARIANT 25: note = Azide modified lysine, 2-amino-6-(2-(cyclooct-2-yn-1-yloxy)acetamido)hexanamide, acetyl-2'-maleimido modified lysine, or propanoyl-3'-maleimido modified lysine
  VARIANT 1..24: note = This region may encompass 1-6 GGGS repeating units
GGGSGGGSGGGSGGGSGGGSGGGSK

SEQ ID NO: 87 - protein, 31 aa, synthetic construct
  VARIANT 31: note = Azide modified lysine, 2-amino-6-(2-(cyclooct-2-yn-1-yloxy)acetamido)hexanamide, acetyl-2'-maleimido modified lysine, or propanoyl-3'-maleimido modified lysine
  VARIANT 1..30: note = This region may encompass 1-6 GGGGS repeating units
GGGGSGGGGSGGGGSGGGGSGGGGSGGGGSK

SEQ ID NO: 88 - protein, 6 aa, synthetic construct
GGGGSK

SEQ ID NO: 89 - protein, 25 aa, synthetic construct
  VARIANT 25: note = Azide modified lysine, 2-amino-6-(2-(cyclooct-2-yn-1-yloxy)acetamido)hexanamide, acetyl-2'-maleimido modified lysine, or propanoyl-3'-maleimido modified lysine
  VARIANT 1..24: note = This region may encompass 1-6 GGGS repeating units
GGGSGGGSGGGSGGGSGGGSGGGSK

SEQ ID NO: 90 - protein, 31 aa, synthetic construct; identical to SEQ ID NO: 87
  VARIANT 31: note = Azide modified lysine, 2-amino-6-(2-(cyclooct-2-yn-1-yloxy)acetamido)hexanamide, acetyl-2'-maleimido modified lysine, or propanoyl-3'-maleimido modified lysine
  VARIANT 1..30: note = This region may encompass 1-6 GGGGS repeating units

SEQ ID NO: 91 - protein, 30 aa, synthetic construct
  SITE 2: note = 2-methyl alanine
  SITE 29: note = 2-methyl alanine
  SITE 30: note = Amidated residue
HAEGTFTSDVSSYLEGQAAKEFIAWLVKAR

SEQ ID NO: 92 - protein, 25 aa, synthetic construct
  VARIANT 25: note = Azide modified lysine, 2-amino-6-(2-(cyclooct-2-yn-1-yloxy)acetamido)hexanamide, acetyl-2'-maleimido modified lysine, or propanoyl-3'-maleimido modified lysine
  VARIANT 1..24: note = This region may encompass 1-6 GGGS repeating units
GGGSGGGSGGGSGGGSGGGSGGGSK

SEQ ID NO: 93 - protein, 47 aa, synthetic construct; identical to SEQ ID NO: 13
  SITE 2: note = 2-Aminoisobutyric acid
  SITE 47: note = Amidated bromo acetyl modified residue

SEQ ID NO: 94 - protein, 47 aa, synthetic construct; identical to SEQ ID NO: 13
  SITE 2: note = 2-Aminoisobutyric acid
  SITE 47: note = Amidated 2-amino-6-(2-(cyclooct-2-yn-1-yloxy)acetamido)hexanamide

SEQ ID NO: 95 - protein, 42 aa, synthetic construct; identical to SEQ ID NO: 14
  SITE 2: note = 2-Aminoisobutyric acid
  SITE 42: note = Amidated 2-amino-6-(2-(cyclooct-2-yn-1-yloxy)acetamido)hexanamide

SEQ ID NO: 96 - protein, 37 aa, synthetic construct; identical to SEQ ID NO: 15
  SITE 2: note = 2-Aminoisobutyric acid
  SITE 37: note = Amidated 2-amino-6-(2-(cyclooct-2-yn-1-yloxy)acetamido)hexanamide

SEQ ID NO: 97 - protein, 47 aa, synthetic construct; identical to SEQ ID NO: 13
  SITE 2: note = 2-Aminoisobutyric acid
  SITE 47: note = Amidated azido lysine modified residue

SEQ ID NO: 98 - protein, 47 aa, synthetic construct; identical to SEQ ID NO: 13
  SITE 2: note = 2-Aminoisobutyric acid
  SITE 47: note = Amidated acetyl-2'-maleimido modified residue

SEQ ID NO: 99 - protein, 32 aa, synthetic construct; identical to SEQ ID NO: 18
  SITE 2: note = 2-Aminoisobutyric acid
  SITE 32: note = Amidated residue linked to another sequence via the side chain

SEQ ID NO: 100 - protein, 32 aa, synthetic construct; identical to SEQ ID NO: 18
  SITE 2: note = 2-Aminoisobutyric acid
  SITE 32: note = Amidated azide modified residue

SEQ ID NO: 101 - protein, 15 aa, synthetic construct
  SITE 1: note = Pent-4-ynoyl modified residue
  SITE 15: note = Amidated 2-amino-6-(2-(cyclooct-2-yn-1-yloxy)acetamido)hexanamide
GGGSGGGGSGGGGSK

SEQ ID NO: 102 - protein, 45 aa, synthetic construct
  SITE 2: note = 2-Aminoisobutyric acid
HXEGTFTSDYSSYLEEQAAKEFIAWLVKGGGGGGGSGGGGSGGGG

SEQ ID NO: 103 - protein, 47 aa, synthetic construct; identical to SEQ ID NO: 13
  SITE 2: note = 2-Aminoisobutyric acid
  SITE 47: note = Amidated bromo acetyl modified residue

SEQ ID NO: 104 - protein, 47 aa, synthetic construct; identical to SEQ ID NO: 13
  SITE 2: note = 2-Aminoisobutyric acid
  SITE 47: note = Amidated 2-amino-6-(2-(cyclooct-2-yn-1-yloxy)acetamido)hexanamide

SEQ ID NO: 105 - protein, 42 aa, synthetic construct; identical to SEQ ID NO: 14
  SITE 2: note = 2-Aminoisobutyric acid
  SITE 42: note = Amidated 2-amino-6-(2-(cyclooct-2-yn-1-yloxy)acetamido)hexanamide

SEQ ID NO: 106 - protein, 37 aa, synthetic construct; identical to SEQ ID NO: 15
  SITE 2: note = 2-Aminoisobutyric acid
  SITE 37: note = Amidated 2-amino-6-(2-(cyclooct-2-yn-1-yloxy)acetamido)hexanamide

SEQ ID NO: 107 - protein, 47 aa, synthetic construct; identical to SEQ ID NO: 13
  SITE 2: note = 2-Aminoisobutyric acid
  SITE 47: note = Amidated azido lysine modified residue

SEQ ID NO: 108 - protein, 47 aa, synthetic construct; identical to SEQ ID NO: 13
  SITE 2: note = 2-Aminoisobutyric acid
  SITE 47: note = Amidated acetyl-2'-maleimido modified residue

SEQ ID NO: 109 - protein, 32 aa, synthetic construct; identical to SEQ ID NO: 18
  SITE 2: note = 2-Aminoisobutyric acid
  SITE 32: note = Amidated residue linked to another sequence via the side chain

SEQ ID NO: 110 - protein, 32 aa, synthetic construct; identical to SEQ ID NO: 18
  SITE 2: note = 2-Aminoisobutyric acid
  SITE 32: note = Azide modified residue

SEQ ID NO: 111 - protein, 15 aa, synthetic construct
  SITE 1: note = Pent-4-ynoyl modified residue
  SITE 15: note = Amidated 2-amino-6-(2-(cyclooct-2-yn-1-yloxy)acetamido)hexanamide
GGGSGGGGSGGGGSK

SEQ ID NO: 112 - protein, 16 aa, synthetic construct
FLIYAASSLQSGVPSR

SEQ ID NO: 113 - protein, 16 aa, synthetic construct
  SITE 10: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
LLIYTTSTLQSGVPSR

SEQ ID NO: 114 - protein, 47 aa, synthetic construct
  SITE 1: note = 2-amino-6-(2-mercaptoacetamido)hexanamide modified residue
  SITE 46: note = 2-Aminoisobutyric acid
KSGGGGSGGGGSGGGGGGGKVLWAIFEKAAQEELYSSYDSTFTGEXH

SEQ ID NO: 115 - protein, 47 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 46: note = 2-Aminoisobutyric acid
QSGGGGSGGGGSGGGGGGGKVLWAIFEKAAQEELYSSYDSTFTGEXH

SEQ ID NO: 116 - protein, 47 aa, synthetic construct; identical to SEQ ID NO: 115
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
  SITE 46: note = 2-Aminoisobutyric acid

SEQ ID NO: 117 - protein, 42 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 41: note = 2-Aminoisobutyric acid
QSGGGGSGGGGGGGKVLWAIFEKAAQEELYSSYDSTFTGEXH

SEQ ID NO: 118 - protein, 42 aa, synthetic construct; identical to SEQ ID NO: 117
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
  SITE 41: note = 2-Aminoisobutyric acid

SEQ ID NO: 119 - protein, 37 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 36: note = 2-Aminoisobutyric acid
QSGGGGGGGKVLWAIFEKAAQEELYSSYDSTFTGEXH

SEQ ID NO: 120 - protein, 37 aa, synthetic construct; identical to SEQ ID NO: 119
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
  SITE 36: note = 2-Aminoisobutyric acid

SEQ ID NO: 121 - protein, 47 aa, synthetic construct; identical to SEQ ID NO: 114
  SITE 1: note = 2-amino-6-(2-((1-(4-(2-(2-(2-(2-aminoethoxy)ethoxy) ethoxy)ethoxy)phenyl)-4-methyl-5,6,7,8,9,10-hexahydrocyclo octa[d]pyridazin-5-yl)oxy)acetamido)hexanamide modified residue
  SITE 46: note = 2-Aminoisobutyric acid

SEQ ID NO: 122 - protein, 47 aa, synthetic construct; identical to SEQ ID NO: 114
  SITE 1: note = 2-amino-6-(2-((4-(4-(2-(2-(2-(2-aminoethoxy)ethoxy) ethoxy)ethoxy)phenyl)-1-methyl-5,6,7,8,9,10-hexahydrocyclo octa[d]pyridazin-5-yl)oxy)acetamido)hexanamide modified residue
  SITE 46: note = 2-Aminoisobutyric acid

SEQ ID NO: 123 - protein, 47 aa, synthetic construct; identical to SEQ ID NO: 114
  SITE 1: note = 1-amino-23-(3-(5-amino-6-((5,6-diamino-6-oxohexyl)a mino)-6-oxohexyl)-3,9-dihydro-8H-dibenzo[b,f][1,2,3]triazo lo[4,5-d]azocin-8-yl)-16,19,23-trioxo-3,6,9,12-tetraoxa-15 ,20-diazatricosane-18-sulfonic acid modified residue
  SITE 46: note = 2-Aminoisobutyric acid

SEQ ID NO: 124 - protein, 47 aa, synthetic construct; identical to SEQ ID NO: 114
  SITE 1: note = 1-amino-23-(1-(5-amino-6-((5,6-diamino-6-oxohexyl)a mino)-6-oxohexyl)-1,9-dihydro-8H-dibenzo[b,f][1,2,3]triazo lo[4,5-d]azocin-8-yl)-16,19,23-trioxo-3,6,9,12-tetraoxa-15 ,20-diazatricosane-18-sulfonic acid modified residue
  SITE 46: note = 2-Aminoisobutyric acid

SEQ ID NO: 125 - protein, 47 aa, synthetic construct; identical to SEQ ID NO: 114
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 46: note = 2-Aminoisobutyric acid

SEQ ID NO: 126 - protein, 47 aa, synthetic construct; identical to SEQ ID NO: 114
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
  SITE 46: note = 2-Aminoisobutyric acid

SEQ ID NO: 127 - protein, 47 aa, synthetic construct; identical to SEQ ID NO: 114
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 46: note = 2-Aminoisobutyric acid

SEQ ID NO: 128 - protein, 47 aa, synthetic construct; identical to SEQ ID NO: 114
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
  SITE 46: note = 2-Aminoisobutyric acid

SEQ ID NO: 129 - protein, 47 aa, synthetic construct; identical to SEQ ID NO: 114
  SITE 1: note = 2-amino-6-(2-mercaptoacetamido)hexanamide modified residue
  SITE 46: note = 2-Aminoisobutyric acid

SEQ ID NO: 130 - protein, 47 aa, synthetic construct; identical to SEQ ID NO: 114
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 46: note = 2-Aminoisobutyric acid

SEQ ID NO: 131 - protein, 47 aa, synthetic construct; identical to SEQ ID NO: 114
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
  SITE 46: note = 2-Aminoisobutyric acid

SEQ ID NO: 132 - protein, 47 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 16: note = 3-(1-(5,6-diamino-6-oxohexyl)-1H-1,2,3-triazol-4-yl )propanoic acid
  SITE 47: note = 2-Aminoisobutyric acid
KGGGGSGGGGSGGGGXGGGKVLWAIFEKAAQEELYSSYDSTFTGEXH

SEQ ID NO: 133 - protein, 48 aa, synthetic construct
  SITE 1: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
  SITE 16: note = 3-(1-(5,6-diamino-6-oxohexyl)-1H-1,2,3-triazol-4-yl )propanoic acid
  SITE 47: note = 2-Aminoisobutyric acid
KSGGGGSGGGGSGGGGXGGGKVLWAIFEKAAQEELYSSYDSTFTGEXH

SEQ ID NO: 134 - protein, 47 aa, synthetic construct
  SITE 1: note = 2-amino-6-(2-((1-(4-(aminomethyl)phenyl)-4-methyl-5 ,6,7,8,9,10-hexahydrocycloocta[d]pyridazin-5-yl)oxy)acetam ido)hexanamide modified residue
  SITE 16: note = 3-(1-(5,6-diamino-6-oxohexyl)-1H-1,2,3-triazol-4-yl )propanoic acid
  SITE 46: note = 2-Aminoisobutyric acid
QSGGGGSGGGGSGGGGXGGKVLWAIFEKAAQEELYSSYDSTFTGEXH

SEQ ID NO: 135 - protein, 19 aa, synthetic construct
  SITE 19: note = Residue linked to another sequence via the side chain
GGGGGGGSGGGGSGGGGSK

SEQ ID NO: 136 - protein, 18 aa, synthetic construct
  SITE 18: note = 2-amino-6-(2-(cyclooct-2-yn-1-yloxy)acetamido)hexan amide
TAGGGGSGGGGSGGGGSK

SEQ ID NO: 137 - protein, 17 aa, synthetic construct
  SITE 17: note = 2-amino-6-(2-(cyclooct-2-yn-1-yloxy)acetamido)hexan amide modified residue
TGGGGSGGGGSGGGGSK

SEQ ID NO: 138 - protein, 17 aa, synthetic construct
  SITE 17: note = 2-amino-6-(2-(cyclooct-2-yn-1-yloxy)acetamido)hexan amide
TAGGGSGGGGSGGGGSK

SEQ ID NO: 139 - protein, 17 aa, synthetic construct
  SITE 17: note = 2-amino-6-(2-(cyclooct-2-yn-1-yloxy)acetamido)hexan amide
TAGGGSGGGGSGGGGSK

SEQ ID NO: 140 - protein, 47 aa, synthetic construct; identical to SEQ ID NO: 13
  SITE 2: note = 2-Aminoisobutyric acid
  SITE 47: note = Amidated bromo acetyl modified residue

SEQ ID NO: 141 - protein, 16 aa, synthetic construct
  SITE 10: note = 2-amino-6-(2-((4-(4-(aminomethyl)phenyl)-1-methyl-5 ,6,7,8,9,10-hexahydrocycloocta[d]pyridazin-5-yl)oxy)acetam ido)hexanamide modified residue
LLIYTTSTLQSGVPSR

SEQ ID NO: 142 - protein, 19 aa, synthetic construct
  SITE 19: note = Residue linked to another sequence via the side chain
GGGGGGGSGGGGSGGGGSK

(19) United States
(12) Patent Application Publication
(10) Pub. No.: US 2026/0199503 A1
(43) Pub. Date: Jul. 16, 2026

(54) GLP1R AGONIST-TETHERED GDF8 ANTIBODY CONJUGATES AND USES THEREOF

(71) Applicant: Regeneron Pharmaceuticals, Inc., Tarrytown, NY (US)

(72) Inventors: Amy HAN, Hockessin, DE (US); Jason Mastaitis, Yorktown Heights, NY (US); Xiang Zheng, Allendale, NJ (US); Se Hyun Kim, Fair Lawn, NJ (US); Jean Yanolatos, Bronx, NY (US); Judith Altarejos, Chappaqua, NY (US); Mark Sleeman, New York, NY (US); Chester A. Metcalf, III, Ardsley, NY (US); William Olson, Yorktown Heights, NY (US); Andrew J. Murphy, Croton-on-Hudson, NY (US); George D. Yancopoulos, Yorktown Heights, NY (US)

(73) Assignee: Regeneron Pharmaceuticals, Inc., Tarrytown, NY (US)

(21) Appl. No.: 19/445,230

(22) Filed: Jan. 9, 2026

Related U.S. Application Data
(60) Provisional application No. 63/744,076, filed on Jan. 10, 2025.
(60) Provisional application No. 63/770,235, filed on Mar. 11, 2025.
(60) Provisional application No. 63/862,384, filed on Aug. 12, 2025.

(51) Int. Cl. A61K 47/68; A61P 3/04; C07K 14/00; C07K 16/22

(52) U.S. Cl. CPC A61K 47/6845; A61K 47/6811; A61K 47/6889; A61P 3/04; C07K 14/001; C07K 16/22

(57) ABSTRACT

Described herein are protein-drug conjugates and compositions thereof that are useful, for example, for targeting glucagon-like peptide 1 receptor (GLP1R). In certain embodiments, provided are peptidomimetic payloads and linker-payloads and methods of making same. More specifically, GLP1 peptidomimetics, antibody-drug conjugates, and compositions which comprise anti-GLP1R antibodies and GLP1 peptidomimetic payloads and methods of treating GLP1R-associated conditions are provided.

### CROSS-REFERENCE TO RELATED APPLICATIONS

This application claims benefit to Provisional Application Nos. 63/744,076, filed Jan. 10, 2025, 63/770,235, filed Mar. 11, 2025, and 63/862,384, filed Aug. 12, 2025, which are hereby incorporated by reference in their entirety.

### SEQUENCE LISTING

The instant application contains a Sequence Listing which has been submitted electronically in XML format and is hereby incorporated by reference in its entirety. Said XML copy, created on Dec. 30, 2025, is named 250298_001183_SL.xml. and is 216,975 bytes in size.

### FIELD OF THE DISCLOSURE

The present disclosure relates to protein-ligand conjugates (e.g., antibody-ligand conjugates), processes of manufacturing, pharmaceutical compositions, and methods of treating diseases therewith. More specifically, the present disclosure relates to an antibody-tethered GLP1R agonist ligand (ATL) conjugates, processes of manufacturing, and methods of treating GDF8 and GLP1R-associated conditions therewith.

### BACKGROUND OF THE DISCLOSURE

Obesity and the closely associated type 2 diabetes mellitus are a global problem for over a third of the world population. In the United States of America, the average obesity rate is over 20%. The costs of obesity-related illness are staggering, amounting to $190.2 billion, roughly 21% of annual medical costs in the U.S. Obesity is an epidemic disease characterized by chronic low-grade inflammation associated with dysfunctional (elevated) fat mass. Obesity is an important underlying risk factor for developing other diseases such as heart disease, stroke, and diabetes. Even a modest decrease in body weight (5-10% of initial body weight) lowers the risk for developing obesity-associated diseases such as heart disease and diabetes.

Glucagon-Like Peptide 1 Receptor (GLP1R) is the receptor for glucagon-like peptide 1 (GLP1) and is expressed in the pancreatic beta cells. GLP1R is also expressed in the brain where it functions in the control of appetite, memory, and learning. GLP1R is a member of the secretin family (Class B) of G protein-coupled receptors (GPCRs). Upon binding of its ligand, GLP1, GLP1R initiates a downstream signaling cascade through Gαs G-proteins that raises intracellular cyclic AMP (cAMP) levels, which leads to the transcriptional regulation of genes (Donnelly, Br J Pharmacol, 166(1):27-41 (2011)). Activation of GLP1R results in increased insulin synthesis and release of insulin.

GLP-1 analogues, fusion proteins and GLP-1 receptor agonists are disclosed, for example, in U.S. Pat. Nos. 7,452,966, 8,389,689, 8,496,149, 8,497,240, 8,557,769, 8,883,447, 8,895,694, 9,409,966, US20160194371, US20140024586, US20140073563, US20120148586, US20170114115, US20170112904, US20160361390, US20150313908, US20150259416, WO2017074715, WO2016127887, WO2015021871, WO2014113357, EP3034514, EP2470198, and EP2373681.

GLP1R and GLP1 are highly validated targets for the treatment of obesity and type 2 diabetes mellitus. Marketed GLP1R agonists have shown a remarkable efficacy in reducing bodyweight. However, those therapeutics, while effective in reducing the bodyweight, are also accompanied with significant loss of lean muscle mass. And the lost weights might return once the medication is withdrawn. There are still unmet needs for efficient management of obesity and diabetes using GLP1R agonist.

Growth and Differentiation Factor-8 (GDF8), also known as myostatin, is a member of the TGF-β superfamily of growth factors. GDF8 plays a central role in the development and maintenance of skeletal muscle, acting as a negative regulator of muscle mass. GDF8 is highly conserved across species, and the amino acid sequences of murine and human GDF8 are identical (human GDF8 nucleic acid sequence and amino acid sequence shown in SEQ ID NO:338-339) (McPherron et al. 1977 Nature 387:83-90).

A number of human diseases are associated with loss or impairment of muscle tissue, for example, muscular dystrophy, muscle atrophy, muscle wasting syndrome, sarcopenia and cachexia, and inhibitors of GDF8 are applicable treating these diseases or disorders. While the myostatin null mouse phenotype demonstrates the importance of GDF8 in the control of muscle size during development, muscle hypertrophy can also be elicited in adult muscle through inhibition of GDF8 with neutralizing antibodies, decoy receptors, or other antagonists.

Administration of GDF8 neutralizing antibodies has been reported to result in muscle mass increases of between 10 and 30%. The increased muscle mass seen is due to increased fiber diameter as opposed to myofiber hyperplasia (fiber number). A number of studies have also reported increases in muscle strength or performance commensurate with increased size including twitch and tetanic force.

Antibodies to Growth and Differentiation Factor-8 (GDF8), also known as myostatin, are known in the art. Antibodies to GDF8 and therapeutic methods are disclosed in, e.g., U.S. Pat. No. 8,840,894. Anti-GDF8 antibodies are also mentioned in, e.g., U.S. Pat. Nos. 6,096,506; 7,320,789; 7,261,893; 7,807,159; 7,888,486; 7,635,760; 7,632,499; in US Patent Appl. Publ. Nos. 2006/0263354; 2007/0178095; 2008/0299126; 2010/0166764; 2009/0148436; and International Patent Appl. Publ. Nos. WO2004/037861; WO2006/116269; WO2012/024242; WO2014/144903; WO2007/047112; WO 2010/070094; WO2011/151432; WO2010/070094; WO2023/187022. Antibodies to GDF8 and therapeutic methods are disclosed in, e.g., U.S. Pat. Nos. 6,096,506, 7,320,789, 7,807,159, WO 2007/047112, WO 2005/094446, US 2007/0087000, U.S. Pat. No. 7,261,893, WO 2010/070094, U.S. Pat. No. 8,840,894, WO2013006437, and U.S. Pat. No. 9,260,515.

Additional examples include Abrilumab (AMG 181, WO2010107752), Apitegromab (SRK-015, WO2016073879 and WO2016073879), Bimagrumab (BYM 338, WO2017081624, WO2016092439, and WO2010125003), Domagrozumab (PF-06252616, WO2013186719), GYM329 (Chugai Pharmaceutical Co. and Hoffmann-La Roche, Inc., RO7204239), Landogrozumab (LY2495655, WO2005094446, WO2007044411, WO2007047112, and WO2008030706), Stamulumab (MYO-029, WO2003027248, WO2004037861, WO2009058346, and WO2007024535), and Trevogrumab (REGN1033, WO2011150008).

The above-noted patents and patent applications are incorporated herein by reference in their entirety.

The current disclosure GLP1R agonist ligand tethered GDF8 antibody may provide an effective solution to obesity management without significant loss of lean mass and offer much improvement in clinical outcomes.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE DISCLOSURE

Various non-limiting aspects and embodiments of the disclosure are described below.

The present disclosure relates to protein-ligand conjugates (e.g., antibody-ligand conjugates), pharmaceutical compositions, and methods of treating disease therewith. More specifically, the present disclosure relates to GDF8 antibody-tethered GLP1R agonist ligand (ATL) and methods of treating GDF8 and GLP1R-associated conditions therewith including diabetes and obesity.

In one aspect, provided herein is a compound of Formula (A): H-Aib-AA₁-G-T-AA₂-T-S-D-AA₃-AA₄-S-Y-L-E-E-Q-A-A-AA₅-E-AA₆-I-A-W-L-V-AA₇-G-G-G (SEQ ID NO: 71) (A),

where
H is His;
Aib is 2-Aminoisobutyric acid;
AA₁ is E or

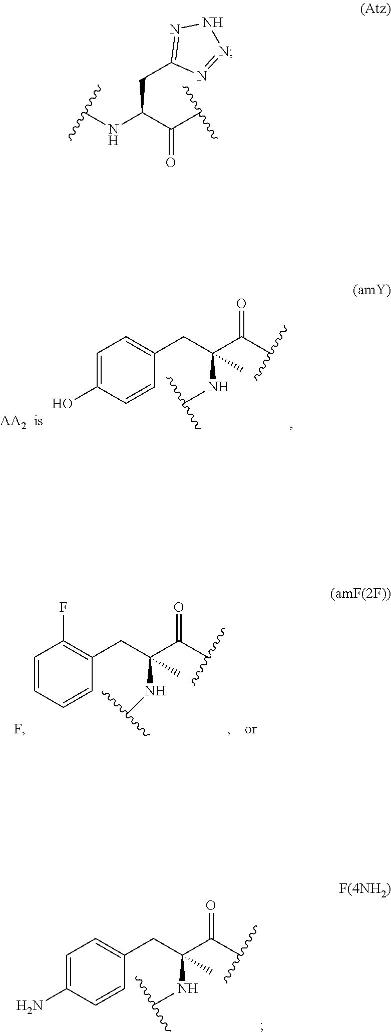 [SMILES: *N[C@@H](Cc1nn[nH]n1)C(*)=O ; *N[C@@](C)(Cc1ccc(O)cc1)C(*)=O ; *N[C@@](C)(Cc1ccccc1F)C(*)=O ; *N[C@@](C)(Cc1ccc(N)cc1)C(*)=O]

AA₂ is

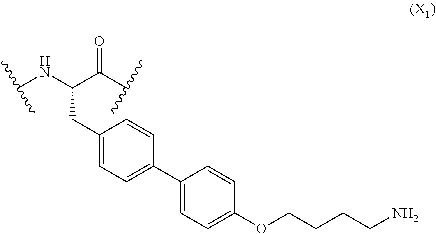 [SMILES: *N[C@@H](Cc1ccc(-c2ccc(OCCCCN)cc2)cc1)C(*)=O]

or Y;
AA₄ is S or

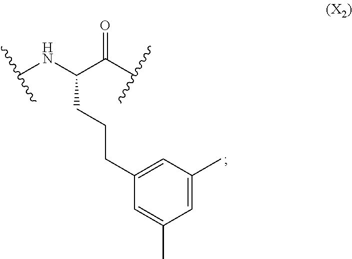 [SMILES: *N[C@@H](CCCc1cc(C)cc(C)c1)C(*)=O]

AA₅ is K or

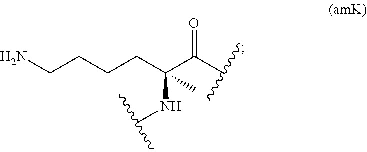 [SMILES: *N[C@@](C)(CCCCN)C(*)=O]

AA₆ is F or

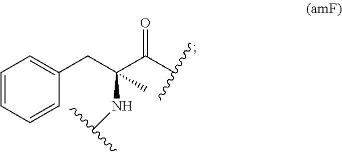 [SMILES: *N[C@@](C)(Cc1ccccc1)C(*)=O]

and
AA₇ is K or amK.

In some embodiments, AA₁ is E.

In some embodiments, AA₂ is F. In other embodiments, AA₂ is amY.

In some embodiments, AA₃ is Y.

In some embodiments, AA₄ is S.

In some embodiments, AA₅ is K.

In some embodiments, AA₆ is F.

In some embodiments, AA₇ is K.

In some embodiments, the compound is selected form the group consisting of:

(SEQ ID NO: 3)
H(Aib)EGTFTSDYSSYLEEQAAKEFIAWLVKGGG,
(SEQ ID NO: 38)
H[Aib]EGT-amY-TSD-X1-SSYLEEQAAKEFIAWLVKGGG,
(SEQ ID NO: 39)
H[Aib]EGT-amY-TSD-X1-SSYLEEQAA-amK-E-amF-
IAWLV-amK-GGG,
(SEQ ID NO: 40)
H[Aib]EGT-amF(2F)-TSDYSSYLEEQAAKEFIAWLVKGGG,
(SEQ ID NO: 41)
H[Aib]Atz-GTFTSDYSSYLEEQAAKEFIAWLVKGGG,
(SEQ ID NO: 43)
H[Aib]EGT-amY-TSD-X1-SSYLEEQAA-amK-E-amF-IAWLV-
amK-GGG,
(SEQ ID NO: 44)
H[Aib]EGT-amF(2F)-TSDYSSYLEEQAAKEFIAWLVKGGG,
(SEQ ID NO: 45)
H[Aib]EGT-F(4NH2)-TSDYSSYLEEQAAKEFIAWLVKGGG,
(SEQ ID NO: 46)
H[Aib]EGTFTSDY-X2-SYLEEQAAKEFIAWLVKGGG,
(SEQ ID NO: 42)
H[Aib]EGT-amF(2F)-TSDYSSYLEEQAAKEFIWLVKGGG,
and
(SEQ ID NO: 12)
H[Aib]Atz-GTFTSDYSSYLEEQAAKEFIAWLVKGGG.

In another aspect, provided herein is a composition comprising a GLP1R agonist-tethered GDF8 antibody conjugate, or a pharmaceutically acceptable salt thereof, where the conjugate comprises
a) an antibody, or an antigen-binding fragment thereof, that specifically binds to and/or blocks the biological activity of, Growth and Differentiation Factor-8 (GDF8, SEQ ID NO: 1);
b) at least one Glucagon-like peptide-1 (GLP-1) receptor (GLP1R) agonist; and
c) at least one linker that covalently connects the at least one GLP1R agonist to the GDF8 antibody, or the antigen-binding fragment thereof.

In some embodiments, the GLP1R agonist is linked to the GDF8 antibody, or the antigen-binding fragment thereof, through a side chain of a cysteine residue (Cys) of the GDF8 antibody, or the antigen-binding fragment thereof.

In some embodiments, the Cys is a naturally occurring Cys or an engineered Cys.

In some embodiments, the engineered Cys is introduced to the GDF8 antibody, or the antigen-binding fragment thereof, by site-specific modification of one or more amino acid residues.

In some embodiments, the GLP1R agonist is covalently attached to the GDF8 antibody, or the antigen-binding fragment thereof, through the side chain of a glutamine residue (Gln) of the GDF8 antibody, or the antigen-binding fragment thereof.

In some embodiments, the GLP1R agonist is covalently attached to the GDF8 antibody, or the antigen-binding fragment thereof, via a microbial transglutaminase assisted reaction, a Click or Diels-Alder reaction, or a combination thereof.

In some embodiments, the Gln is a naturally occurring Gln or an engineered Gln.

In some embodiments, the Gln is Gln55 (Q55), Gln295 (Q295), or a combination thereof.

In some embodiments, the GLP1R agonist is selected from the group consisting of GLP-1(7-37) (SEQ ID NO: 2); the compound according to any of the embodiments described herein; exenatide, liraglutide, lixisenatide, albiglutide, dulaglutide, semaglutide, taspoglutide, and tirzepatide.

In some embodiments, the GLP1R agonist is the compound according to any of the embodiments described herein.

In some embodiments, the GLP1R agonist is selected from the group consisting of GLP-1(7-37) (SEQ ID NO: 2); GLP1R agonist (SEQ ID NO: 3), exenatide, liraglutide, lixisenatide, albiglutide, dulaglutide, semaglutide, taspoglutide, and tirzepatide.

In some embodiments, the linker comprises

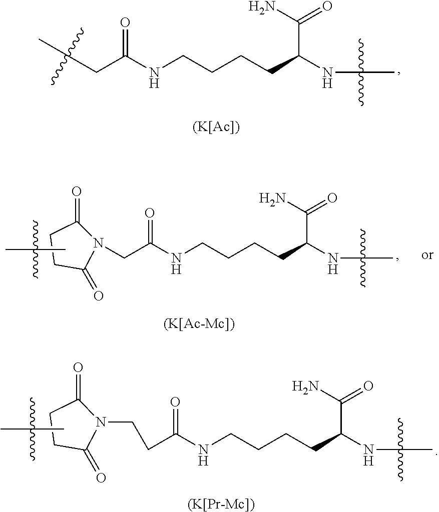

In some embodiments, the linker comprises

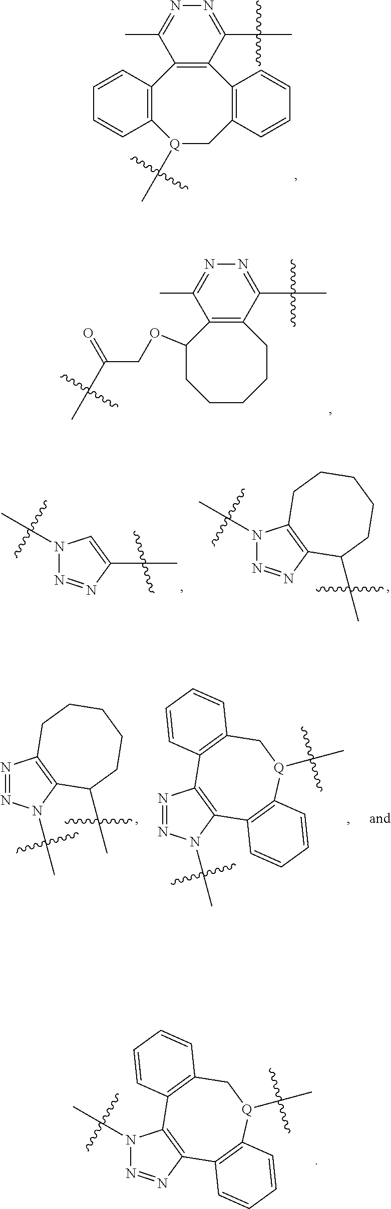

In some embodiments, the linker comprises

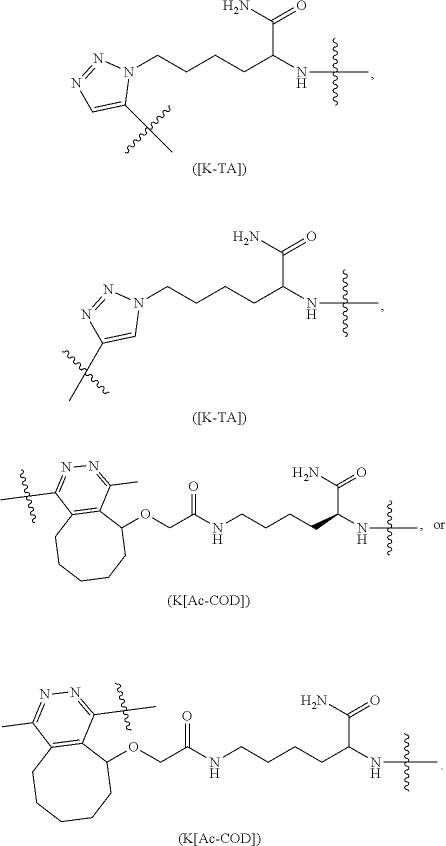

In some embodiments, the reactive moiety for the Click reaction comprises

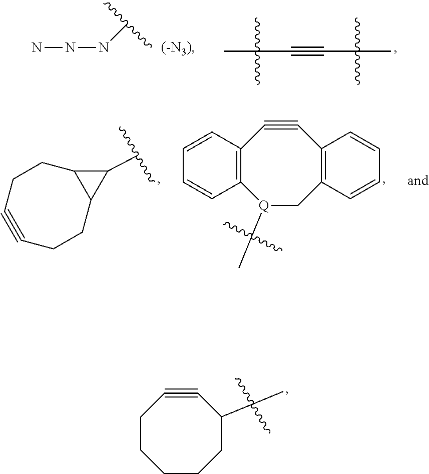

where Q is CH or N.

In some embodiments, the reactive moiety for the Diels-Alder reaction comprises

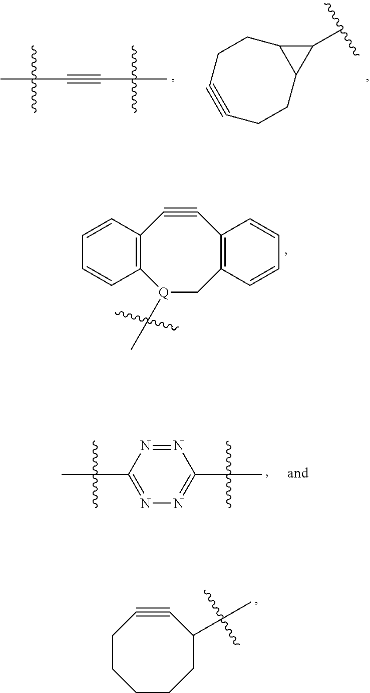

where Q is CH or N.

In some embodiments, the compound comprises between about one and about sixteen GLP1R agonist moieties.

In some embodiments, the GDF8 antibody or the antigen binding fragment thereof is an antibody, or the antigen binding fragment thereof that binds to, and blocks or substantially reduce the activities of GDF8. In some embodiments, the GDF8 antibody or the antigen binding fragment thereof comprises 1A2, 21-E5, 8D12, H4H1657N2 (REGN1033), and H4H1669P. In some embodiments, the GDF8 antibody comprises Abrilumab (AMG 181, WO2010107752), Apitegromab (SRK-015, WO2016073879 and WO2016073879), Bimagrumab (BYM 338, WO2017081624, WO2016092439, and WO2010125003), Domagrozumab (PF-06252616, WO2013186719), GYM329 (Chugai Pharmaceutical Co. and Hoffmann-La Roche, Inc., RO7204239), Landogrozumab (LY2495655, WO2005094446, WO2007044411, WO2007047112, and WO2008030706), Stamulumab (MYO-029, WO2003027248, WO2004037861, WO2009058346, and WO2007024535), and Trevogrumab (REGN1033, WO2011150008).

In some embodiments, the GDF8 antibody, or the antigen-binding fragment thereof, comprises heavy chain complementarity determining regions (HCDRs) of a heavy chain variable region (HCVR) comprising SEQ ID NO:4, and light chain complementarity determining regions (LCDRs) of a light chain variable region (LCVR) comprising SEQ ID NO:5.

In some embodiments, the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively, and the GLP1R agonist is the compound according to any of the embodiments described herein.

In some embodiments, the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively, and the GLP1R agonist has a sequence of H(Aib)EGTFTSDYSSYLEEQAAKEFIAWLVKGGG (SEQ ID NO: 3), where Aib is 2-Aminoisobutyric acid.

In some embodiments, the GLP1R agonist comprises the compound according to any of the embodiments described herein.

In some embodiments, the GLP1R agonist comprises the sequence H(Aib)EGTFTSDYSSYLEEQAAKEFIAWLVKGGG (SEQ ID NO: 3), where Aib is 2-Aminoisobutyric acid.

In some embodiments, the GLP1R agonist consists of the compound according to any of the embodiments described herein.

In some embodiments, the GLP1R agonist consists of the sequence H(Aib)EGTFTSDYSSYLEEQAAKEFIAWLVKGGG (SEQ ID NO: 3), where Aib is 2-Aminoisobutyric acid.

In some embodiments, the linker comprises a sequence selected from the group consisting of Gly₃Ser (SEQ ID NO: 72), Gly₄Ser, (SEQ ID NO: 73) (Gly₃Ser)₂ (SEQ ID NO: 74), (Gly₄Ser)₂ (SEQ ID NO: 75), (Gly₃Ser)₃, (SEQ ID NO: 76) (Gly₄Ser)₃ (SEQ ID NO: 77), (Gly₃Ser)₄ (SEQ ID NO: 78), (Gly₄Ser)₄ (SEQ ID NO: 79), (Gly₃Ser)₅ (SEQ ID NO: 80), (Gly₄Ser)₅ (SEQ ID NO: 81), (Gly₃Ser)₆ (SEQ ID NO: 82), and (Gly₄Ser)₆ (SEQ ID NO: 83).

In some embodiments, the composition according to the present disclosure further comprises one or more pharmaceutically acceptable salts, excipients, and/or diluents.

In another aspect, provided herein is a pharmaceutical dosage form comprising the composition of any of the embodiments described herein.

In another aspect, provided herein is a GLP1R agonist-tethered GDF8 antibody conjugate, or a pharmaceutically acceptable salt thereof, having a Formula (I):

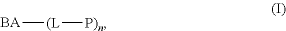

where
BA is an antibody, or an antigen-binding fragment thereof, that specifically binds to and/or blocks the biological activity of, human Growth and Differentiation Factor-8 (GDF8, SEQ ID NO: 1);
P is a Glucagon-like peptide-1 (GLP-1) receptor (GLP1R) agonist;
L is a linker that covalently links the P to the BA; and
n ranges from about 1 to about 16.

In some embodiments, the GLP1R agonist-tethered GDF8 antibody conjugate has a Formula (II):

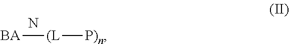

where [symbol] represents that the connection between the BA and the L is through the side chain of a glutamine residue (Gln) of the BA.

In some embodiments, the Gln is a naturally occurring Gln or an engineered Gln.

In some embodiments, the Gln is Gln55 (Q55). In other embodiments, the Gln is Gln295 (Q295).

In some embodiments, the linker in the GLP1R agonist-tethered GDF8 antibody conjugate comprises (a) a Click or Diels-Alder reaction adduct, and (b) an amide bond formed via a transglutaminase reaction between a primary amine and the side chain of a Gln of the BA.

In some embodiments, a handle for Click or Diels-Alder reaction is conjugated to the GDF8 antibody via microbial transglutaminase prior to the conjugation with the GLP1R agonist, where the GLP1R agonist comprises a reactive moiety for Click reaction, selected from the group consisting of from

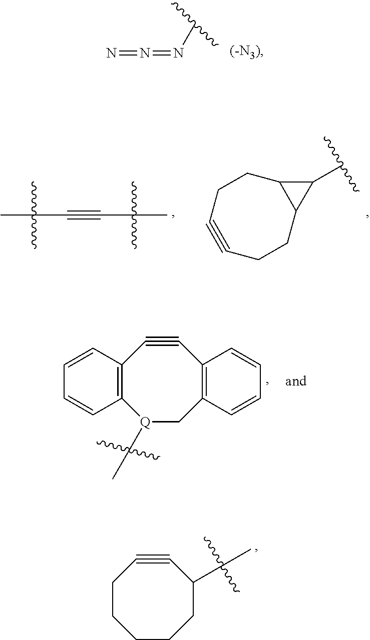

where Q is CH or N.

In some embodiments, a handle for Click or Diels-Alder reaction is conjugated to the GDF8 antibody via microbial transglutaminase prior to the conjugation with the GLP1R agonist, where the GLP1R agonist comprises a reactive moiety for Diels-Alder reaction, selected from the group consisting of from

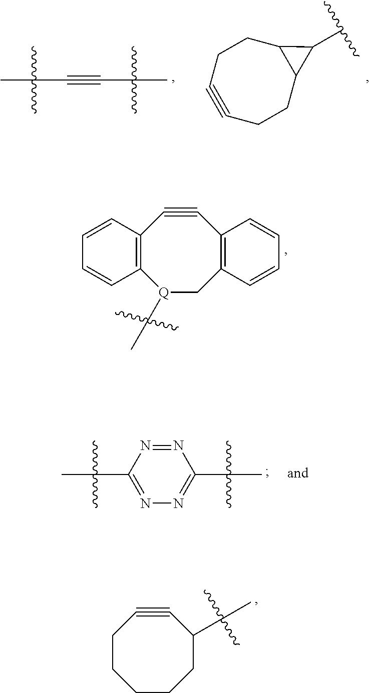

where Q is CH or N.

In some embodiments, the handle for Click or Diels-Alder reaction, is selected from the group consisting of

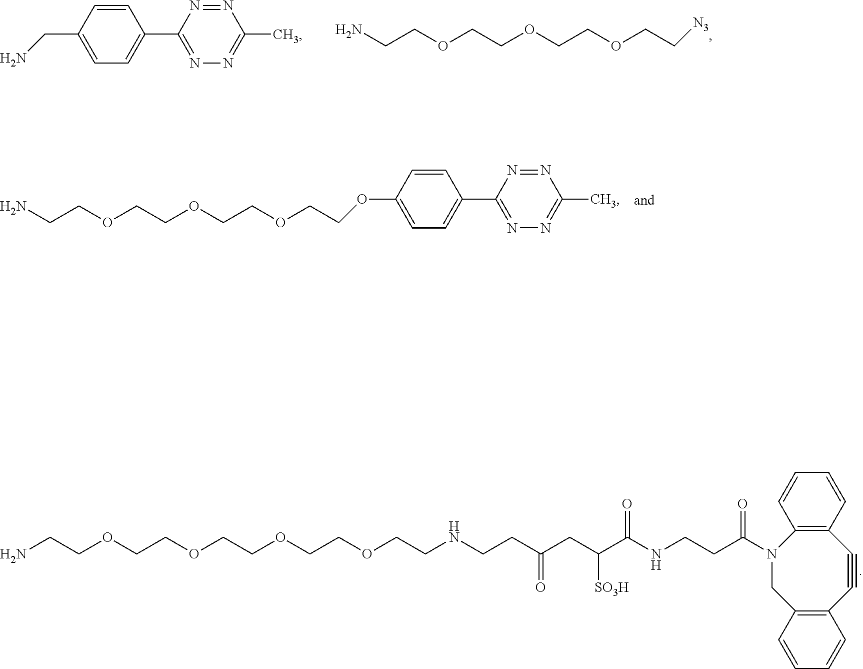 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the linker L comprises a Click or Diels-Alder reaction adduct selected from the group consisting of:

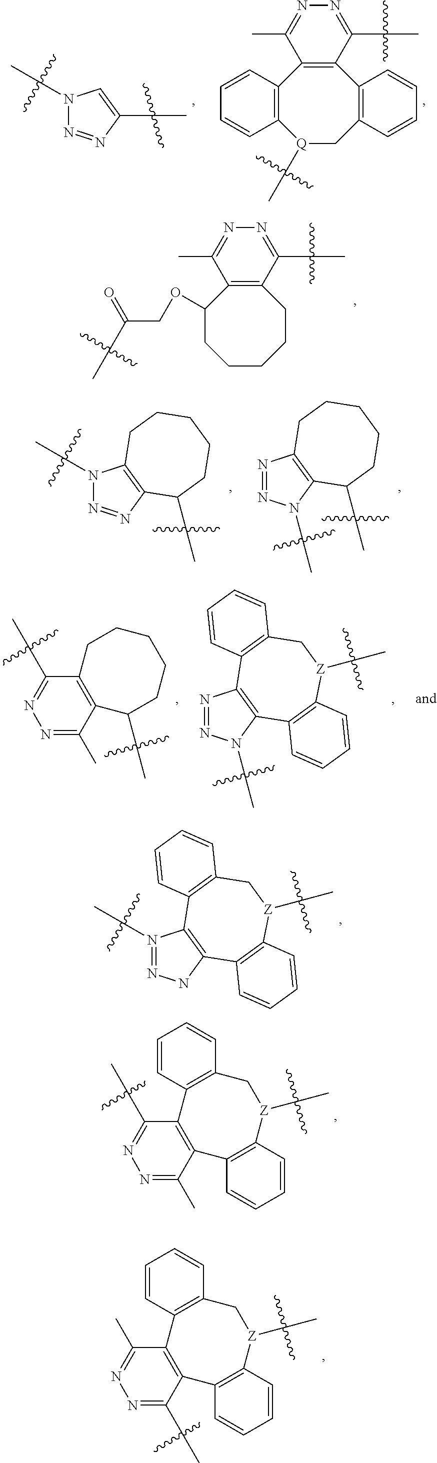

where Z is CH or N.

In some embodiments, the linker L comprises

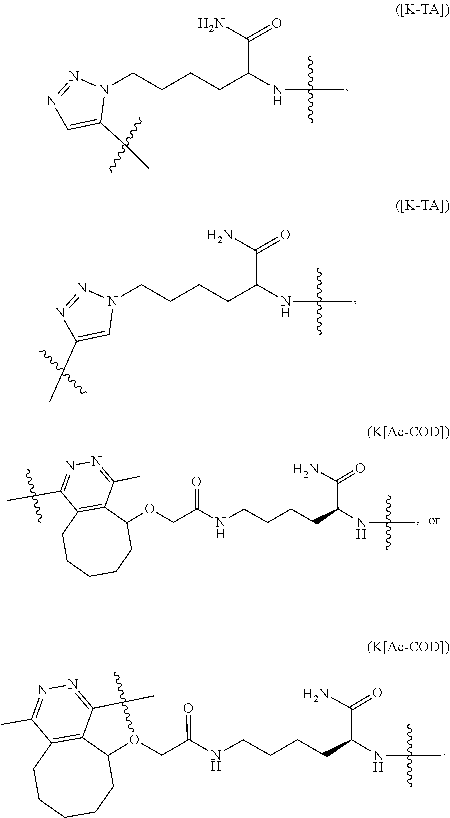 [SMILES: *NC(CCCCn1nncc1*)C(N)=O ; *NC(CCCCn1cc(*)nn1)C(N)=O ; *N[C@@H](CCCCNC(=O)COC1CCCCCc2c(*)nnc(C)c21)C(N)=O ; *N[C@@H](CCCCNC(=O)COC1CCCCCc2c(C)nnc(*)c21)C(N)=O]

In some embodiments, the GLP1R agonist-tethered GDF8 antibody conjugate has a Formula (III):

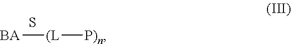

where [symbol] represents that the connection between the BA and the L is via the side chain of a cysteine residue (Cys) of the BA.

In some embodiments, the Cys of the BA is a naturally occurring Cys or an engineered Cys. In some embodiments, the Cys of GDF8 antibody is a naturally occurring Cys. In some embodiments, the Cys of the BA is an engineered Cys introduced by site-specific mutation.

In some embodiments, L comprises a moiety resulted from chemical conjugation of a reactive group (RG) on the L-P with the side chain of Cys of the BA.

In some embodiments, the reactive group is a bromo acetyl or a maleimide group.

In some embodiments, the linker L comprises

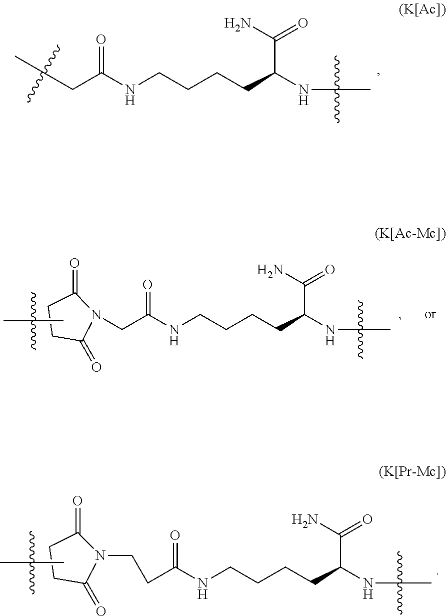

In some embodiments, the GDF8 antibody or the antigen binding fragment thereof comprises 1A2, 21-E5, 8D12, H4H1657N2 (REGN1033), and H4H1669P.

In some embodiments, the BA comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, the P comprises the compound according to any of the embodiments described herein.

In some embodiments, the P comprises the sequence of H(Aib)EGTFTSDYSSYLEEQAAKEFIAWLVKGGG (SEQ ID NO: 3), where Aib is 2-Aminoisobutyric acid.

In some embodiments, the P consists of the compound according to any of the embodiments described herein.

In some embodiments, the P consists of the sequence of H(Aib)EGTFTSDYSSYLEEQAAKEFIAWLVKGGG (SEQ ID NO: 3), where Aib is 2-Aminoisobutyric acid.

In some embodiments, the P is selected from the group consisting of exenatide, liraglutide, lixisenatide, albiglutide, dulaglutide, semaglutide, taspoglutide, and tirzepatide.

In some embodiments, n ranges from about 2 to about 4.

In some embodiments, the linker L comprises a sequence selected from the group consisting of -(Gly₃Ser)ₘ- (SEQ ID NO: 84) and -(Gly₄Ser)ₘ- (SEQ ID NO: 85), where m is an integer from one to six.

In some embodiments, the linker L comprises a sequence selected from the group consisting of -(Gly₃Ser)ₘ-Lys- (SEQ ID NO: 86) and -(Gly₄Ser)ₘ-Lys- (SEQ ID NO: 87), where m is an integer from one to six; and where the side chain of Lys is further functionalized with a reactive moiety for conjugation with the BA.

In some embodiments, the side chain of the Lys is further functionalized with a reactive moiety for conjugation with the BA having a structure of

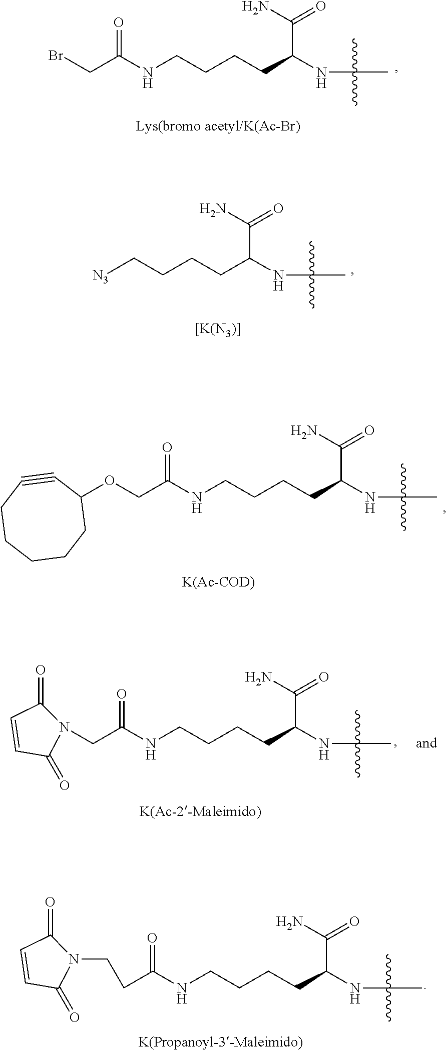

In some embodiments, a handle comprising a reactive moiety for Click or Diels-Alder reaction is first conjugated to the BA via mTG for the following conjugation with a reactive moiety on the side chain of Lys of linker -(Gly₄Ser)ₙ-Lys- (SEQ ID NO: 88), where the handle has structure of consisting of

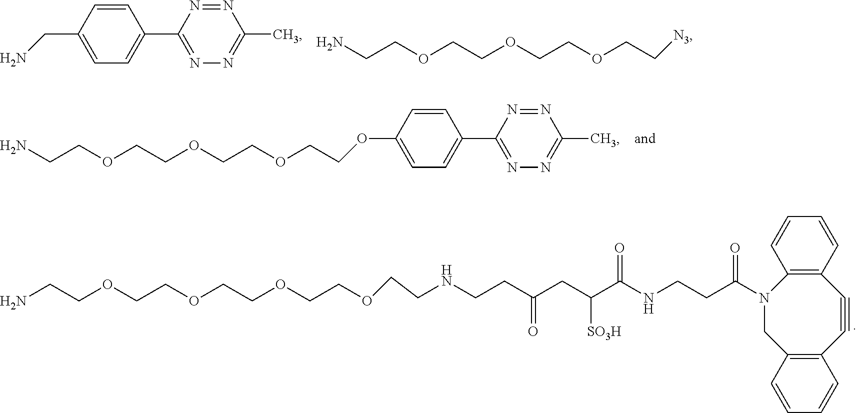

In some embodiments, the GLP1R agonist-tethered GDF8 antibody conjugate has the structure selected from the group consisting of:

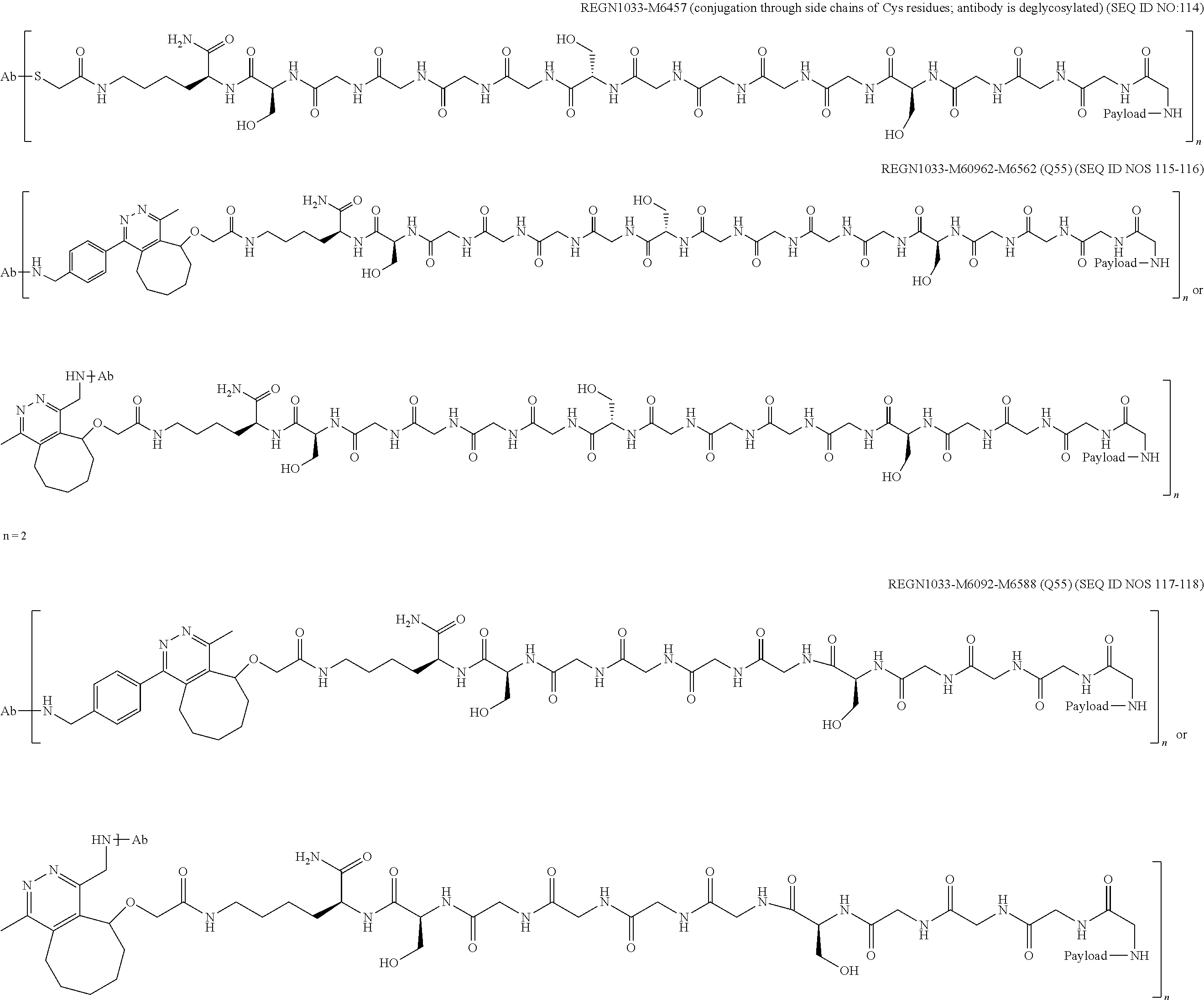

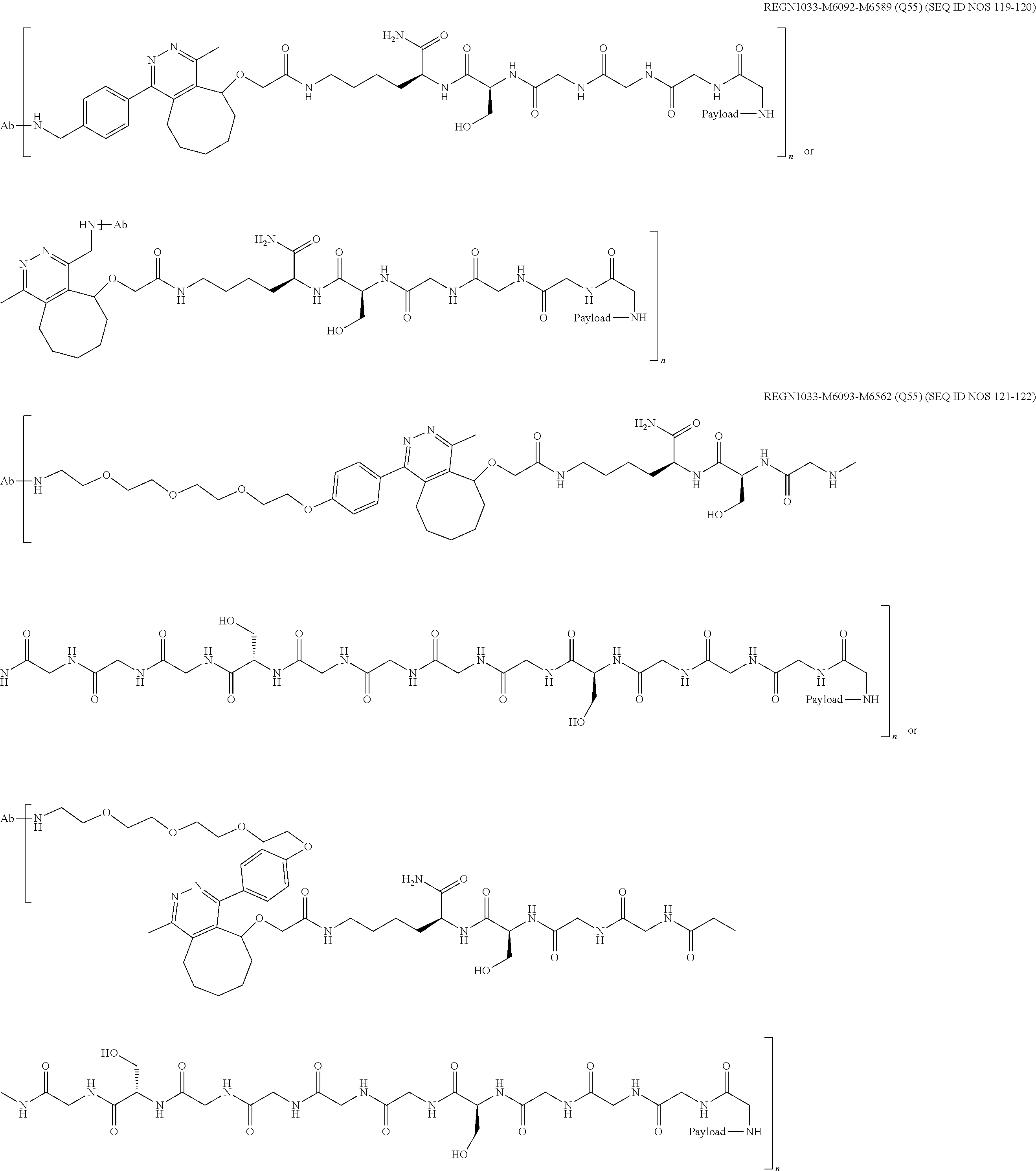

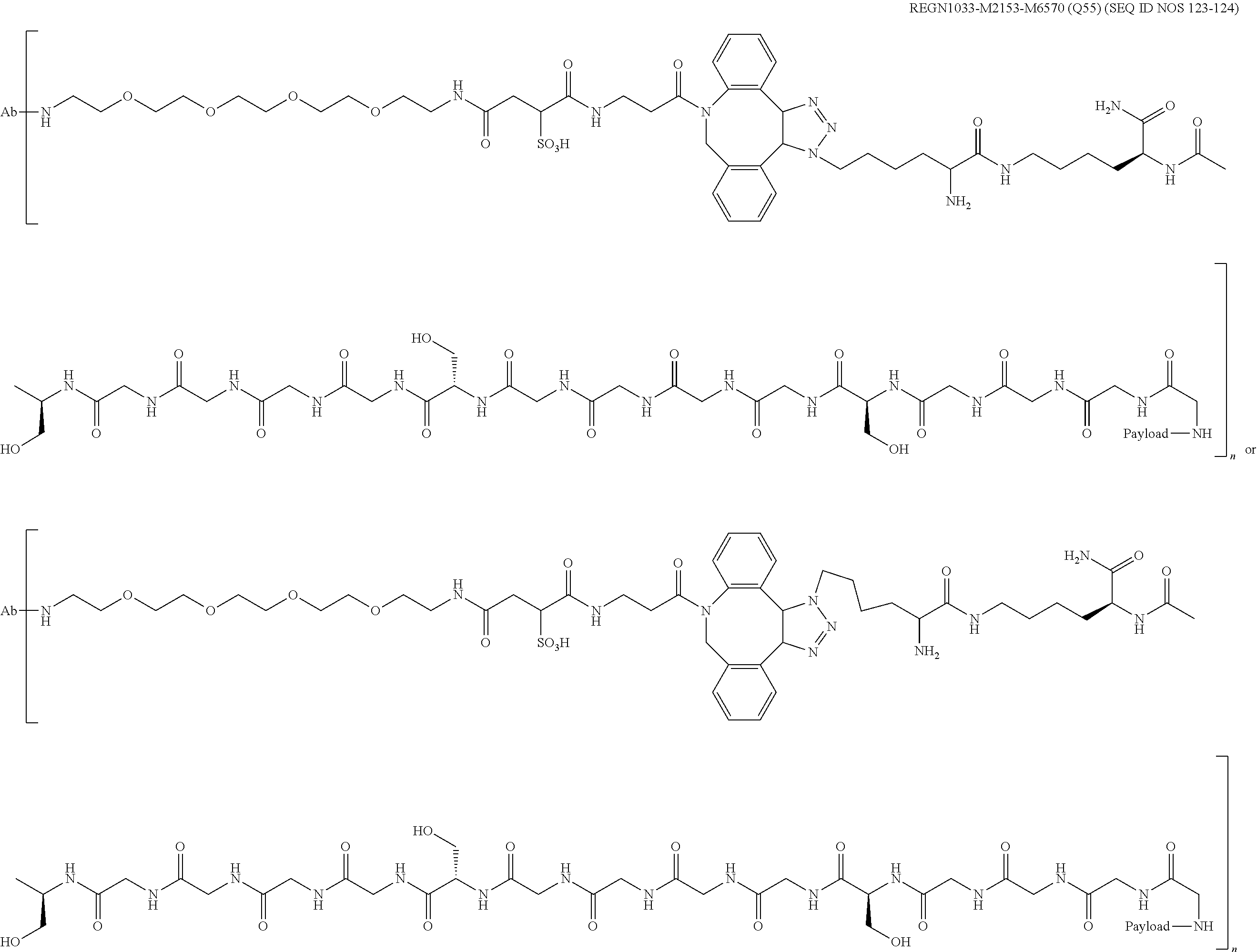

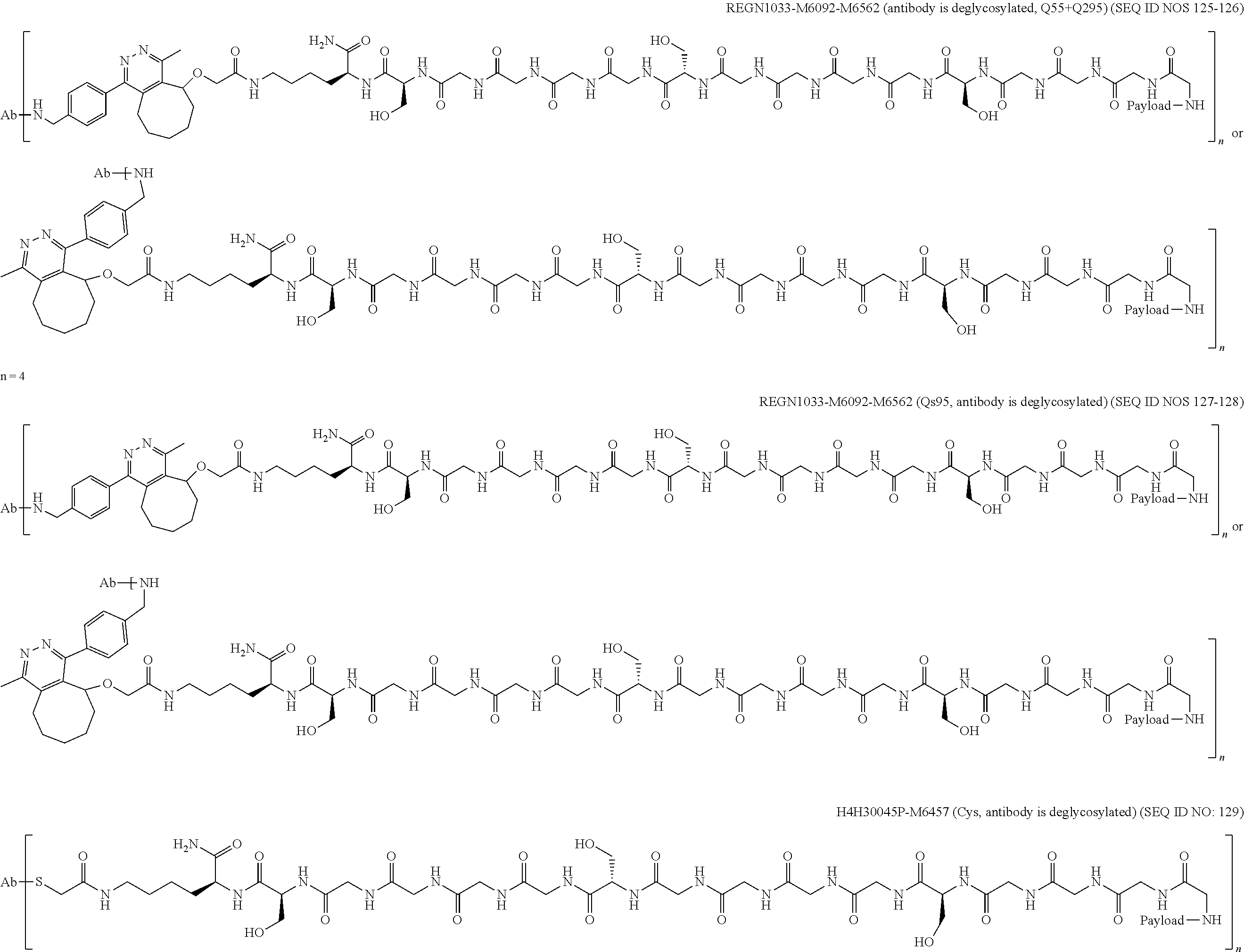

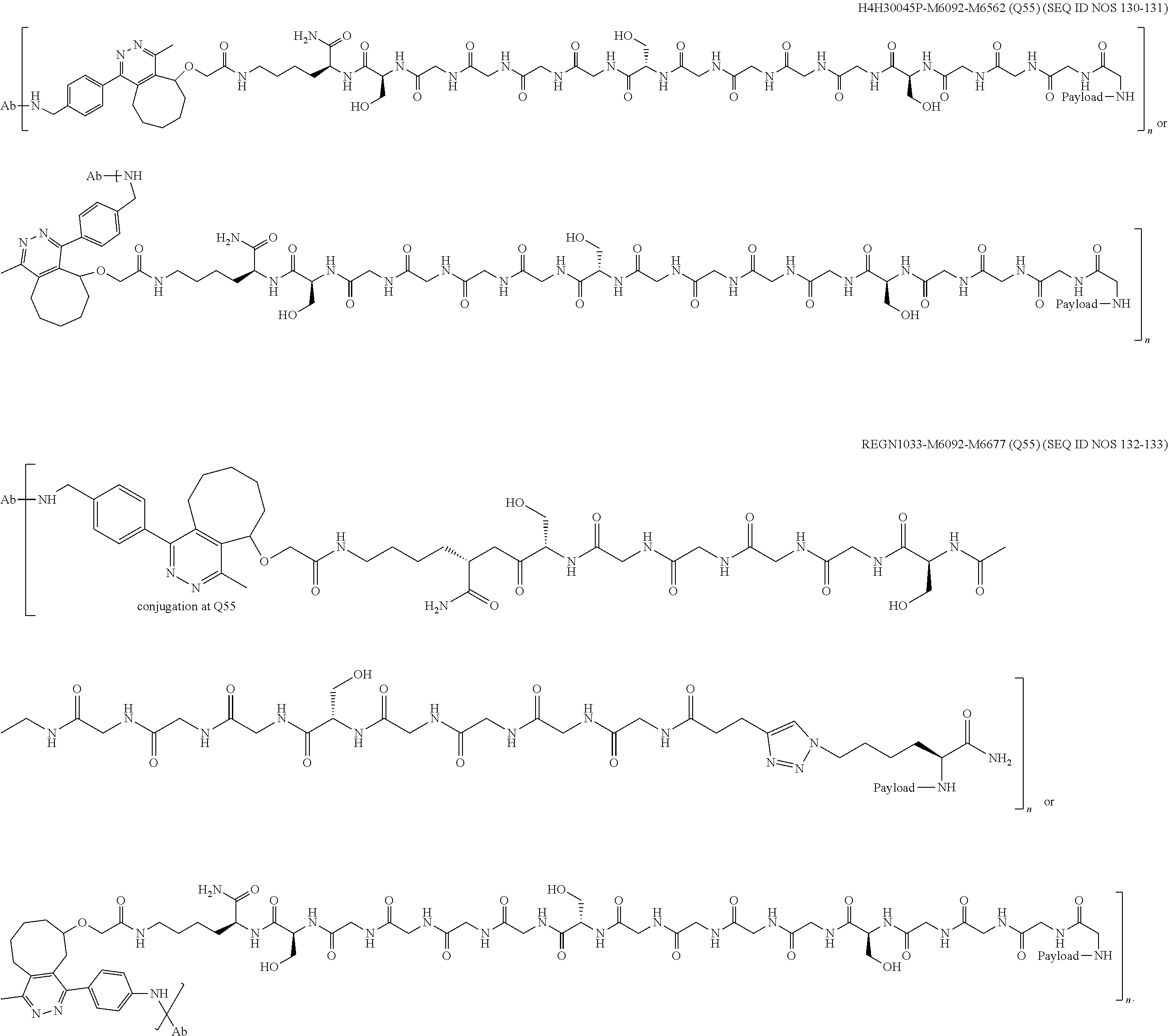

or a pharmaceutically acceptable salt thereof, where the Payload is the compound according to any of the embodiments described herein, and where n is about 1 to 10, and Ab is the GDF8 antibody, or the antigen-binding fragment thereof.

In some embodiments, the GLP1R agonist-tethered GDF8 antibody conjugate has the structure selected from the group consisting of:

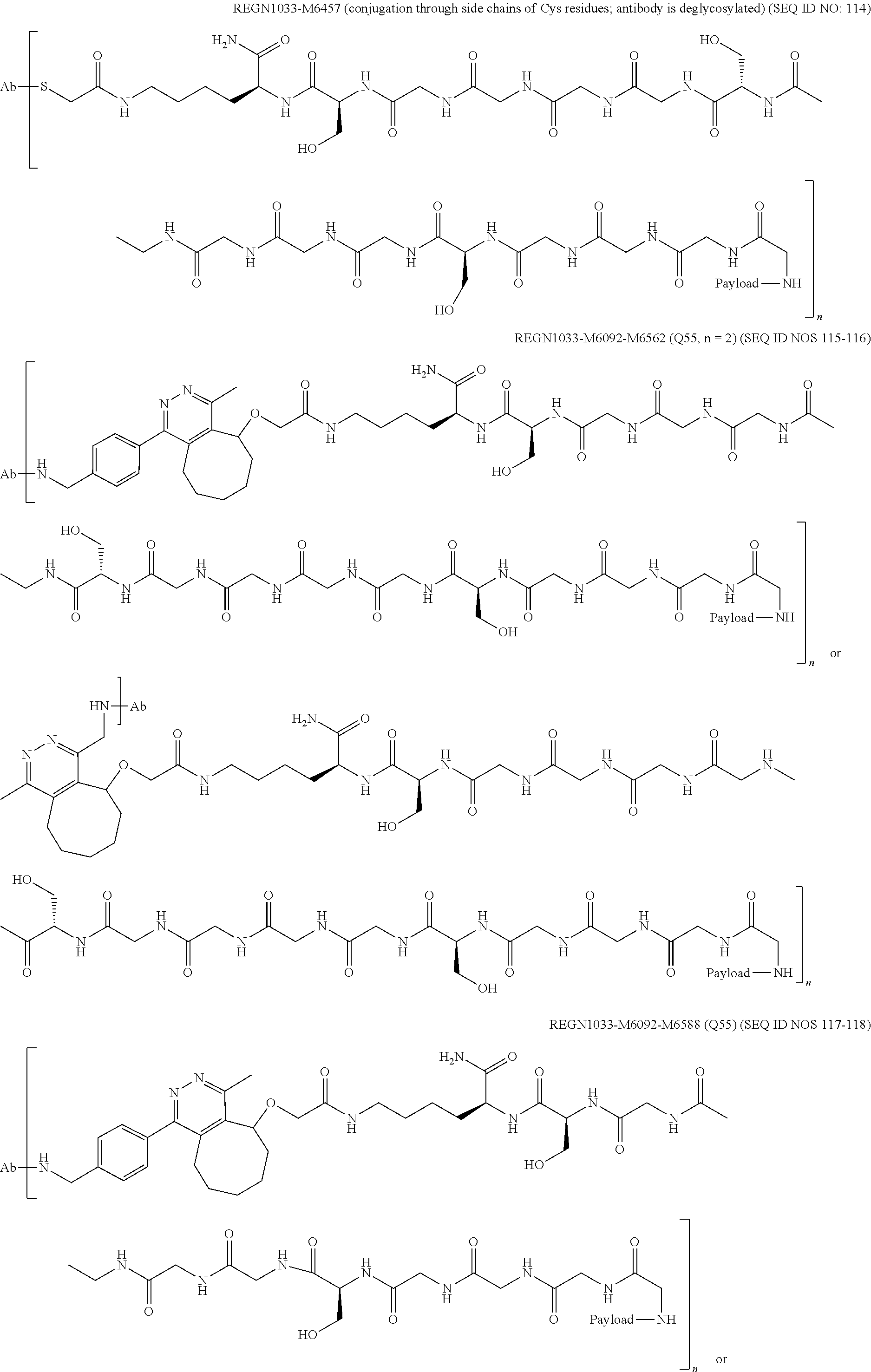

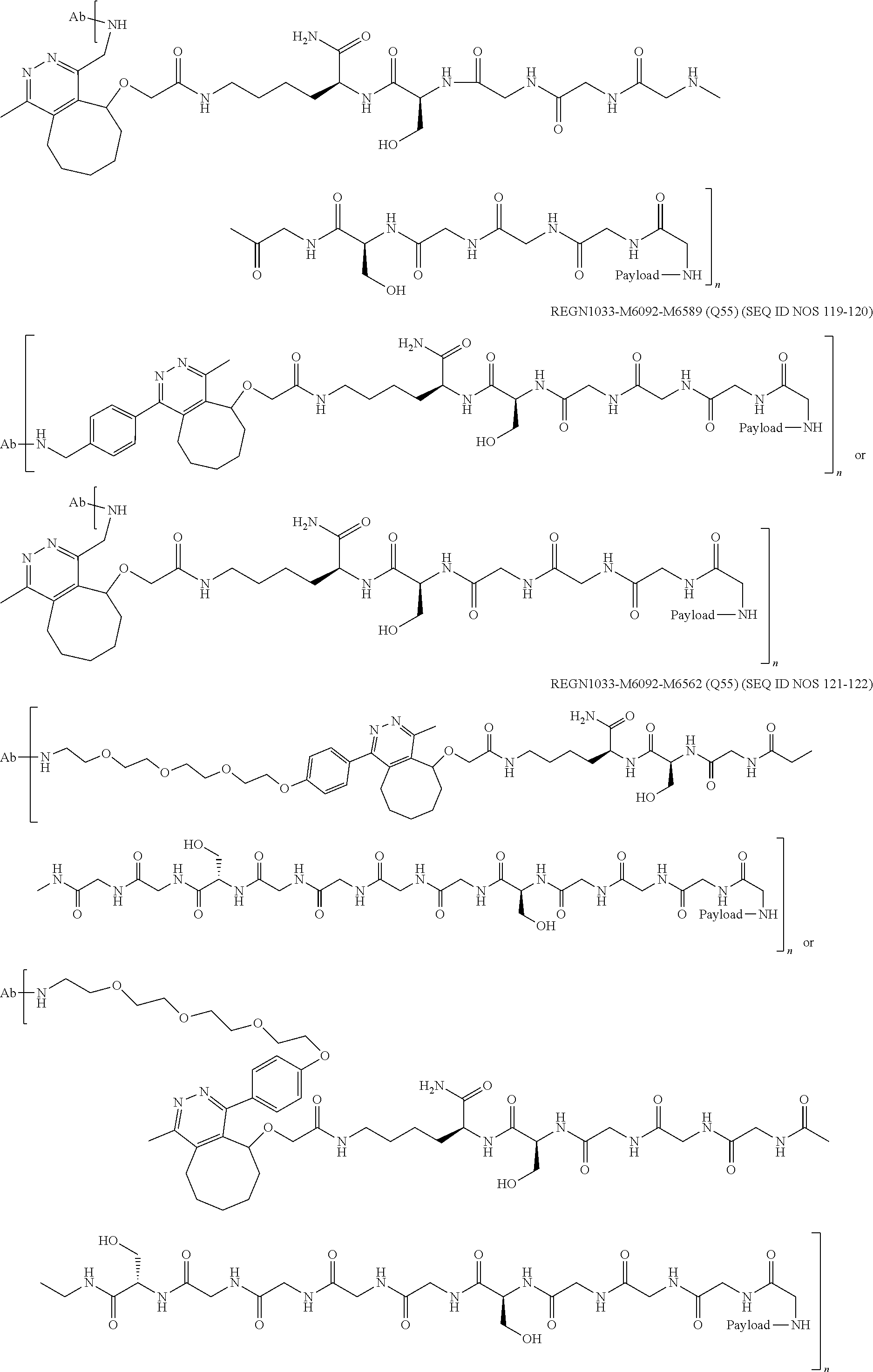

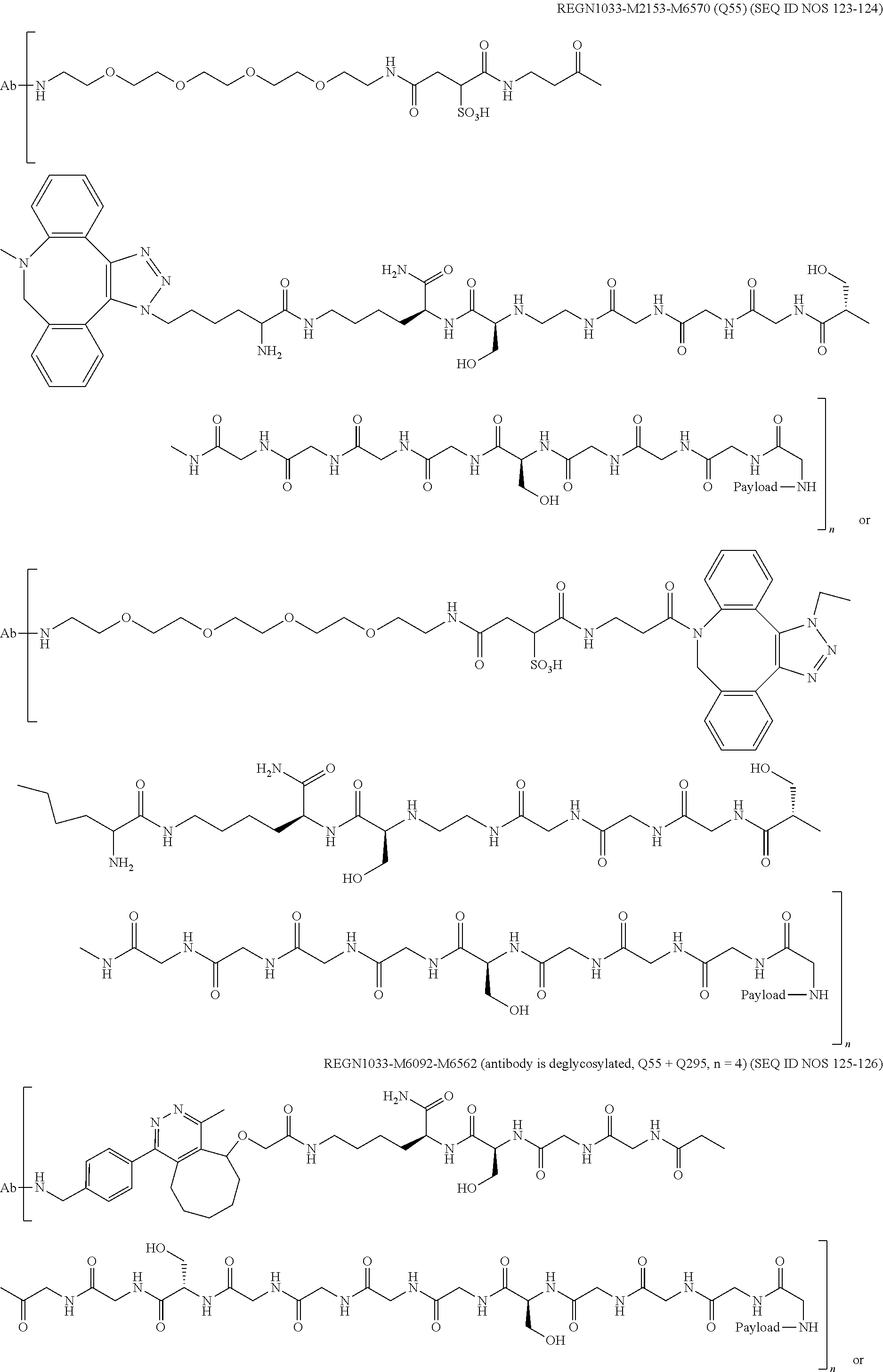

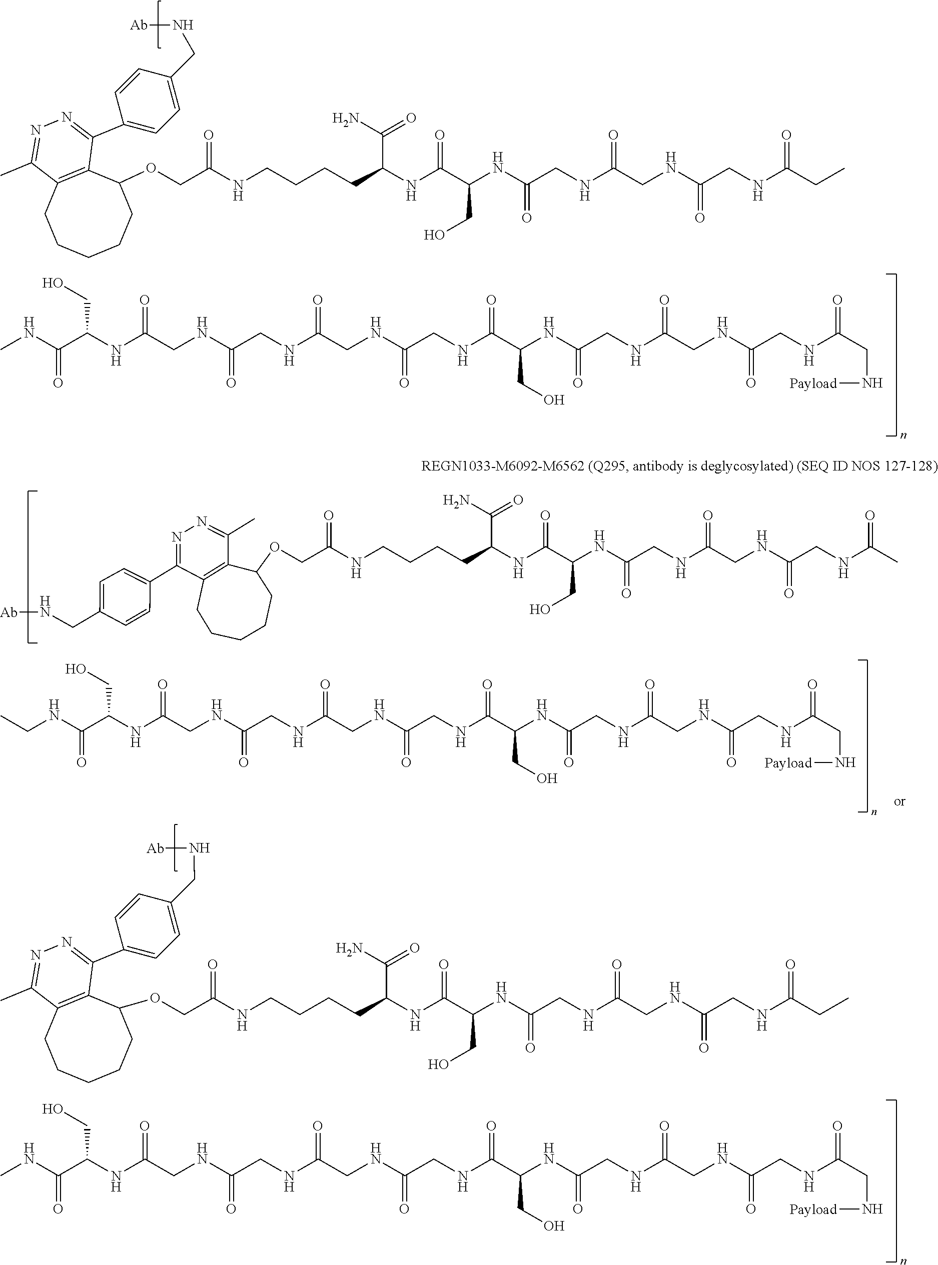

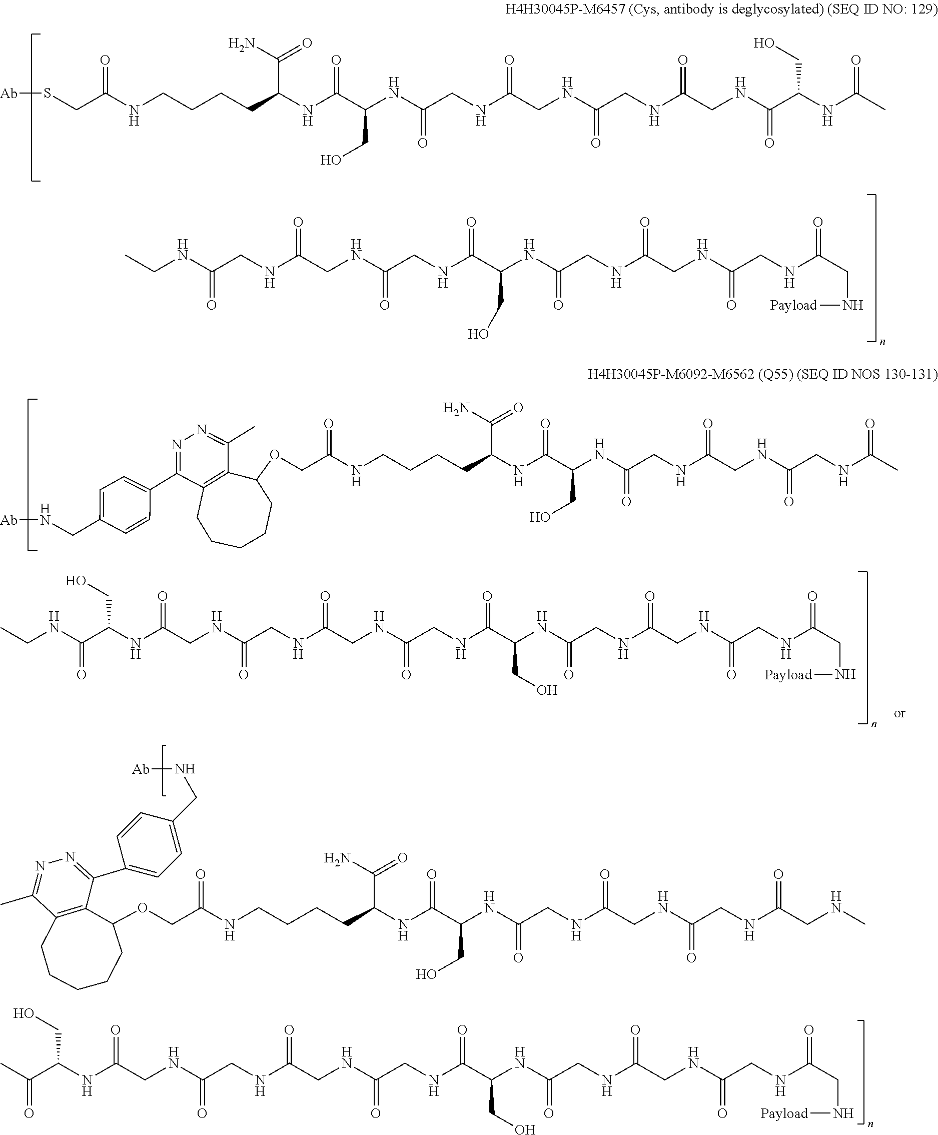

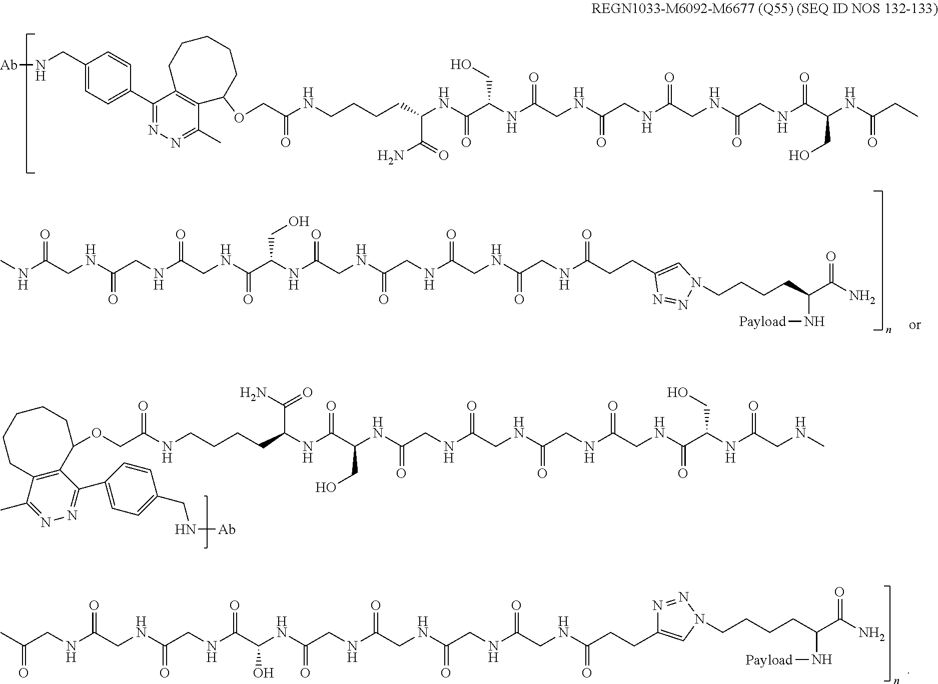

or a pharmaceutically acceptable salt thereof, where the Payload is H[Aib]EGTFTSDYSSYLEEQAAKEFIAWLVKGGG (SEQ ID NO: 3), and where n is about 1 to 10, and Ab is the GDF8 antibody, or the antigen-binding fragment thereof.

In another aspect, provided herein is a pharmaceutical composition comprising any GLP1R agonist-tethered GDF8 antibody conjugate of any of the embodiments described herein, or a pharmaceutically acceptable salt thereof.

In some embodiments, the pharmaceutical composition further comprises one or more pharmaceutically accept salts, excipients, and/or diluents.

In another aspect, provided herein is a pharmaceutical dosage form comprising the GLP1R agonist-tethered GDF8 antibody according to any of the embodiments described herein, or a pharmaceutically acceptable salt thereof, or the pharmaceutical composition according to any of the embodiments described herein.

In another aspect, provided herein is a compound, or a pharmaceutically acceptable salt thereof, having the structure of Formula (IV):

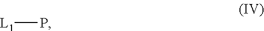

where
L1 is a linker comprising a sequence selected from the group consisting of -(Gly₃Ser)ₙ-Lys(X)- (SEQ ID NO: 89) and -(Gly₄Ser)ₙ-Lys(X)- (SEQ ID NO: 90), where n is an integer from one to six; and where the side chain of Lys is functionalized with a reactive moiety X for conjugation with a target antibody or antigen binding fragment thereof, and P is a Glucagon-like peptide-1 (GLP-1) receptor (GLP1R) agonist.

In some embodiments, the reactive moiety X is a moiety reactive toward cysteine thiol or a moiety for Click or Diels-Alder reaction.

In some embodiments, Lys(X) has a formula

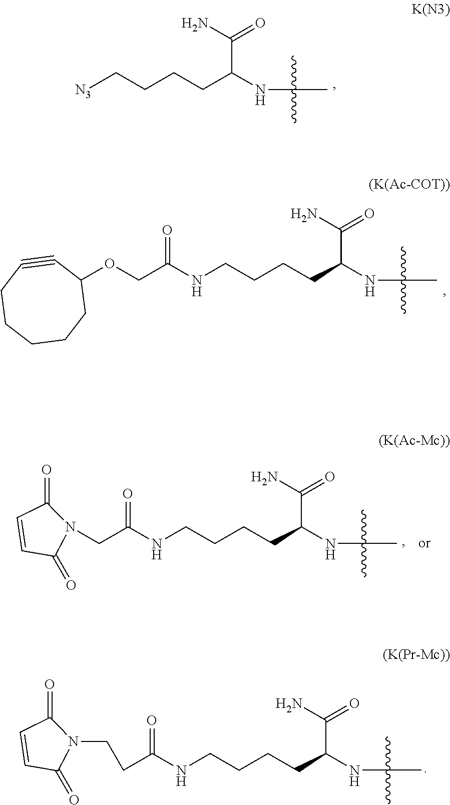 [SMILES: *NC(CCCCN=[N+]=[N-])C(N)=O ; *N[C@@H](CCCCNC(=O)COC1C#CCCCCC1)C(N)=O ; *N[C@@H](CCCCNC(=O)CN1C(=O)C=CC1=O)C(N)=O ; *N[C@@H](CCCCNC(=O)CCN1C(=O)C=CC1=O)C(N)=O]

In some embodiments, P (Payload) comprises the compound according to any of the embodiments described herein.

In some embodiments, P (Payload) comprises the sequence H(Aib)EGTFTSDYSSYLEEQAAKEFIAWLVKGGG (SEQ ID NO: 3), where Aib is 2-Aminoisobutyric acid.

In some embodiments, P (Payload) consists of the compound according to any of the embodiments described herein.

In some embodiments, P (Payload) consists of the sequence H(Aib)EGTFTSDYSSYLEEQAAKEFIAWLVKGGG (SEQ ID NO: 3), where Aib is 2-Aminoisobutyric acid.

In some embodiments, the compound has the structure selected from the group consisting of:

(SEQ ID NO: 15)
H(Aib)EGTFTSDYSSYLEEQAAKEFIAWLVKGGG-GGGGS-K[Ac-COT]amide
(Payload -(G4S)-K[Ac-COT]),
(SEQ ID NO: 14)
H(Aib)EGTFTSDYSSYLEEQAAKEFIAWLVKGGG-GGGGSGGGGS-K[Ac-COT]amide
(Payload -(G4S)2-K[Ac-COT]),
(SEQ ID NO: 13)
H(Aib)EGTFTSDYSSYLEEQAAKEFIAWLVKGGG-GGGGSGGGGSGGGGS-K[Ac-
COT]amide (Payload -(G₄S)₃-K[Ac-COT]),
(SEQ ID NO: 16)
H(Aib)EGTFTSDYSSYLEEQAAKEFIAWLVKGGG-GGGGSGGGGSGGGGS-K[KN3]
amide (Payload -(G₄S)₃-K[KN3]),
(SEQ ID NO: 17)
H(Aib)EGTFTSDYSSYLEEQAAKEFIAWLVKGGG-GGGGSGGGGSGGGGS-K[Ac-Mc]
amide (Payload -(G₄S)₃-K[Ac-Mc]),
(SEQ ID NO: 20)
H(Aib)EGTFTSDYSSYLEEQAAKEFIAWLVKGGG-GGGGSGGGGSGGGGS-
K[propanoyl-Mc]amide (Payload -(G₄S)₃-K[Pr-Mc]),
(SEQ ID NOS 18 & 136)
H[Aib]EGTFTSDYSSYLEEQAAKEFIAWLVKGGGK(TA-GGGGSGGGGSGGGGS-
K(Ac-COT)) amide (M6677) (Payload -K(TA-(G₄S)₃-K[Ac-COT]),
(SEQ ID NO: 19)
H[Aib]EGTFTSDYSSYLEEQAAKEFIAWLVKGGGK(N3) amide
(M6675)(Payload -K(N3)),

or a pharmaceutically acceptable salt thereof.

In some embodiments, the compound has the structure selected from the group consisting of:

(SEQ ID NO: 47)
H[Aib]EGT-amY-TSD-X1-SSYLEEQAAKEFIAWLVKGGGK
(N3) amide (M6618) (Payload -K(N3)),
(SEQ ID NO: 48)
H[Aib]EGT-amY-TSD-X1-SSYLEEQAA-amK-E-amF-IAWLV-amK-
GGGK(N3) amide (Payload -K(N3)),
(SEQ ID NO: 49)
H[Aib]EGT-amF(2F)-TSDYSSYLEEQAAKEFIAWLVKGGGK(N3)
amide (Payload -K(N3)),
SEQ ID NO: 50)
H[Aib]Atz-GTFTSDYSSYLEEQAAKEFIAWLVKGGGK(KN3) amide
(Payload -K(K(N3))),
(SEQ ID NOS 51 & 137)
H[Aib]EGT-amY-TSD-X1-SSYLEEQAA-amK-E-amF-IAWLV-amK-GGGK(T-
GGGGSGGGGSGGGGS-K(ALO)) amide (Payload -K(T-(G₄S)₃-K[ALO]),
(SEQ ID NO: 52)
H[Aib]EGT-amF(2F)-TSDYSSYLEEQAAKEFIAWLVKGGGGGGGSGGGG
SGGGGSK amide (Payload -(G₄S)₃-K),
(SEQ ID NO: 53)
H[Aib]EGT-F(4NH2)-TSDYSSYLEEQAAKEFIAWLVKGGGGGGGSGGGG
SGGGGSK amide (Payload -(G₄S)₃-K),
(SEQ ID NO: 54)
H[Aib]EGTFTSDY-X2-SYLEEQAAKEFIAWLVKGGGGGGGSGGGGSGGGG
SK (Payload -(G₄S)₃-K),
(SEQ ID NO: 55)
H[Aib]EGT-amF(2F)-TSDYSSYLEEQAAKEFIAWLVKGGGGGGGSGGGGSG
GGGSK[K(N3)]-CONH2 (M6571)(Payload -(G₄S)₃-K[K(N3)]),
(SEQ ID NO: 56)
H[Aib]Atz-GTFTSDYSSYLEEQAAKEFIAWLVKGGGGGGGSGGGGSGGG
GSK[K(N3)]-CONH2 (M6560) (Payload -(G₄S)₃-K[K(N3)]),
(SEQ ID NO: 17)
H[Aib]EGTFTSDYSSYLEEQAAKEFIAWLVKGGG (G₄S)₃-
K(Ac-Mc)-amide (M6557)
(Payload -(G₄S)₃-K(Ac-Mc)),
(SEQ ID NO: 13)
H[Aib]EGTFTSDYSSYLEEQAAKEFIAWLVKGGG (G₄S)₃-
K(Ac-COT)-amide (M6562)(Payload -(G₄S)₃-K(Ac-COT)),
(SEQ ID NO: 57)
H[Aib]EGTFTSDYSSYLEEQAAKEFIAWLVKGGG-K[K(N3)]-amide
(Payload -K[K(N3)]),
(SEQ ID NO: 58)
H[Aib]EGTFTSDYSSYLEEQAAKEFIAWLVKGGG-K(T-CH2-ALO)-amide
(M6676) (Payload -K(T-CH2-ALO)),
(SEQ ID NO: 59)
H[Aib]EGTFTSDYSSYLEEQAAKEFIAWLVKGGG(G₄S)₃-K(N3)-amide
(Payload -(G₄S)₃-K(N3)),
(SEQ ID NO: 60)
H[Aib]EGTFTSDYSSYLEEQAAKEFIAWLVKGGG(G₄S)₃-
K(T-CH2-ALO)-amide (M6651)(Payload -(G₄S)₃-K(T-CH2-ALO)),

or a pharmaceutically acceptable salt thereof.

In some embodiments, the GLP1R agonist P is selected from the group consisting of exenatide, liraglutide, lixisenatide, albiglutide, dulaglutide, semaglutide, taspoglutide, and tirzepatide.

In another aspect, provided herein is an antibody conjugate comprising a compound according to any of the embodiments described herein conjugated to GDF8 antibody that specifically binds to and/or blocks the activity of GDF8 (SEQ ID NO: 1).

In another aspect, provided herein is a method for treating obesity by reducing body weight while maintaining or increasing lean body mass in a subject, comprising administering a therapeutically effective amount of a GLP-1 agonist-tethered GDF8 antibody conjugate disclosed herein to the subject in need thereof.

In another aspect, provided herein is a method for treating obesity by reducing fat mass while maintaining or increasing lean body mass in a subject, comprising administering a therapeutically effective amount of a GLP-1 agonist-tethered GDF8 antibody conjugate disclosed herein to the subject in need thereof.

In another aspect, provided herein is a method for treating Type 2 diabetes by improving glycemic control and maintaining or increasing lean body mass while reducing fat mass in a subject, comprising administering a therapeutically effective amount of a GLP-1 agonist-tethered GDF8 antibody conjugate disclosed herein to the subject in need thereof.

In another aspect, provided herein is a method for treating obesity and Type 2 diabetes by improving glycemic control and maintaining or increasing lean body mass while reducing fat mass in a subject, comprising administering a therapeutically effective amount of a GLP-1 agonist-tethered GDF8 antibody conjugate disclosed herein to the subject in need thereof.

In another aspect, provided herein is a method for treating obesity, diabetes, and/or liver diseases associated with increased fat mass in a subject, comprising administering a therapeutically effective amount of a GLP-1 agonist-tethered GDF8 antibody conjugate disclosed herein to the subject in need thereof.

In another aspect, provided herein is a method for treating obesity, diabetes, and/or liver diseases associated with increased fat mass in a subject, comprising administering a therapeutically effective amount of a GLP-1 agonist-tethered GDF8 antibody conjugate disclosed herein, together with one or more other therapeutic agents, to the subject in need thereof.

In another aspect, provided herein is a method for treating a subject of metabolic syndrome by improving glycemic control, maintaining or increasing lean body mass while reducing fat mass in a subject, comprising administering a therapeutically effective amount of a GLP-1 agonist-tethered GDF8 antibody conjugate disclosed herein to the subject in need thereof.

In another aspect, provided herein is a method for treating a subject of metabolic syndrome by improving glycemic control, maintaining or increasing lean body mass while reducing fat mass in a subject, comprising administering a therapeutically effective amount of a GLP-1 agonist-tethered GDF8 antibody conjugate disclosed herein, together with one or more other therapeutic agents, to the subject in need thereof.

In some embodiments, the GLP-1 agonist-tethered GDF8 antibody conjugate comprises
a) an antibody, or an antigen-binding fragment thereof, that specifically binds to and/or blocks the biological activity of, Growth and Differentiation Factor-8 (GDF8, SEQ ID NO: 1);
b) at least one Glucagon-like peptide-1 (GLP-1) receptor (GLP1R) agonist; and
c) at least one linker that covalently connects the at least one GLP1R agonist to the GDF8 antibody, or the antigen-binding fragment thereof.

In some embodiments, the GDF8 antibody, or the antigen-binding fragment thereof, comprises heavy chain complementarity determining regions (HCDRs) of a heavy chain variable region (HCVR) comprising SEQ ID NO: 4, and light chain complementarity determining regions (LCDRs) of a light chain variable region (LCVR) comprising SEQ ID NO: 5.

In some embodiments, where the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively, and the GLP1R agonist is the compound according to any of the embodiments described herein.

In some embodiments, the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively, and the GLP1R agonist has a sequence of H(Aib)EGTFTSDYSSYLEEQAAKEFIAWLVKGGG (SEQ ID NO: 3), where Aib is 2-Aminoisobutyric acid.

In some embodiments, the GLP1R agonist is selected from the group consisting of GLP-1(7-37) (SEQ ID NO: 2); the compound according to any of the embodiments described herein; exenatide, liraglutide, lixisenatide, albiglutide, dulaglutide, semaglutide, taspoglutide, and tirzepatide.

In some embodiments, the GLP1R agonist is selected from the group consisting of GLP-1(7-37) (SEQ ID NO: 2); GLP1R agonist (SEQ ID NO: 3), exenatide, liraglutide, lixisenatide, albiglutide, dulaglutide, semaglutide, taspoglutide, and tirzepatide.

In some embodiments, the GLP1R agonist is the compound according to any of the embodiments described herein.

In some embodiments, the GLP1R agonist has a sequence of H(Aib)EGTFTSDYSSYLEEQAAKEFIAWLVKGGG (SEQ ID NO: 3), where Aib is 2-Aminoisobutyric acid.

In some embodiments, the conjugate of GLP1R agonist tethered GFD8 antibody the structure selected from the group consisting of:

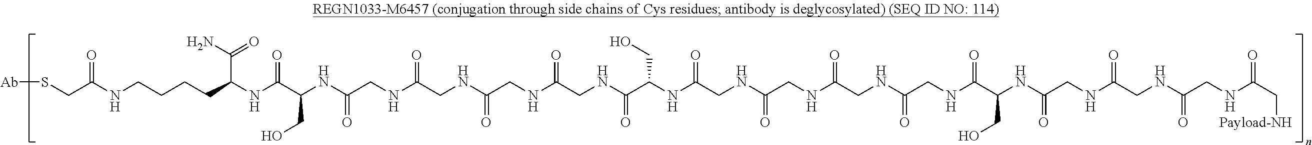

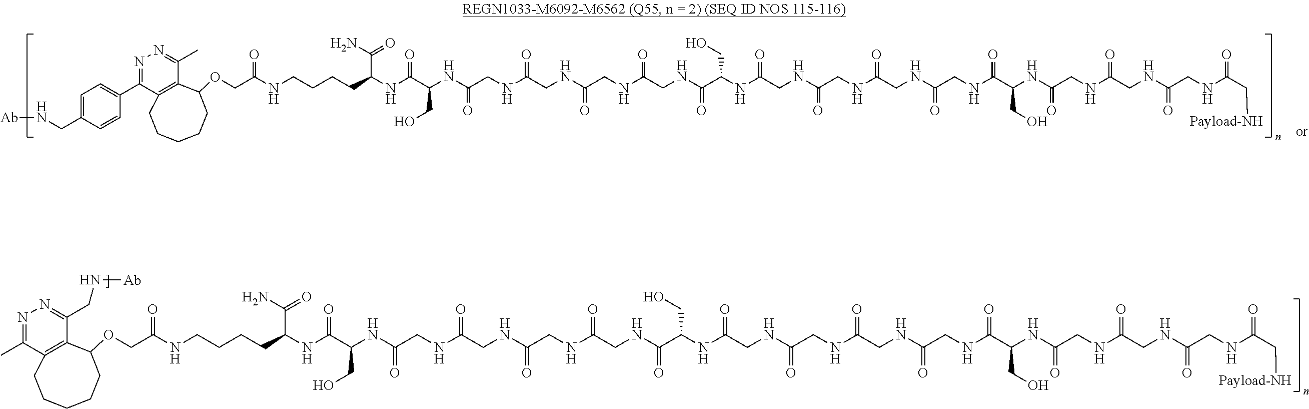

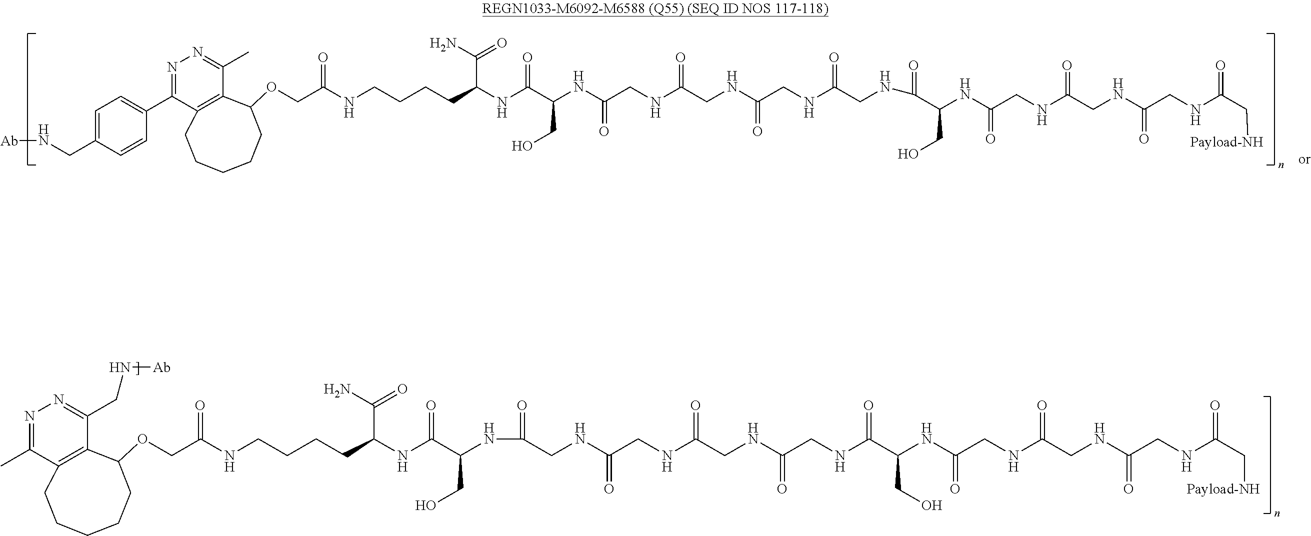

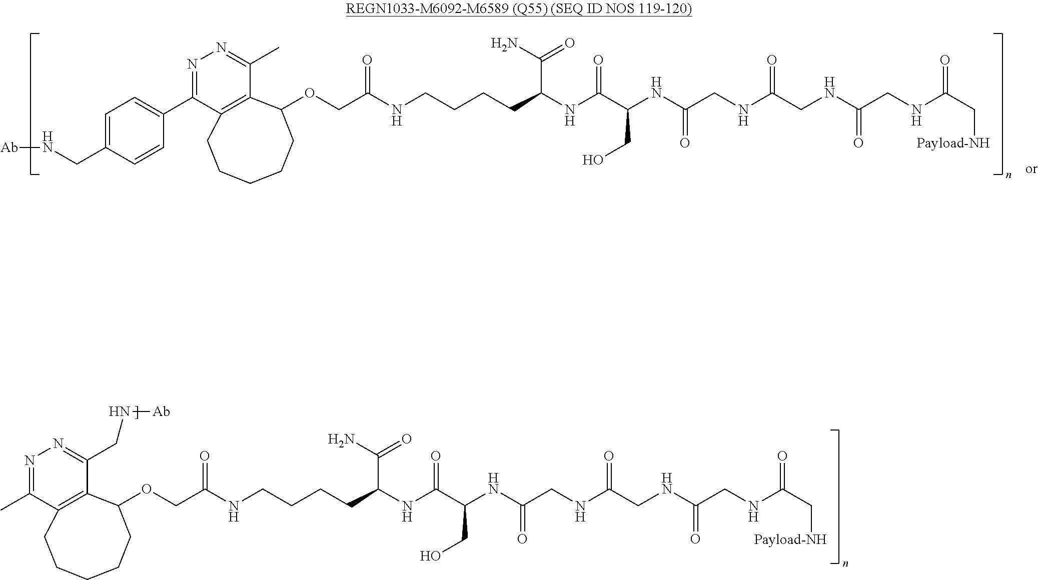

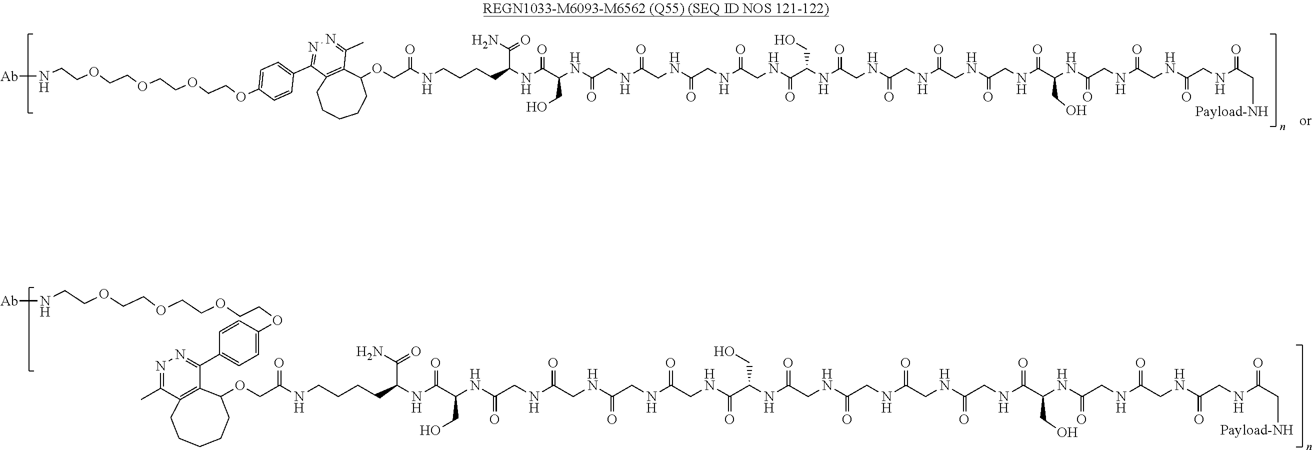

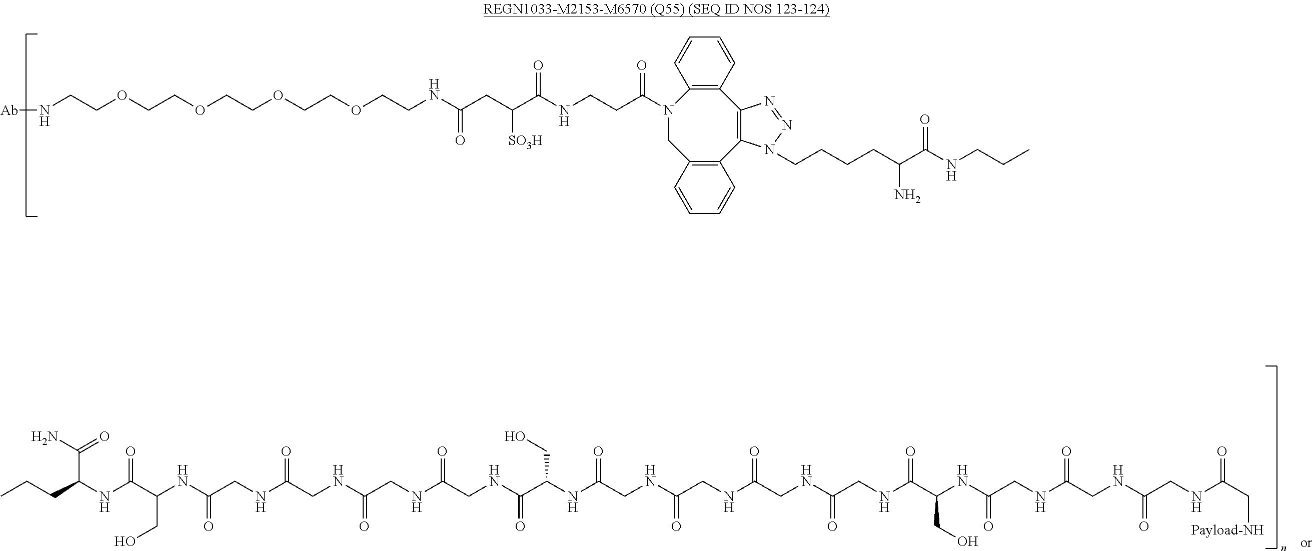

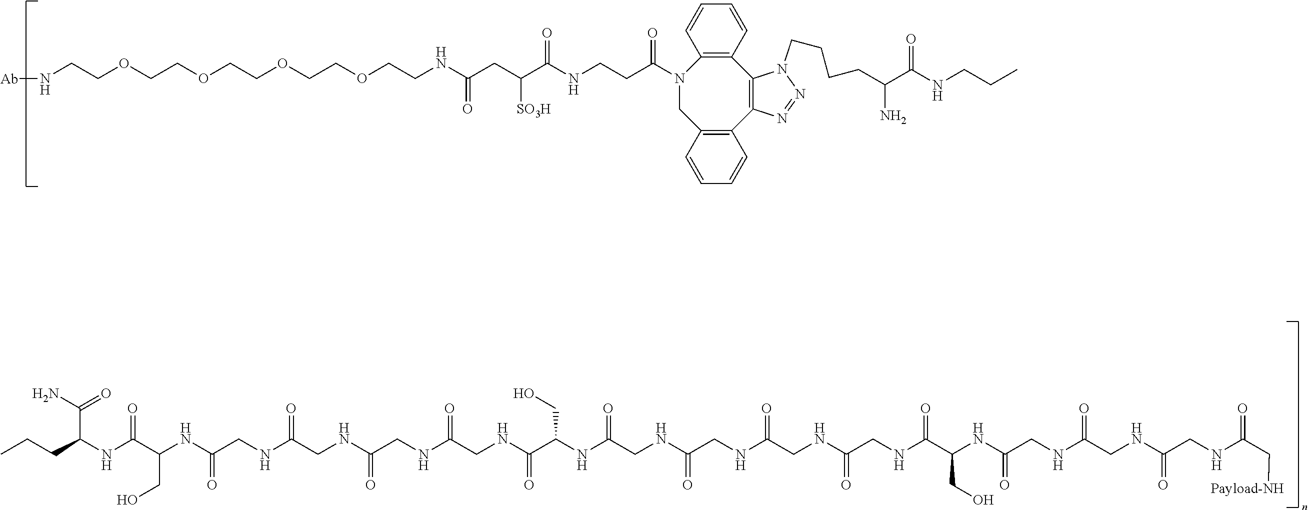

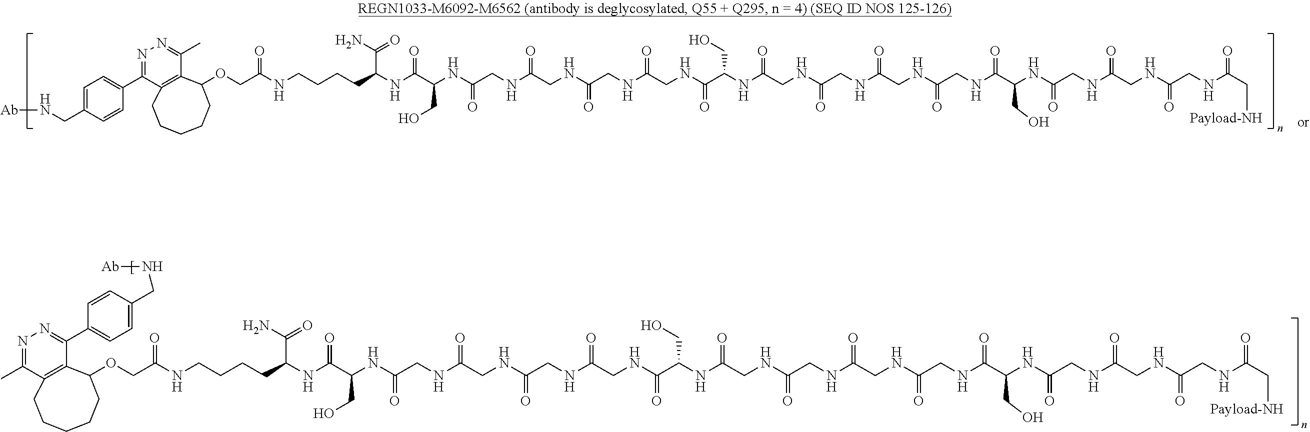

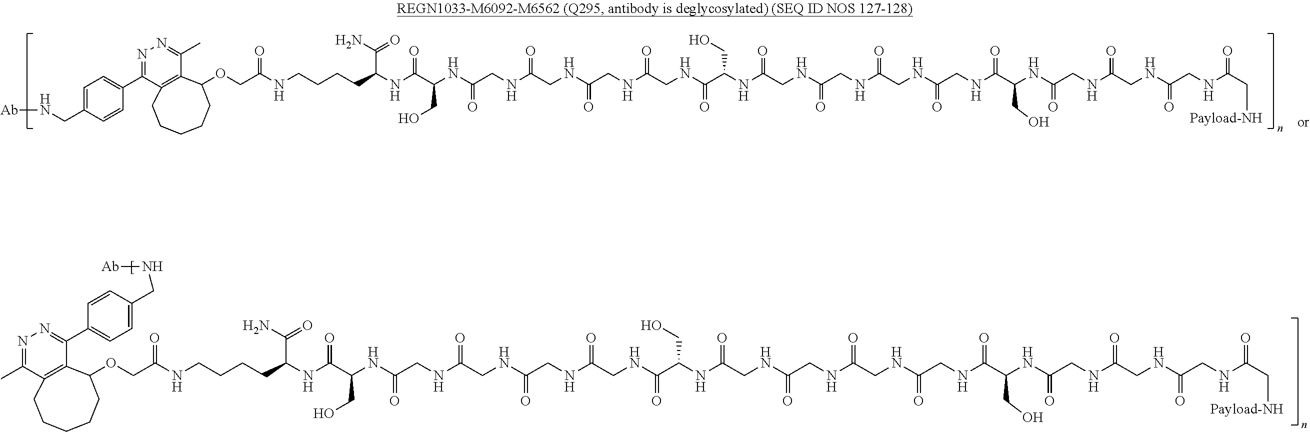

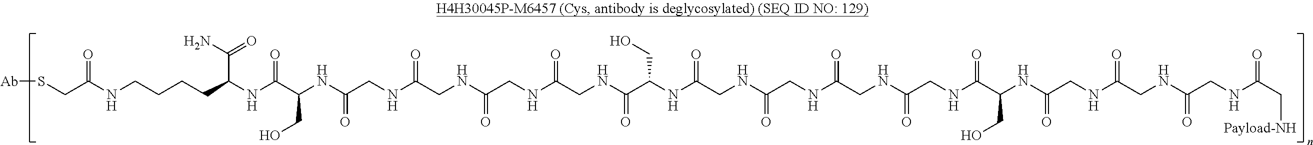

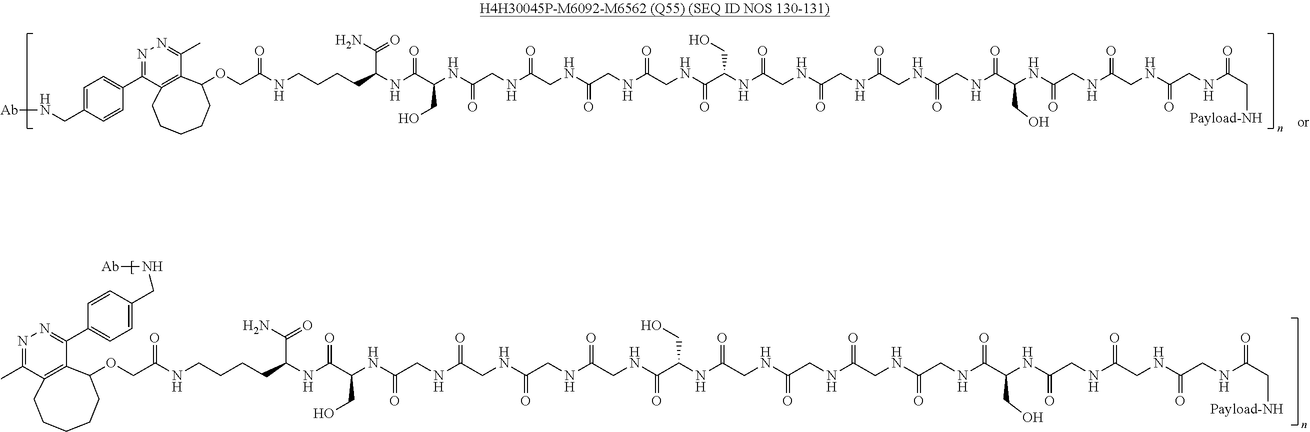

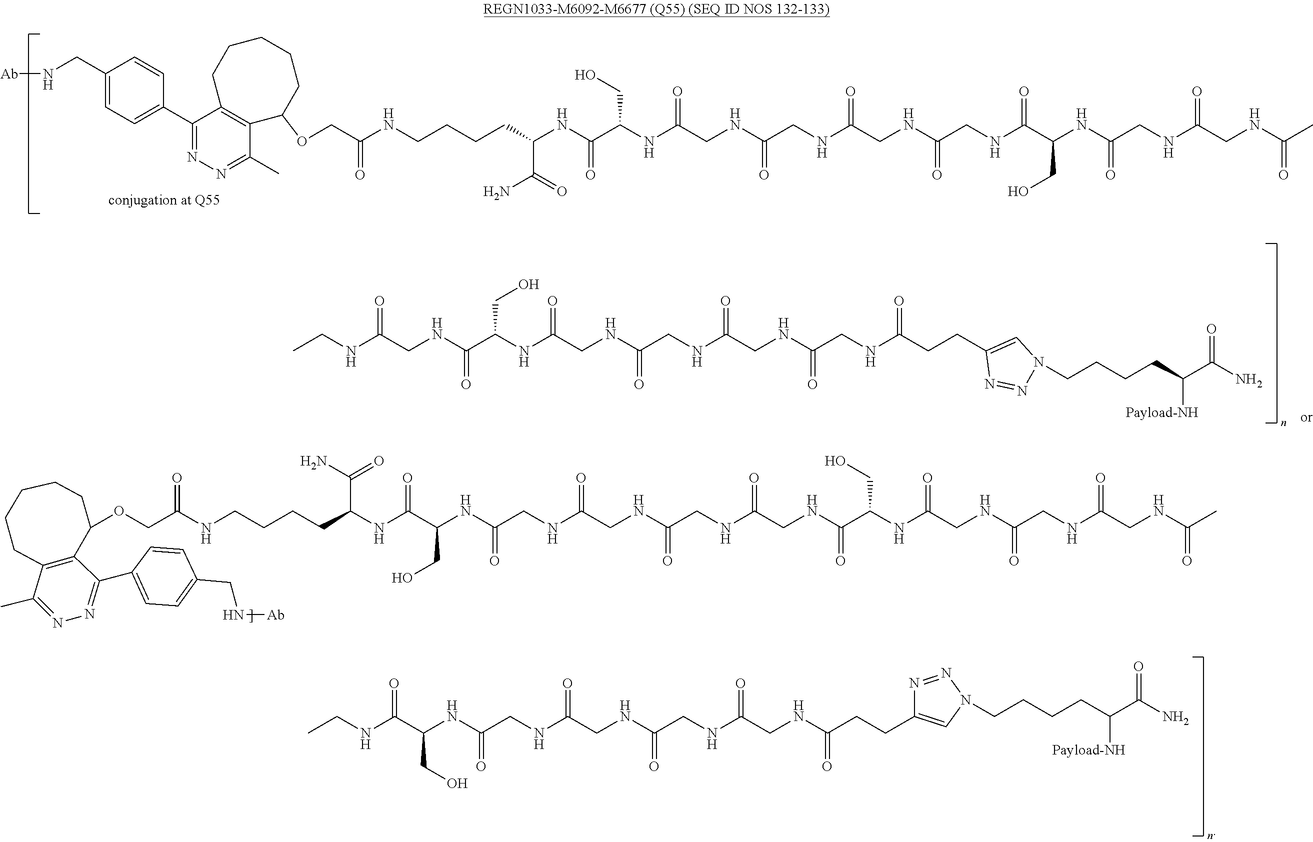

or a pharmaceutically acceptable salt thereof, where the Payload is the compound according to any of the embodiments described herein, and where n is about 1 to 10, and Ab is the GDF8 antibody, or the antigen-binding fragment thereof.

In some embodiments, the conjugate of GLP1R agonist tethered GFD8 antibody the structure selected from the group consisting of:

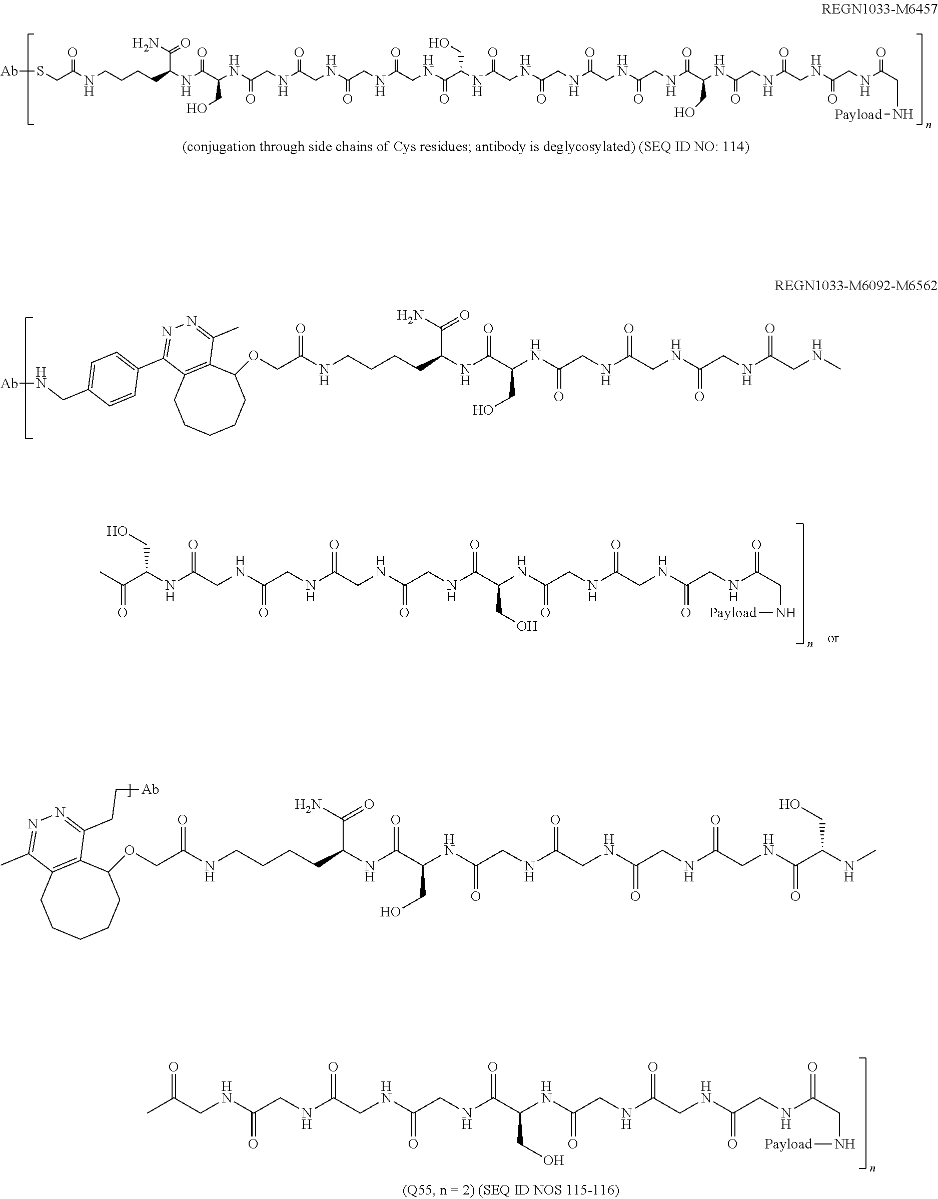

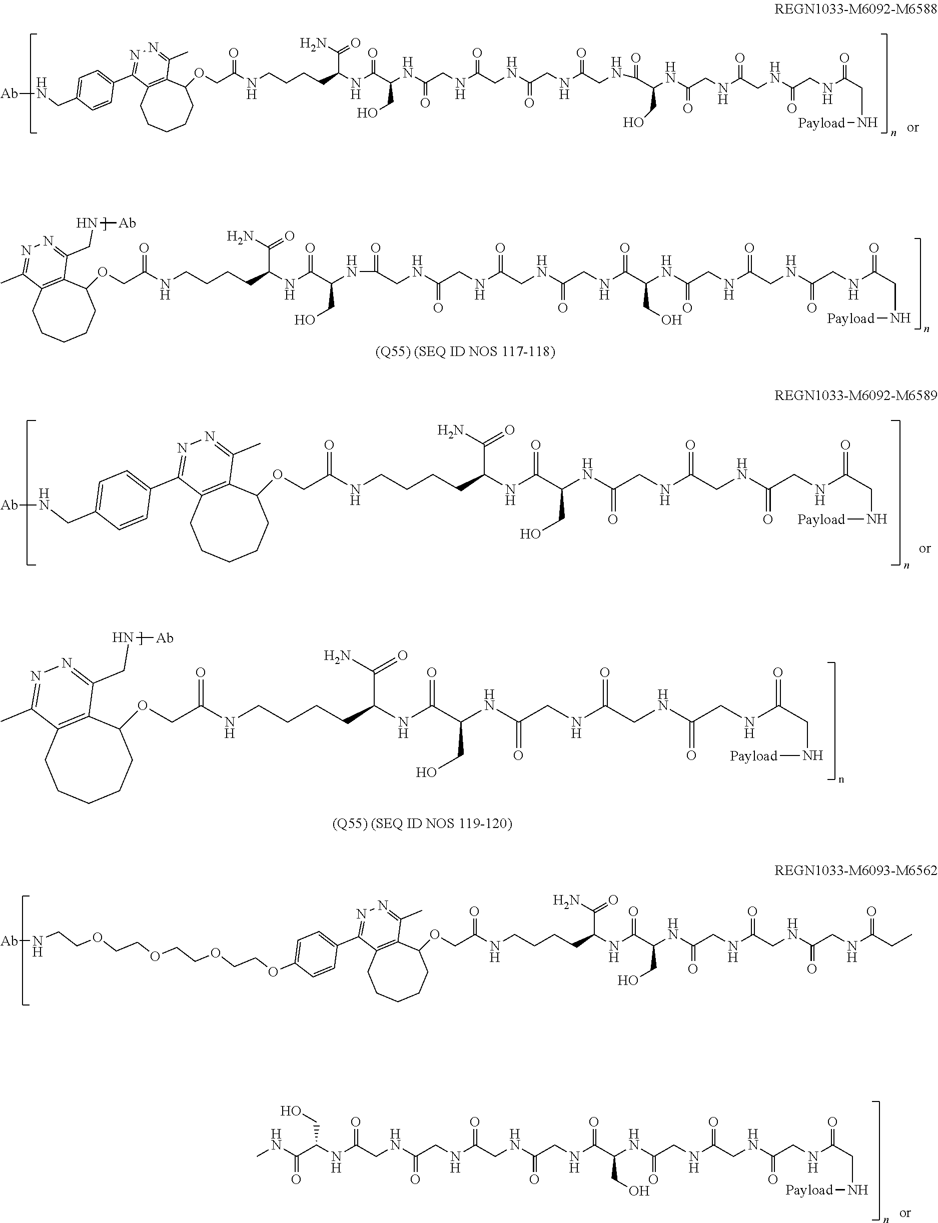

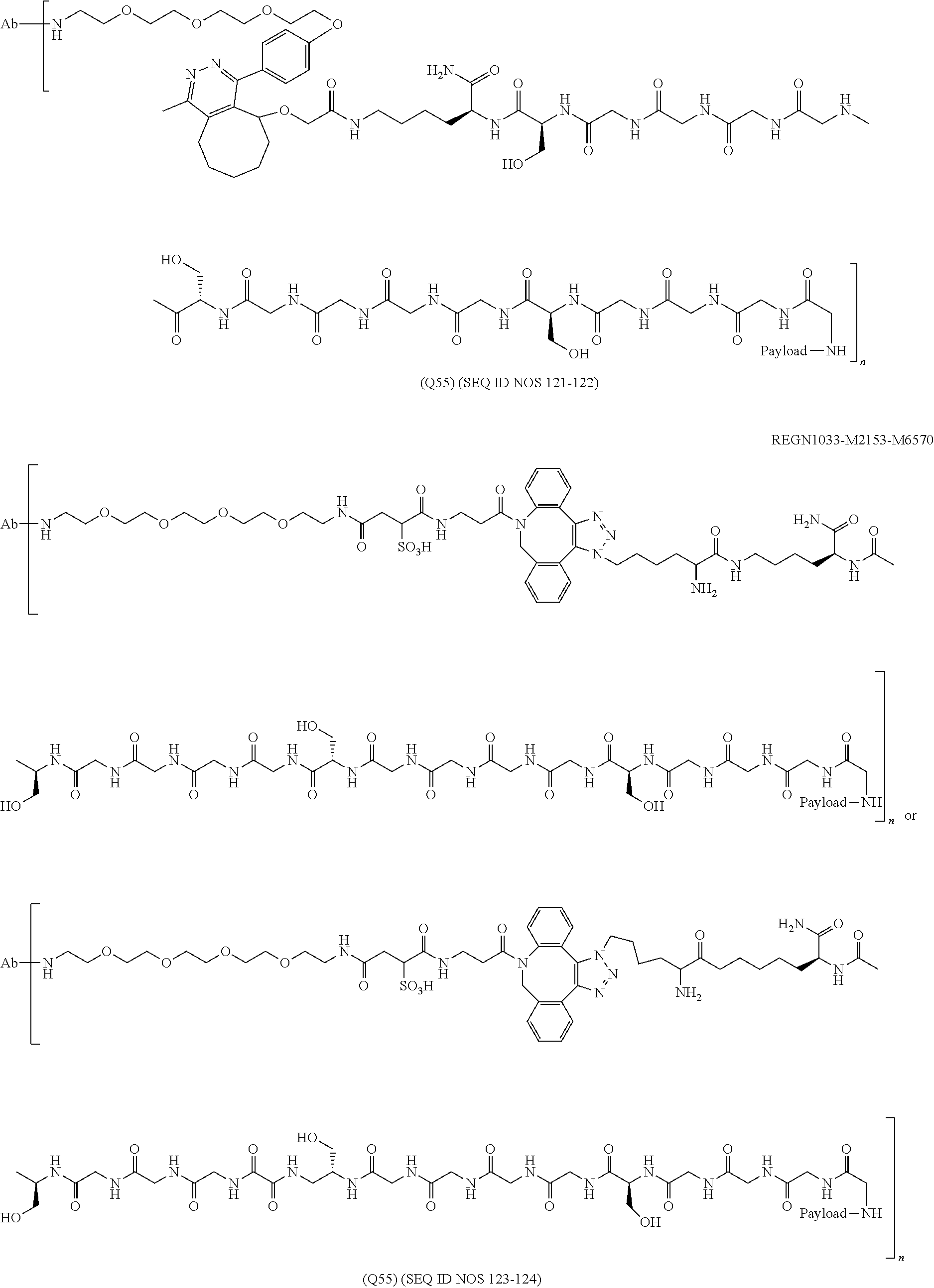

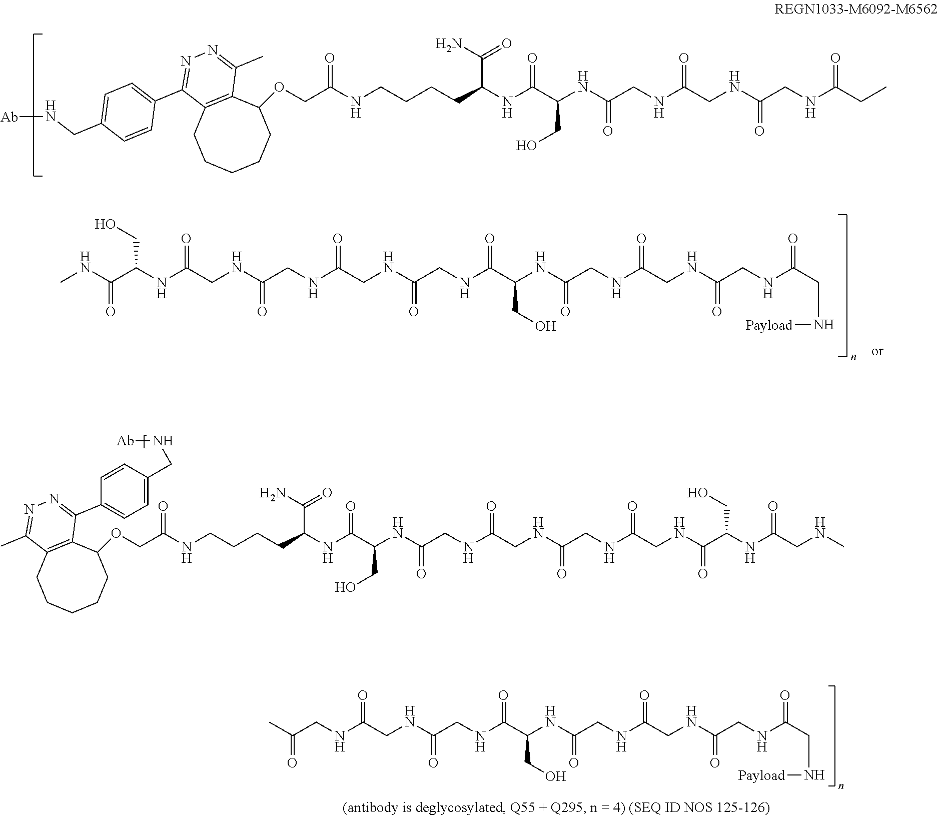

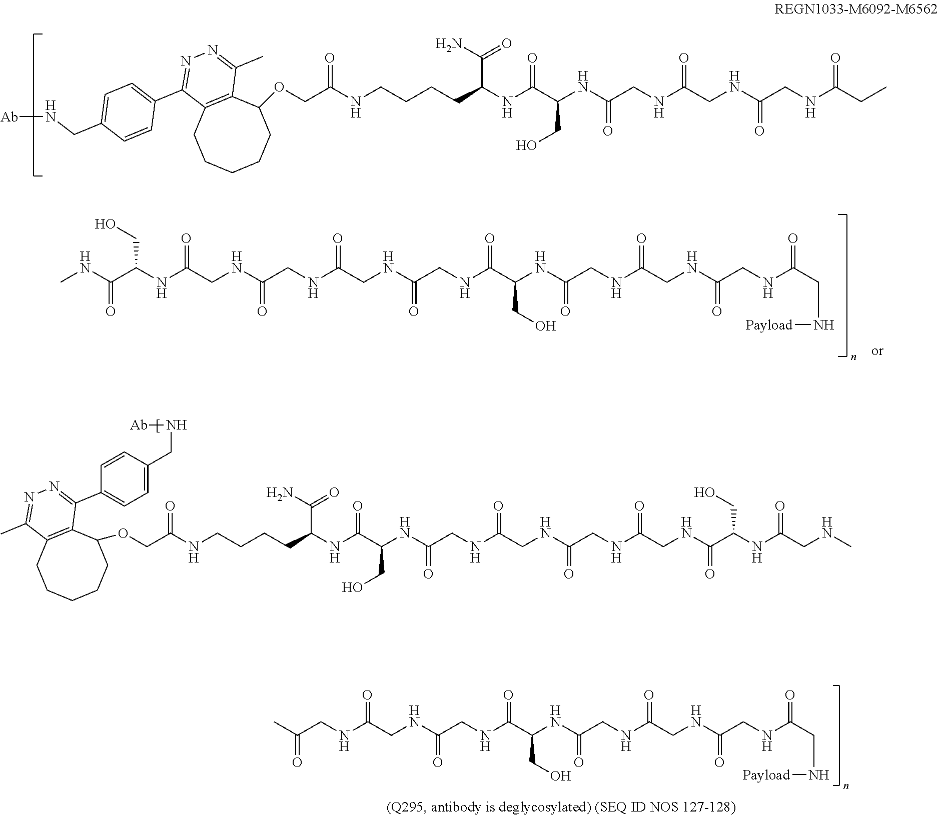

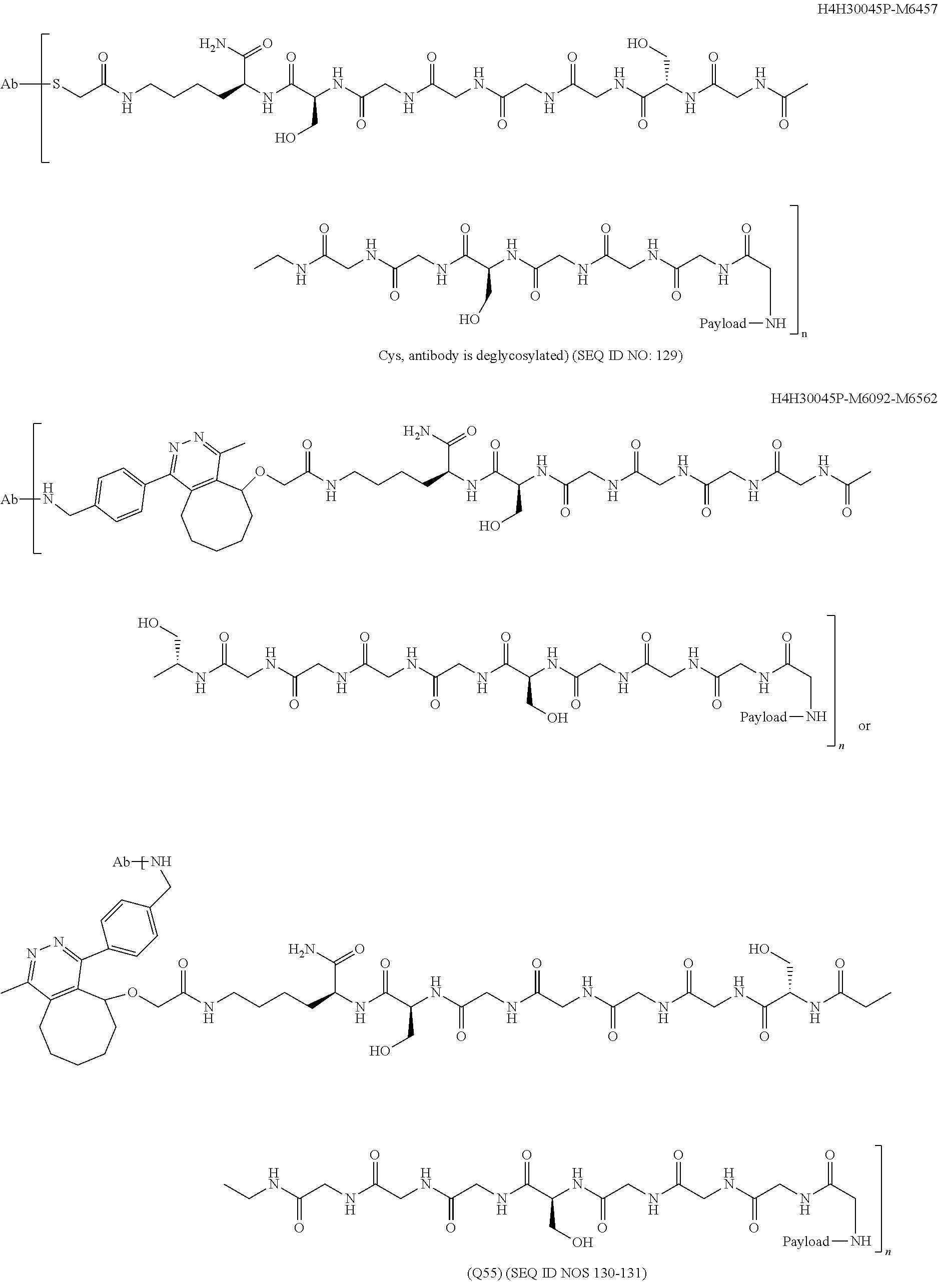

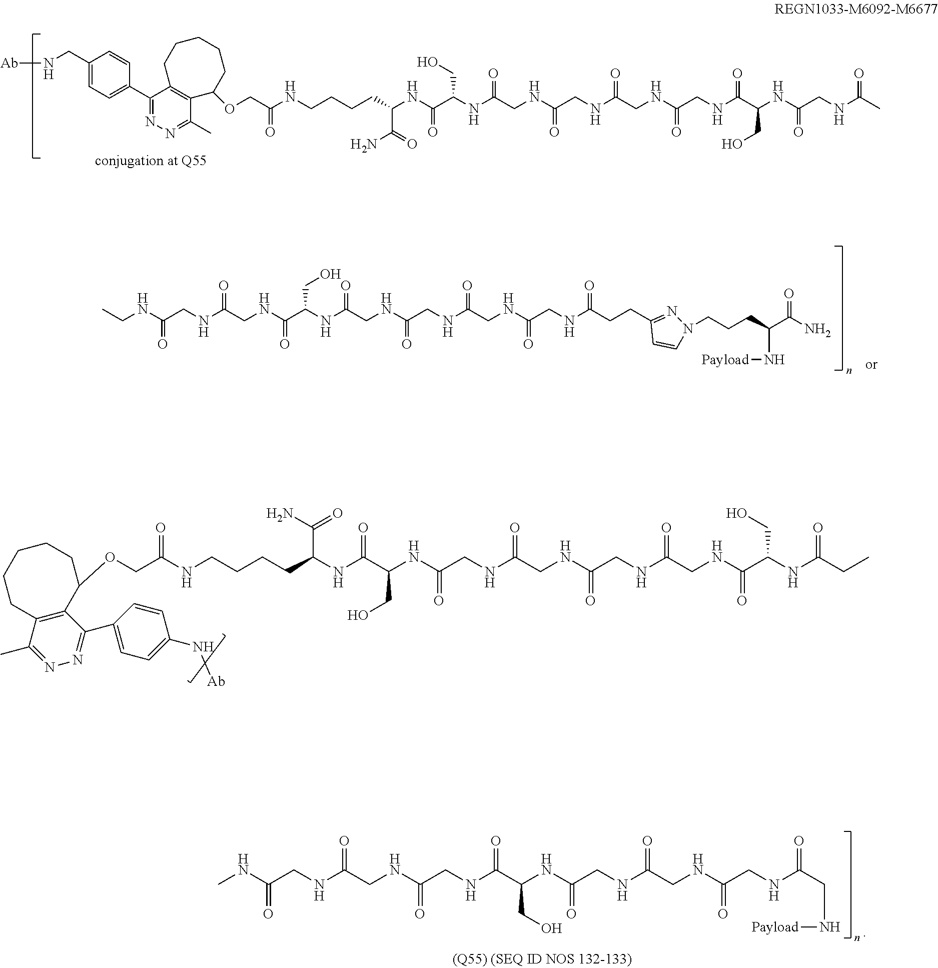

or a pharmaceutically acceptable salt thereof, where the Payload is H[Aib]EGTFTSDYSSYLEEQAAKEFIAWLVKGGG (SEQ ID NO: 3), and where n is about 1 to 10, and Ab is the GDF8 antibody, or the antigen-binding fragment thereof.

In some embodiments, the GLP1R agonist is conjugated to the GDF8 antibody or antigen binding fragment thereof via Q55 on the light chain or Q295 of the heavy chain.

In another aspect, provided herein is a process for manufacturing a conjugate of GLP1R agonist tethered GDF8 antibody or antigen binding fragment thereof. This proses comprises a) covalently attaching a handle comprising a first reactive moiety for Click or Diels-Alder reaction, in the presence of microbial transglutaminase; b) exposing a GLP1R agonist comprising a second reactive moiety for Click or Diels-Alder reaction, where the first and the second reactive moieties are complimentary to each other and form a stable conjugate; and c) isolating or purifying the conjugate of GLP1R agonist tethered GDF8 antibody or antigen binding fragment thereof.

In some embodiments, the reactive moiety for the Click reaction, comprises —N₃,

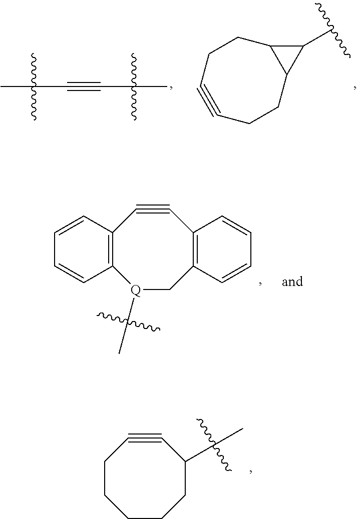

where Q is CH or N.

In some embodiments, the reactive moiety for the Diels-Alder reaction, comprises

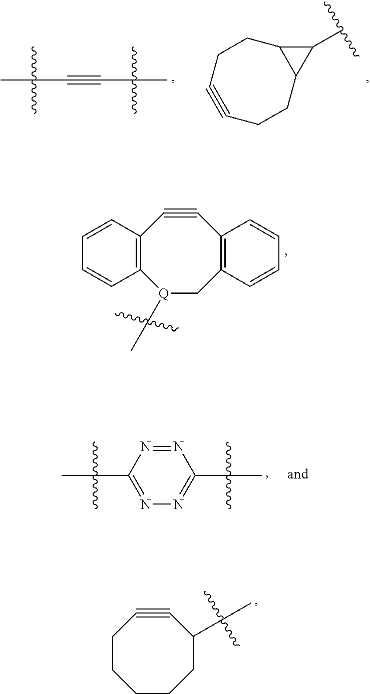

where Q is CH or N.

In some embodiments, the handle comprises

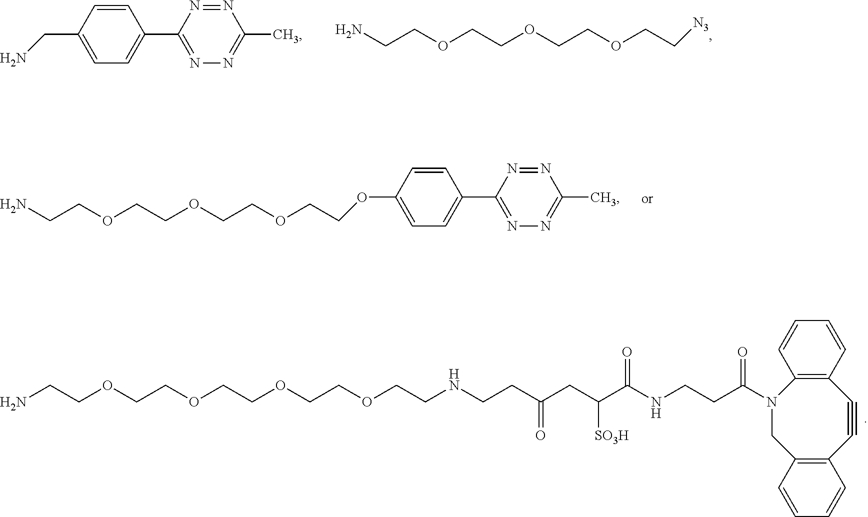 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the reactive moiety of the GDF8 antibody and the reactive moiety of the GLP1R agonist form a linker adduct having a structure of

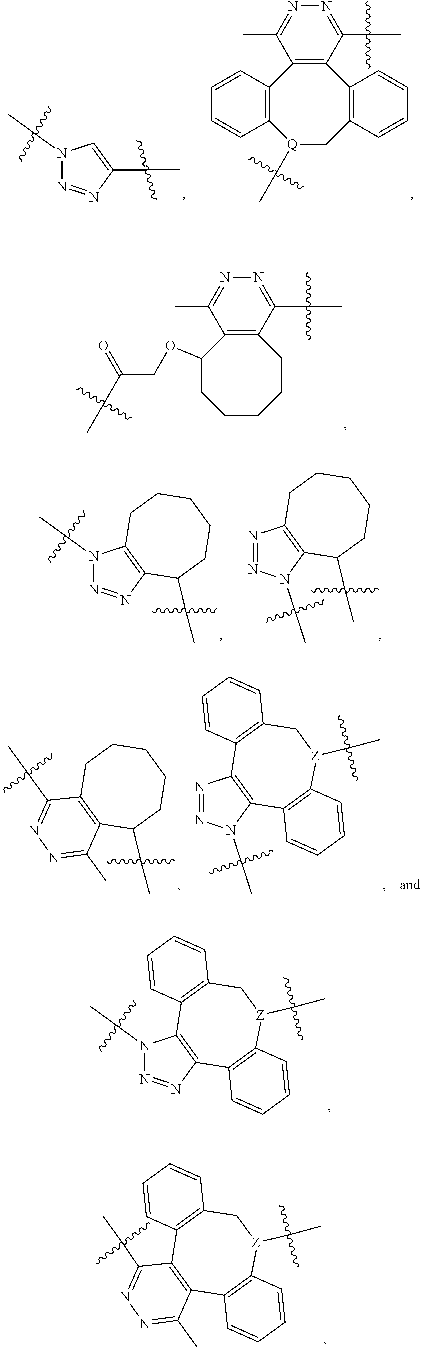

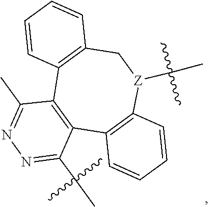

where Z is CH or N.

In some embodiments, the GLP1R agonist is conjugated to the GDF8 antibody or antigen binding fragment thereof via Q55 on the light chain or Q295 of the heavy chain of the antibody.

In another aspect, provided herein is a product of any of the process disclosed herein.

In another aspect, provided herein is a conjugate, or a pharmaceutically acceptable salt thereof, comprising an antigen-binding protein (BA) and a GLP1R agonist, where the BA specifically binds to and/or blocks the activity of GDF8 (SEQ ID NO: 1), and the GLP1R agonist is conjugated to the BA through a linker.

In some embodiments, the antigen-binding protein (BA) is an antibody or antigen-binding fragment thereof, where the BA is modified with a handle with the assistance of mTG, where the handle comprises a primary amino group and a reactive moiety for Click or Diels-Alder reaction for conjugation with the GLP1R agonist.

In some embodiments, the BA is an anti-GDF8 antibody trevogrumab comprising the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO: 5.

In some embodiments, the BA is an anti-GDF8 antibody trevogrumab comprising heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In some embodiments, the GLP1R agonist is a compound according to any of the embodiments described herein.

In some embodiments, the GLP1R agonist is a peptide comprising the amino acid sequence of H(Aib)EGTFTSDYSSYLEEQAAKEFIAWLVKGGG (SEQ ID NO: 3).

In some embodiments, the GLP1R agonist is selected from the group consisting of exenatide, liraglutide, lixisenatide, albiglutide, dulaglutide, semaglutide, taspoglutide, and tirzepatide.

In some embodiments, the linker comprises a conjugation reaction product of a reactive moiety from the GLP1R agonist and a reactive moiety from the BA.

In some embodiments, the reactive moiety comprises thiol, maleimido, 2-bromo acetyl, —N₃,

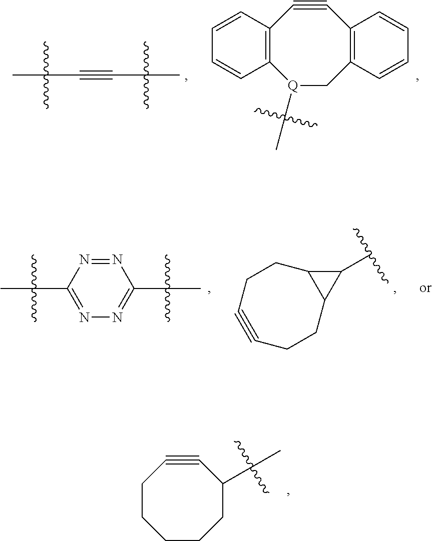

where Q is CH or N.

In some embodiments, the reactive moiety comprises

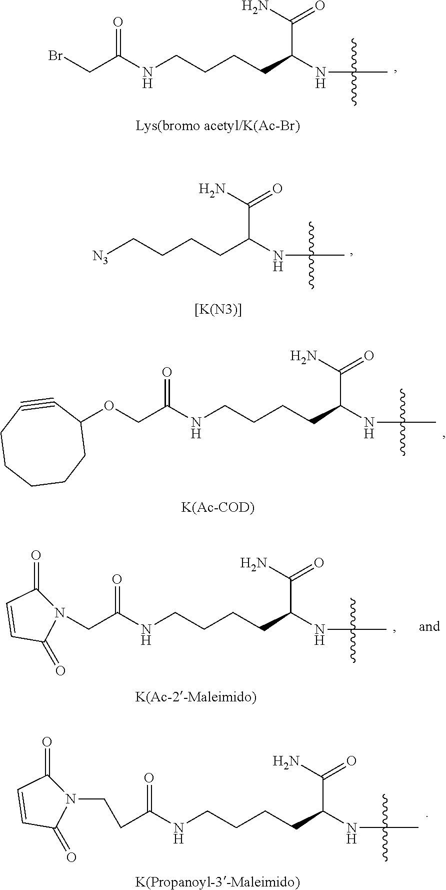

In some embodiments, the linker has a structure of

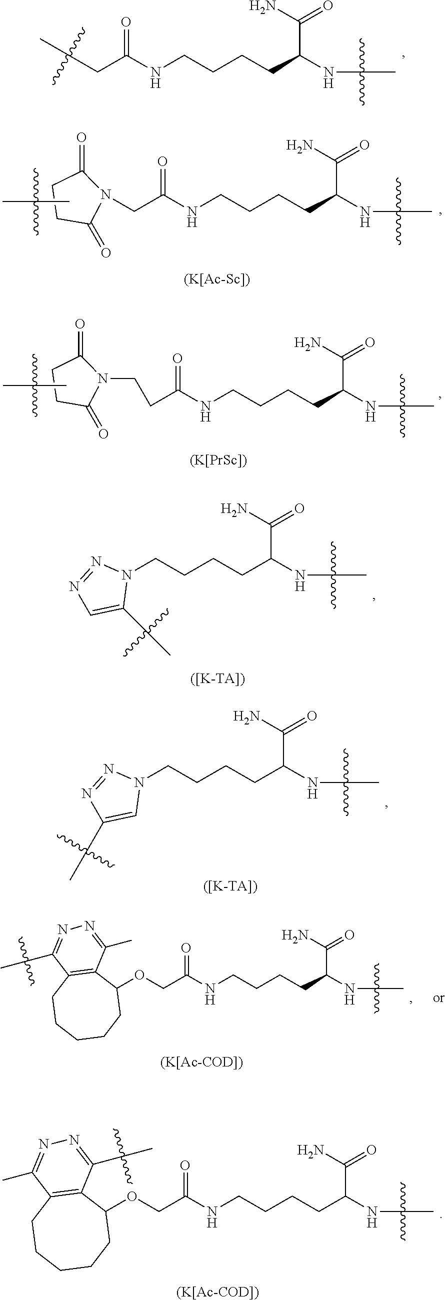

In some embodiments, the linker comprises a moiety formed by Click or Diels-Alder reaction, having the structure of have a structure of

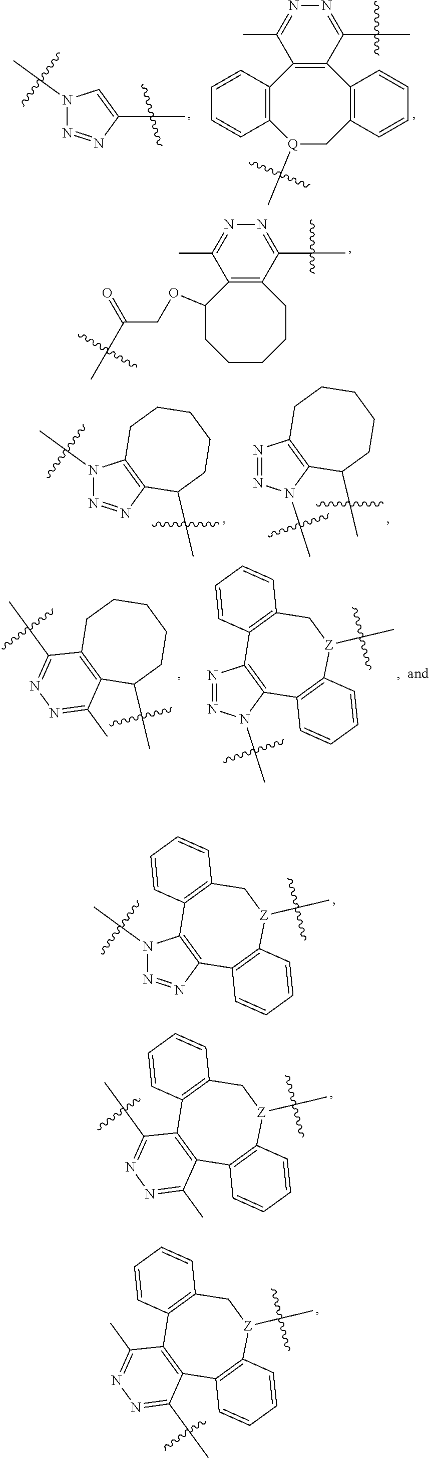

where Z is CH or N.

In some embodiments, the linker further comprises a sequence of -(Gly₃Ser)ₙ-Lys(X)- (SEQ ID NO: 89) or -(Gly₄Ser)ₙ-Lys(X)- (SEQ ID NO: 90), where n is an integer from one to six; and where X is a reactive moiety attached to the side chain of Lys for conjugation with the BP.

In another aspect, provided herein is a pharmaceutical composition comprising the conjugate according to any of the embodiments described herein, or a pharmaceutically acceptable salt thereof.

In some embodiments, the pharmaceutical composition further comprises one or more pharmaceutically accept salts, excipients, and/or diluents.

In another aspect, provided herein is a process for conjugating a drug, a ligand, or a handle, site-specifically to an isolated antibody or antigen binding fragment thereof, where the site of conjugation is at Gln/Q55 of the light chain of the antibody, and where the site-specific conjugation is assisted by microbial transglutaminase (mTG).

In some embodiments, the drug, ligand, or handle comprises a primary amino group for the conjugation assisted by mTG to the side chain of Q55 of the antibody.

In some embodiments, the handle comprises a primary amino group for conjugation assisted by mTG to the side chain of Q55 of the antibody and a reactive moiety for conjugation with a drug or a ligand.

In some embodiments, the reactive moiety is for the Click or the Diels-Alder reaction.

In some embodiments, the reactive moiety for the Click reaction comprises —N₃,

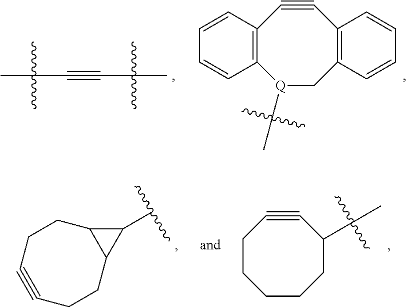

where Q is CH or N.

In some embodiments, the reactive moiety for the Diels-Alder reaction comprises

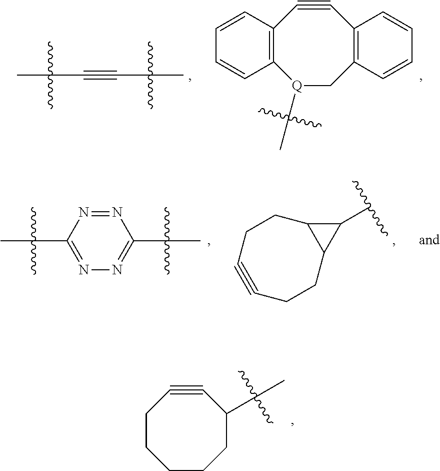

where Q is CH or N.

In some embodiments, the handle comprises

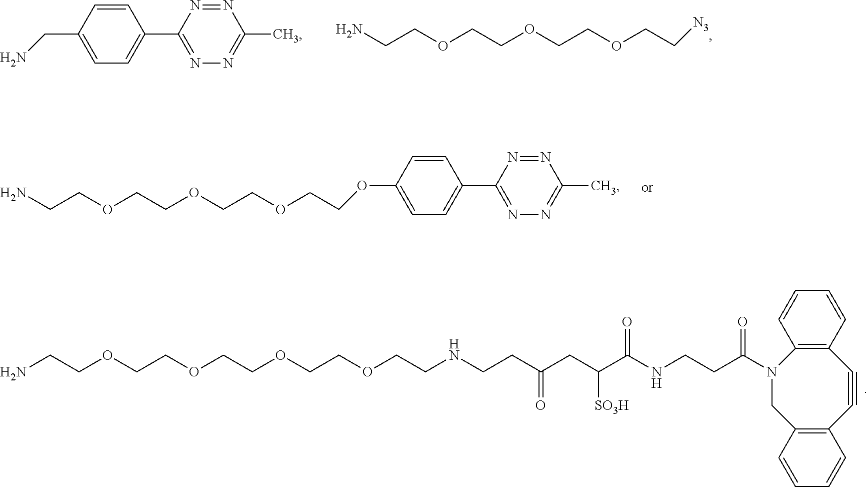 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the isolated antibody or antigen binding fragment thereof comprises REGN1033 (anti-GDF8 antibody), REGN4320 (anti-MSR1 N297Q), REGN4322 (H1H21231N, anti-MSR1, N297Q), H2aM21339N (anti-HLA-A2/CMV), H4H11283N (anti-HLA-B27), H4sH14137N (anti-CD20), H1H20918P (CD226), H4H20122P (HLA-B27), H4H13767P (HFE2), H4H12587P (IL-6R), H4H11281N (HLA-B27), H1H15208P (MERS), H4H11283N, H4H11924N, H2aM14137N, H4sH14137N, H4H6073N2, H2aM14140N, and H1H10154P.

In some embodiments, the isolated antibody or antigen binding fragment thereof preferably has a phenylalanine residue at position 46 of the light chain.

In some embodiments, the isolated antibody or antigen binding fragment thereof is a GDF8 antibody or antigen-binding fragment thereof that specifically binds to and/or blocks the biological activity of wild-type mature human GDF8 comprising SEQ ID NO: 1.

In another aspect, provided herein is an antibody conjugate or a pharmaceutically acceptable salt thereof manufactured according to any of the processes described herein.

In another aspect, provided herein is a GLP1R agonist-tethered antibody conjugate, or a pharmaceutically acceptable salt thereof, wherein the conjugate comprises
a) an antibody, or an antigen-binding fragment thereof,
b) at least one Glucagon-like peptide-1 (GLP-1) receptor (GLP1R) agonist according to any of the embodiments described herein; and
c) one linker or a bond that covalently connects the at least one said GLP1R agonist to the antibody, or the antigen-binding fragment thereof.

In some embodiments, the antibody is a PCSK9 antibody or antigen-binding fragment thereof.

In some embodiments, the antibody is a GLP1R antibody or antigen-binding fragment thereof.

In some embodiments, the antibody is a GIPR antibody or antigen-binding fragment thereof.

In some embodiments, the antibody is an activin receptor type 2 antibody or antigen-binding fragment thereof.

In some embodiments, the antibody is a Growth and Differentiation Factor-8 (GDF8, myostatin) antibody or antigen-binding fragment thereof.

In some embodiments, the GDF8 antibody, or an antigen-binding fragment thereof, binds to and/or blocks the biological activity of Growth and Differentiation Factor-8 (GDF8, SEQ ID NO: 1).

In some embodiments, the GDF8 antibody or the antigen binding fragment thereof comprises 1A2, 21-E5, 8D12, H4H1669P, AMG 181, AMG 745, Apitegromab (SRK-015), Bimagrumab (BYM338), Domagrozumab (PF-06252616), GYM329 (RO7204239), Landogrozumab (LY2495655), Stamulumab (MYO-029), and Trevogrumab (REGN1033).

In another aspect, provided herein is a pharmaceutical composition comprising an antibody conjugate manufactured according to any of the processes described herein together with one or more pharmaceutically acceptable excipients.

These and other aspects of the present disclosure will become apparent to those skilled in the art after a reading of the following detailed description of the disclosure, including the appended claims.

### BRIEF DESCRIPTIONS OF DRAWINGS

FIGS. 1A-1C show three different processes for site-specific conjugation mediated by mTG: conjugation at Q55 using a wild-type mTG without deglycosylation (FIG. 1A), conjugation at Q55 and Q295 using a mutated mTG without deglycosylation (FIG. 1B), and conjugation at Q295 by blocking Q55 first using a wild-type mTG with a deglycosylated antibody (FIG. 1C).

FIG. 2 shows preparative size exclusion chromatography (SEC) chromatogram of an ATL conjugation mixture.

FIG. 3 shows analytical SEC chromatogram of a purified anti-GDF8 ATL.

FIGS. 4A-4D show characterization of the crude conjugate REGN1033-M6092-M6562_L100. Reaction conditions: 5×LP, 37° C., 20.2 mg/mL, 14% DMSO.

FIG. 5 shows interchain cysteine conjugation.

FIG. 6 shows that REGN1033-M6562 reduces body weight better than REGN1033-M6457. DIO mice were dosed subcutaneously at 10 mg/kg weekly with the labeled treatments. Body weights were measured twice weekly throughout the study. Each data point is mean±SEM. *P<0.05 vs. Control mAb, #P<0.05 vs. REGN1033-Q55, \hat{ }P<0.05 vs. Isotype-Q55. Stats by two-way RM ANOVA.

FIGS. 7A-7B shows that REGN1033-M6562 reduces body fat to a greater degree than REGN1033-M6457. Body composition was measured by qNMR at baseline and on D14 and D23 of the study. Data expressed as percent change from the baseline reading. Each data point is mean±SEM. *P<0.05 vs. Control mAb, #P<0.05 vs. REGN1033-Q55, \hat{ }P<0.05 vs. Isotype-Q55. Stats by two-way RM ANOVA.

FIGS. 8A-8F show that both REGN1033 conjugated to GLP1R agonists via Q55 or cysteines reduce mesenteric fat while reversing GLP-1 induced lean mass loss in DIO mice. Terminal fat and skeletal muscle weights. Data expressed as percent change from Control mAb. Each data point is mean±SEM. *P<0.05, **P<0.01, ***P<0.001, ****P<0.0001 vs. designated group. Stats by one-way ANOVA.

FIGS. 9A-9C show that both REGN1033 conjugated to GLP1R agonists via Q55 or cysteines reduce fed glucose and liver weight in DIO mice. Fed blood glucose and terminal liver and pancreas weights. Fed glucose expressed as percent change from baseline, liver and pancreas as percent change from REGN1945. Each data point is mean±SEM. Panel A: *P<0.05 vs. REGN1945, #P<0.05 vs. REGN1033. Stats by two-way RM ANOVA. Panels B-C: *P<0.05 vs. REGN1945, #P<0.05 vs. REGN1033. Stats by two-way RM ANOVA.

FIG. 10 shows reduction of body weight with REGN1033-M6562 and REGN1033-M6677. DIO mice were dosed subcutaneously at 10 mg/kg weekly with the labeled treatments. Body weights were measured twice weekly throughout the study. Each data point is mean±SEM. ****P<0.0001 vs. Control mAb. Stats by two-way RM ANOVA.

FIGS. 11A-11B show reduction of body fat with REGN1033-M6562 and REGN1033-M6677. Body composition was measured by qNMR at baseline and on D14 and D23 of the study. Data expressed as percent change from the baseline reading. Each data point is mean±SEM. ****P<0.0001 vs. Control mAb. Stats by two-way RM ANOVA.

FIGS. 12A-12F show individual depot weights with REGN1033-M6562 and REGN1033-M6677. Terminal fat and skeletal muscle weights. Data expressed as percent change from Control mAb. Each data point is mean±SEM. *P<0.05, ***P<0.001, ****P<0.0001 vs. designated group. Stats by one-way ANOVA.

FIGS. 13A-13C shows fed glucose and liver weight effects with REGN1033-M6562 and REGN1033-M6677. Fed blood glucose and terminal liver and pancreas weights. Fed glucose expressed as percent change from baseline, liver, and pancreas as percent change from Control mAb. Each data point is mean±SEM. Panel A: *P<0.05 vs. Control. Stats by two-way RM ANOVA. Panels B-C: ***P<0.001, ****P<0.0001 vs. designated group. Stats by two-way RM ANOVA.

FIG. 14 shows the locations of conjugations (as indicated by a star) for antibodies REGN4320 (no conjugation at Q55) and REGN4322 (efficient conjugation at Q55).

FIGS. 15A and 15B show the four stereo isomers formed during conjugation process, one chiral center of R- and S- configurations together with the cis- and trans- isomers, e.g. cis-R, cis-S, trans-R, and trans-S. See also Table 46 for the detected mass signals. Figures disclose SEQ ID NOS 113, 135, 141, and 142, respectively, in order of appearance.

FIG. 16A depicts UV (254 nm, 16A) and FIG. 16B shows the base-peak ion (BPI, 16B) chromatogram of four isomeric peaks between retention time (RT) 46.0-47.5 min. See also Table 46 for more detailed information.

FIG. 17 shows representative MS/MS spectra of corresponding light chain peptide conjugated with linker/payload M6092-M6562 after trypsin digestion. See also Table 45 for more details.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive. Therefore, specific structural and functional details disclosed herein are not to be interpreted as limiting, but merely as a representative basis for teaching one skilled in the art to variously employ the present disclosure.

### Definitions

Unless defined otherwise, all technical and scientific terms used herein have the same meaning as commonly understood by one of ordinary skill in the art to which this disclosure belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder, or condition developing in a subject that may be afflicted with or predisposed to the state, disorder, or condition but does not yet experience or display clinical or subclinical symptoms of the state, disorder, or condition; or (2) inhibiting the state, disorder, or condition, i.e., arresting, reducing or delaying the development of the disease or a relapse thereof or at least one clinical or sub-clinical symptom thereof; or (3) relieving the disease, i.e., causing regression of the state, disorder, or condition or at least one of its clinical or sub-clinical symptoms. The benefit to a subject to be treated is either statistically significant or at least perceptible to the patient or to the physician. In some embodiments, treatment comprises methods where cells are ablated in such manner where disease is indirectly impacted. In certain embodiments, treatment comprises depleting immune cells as a hematopoietic conditioning regimen prior to therapy.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable salt”, as used in connection with compositions of the disclosure, refers to any salt suitable for administration to a patient. Suitable salts include, but are not limited to, those disclosed in. Berge et al., “Pharmaceutical Salts”, J. Pharm. Sci., 1977, 66:1, incorporated herein by reference. Examples of salts include, but are not limited to, acid derived, base derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, para-toluene sulfonic acid, salicylic acid, and the like. In some examples, a payload described herein comprises a tertiary amine, where the nitrogen atom in the tertiary amine is the atom through which the payload is bonded to a linker or a linker-spacer. In such instances, bonding to the tertiary amine of the payload yields a quaternary amine in the linker-payload molecule. The positive charge on the quaternary amine can be balanced by a counter ion (e.g., chloro, bromo, iodo, or any other suitably charged moiety such as those described herein).

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.). Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “adduct” of the present disclosure encompasses any moiety comprising the product of an addition reaction, independent of the synthetic steps taken to produce the moiety.

The term “covalent attachment” means formation of a covalent bond, i.e., a chemical bond that involves sharing of one or more electron pairs between two atoms. Covalent bonding may include different interactions, including but not limited to a-bonding, 7z-bonding, metal-to-metal bonding, agostic interactions, bent bonds, and three-center two-electron bonds. When a first group is said to be “capable of covalently attaching” to a second group, this means that the first group is capable of forming a covalent bond with the second group, directly or indirectly, e.g., through the use of a catalyst or under specific reaction conditions. Non-limiting examples of groups capable of covalently attaching to each other may include, e.g., an amine and a carboxylic acid (forming an amide bond), a maleimide and a thiol (forming a thio-maleimide), and an azide and an alkyne (forming a triazole via a 1,3-cycloaddition reaction).

The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

Unless otherwise stated, cyclic adducts, e.g., products of a cycloaddition reaction, e.g., an azide-acetylene cycloaddition reaction, also referred to as Click reaction, depicted herein include all regioisomers, i.e., structural isomers that differ only in the position of a functional group or a substituent. By way of an example, the following structures represent triazole regioisomers, which differ only in the position of the substituent on the triazole ring:

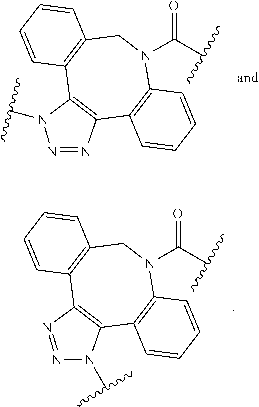 [SMILES: *C(=O)N1Cc2ccccc2-c2c(nnn2*)-c2ccccc21 ; *C(=O)N1Cc2ccccc2-c2nnn(*)c2-c2ccccc21]

Triazole regioisomers may also be represented by the following structure:

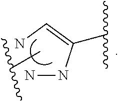

As disclosed herein, a Click reaction, Click chemistry, or Diels-Alder reaction, is a class of bioorthogonal organic reactions that could be used to selectively and/or site-specifically modify a protein or an antibody in an aqueous environment. Diels-Alder reactions as disclosed herein comprise not only the traditional Diels-Alder reaction between a diene and a dienophile but also include those newly developed the IEDDA (inverse electron demand Diels-Alder reaction). Due to its fast, efficient, and selective nature of this class of reactions, IEDDA has also been classified as a Click reaction. For more details, please see R. Bird, et al., Bioconjugate Chem. 2021, 32, 12, 2457-2479; Knall and Slugovc, Chem. Soc. Rev., 2013, 42, 5131; Baalmann, et al., Angew. Chem. Int. Ed. 2020, 59, 12885-12893; and Deb, et al., Chem. Rev. 2021, 121, 6850-6914.

Unless otherwise stated, all tautomeric forms of the compounds of the disclosure are within the scope of the disclosure.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this disclosure.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the disclosure and salts thereof are also within the scope of the disclosure. The compounds of the disclosure may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the disclosure are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the disclosure are substantially isolated. By “substantially isolated” is meant that a particular compound is at least partially isolated from impurities. For example, in some embodiments a compound of the disclosure comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

Certain groups, moieties, substituents, and atoms are depicted with a wavy line. The wavy line can intersect or cap a bond or bonds. The wavy line indicates the atom through which the groups, moieties, substituents, or atoms are bonded. For example, a phenyl group that CH₃ CH₃ is substituted with a propyl group depicted as:

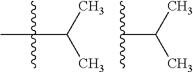

has the following structure:

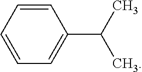

All amino acid abbreviations used in this disclosure are those accepted by the United States Patent and Trademark Office as set forth in 37 C.F.R. § 1.822 (B)(J).

The term “protein” means any amino acid polymer having more than about 20 amino acids covalently linked via amide bonds. As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins can be produced using recombinant cell-based production systems, such as the insect bacculovirus system, yeast systems (e.g, Pichia sp.), mammalian systems (e.g., CHO cells and CHO derivatives like CHO-K1 cells).

All references to proteins, polypeptides and protein fragments herein are intended to refer to the human version of the respective protein, polypeptide or protein fragment unless explicitly specified as being from a non-human species. Thus, the expression “GLP1R” means human GLP1R unless specified as being from a non-human species, e.g., “mouse GLP1R,” “monkey GLP1R,” etc.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

The term “glutaminyl-modified antibody” refers to an antibody with at least one covalent linkage from a glutamine side chain to a primary amine compound of the present disclosure. In particular embodiments, the primary amine compound is linked through an amide linkage on the glutamine side chain. In certain embodiments, the glutamine is an endogenous glutamine. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags, or TGase recognition tag).

The term “antibody,” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2, and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding portion thereof) can be identical to the human germline sequences, or can be naturally or artificially modified. An amino acid consensus sequence can be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody can be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain can be of any size or amino acid composition and will generally comprise at least one CDR which is adjacent to or in frame with one or more framework sequences. In antigen-binding fragments having a VH domain associated with a VL domain, the VH and VL domains can be situated relative to one another in any suitable arrangement. For example, the variable region can be dimeric and contain VH-VH, VH-VL or VL-VL dimers.

Alternatively, the antigen-binding fragment of an antibody can contain a monomeric VH or VL domain.

In certain embodiments, an antigen-binding fragment of an antibody can contain at least one variable domain covalently linked to at least one constant domain. Non-limiting, exemplary configurations of variable and constant domains that can be found within an antigen-binding fragment of an antibody of the present description include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed herein, the variable and constant domains can be either directly linked to one another or can be linked by a full or partial hinge or linker region. A hinge region can consist of at least 2 (e.g., 5, 10, 15, 20, 40, 60, or more) amino acids which result in a flexible or semi-flexible linkage between adjacent variable and/or constant domains in a single polypeptide molecule.

Moreover, an antigen-binding fragment of an antibody of the present description can comprise a homo-dimer or hetero-dimer (or other multimer) of any of the variable and constant domain configurations listed herein in non-covalent association with one another and/or with one or more monomeric VH or VL domain (e.g., by disulfide bond(s)).

As with full antibody molecules, antigen-binding fragments can be monospecific or multispecific (e.g., bispecific). A multispecific antigen-binding fragment of an antibody will typically comprise at least two different variable domains, where each variable domain is capable of specifically binding to a separate antigen or to a different epitope on the same antigen. Any multispecific antibody format, including the exemplary bispecific antibody formats disclosed herein, can be adapted for use in the context of an antigen-binding fragment of an antibody of the present description using routine techniques available in the art.

In certain embodiments, the antibodies of the description, e.g., anti-GLP1R antibodies, are human antibodies. The term “human antibody,” as used herein, is intended to include antibodies having variable and constant regions derived from human germline immunoglobulin sequences. The human antibodies of the description can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies can, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (See, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30: 105) to levels typically observed using a human IgG1 hinge. The instant description encompasses antibodies having one or more mutations in the hinge, CH2 or CH3 region which can be desirable, for example, in production, to improve the yield of the desired antibody form.

The antibodies of the description can be isolated or purified antibodies. An “isolated antibody” or “purified antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present description. For example, an antibody that has been purified from at least one component of a reaction or reaction sequence, is a “purified antibody” or results from purifying the antibody. An isolated antibody also includes an antibody in situ within a recombinant cell. Isolated antibodies are antibodies that have been subjected to at least one purification or isolation step. According to certain embodiments, an isolated antibody or purified antibody can be substantially free of other cellular material and/or chemicals.

The antibodies disclosed herein can comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present description includes antibodies, and antigen-binding fragments thereof, which are derived from any of the amino acid sequences disclosed herein, where one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with given heavy and light chain variable region sequences, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2, or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies of the present description can contain any combination of two or more germline mutations within the framework and/or CDR regions, e.g., where certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, improved drug-to-antibody ratio (DAR) for antibody-drug conjugates, etc. Antibodies and antigen-binding fragments obtained in this general manner are encompassed within the present description.

The term “aglycosylated antibody” refers to an antibody that does not comprise a glycosylation sequence that might interfere with a transglutamination reaction, for instance an antibody that does not have saccharide group at N297 on one or more heavy chains. In particular embodiments, an antibody heavy chain has an N297 mutation. In other words, the antibody is mutated to no longer have an asparagine residue at position 297 according to the EU numbering system as disclosed by Kabat et al. In particular embodiments, an antibody heavy chain has an N297Q or an N297D mutation. Such an antibody can be prepared by site-directed mutagenesis to remove or disable a glycosylation sequence or by site-directed mutagenesis to insert a glutamine residue at site apart from any interfering glycosylation site or any other interfering structure. Such an antibody also can be isolated from natural or artificial sources. Aglycosylated antibodies also include antibodies comprising a T299 or S298P or other mutations, or combinations of mutations that result in a lack of glycosylation.

The term “deglycosylated antibody” refers to an antibody in which a saccharide group at is removed to facilitate transglutaminase-mediated conjugation. Saccharides include, but are not limited to, N-linked oligosaccharides. In some embodiments, deglycosylation is performed at residue N297. In some embodiments, removal of saccharide groups is accomplished enzymatically, included but not limited to via PNGase.

The term “epitope” refers to an antigenic determinant that interacts with a specific antigen binding site in the variable region of an antibody molecule known as a paratope. A single antigen can have more than one epitope. Thus, different antibodies can bind to different areas on an antigen and can have different biological effects. Epitopes can be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope can include moieties of saccharides, phosphoryl groups, or sulfonyl groups on the antigen.

The terms “conjugated protein” or “conjugated antibody” as used herein refers to a protein or an antibody covalently linked to one or more chemical moieties. The chemical moiety can include an amine compound of the present disclosure. Linkers (L) and payloads (P) suitable for use with the present disclosure are described in detail herein. In particular embodiments, a conjugated antibody comprising a therapeutic moiety is an antibody-drug conjugate (ADC), or antibody-tethered ligand (ATL), or an antibody-tethered drug conjugate (ATDC), also referred to as an antibody-payload conjugate, or an antibody-linker-payload conjugate.

The term “Drug-to-Antibody Ratio” or (DAR) is the average number of therapeutic moieties, e.g., drugs, conjugated to a binding agent of the present disclosure.

The term “Linker Antibody Ratio” or (LAR), also denoted as the lower case, in some embodiments, is the average number of reactive primary amine compounds conjugated to a binding agent of the present disclosure. Such binding agents, e.g., antibodies, can be conjugated with primary amine compounds comprising, e.g., a suitable azide or alkyne. The resulting binding agent, which is functionalized with an azide or an alkyne can subsequently react with a therapeutic moiety comprising the corresponding azide or alkyne via the 1,3-cycloaddition reaction.

The phrase “pharmaceutically acceptable amount” refers to an amount effective or sufficient in treating, reducing, alleviating, or modulating the effects or symptoms of at least one health problem in a subject in need thereof. For example, a pharmaceutically acceptable amount of an antibody or antibody-drug conjugate is an amount effective for modulating a biological target using the antibody or antibody-drug-conjugates provided herein. Suitable pharmaceutically acceptable amounts include, but are not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% of an antibody or antibody-drug-conjugate provided herein.

The phrase “reaction pH” refers to the pH of a reaction after all reaction components or reactants have been added.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98%, or 99% of the nucleotide bases, as measured by any well-known algorithm of sequence identity, such as FASTA, BLAST or Gap, as discussed below. A nucleic acid molecule having substantial identity to a reference nucleic acid molecule can, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity can be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another particular algorithm when comparing a sequence of the description to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

### GLP1R Agonist-Tethered GDF8 Antibody Conjugates

In one aspect, the present disclosure provides is a GLP1R agonist-tethered antibody conjugate, or a pharmaceutically acceptable salt thereof, wherein the conjugate comprises
a) an antibody, or an antigen-binding fragment thereof,
b) at least one Glucagon-like peptide-1 (GLP-1) receptor (GLP1R) agonist according to any of the embodiments described herein; and
c) one linker or a bond that covalently connects the at least one said GLP1R agonist to the antibody, or the antigen-binding fragment thereof.

In another aspect, the present disclosure provides a composition comprising an GLP1R agonist-tethered GDF8 antibody conjugate, or a pharmaceutically acceptable salt thereof. This conjugate comprises
a) an antibody, or an antigen-binding fragment thereof, that specifically binds to and/or blocks the biological activity of, Growth and Differentiation Factor-8 (GDF8, SEQ ID NO: 1);
b) at least one Glucagon-like peptide-1 (GLP-1) receptor (GLP1R) agonist; and
c) a linker that covalently connects the at least one GLP1R agonist to the GDF8 antibody, or the antigen-binding fragment thereof.

The Growth and Differentiation Factor-8 has a sequence of Asp Phe Gly Leu Asp Cys Asp Glu His Ser Thr Glu Ser Arg Cys Cys Arg Tyr Pro Leu Thr Val Asp Phe Glu Ala Phe Gly Trp Asp Trp Ile Ile Ala Pro Lys Arg Tyr Lys Ala Asn Tyr Cys Ser Gly Glu Cys Glu Phe Val Phe Leu Gln Lys Tyr Pro His Thr His Leu Val His Gln Ala Asn Pro Arg Gly Ser Ala Gly Pro Cys Cys Thr Pro Thr Lys Met Ser Pro Ile Asn Met Leu Tyr Phe Asn Gly Lys Glu Gln Ile Ile Tyr Gly Lys Ile Pro Ala Met Val Val Asp Arg Cys Gly Cys Ser (SEQ ID NO: 1).

In some embodiments, the GLP1R agonist is linked to the GDF8 antibody, or the antigen-binding fragment thereof, through a side chain of a cysteine residue (Cys) of the GDF8 antibody, or the antigen-binding fragment thereof.

In some embodiments, the Cys is a naturally occurring Cys. In some embodiments, the Cys is an engineered Cys.

In some embodiments, the engineered Cys is introduced to the GDF8 antibody, or the antigen-binding fragment thereof, by site-specific modification of one or more amino acid residues.

In some embodiments, the GLP1R agonist is covalently attached to the GDF8 antibody, or the antigen-binding fragment thereof, through the side chain of a glutamine residue (Gln) of the GDF8 antibody, or the antigen-binding fragment thereof.

In some embodiments, GLP1R agonist is covalently attached to the GDF8 antibody, or the antigen-binding fragment thereof, via a microbial transglutaminase assisted reaction, a Click or Diels-Alder reaction, or a combination thereof.

In some embodiments, the Gln is a naturally occurring Gln. In some embodiments, the Gln is an engineered Gln. In some embodiments, the Gln is Gln55 (Q55), Gln295 (Q295), or a combination thereof.

In some embodiments, the linker comprises

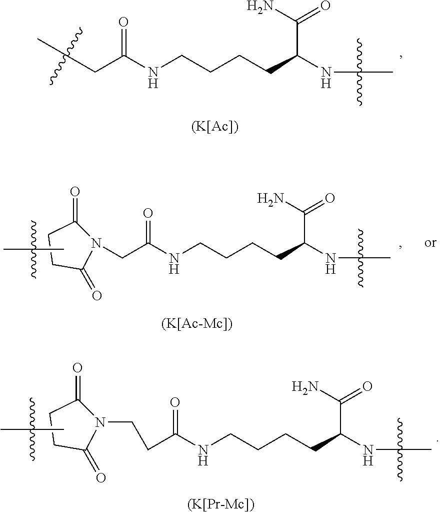

In some embodiments, the compound comprises between about one and about sixteen GLP1R agonist moieties.

In some embodiments, the GLP1R agonist is the compound according to any of the embodiments described herein.

In some embodiments, the GDF8 antibody, or the antigen-binding fragment thereof, comprises three heavy chain complementarity determining regions (HCDR1, HCDR2, and HCDR3) comprising Gly Phe Thr Phe Ser Ala Tyr Ala (SEQ ID NO: 6), Ile Ser Gly Ser Gly Gly Ser Ala (SEQ ID NO: 7), and Ala Lys Asp Gly Ala Trp Lys Met Ser Gly Leu Asp Val (SEQ ID NO: 8), respectively, and three light chain complementarity determining regions (LCDR1, LCDR2, and LCDR3) comprising Gln Asp Ile Ser Asp Tyr (SEQ ID NO: 9), Thr Thr Ser, and Gln Lys Tyr Asp Ser Ala Pro Leu Thr (SEQ ID NO: 11), respectively, and the GLP1R agonist has a sequence of H(Aib)EGTFTSDYSSYLEEQAAKEFIAWLVKGGG (SEQ ID NO: 3), where Aib is 2-Aminoisobutyric acid.

In some embodiments, the GDF8 antibody, or the antigen-binding fragment thereof, comprises three heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively, and the GLP1R agonist is the compound according to any of the embodiments described herein.

In some embodiments, the composition comprising an GLP1R agonist-tethered GDF8 antibody conjugate further comprises one or more pharmaceutically accept salts, excipients, and/or diluents.

In another aspect, the present disclosure provides a GLP1R agonist-tethered GDF8 antibody conjugate, or a pharmaceutically acceptable salt thereof, having a Formula (I):

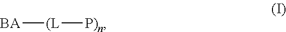

where
BA is an antibody, or an antigen-binding fragment thereof, that specifically binds to and/or blocks the biological activity of, human Growth and Differentiation Factor-8 (GDF8, SEQ ID NO: 1);
P is a Glucagon-like peptide-1 (GLP-1) receptor (GLP1R) agonist;
L is a linker that covalently links the P to the BA; and
n ranges from about 1 to about 16.

In some embodiments, the GLP1R agonist-tethered GDF8 antibody conjugate has a Formula (II):

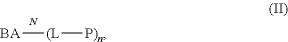

where [symbol] represents that the connection between the BA and the L is through the side chain of a glutamine residue (Gln) of the BA.

In some embodiments, the Gln is a naturally occurring Gln. In some embodiments, the Gln is an engineered Gln. In some embodiments, the Gln is Gln55 (Q55). In some embodiments, the Gln is Gln295 (Q295).

In some embodiments, the linker comprises (a) a Click or Diels-Alder reaction adduct, and (b) an amide bond formed via a transglutaminase reaction between a primary amine and the side chain of a Gln of the BA.

In some embodiments, a handle for Click or Diels-Alder reaction is conjugated to the GDF8 antibody via microbial transglutaminase prior to the conjugation with the GLP1R agonist, where the GLP1R agonist comprises a reactive moiety for Click or Diels-Alder reaction, wherein the reactive moiety is selected from the group consisting of from

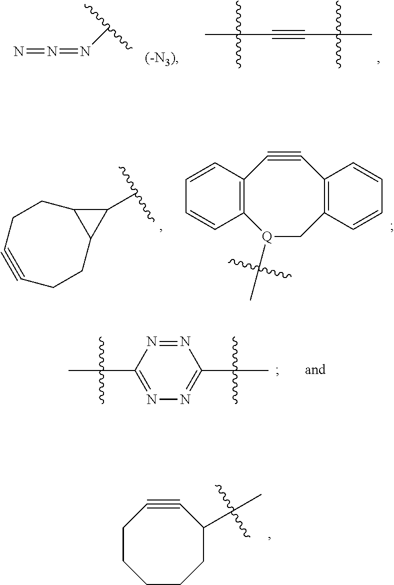

where Q is CH or N.

In some embodiments, the handle for Click or Diels-Alder reaction, is selected from the group consisting of

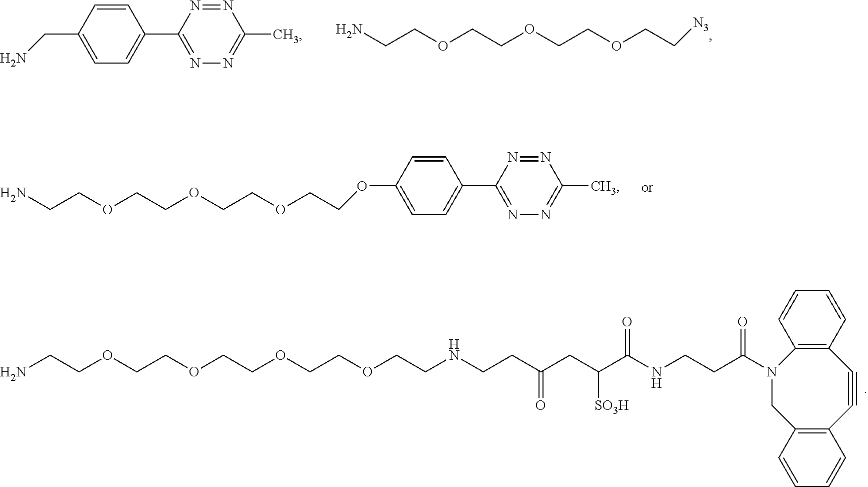 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the linker L comprises a Click or Diels-Alder reaction adduct selected from the group consisting of:

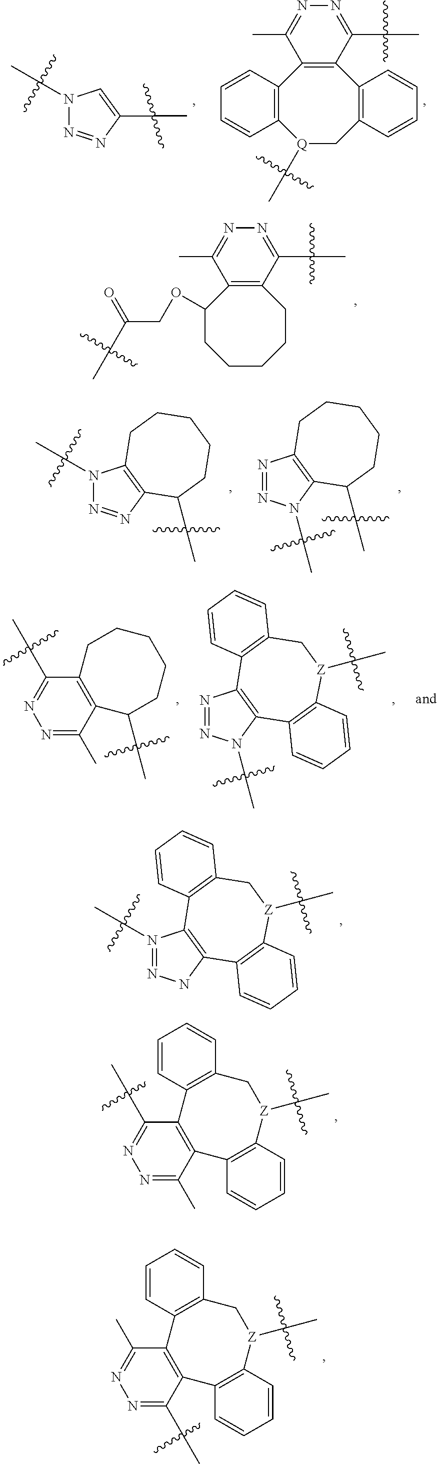

where Z is CH or N.

In some embodiments, the linker L comprises

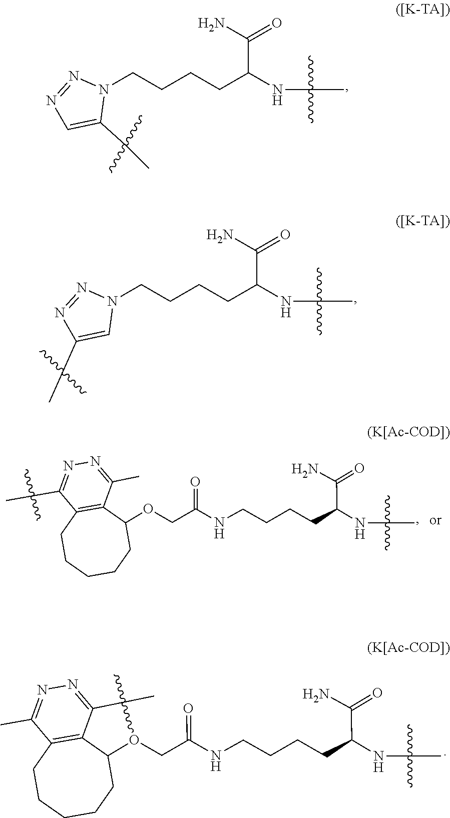 [SMILES: *NC(CCCCn1nncc1*)C(N)=O ; *NC(CCCCn1cc(*)nn1)C(N)=O ; *N[C@@H](CCCCNC(=O)COC1CCCCCc2c(*)nnc(C)c21)C(N)=O ; *N[C@@H](CCCCNC(=O)COC1CCCCCc2c(C)nnc(*)c21)C(N)=O]

In some embodiments, the GLP1R agonist-tethered GDF8 antibody conjugate has a Formula (III):

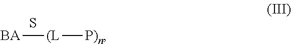

where [symbol] represents that the connection between the BA and the L is via the side chain of a cysteine residue (Cys) of the BA.

In some embodiments, the Cys of the BA is a naturally occurring Cys. In some embodiments, the Cys of the BA is an engineered Cys. In some embodiments, the Cys of GDF8 antibody is a naturally occurring Cys. In some embodiments, the Cys of the BA is an engineered Cys introduced by site-specific mutation.

In some embodiments, the L comprises a moiety resulted from chemical conjugation of a reactive group (RG) on the L-P with the side chain of Cys of the BA.

In some embodiments, the reactive group is a bromo acetyl or a maleimide group.

In some embodiments, n ranges from about 2 to about 4.

In some embodiments, the GLP1R agonist-tethered GDF8 antibody conjugate is selected from the group consisting of:

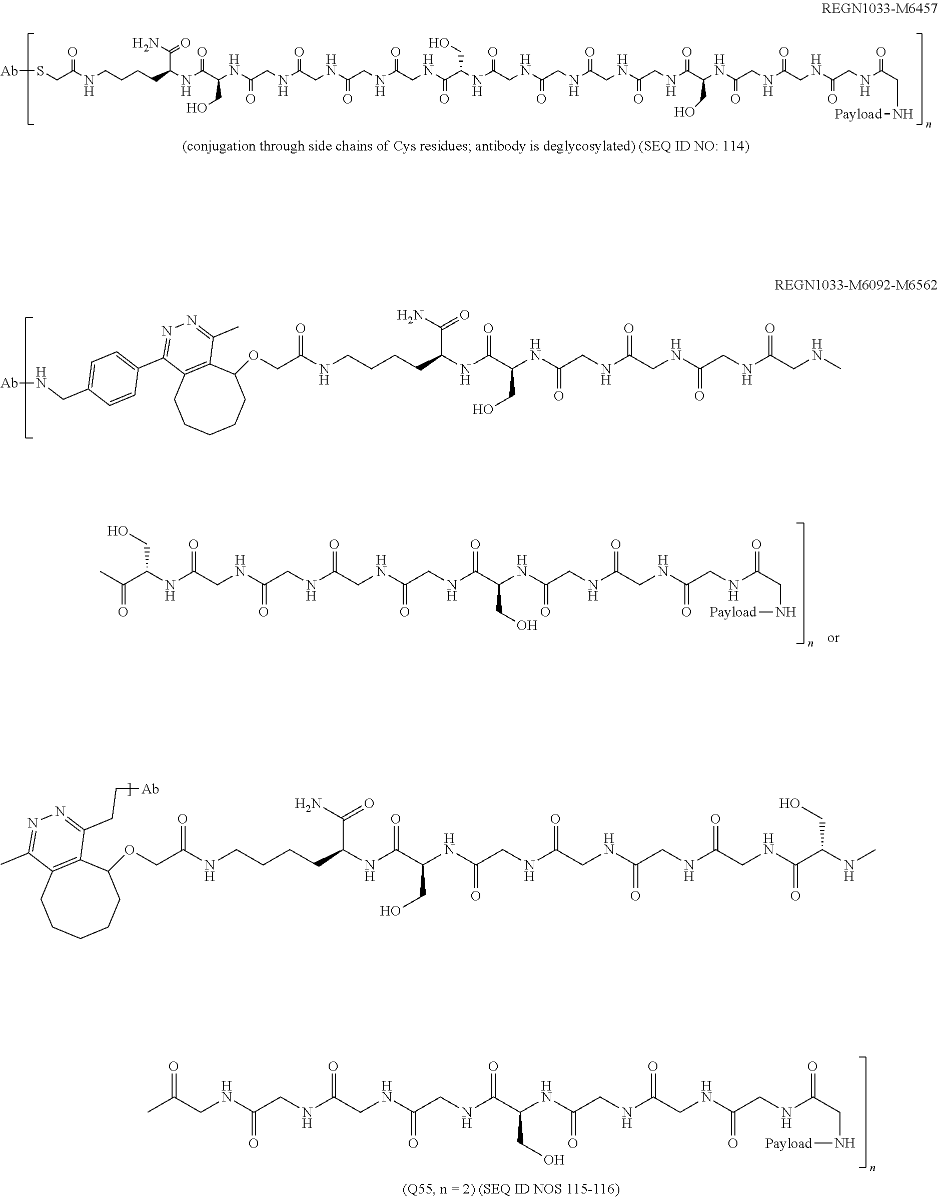

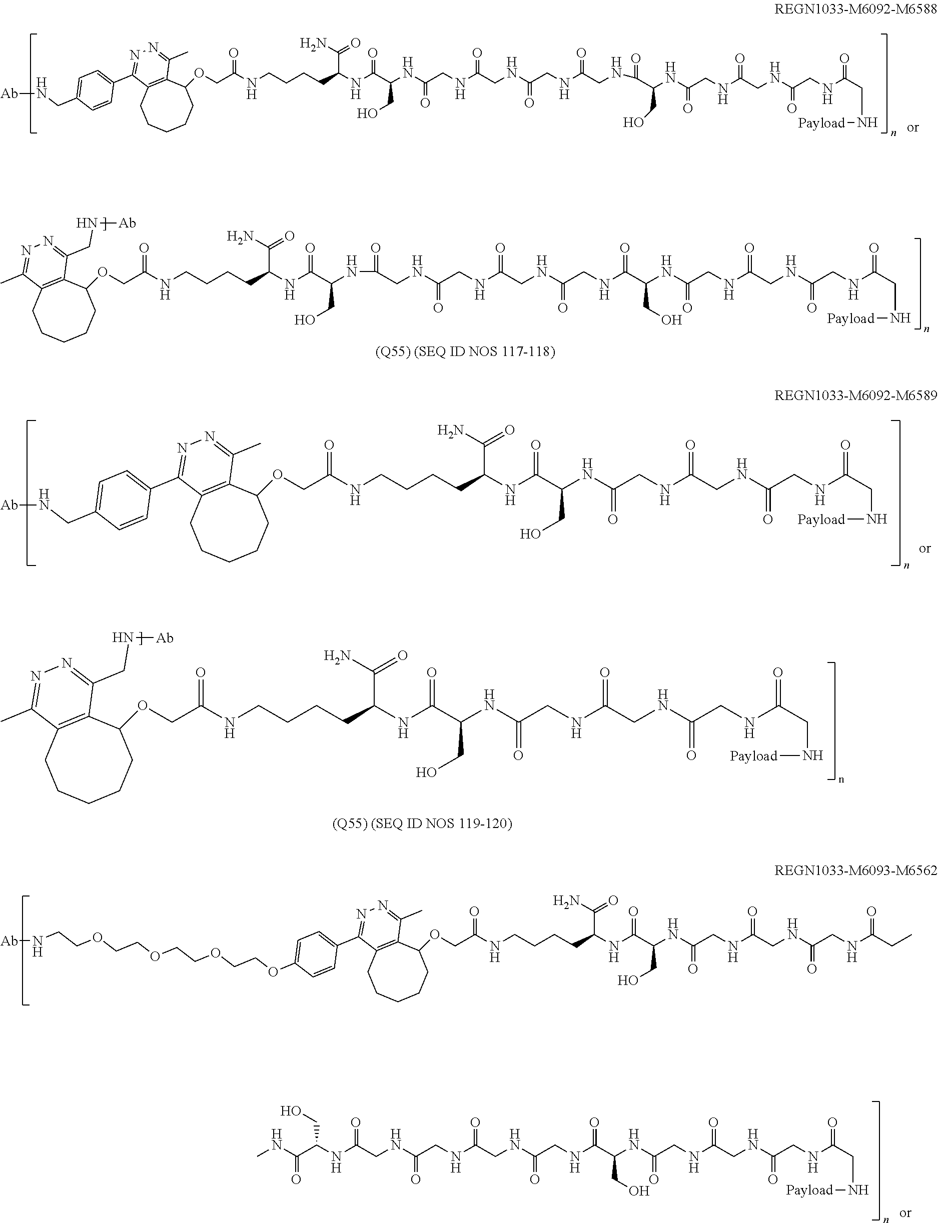

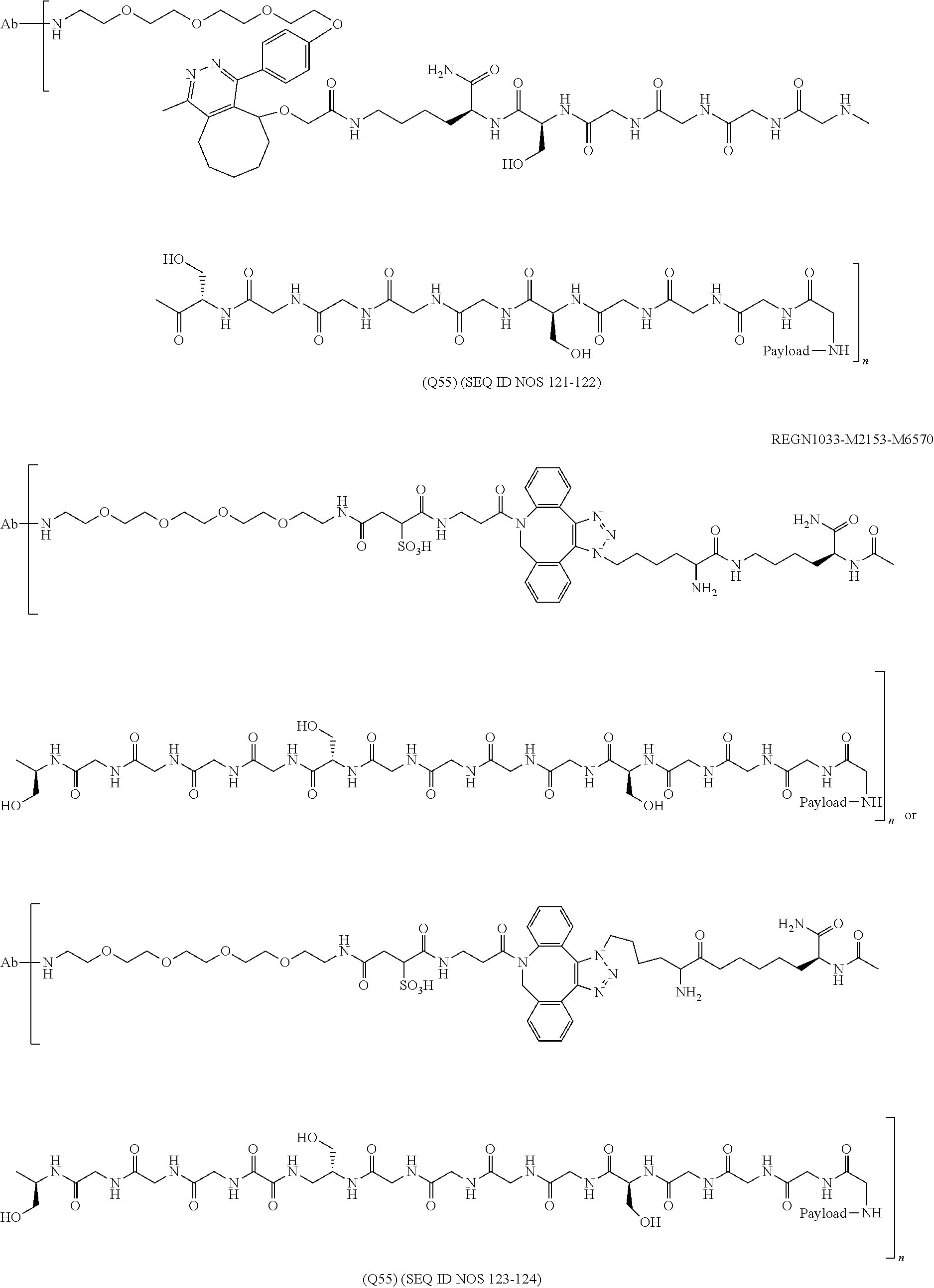

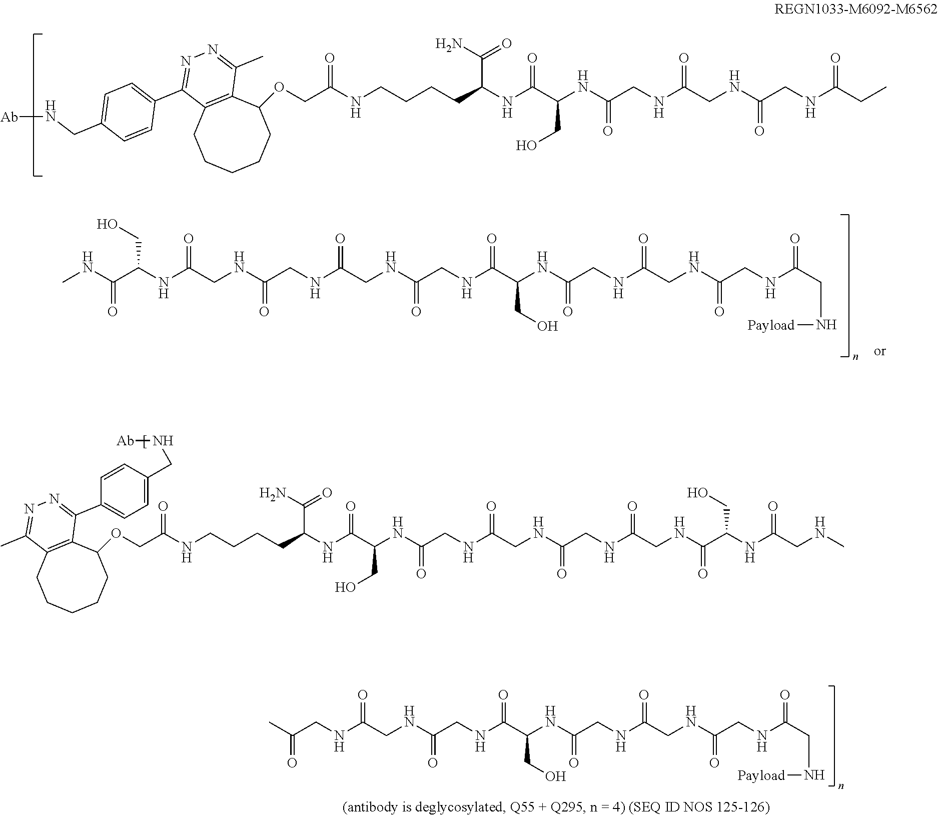

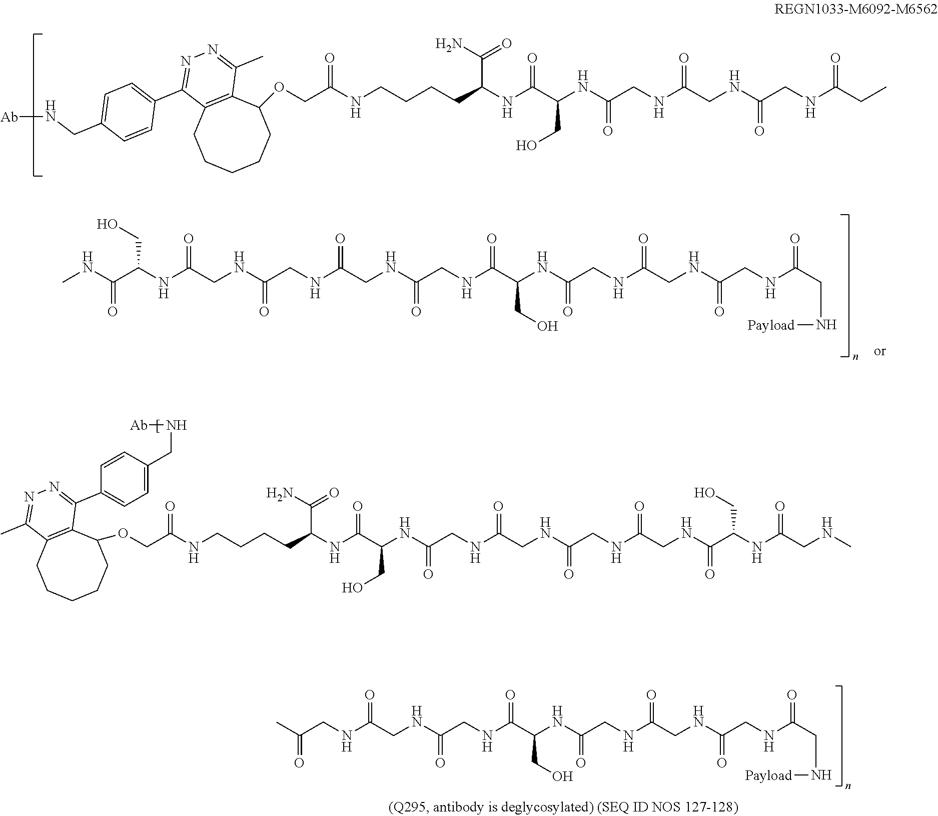

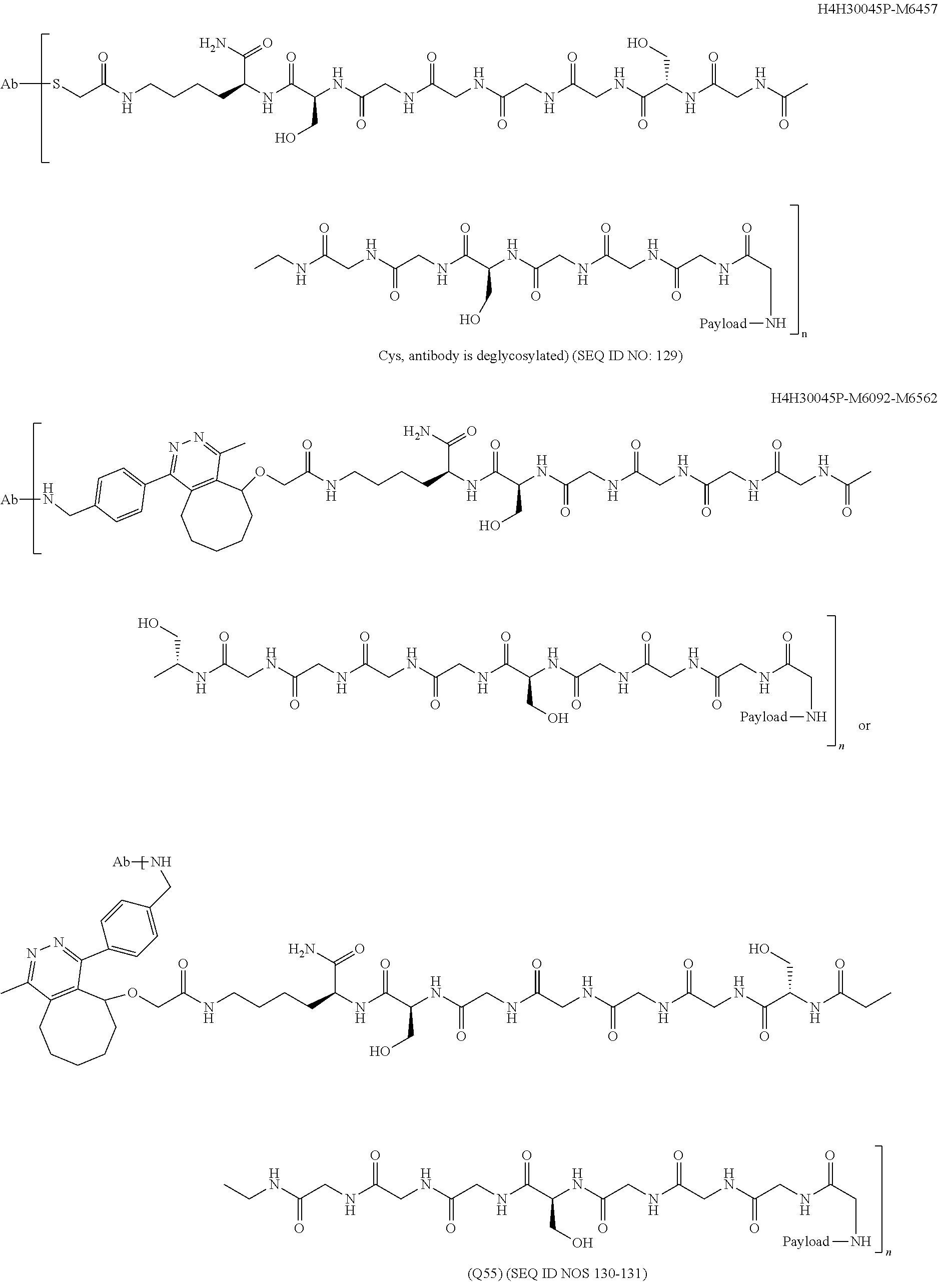

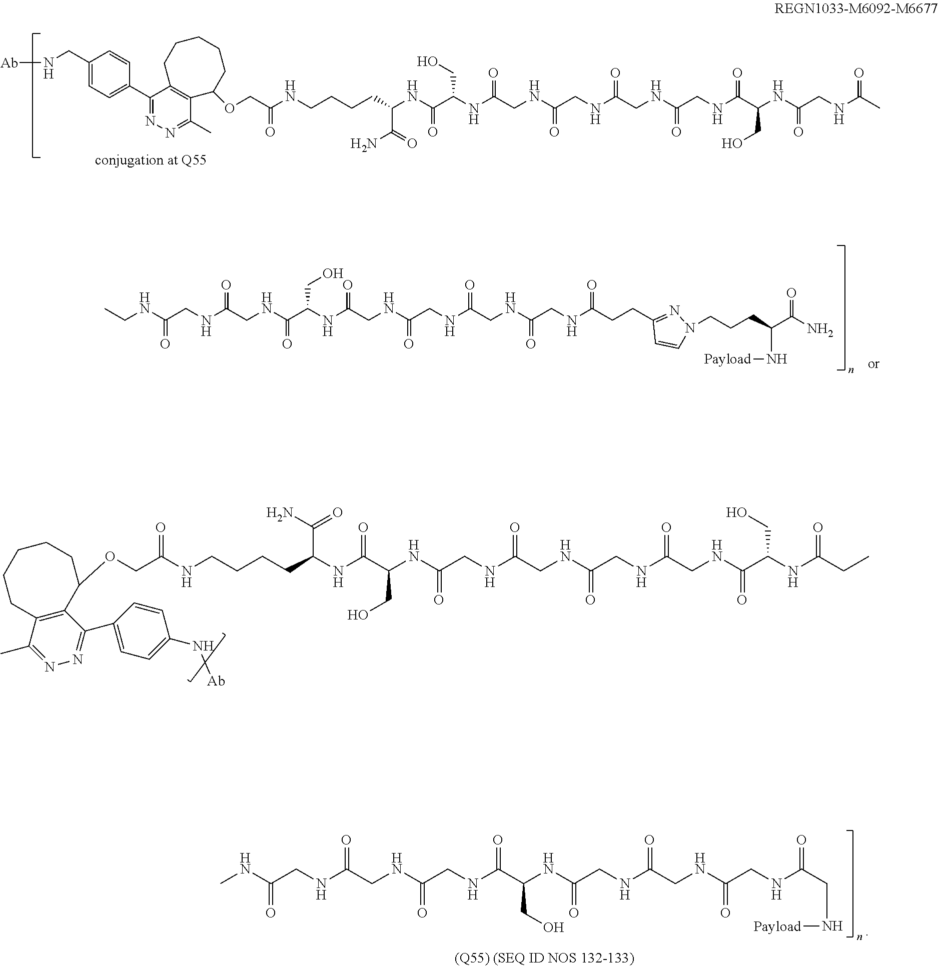

or a pharmaceutically acceptable salt thereof, where the Payload is the compound according to any of the embodiments described herein, and where n is about 1 to 10, and Ab is the GDF8 antibody, or the antigen-binding fragment thereof.

In some embodiments, the GLP1R agonist-tethered GDF8 antibody conjugate is selected from the group consisting of:

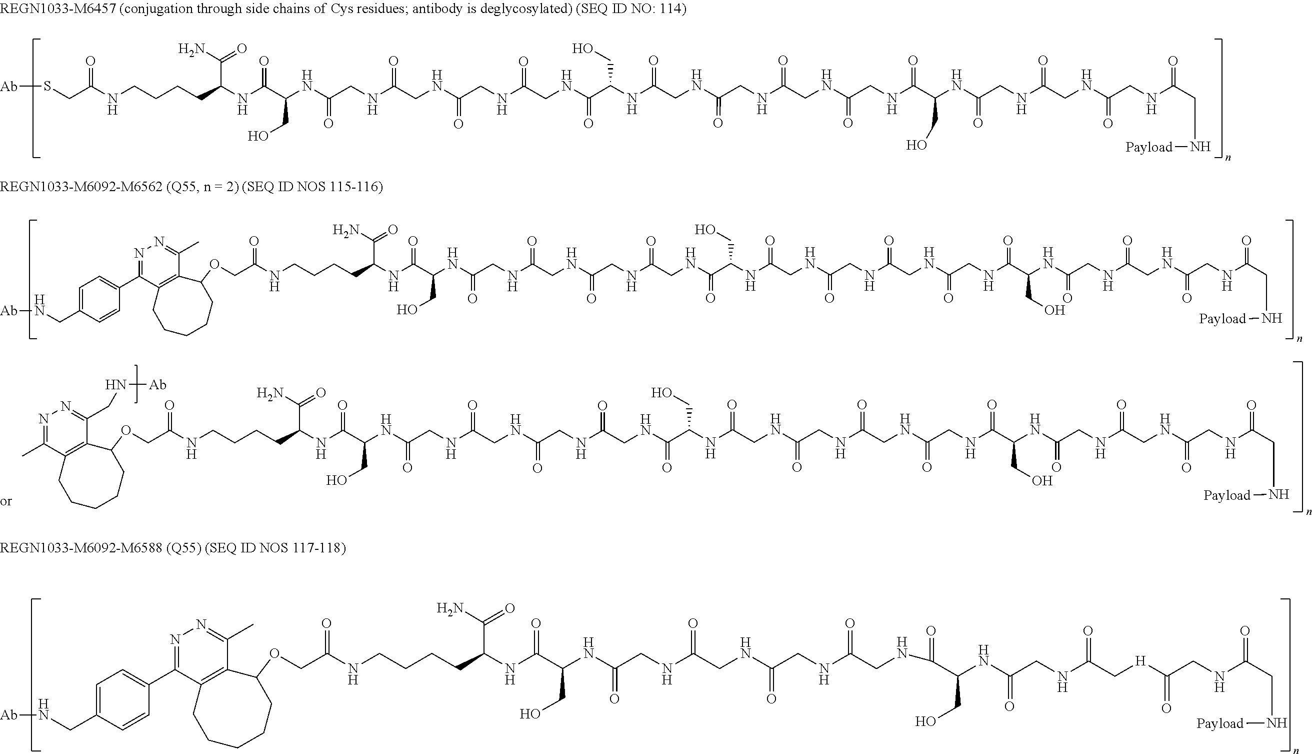

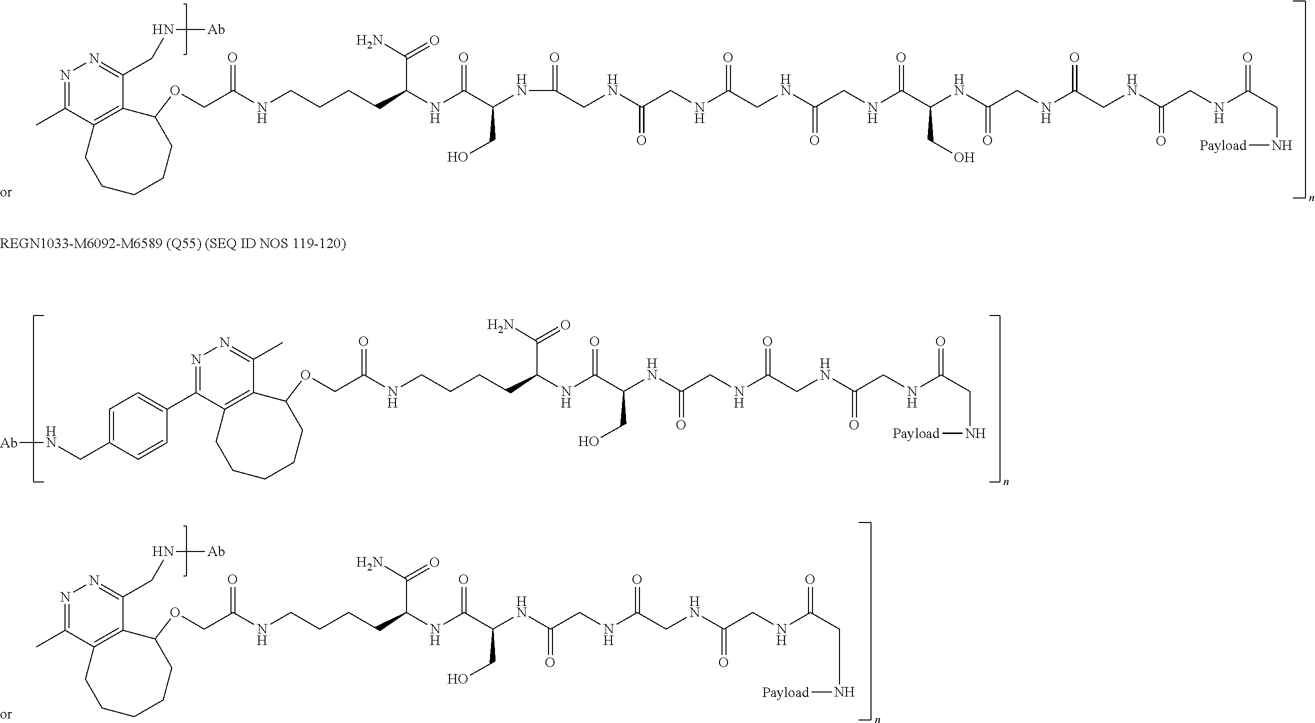

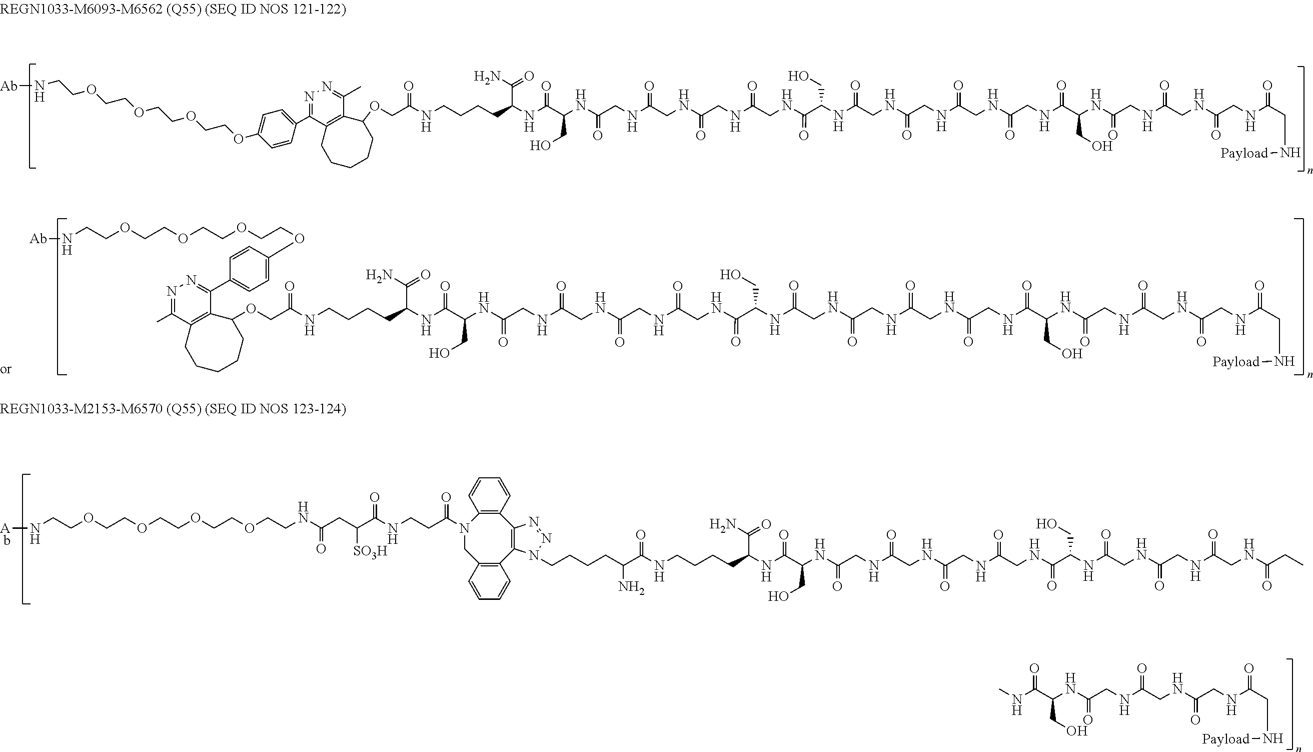

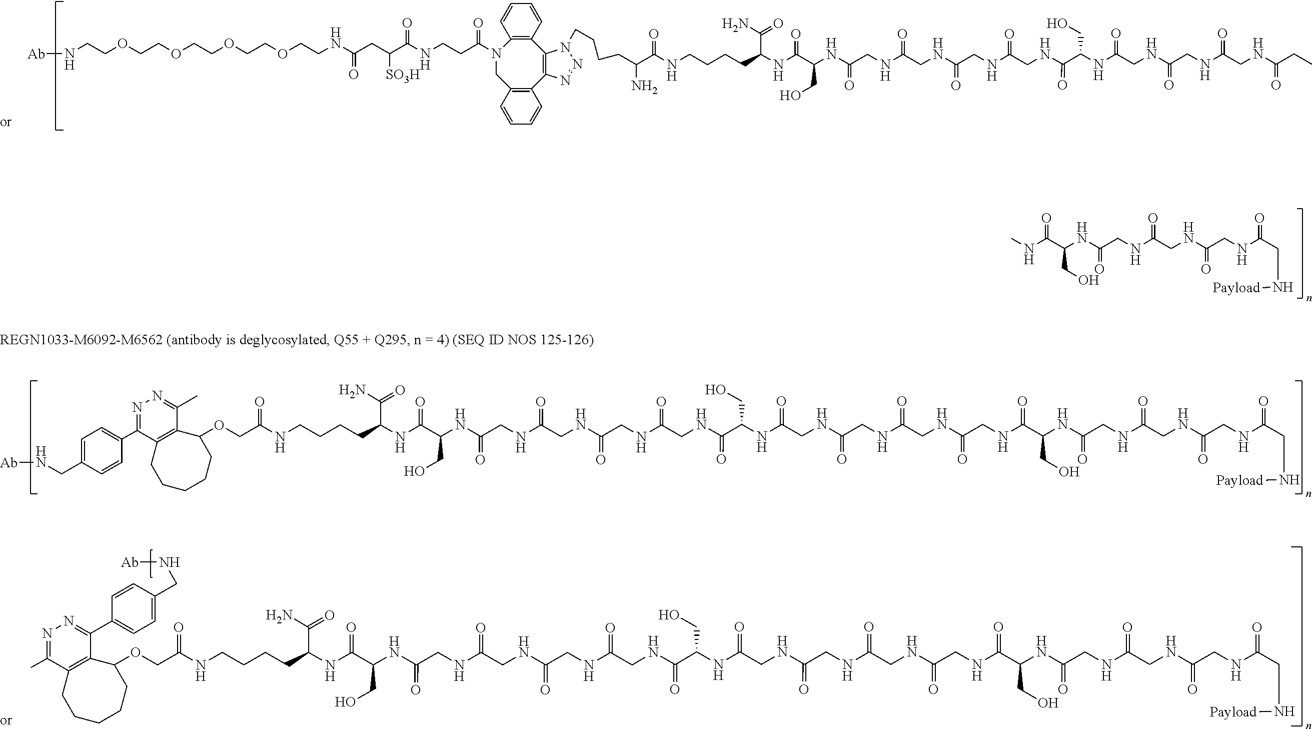

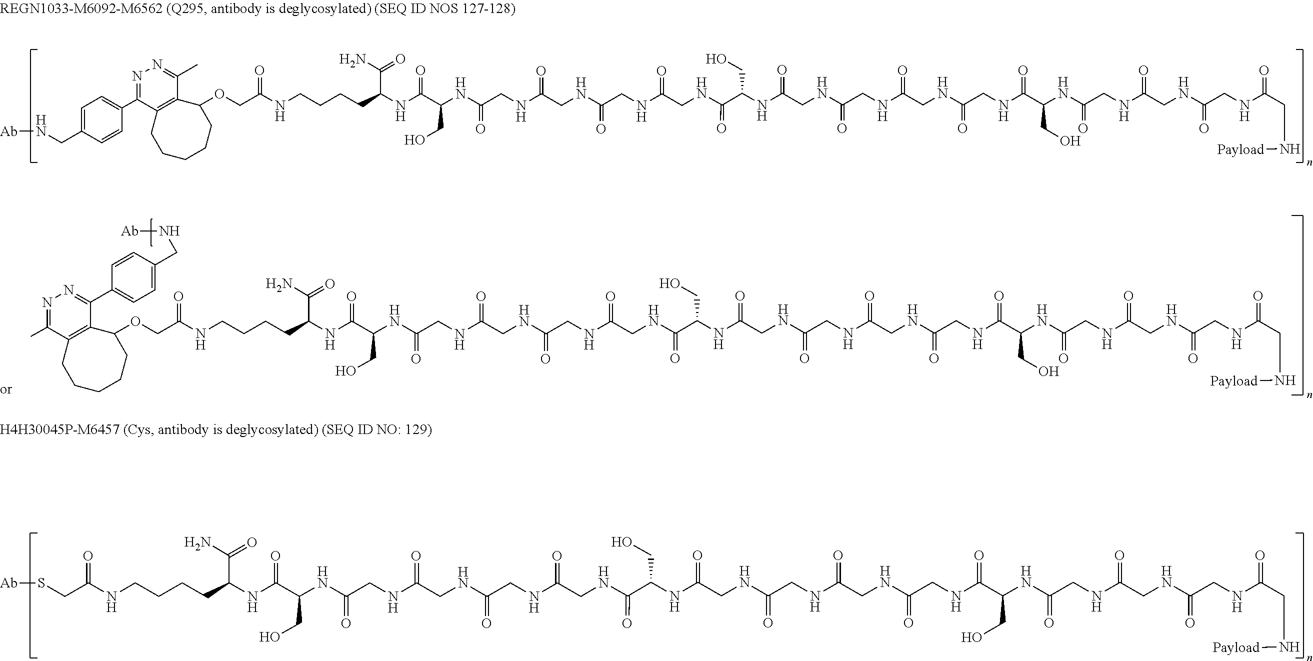

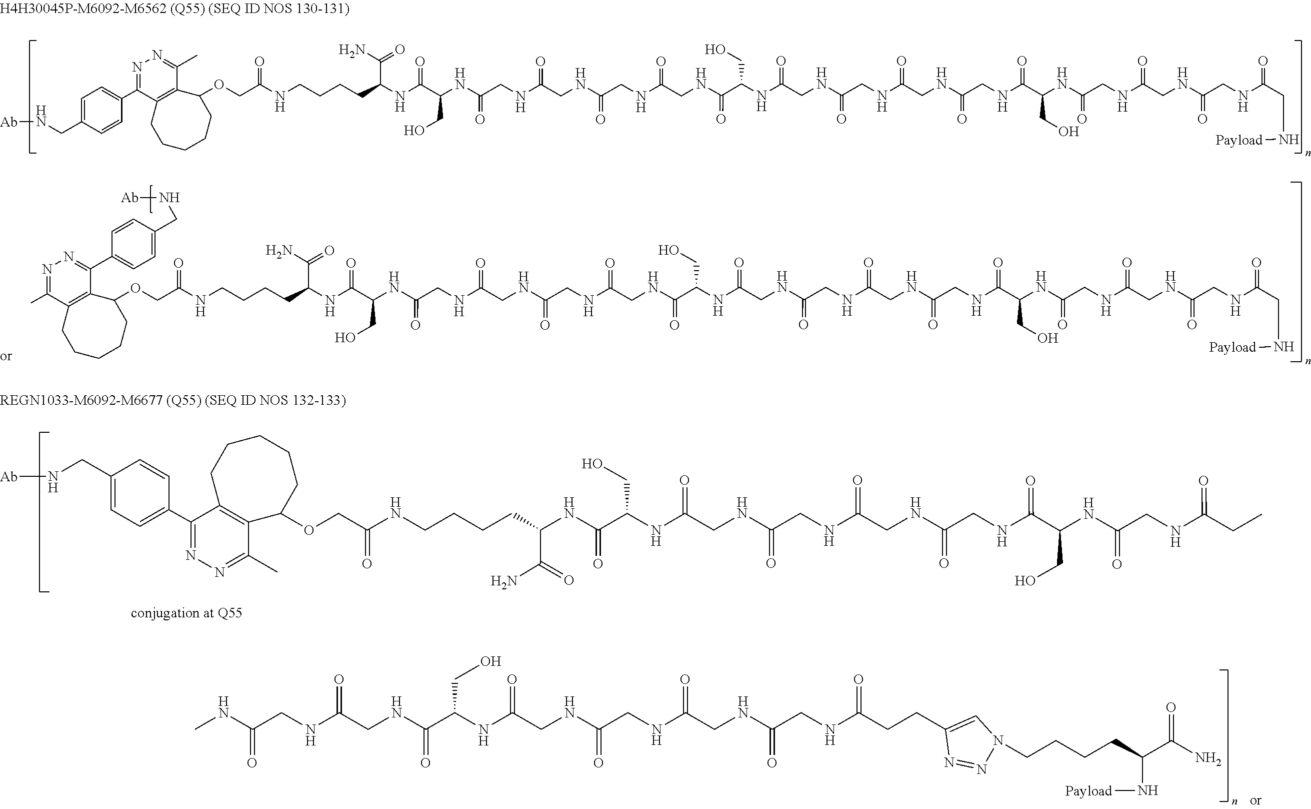

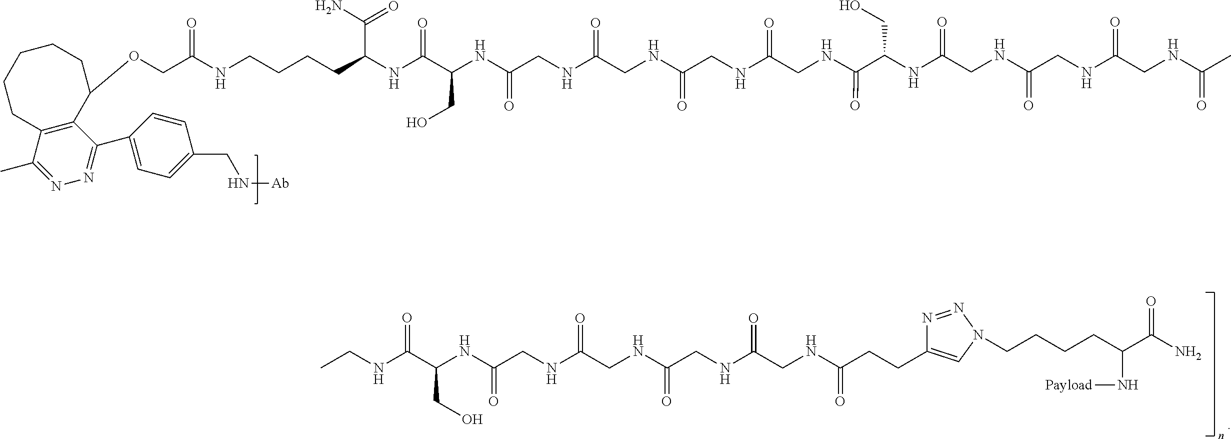

or a pharmaceutically acceptable salt thereof, where the Payload is H[Aib]EGTFTSDYSSYLEEQAAKEFIAWLVKGGG (SEQ ID NO: 3), and where n is about 1 to 10, and Ab is the GDF8 antibody, or the antigen-binding fragment thereof.

In another aspect, the composition according to the disclosure comprises an GLP1R agonist-tethered GDF8 antibody, or a pharmaceutically acceptable salt thereof, where the GDF8 antibody is a GDF-8 inhibitor. In another embodiment, the GDF8 inhibitor is an antibody or antigen-binding fragment thereof that specifically binds GDF-8. In a further embodiment, the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO:5. In still a further embodiment, the anti-GDF8 antibody or antigen-binding fragment thereof comprises heavy chain complementarity determining regions (HCDR1, HCDR2, and HCDR3) comprising the amino acid sequences of SEQ ID NO: 6, SEQ ID NO: 7, and SEQ ID NO: 8, respectively, and three light chain complementarity determining regions (LCDR1, LCDR2, and LCDR3) comprising the amino acid sequences of SEQ ID NO: 9, SEQ ID NO: 10, and SEQ ID NO: 11, respectively.

In another aspect, the composition according to the disclosure comprises an GLP1R agonist-tethered GDF8 antibody, or a pharmaceutically acceptable salt thereof, where the GLP1R agonist is selected from the group consisting of the compound according to any of the embodiments described herein, Exenatide (long-acting), Dulaglutide, Liraglutide, Tirzepatide, and Semaglutide. In a further embodiment, the GLP-1 agonist is a GLP-1-specific binding protein. In still a further embodiment, the GLP-1 agonist is an antibody or antigen-binding fragment thereof that specifically binds GLP-1.

In another aspect, the composition according to the disclosure comprises an GLP1R agonist-tethered GDF8 antibody, or a pharmaceutically acceptable salt thereof, where the GLP1R agonist is selected from the group consisting of Exenatide (long-acting), Dulaglutide, Liraglutide, Tirzepatide, and Semaglutide. In a further embodiment, the GLP-1 agonist is a GLP-1-specific binding protein. In still a further embodiment, the GLP-1 agonist is an antibody or antigen-binding fragment thereof that specifically binds GLP-1.

In another aspect, the present disclosure provides a process for manufacturing a conjugate of GLP1R agonist tethered GDF8 antibody or antigen binding fragment thereof. This process comprises a) covalently attaching a handle comprising a first reactive moiety for Click or Diels-Alder reaction, in the presence of microbial transglutaminase; b) exposing a GLP1R agonist comprising a second reactive moiety for Click or Diels-Alder reaction, where the first and the second reactive moieties are complimentary to each other and form a stable conjugate; and c) isolating or purifying the conjugate of GLP1R agonist tethered GDF8 antibody or antigen binding fragment thereof.

The first reactive moiety for Click or Diels-Alder reaction is complimentary to the second reactive moiety for Click or Diels-Alder reaction, and the first and the second reactive moieties, when combined, form a stable conjugate.

For a Diels-Alder reaction, one of the reactive moieties is a diene and another one is a dienophile. The term “diene” in the context of the Diels-Alder reaction refers to 1,3-(hetero)dienes, and includes conjugated dienes (R2C═CR—CR═CR2), imines (e.g. R2C═CR N═CR2 or R2C═CR—CR═NR, R2C═N—N═CR2) and carbonyls (e.g. R2C═CR—CR═O or O═CR—CR═O). Hetero-Diels-Alder reactions with N- and O-containing dienes are known in the art. Any diene known in the art to be suitable for Diels-Alder reactions may be used as one of the reactive moieties. In non-limiting embodiments, preferred dienes include tetrazines, 1,2-quinones, and triazines. Although any dienophile known in the art to be suitable for Diels-Alder reactions may be used as another one of the reactive moieties, the dienophile is preferably an alkene or alkyne group, most preferably an alkyne group.

For a Click reaction, one of the reactive moieties is a 1,3-dipole and another one is a dipolarophile. Any 1,3-dipole known in the art to be suitable for Click reaction may be used as one of the reactive moieties. In non-limited embodiments, preferred 1,3-dipoles include azido groups, nitrone groups, nitrile oxide groups, nitrile imine groups, and diazo groups. Although any dipolarophile known in the art to be suitable for Click reaction may be used as the other one of the reactive moieties, the dipolarophile is preferably an alkene or alkyne group, most preferably an alkyne group.

In some embodiments, the reactive moiety for the Click reaction, comprise —N₃,

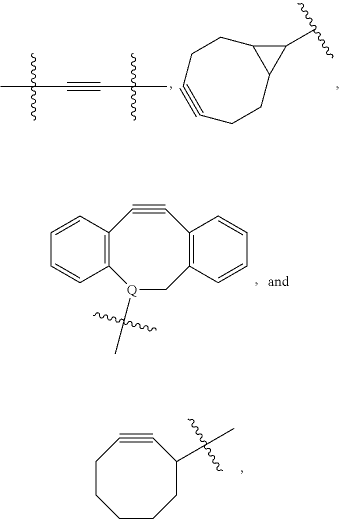

where Q is CH or N.

In some embodiments, the reactive moiety for the Diels-Alder reaction, comprise

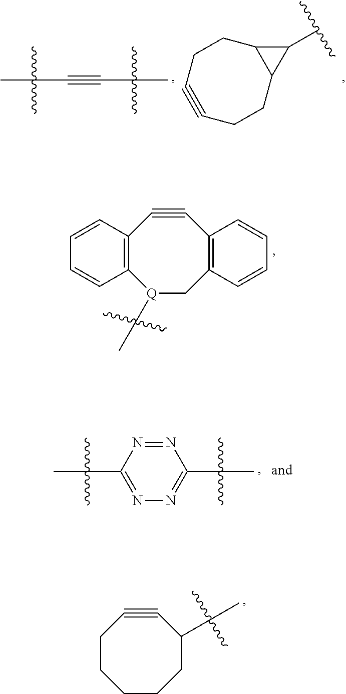

where Q is CH or N.

In some embodiments, the handle comprises

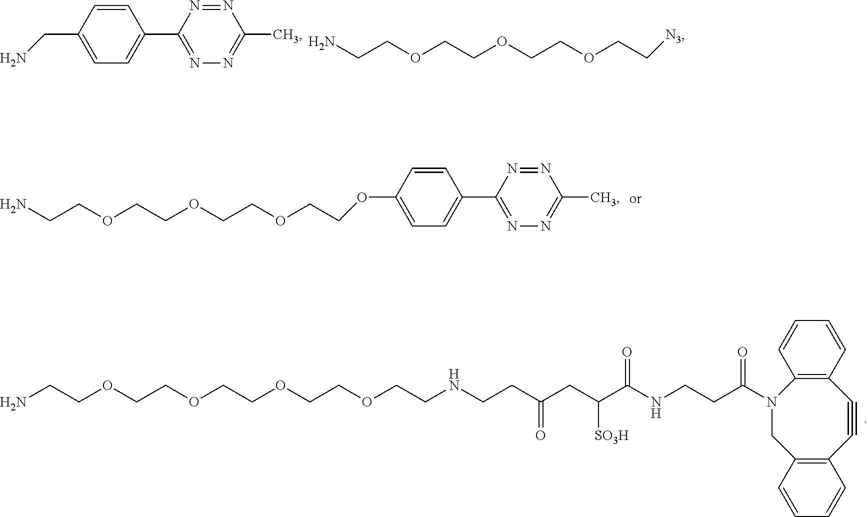 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the reactive moiety of the GDF8 antibody and the reactive moiety of the GLP1R agonist form a linker adduct having a structure of

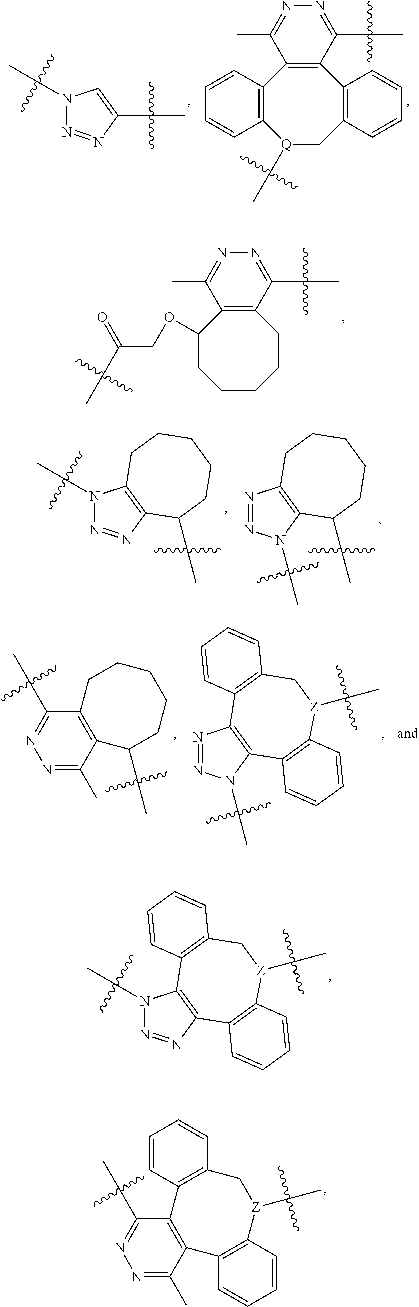

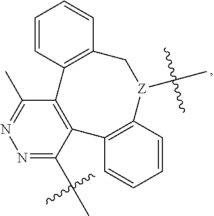

where Z is CH or N.

In some embodiments, the GLP1R agonist is conjugated to the GDF8 antibody or antigen binding fragment thereof via Q55 on the light chain or Q295 of the heavy chain of the antibody.

In another aspect, the present disclosure provides a product prepared according to any of the processes described herein.

In another aspect, the present disclosure provides a conjugate, or a pharmaceutically acceptable salt thereof, comprising an antigen-binding protein (BA) and a GLP1R agonist, where the BA specifically binds to and/or blocks the activity of GDF8 (SEQ ID NO: 1), and the GLP1R agonist is conjugated to the BA through a linker.

In some embodiments, the antigen-binding protein (BA) is an antibody or antigen-binding fragment thereof, where the BA is modified with a handle with the assistance of mTG, where the handle comprises a primary amino group and a reactive moiety for Click or Diels-Alder reaction for conjugation with the GLP1R agonist.

In some embodiments, the BA is an anti-GDF8 antibody trevogrumab comprising the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO: 5, as follows:

(SEQ ID NO: 4)
Glu Val Gln Val Leu Glu Ser Gly Gly Asp Leu
Val Gln Pro Gly Gly Ser Leu Arg Leu Ser Cys
Ala Ala Ser Gly Phe Thr Phe Ser Ala Tyr Ala
Met Thr Trp Val Arg Gln Ala Pro Gly Lys Gly
Leu Glu Trp Val Ser Ala Ile Ser Gly Ser Gly
Gly Ser Ala Tyr Tyr Ala Asp Ser Val Lys Gly
Arg Phe Thr Ile Ser Arg Asp Asn Ser Lys Asn
Thr Val Tyr Leu Gln Met Asn Ser Leu Arg Ala
Glu Asp Thr Ala Val Tyr Tyr Cys Ala Lys Asp
Gly Ala Trp Lys Met Ser Gly Leu Asp Val Trp
Gly Gln Gly Thr Thr Val Ile Val Ser Ser
(SEQ ID NO: 5)
Asp Ile Gln Met Thr Gln Ser Pro Ala Ser Leu
Ser Ala Ser Val Gly Asp Arg Val Thr Ile Thr
Cys Arg Ala Ser Gln Asp Ile Ser Asp Tyr Leu
Ala Trp Tyr Gln Gln Lys Pro Gly Lys Ile Pro
Arg Leu Leu Ile Tyr Thr Thr Ser Thr Leu Gln
Ser Gly Val Pro Ser Arg Phe Arg Gly Ser Gly
Ser Gly Thr Asp Phe Thr Leu Thr Ile Ser Ser
Leu Gln Pro Glu Asp Val Ala Thr Tyr Tyr Cys
Gln Lys Tyr Asp Ser Ala Pro Leu Thr Phe Gly
Gly Gly Thr Lys Val Glu Ile Lys

In some embodiments, the BA is an anti-GDF8 antibody trevogrumab comprising heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

In another aspect, the present disclosure provides an antibody conjugate comprising a linker-payload as described herein conjugated to GDF8 antibody that specifically binds to and/or blocks the activity of GDF8 (SEQ ID NO: 1).

### Linker Payloads

In another aspect, the present disclosure provides a compound, or a pharmaceutically acceptable salt thereof, having the structure of Formula (IV):

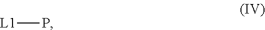

where
L1 is a linker comprising a sequence selected from the group consisting of -(Gly₃Ser)ₙ-Lys(X) (SEQ ID NO: 89)- and -(Gly₄Ser)ₙ-Lys(X)- (SEQ ID NO: 90), where n is an integer from one to six; and where the side chain of Lys is functionalized with a reactive moiety X for conjugation with a target antibody or antigen binding fragment thereof, and
P is a Glucagon-like peptide-1 (GLP-1) receptor (GLP1R) agonist.

In some embodiments, the reactive moiety X is a moiety reactive toward cysteine thiol or a moiety for Click or Diels-Alder reaction.

In some embodiments, Lys(X) has a formula

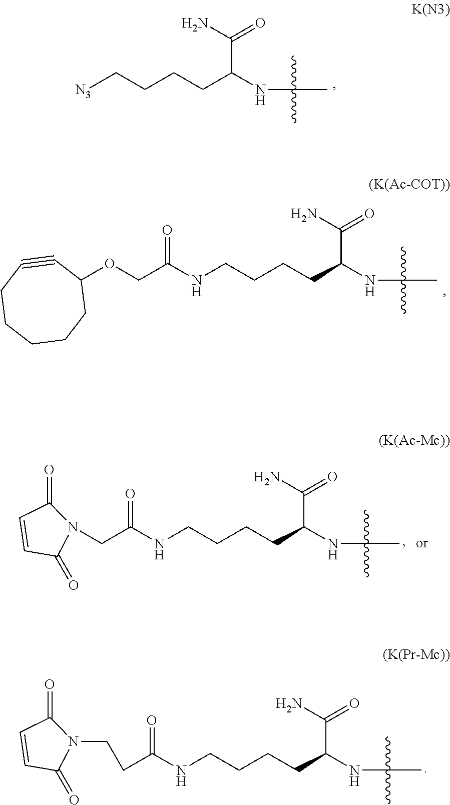 [SMILES: *NC(CCCCN=[N+]=[N-])C(N)=O ; *N[C@@H](CCCCNC(=O)COC1C#CCCCCC1)C(N)=O ; *N[C@@H](CCCCNC(=O)CN1C(=O)C=CC1=O)C(N)=O ; *N[C@@H](CCCCNC(=O)CCN1C(=O)C=CC1=O)C(N)=O]

In some embodiments, the compound has the structure selected from the group consisting of:

(SEQ ID NO: 15)
H(Aib)EGTFTSDYSSYLEEQAAKEFIAWLVKGGG-GGGGS-K[Ac-COT]
amide (Payload -(G4S)-K[Ac-COT]),
(SEQ ID NO: 14)
H(Aib)EGTFTSDYSSYLEEQAAKEFIAWLVKGGG-GGGGSGGGGS-K[Ac-COT]amide
(Payload -(G4S)2-K[Ac-COT]),
(SEQ ID NO: 13)
H(Aib)EGTFTSDYSSYLEEQAAKEFIAWLVKGGG-GGGGSGGGGSGGGGS-K[Ac-
COT]amide (Payload -(G₄S)₃-K[Ac-COT]),
(SEQ ID NO: 16)
H(Aib)EGTFTSDYSSYLEEQAAKEFIAWLVKGGG-GGGGSGGGGSGGGGS-K[KN3]
amide (M6570) (Payload -(G₄S)₃-K[KN3]),
(SEQ ID NO: 17)
H(Aib)EGTFTSDYSSYLEEQAAKEFIAWLVKGGG-GGGGSGGGGSGGGGS-K[Ac-Mc]
amide (Payload -(G₄S)₃-K[Ac-Mc]),
(SEQ ID NO: 20)
H(Aib)EGTFTSDYSSYLEEQAAKEFIAWLVKGGG-GGGGSGGGGSGGGGS-
K[propanoyl-Mc]amide (Payload -(G₄S)₃-K[Pr-Mc]),
H[Aib]EGTFTSDYSSYLEEQAAKEFIAWLVKGGGK(TA-GGGGSGGGGSGGGGS-
(SEQ ID NOS 18 & 136)
K(Ac-COT)) amide (M6677)
(Payload -K(TA-(G₄S)₃-K[Ac-COT]),
(SEQ ID NO: 19)
H[Aib]EGTFTSDYSSYLEEQAAKEFIAWLVKGGGK(N3) amide (M6675)
(Payload -K(N3)),

or a pharmaceutically acceptable salt thereof.

In some embodiments, the compound has the structure selected from the group consisting of:

(SEQ ID NO: 47)
H[Aib]EGT-amY-TSD-X1-SSYLEEQAAKEFIAWLVKGGGK(N3)
amide (M6618) (Payload -K(N3)),
(SEQ ID NO: 48)
H[Aib]EGT-amY-TSD-X1-SSYLEEQAA-amK-E-amF-IAWLV-amK-
GGGK(N3) amide (Payload -K(N3)),
(SEQ ID NO: 49)
H[Aib]EGT-amF(2F)-TSDYSSYLEEQAAKEFIAWLVKGGGK(N3) amide
(Payload -K(N3)),
(SEQ ID NO: 50)
H[Aib]Atz-GTFTSDYSSYLEEQAAKEFIAWLVKGGGK(KN3) amide
(Payload -K(K(N3))),
(SEQ ID NOS 51 & 137)
H[Aib]EGT-amY-TSD-X1-SSYLEEQAA-amK-E-amF-IAWLV-amK-GGGK(T-
GGGGSGGGGSGGGGS-K(ALO)) amide (Payload -K(T-(G₄S)₃-K[ALO]),
(SEQ ID NO: 52)
H[Aib]EGT-amF(2F)-TSDYSSYLEEQAAKEFIAWLVKGGGGGGGSGGGGSGGGGSK
amide (Payload -(G₄S)₃-K),
(SEQ ID NO: 53)
H[Aib]EGT-F(4NH2)-TSDYSSYLEEQAAKEFIAWLVKGGGGGGGSGGGGSGGGGSK
amide (Payload -(G₄S)₃-K),
(SEQ IDNO: 54)
H[Aib]EGTFTSDY-X2-SYLEEQAAKEFIAWLVKGGGGGGGSGGGGS
GGGGSK (Payload -(G₄S)₃-K),
(SEQ ID NO: 55)
H[Aib]EGT-amF(2F)-TSDYSSYLEEQAAKEFIAWLVKGGGGGGGSG
GGGSGGGGSK[K(N3)]-CONH2 (M6571) (Payload -(G₄S)₃-K[K(N3)]),
(SEQ ID NO: 56)
H[Aib]Atz-GTFTSDYSSYLEEQAAKEFIAWLVKGGGGGGGSGGGGSG
GGGSK[K(N3)]-CONH2 (M6560) (Payload -(G₄S)₃-K[K(N3)]),
(SEQ ID NO: 17)
H[Aib]EGTFTSDYSSYLEEQAAKEFIAWLVKGGG (G₄S)₃-K(Ac-Mc)-
amide (M6557) (Payload -(G₄S)₃-K(Ac-Mc)),
(SEQ ID NO: 13)
H[Aib]EGTFTSDYSSYLEEQAAKEFIAWLVKGGG (G₄S)₃-
K(Ac-COT)-amide (M6562) (Payload -(G₄S)₃-K(Ac-COT)),
(SEQ ID NO: 57)
H[Aib]EGTFTSDYSSYLEEQAAKEFIAWLVKGGG-K[K(N3)]-amide
(Payload -K[K(N3)]),
(SEQ ID NO: 58)
H[Aib]EGTFTSDYSSYLEEQAAKEFIAWLVKGGG-K(T-CH2-ALO)-
amide (M6676) (Payload -K(T-CH2-ALO)),
(SEQ ID NO: 59)
H[Aib]EGTFTSDYSSYLEEQAAKEFIAWLVKGGG(G₄S)₃-K(N3)-amide
(Payload -(G₄S)₃-K(N3)),
(SEQ ID NO: 60)
H[Aib]EGTFTSDYSSYLEEQAAKEFIAWLVKGGG(G₄S)₃-K(T-CH2-ALO)-amide
(M6651) (Payload -(G₄S)₃-K(T-CH2-ALO)),

or a pharmaceutically acceptable salt thereof.

### Conjugation of a Drug, a Ligand, or a Handle to an Antibody or Antigen Binding Fragment Thereof

In another aspect, the present disclosure provides a process for conjugating a drug, a ligand, or a handle, site-specifically to an isolated antibody or antigen binding fragment thereof, where the site of conjugation is at Gln/Q55 of the light chain of the antibody, and where the site-specific conjugation is assisted by microbial transglutaminase (mTG).

In some embodiments, the drug, ligand, or handle comprises a primary amino group for the conjugation assisted by mTG to the side chain of Q55 of the antibody.

In some embodiments, the handle comprises a primary amino group for conjugation assisted by mTG to the side chain of Q55 of the antibody and a reactive moiety for conjugation with a drug or a ligand.

In some embodiments, the reactive moiety is for the Click or the Diels-Alder reaction.

In some embodiments, the reactive moiety for the Click reaction comprises —N₃,

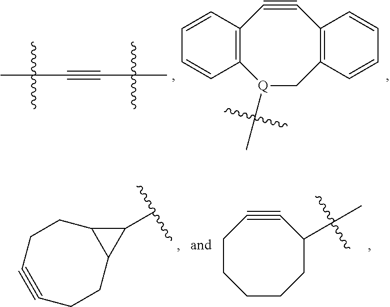

where Q is CH or N.

In some embodiments, the reactive moiety for the Diels-Alder reaction comprises

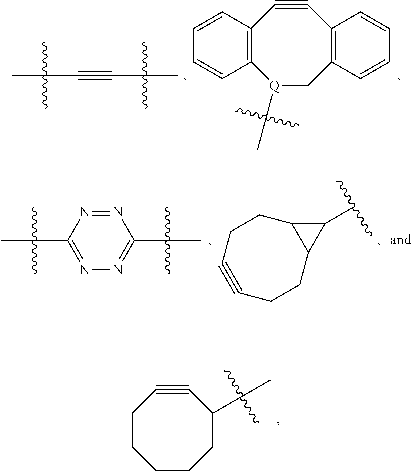

where Q is CH or N.

In some embodiments, the handle comprises

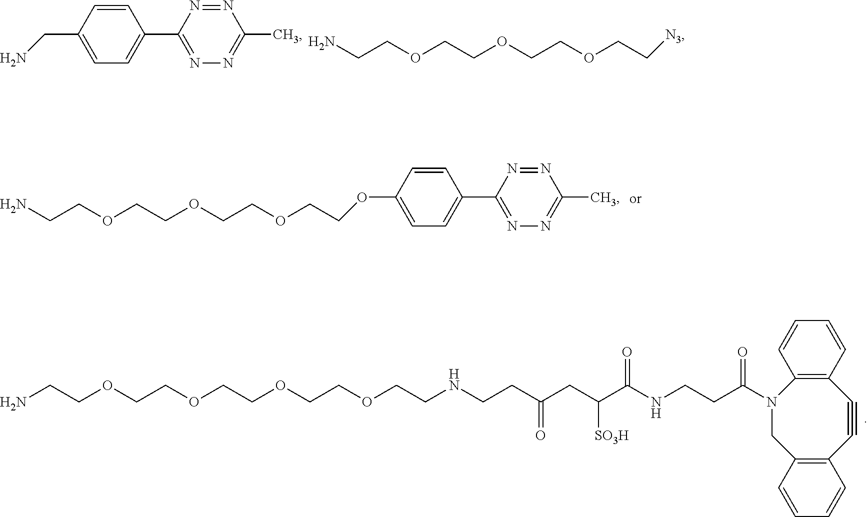 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the isolated antibody or antigen binding fragment thereof comprises REGN1033 (anti-GDF8 antibody), REGN4320 (anti-MSR1 N297Q), REGN4322 (H1H21231N, anti-MSR1, N297Q), H2aM21339N (anti-HLA-A2/CMV), H4H11283N (anti-HLA-B27), and H4sH14137N (anti-CD20), H1H20918P (CD226), H4H20122P (HLA-B27), H4H13767P (HFE2), H4H12587P (IL-6R), H4H11281N (HLA-B27), H1H15208P (MERS), H4H11283N, H4H11924N, H2aM14137N, H4sH14137N, H4H6073N2, H2aM14140N, and H1H10154P.

In some embodiments, the isolated antibody or antigen binding fragment thereof preferably has a phenylalanine residue at position 46 of the light chain.

In some embodiments, the GDF8 antibody, or an antigen-binding fragment thereof, binds to and/or blocks the biological activity of Growth and Differentiation Factor-8 (GDF8, SEQ ID NO: 1).

In some embodiments, the isolated antibody or antigen binding fragment thereof is a GDF8 antibody or antigen-binding fragment thereof that specifically binds to and/or blocks the biological activity of wild-type mature human GDF8 comprising SEQ ID NO: 1.

In another aspect, the present disclosure provides an antibody conjugate or a pharmaceutically acceptable salt thereof manufactured according to any of the processes described herein.

In another aspect, the present disclosure provides a pharmaceutical composition comprising an antibody conjugate manufactured according to any of the processes described herein together with one or more pharmaceutically acceptable excipients.

### Incretins and Glucagon-Like Peptide (GLP)-1 Agonists/Glucagon-Like Peptide (GLP)-1 Receptor Agonists/Payloads

The term “GLP-1”, also called as “glucagon-like peptide 1”, refers to the 31-amino acid peptide hormone released from intestinal L cells following nutrient consumption. GLP-1 binds to GLP-1 receptor and potentiates the glucose-induced secretion of insulin from pancreatic beta cells, increases insulin expression, inhibits beta-cell apoptosis, promotes beta-cell neogenesis, reduces glucagon secretion, delays gastric emptying, promotes satiety and increases peripheral glucose disposal. A “GLP-1 receptor agonist” refers to compounds having glucagon-like peptide-1 (GLP-1) receptor activity. Such exemplary compounds include exendins, exendin analogs, exendin agonists, GLP-1(7-37) (HAEGTFTSDVSSYLEGQAAKEFIAWLVKGRG, SEQ ID NO: 2)), GLP-1(7-37) analogs, and the like. The GLP-1 receptor agonist compounds may optionally be amidated. The terms “GLP-1 receptor (GLP1R) agonist” and “GLP-1 receptor agonist compound” have the same meaning.

As used herein, the term “GLP-1 agonist” refers to a compound that promotes, upregulates, or simulates the activity of GLP-1. GLP-1 agonists can activate GLP-1R and include GLP-1 mimetics, peptides variants, antibodies (including antibodies tethered to ligands), and fusion proteins. GLP-1 agonists include GLP-1 receptor agonists (GLP-1 RAs). The GLP-1 agonists described/used herein are GLP-1 receptor agonists. Indeed, for the purposes of the instant disclosure, the expressions “GLP-1 agonist” and “GLP-1R agonist” are used interchangeably. As used herein, the term “GLP-1 receptor agonist” refers to a compound that binds to GLP-1 receptor. GLP-1 receptor agonists increase glucose-dependent insulin secretion and decrease inappropriate glucagon secretion, delay gastric emptying, and increase satiety (Trujillo, et al., 2021, Ther Adv Endocrinol Metab 12:1-15). GLP-1 agonists may, for example, be selected from small molecule and peptide GLP-1R agonists and allosteric modulators (Graaf, et al., 2016, Pharmacol Rev 68:954-1013).

GLP-1 agonists for use in the instant disclosure include peptide agonists now on the market. In certain embodiments, the GLP-1 agonists mimic the action of glucagon-like peptide 1. Known GLP-1 receptor agonists include Albiglutide, Exenatide (short-acting and long-acting), Efpeglenatide, ITCA650, Lixisenatide, Liraglutide, Dulaglutide, and Semaglutide. In certain embodiments, the GLP-1 agonist is selected from the group consisting of Exenatide (long-acting), Dulaglutide, Liraglutide, and Semaglutide. In a further embodiment of a composition or method according to the disclosure, the GLP-1 agonist is Semaglutide. Semaglutide (sold under the brand name Ozempic, among others) is a glucagon-like peptide-1 receptor agonist that increases the production and secretion of insulin, thus increasing sugar metabolism. In one embodiment, the GLP-1 agonist for us in a method or composition according to the disclosure is a modified peptide drug, for example, Tirzepatide, that activates both the Glucagon-like peptide-1 (GLP-1) and glucose-dependent insulinotropic polypeptide (GIP) receptors.

Exenatide (sold under the brand names Byetta®, Bydureon®, Bydureon BCise®) is commonly used to help lower blood sugar levels in people with type 2 diabetes.

Liraglutide (sold under the brand names Victoza®, Saxenda®) is an anti-diabetic medication used to treat type 2 diabetes and chronic obesity.

Lixisenatide (sold under the brand names Adlyxin®, Lyxumia®) is used to improve blood sugar (glucose) control in adults with type 2 diabetes.

Albiglutide (sold under the brand names Tanzeum® and Eperzan®) is a glucagon-like peptide-1 agonist (GLP-1 agonist) for treatment of type 2 diabetes.

Dulaglutide (sold under the brand names Trulicity® among others) is a medication used for the treatment of type 2 diabetes.

Semaglutide (sold under the brand names Ozempic®, Wegovy®, Rybelsus®) is an anti-diabetic medication used for the treatment of type 2 diabetes and an anti-obesity medication used for long-term weight management.

Taspoglutide is the peptide with the sequence H2N-His-2-methyl-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-2-methyl-Ala-Arg-CONH₂ (SEQ ID NO: 91) and is an analog of human glucagon-like peptide-1 (Sebokova et al., “Taspoglutide, an Analog of Human Glucagon-Like Peptide-1 With Enhanced Stability and in Vivo Potency,” Endocrinology 151(6):2474-82 (2010)).

Tirzepatide (sold under the brand names Mounjaro®, Zepbound®) is an antidiabetic medication used for the treatment of type 2 diabetes and for weight loss.

In another aspect, the present disclosure provides a compound of Formula (A):

(A)
(SEQ ID NO: 71)
H-Aib-AA1-G-T-AA2-T-S-D-AA3-AA4-S-Y-L-E-E-Q-A-
A-AA5-E-AA6-I-A-W-L-V-AA7-G-G-G,

where
H is His;
Aib is 2-Aminoisobutyric acid;
AA₁ is E or

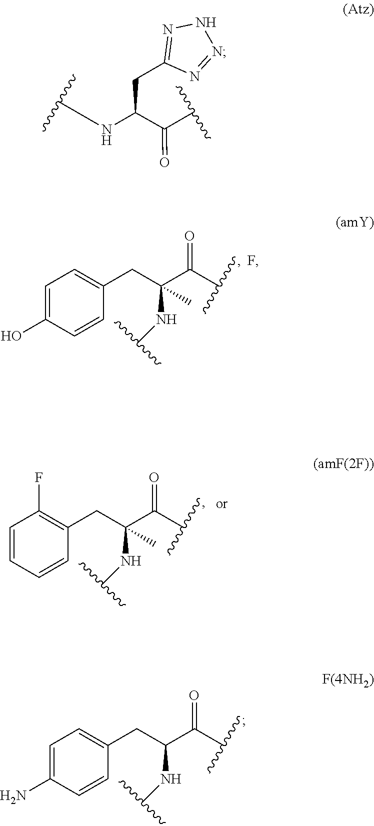 [SMILES: *N[C@@H](Cc1nn[nH]n1)C(*)=O ; *N[C@@](C)(Cc1ccc(O)cc1)C(*)=O ; *N[C@@](C)(Cc1ccccc1F)C(*)=O ; *N[C@@H](Cc1ccc(N)cc1)C(*)=O]

AA₃ is

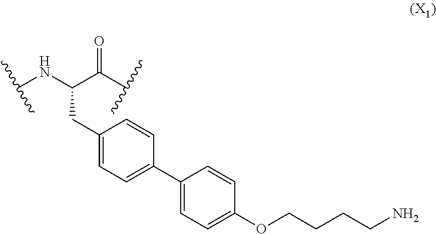 [SMILES: *N[C@@H](Cc1ccc(-c2ccc(OCCCCN)cc2)cc1)C(*)=O]

or Y;
AA₄ is S or

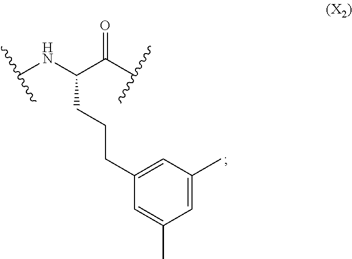 [SMILES: *N[C@@H](CCCc1cc(C)cc(C)c1)C(*)=O]

AA₅ is K or

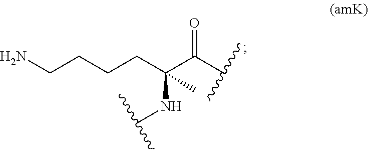 [SMILES: *N[C@@](C)(CCCCN)C(*)=O]

AA₆ is F or

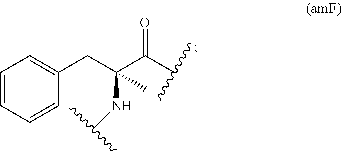 [SMILES: *N[C@@](C)(Cc1ccccc1)C(*)=O]

and
AA₇ is K or amK.

In some embodiments, AA₁ is E.

In some embodiments, AA₂ is F. In other embodiments, AA₂ is amY.

In some embodiments, AA₃ is Y.

In some embodiments, AA₄ is S.

In some embodiments, AA₅ is K.

In some embodiments, AA₆ is F.

In some embodiments, AA₇ is K.

In some embodiments, the compound is selected form the group consisting of:

(SEQ ID NO: 3)
H(Aib)EGTFTSDYSSYLEEQAAKEFIAWLVKGGG,
(SEQ ID NO: 38)
H[Aib]EGT-amY-TSD-X1-SSYLEEQAAKEFIAWLVKGGG,
(SEQ ID NO: 39)
H[Aib]EGT-amY-TSD-X1-SSYLEEQAA-amK-E-amF-
IAWLV-amK-GGG,
(SEQ ID NO: 40)
H[Aib]EGT-amF(2F)-TSDYSSYLEEQAAKEFIAWLVKGGG,
(SEQ ID NO: 41)
H[Aib]Atz-GTFTSDYSSYLEEQAAKEFIAWLVKGGG,
(SEQ ID NO: 43)
H[Aib]EGT-amY-TSD-X1-SSYLEEQAA-amK-E-amF-
IAWLV-amK-GGG,
(SEQ ID NO: 44)
H[Aib]EGT-amF(2F)-TSDYSSYLEEQAAKEFIAWLVKGGG,
(SEQ ID NO: 45)
H[Aib]EGT-F(4NH2)-TSDYSSYLEEQAAKEFIAWLVKGGG,
(SEQ ID NO: 46)
H[Aib]EGTFTSDY-X2-SYLEEQAAKEFIAWLVKGGG,
(SEQ ID NO: 42)
H[Aib]EGT-amF(2F)-TSDYSSYLEEQAAKEFIWLVKGGG,
and
(SEQ ID NO: 12)
H[Aib]Atz-GTFTSDYSSYLEEQAAKEFIAWLVKGGG.

In some embodiments, the GLP1R agonist is selected from the group consisting of GLP-1(7-37) (SEQ ID NO: 2); the compound according to any of the embodiments described herein; exenatide, liraglutide, lixisenatide, albiglutide, dulaglutide, semaglutide, taspoglutide, and tirzepatide.

In one embodiment, the GLP1R agonist is selected from the group consisting of GLP-1(7-37) (SEQ ID NO: 2); GLP1R agonist (SEQ ID NO: 3), exenatide, liraglutide, lixisenatide, albiglutide, dulaglutide, semaglutide, taspoglutide, and tirzepatide.

In another embodiment, the GLP1R agonist is selected from the group consisting of exenatide, liraglutide, lixisenatide, albiglutide, dulaglutide, semaglutide, taspoglutide, and tirzepatide.

In another embodiment, the GPL-1 agonist/receptor agonist for use in a composition or method according to the disclosure is an antibody or antigen-binding fragment thereof that specifically binds GLP-1.

In certain embodiments, the GLP-1 agonist for use in compositions and methods according to the invention is an antibody-drug conjugate (ADC) that specifically binds the glucagon-like peptide 1 receptor (GLP-1R) protein. In a further embodiment, the antibody or antigen-binding fragment thereof of the ADC specifically targets the extracellular domain of GLP-1R, with a GLP-1 peptidomimetic functionally activating GLP-1R.

An antibody-tethered drug conjugate (ATDC) or antibody-drug conjugate (ADC) refers to an antibody or antigen-binding fragments thereof tethered, by a linker or without a linker, to a payload peptide (e.g., a GLP-1 peptidimimetic). An antibody-payload conjugate refers to such an antibody or fragment linked to a payload whereas an antibody-linker-payload conjugate refers to an antibody or fragment conjugated to a payload via a linker. An antibody or antigen-binding fragment referred to herein includes embodiments where the antibody or fragment is be conjugated to a payload or linker-payload.

In some embodiments, the GLP1R agonist is the compound according to any of the embodiments described herein.

In some embodiments, the GLP1R agonist comprises the compound according to any of the embodiments described herein.

In some embodiments, GLP1R agonist (P) comprises the sequence H(Aib)EGTFTSDYSSYLEEQAAKEFIAWLVKGGG (SEQ ID NO: 3), where Aib is 2-Aminoisobutyric acid.

In some embodiments, the GLP1R agonist consists the compound according to any of the embodiments described herein.

In some embodiments, GLP1R agonist (P) consists of the sequence H(Aib)EGTFTSDYSSYLEEQAAKEFIAWLVKGGG (SEQ ID NO: 3), where Aib is 2-Aminoisobutyric acid.

In some embodiments, GLP1R agonist (P) has a sequence of H(Aib)EGTFTSDYSSYLEEQAAKEFIAWLVKGGG (SEQ ID NO: 3), where Aib is 2-Aminoisobutyric acid.

### Antibodies and Antigen-Binding Fragments Thereof

Proprotein convertase subtilisin/kexin type 9 (PCSK9) was initially identified as a new member of the proprotein convertase family and suggested to have a role in liver regeneration and the differentiation of cortical neurons (Peterson et al., “PCSK9 Function and Physiology,” J. Lipid Res. 49(6):1152-1156 (2008), which is incorporated herein by reference in its entirety). PCSK9 is a key player in plasma cholesterol metabolism and regulates the levels of the LDL receptor, which is a plasma membrane glycoprotein that removes cholesterol-rich LDL particles from the plasma.

PCSK9 is a 72-kd protease, expressed highly in liver, with three recognizable domains, an N-terminal prodomain, a catalytic domain, and a carboxyl-terminal domain of unknown function.

In some embodiments, the antibody is a PCSK9 antibody or antigen-binding fragment thereof.

In some embodiments, the antibody is a GLP1R antibody or antigen-binding fragment thereof.

In some embodiments, the antibody is an activin receptor type 2 antibody or antigen-binding fragment thereof.

In some embodiments, the antibody is a Growth and Differentiation Factor-8 (GDF8, myostatin) antibody or antigen-binding fragment thereof.

Growth and Differentiation Factor-8 (GDF8), also known as myostatin, is a member of the TGF-β superfamily of growth factors. GDF8 is a negative regulator of skeletal muscle mass, highly expressed in the developing and adult skeletal muscle.

GDF8 is highly conserved across species, and the amino acid sequences of murine and human GDF8 are identical (human GDF8 nucleic acid sequence and amino acid sequence shown in SEQ ID NO:338-339) (McPherron et al. 1977 Nature 387:83-90).

Antibodies to GDF8 and therapeutic methods are disclosed in, e.g., U.S. Pat. Nos. 6,096,506, 7,320,789, 7,807,159, WO 2007/047112, WO 2005/094446, US 2007/0087000, U.S. Pat. No. 7,261,893, and WO 2010/070094.

These antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al. (2000) J. Immunol. 164:1925-1933).

In certain specific embodiments of the present disclosure, the GDF-8 inhibitor is a GDF8-specific binding protein, and the protein, or the GDF8-specific binding domain, comprises or consists of an anti-GDF8 antibody or antigen-binding fragment thereof. Anti-GDF8 antibodies are mentioned in, e.g., U.S. Pat. Nos. 6,096,506; 7,320,789; 7,261,893; 7,807,159; 7,888,486; 7,635,760; 7,632,499; in US Patent Appl. Publ. Nos. 2007/0178095; 2010/0166764; 2009/0148436; and International Patent Appl. Publ. No. WO 2010/070094. Anti-GDF8 antibodies are also described in U.S. patent application Ser. No. 13/115,170, filed on May 25, 2011, and published as US 20110293630, and the continuation application Ser. No. 14/462,085, filed Aug. 18, 2014, published as US 20140356369, including the antibodies designated 8D12, H4H1657N2, and H4H1669P. In some embodiments, the anti-GDF8 antibodies are selected from the group consisting of AMG 181, AMG 745, Apitegromab (SRK-015), Bimagrumab (BYM338), Domagrozumab (PF-06252616), GYM329 (RO7204239), Landogrozumab (LY2495655), Stamulumab (MYO-029), and Trevogrumab (REGN1033).

In one embodiment, the anti-GDF8 antibody is REGN1033, also known as trevogrumab, or H4H1657N2. Any of the anti-GDF8 antibodies mentioned and/or described in any of the foregoing patents or publications, or antigen-binding fragments thereof, can be used in the context of the present disclosure, so long as such antibodies and/or antigen-binding fragments “specifically bind” GDF8, as that expression is defined herein.

The present invention also includes anti-GDF8 antibodies comprising variants of any of the HCVR, LCVR, and/or CDR amino acid sequences disclosed herein having one or more conservative substitutions. For example, the present invention includes anti-GDF8 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

Suitable GDF8 antibody or the antigen binding fragment thereof that can be used include the ones mentioned in U.S. Pat. No. 8,840,894. In some embodiments, the GDF8 antibody or the antigen binding fragment thereof comprises 1A2, 21-E5, 8D12, H4H1669P, AMG 181, AMG 745, Apitegromab (SRK-015), Bimagrumab (BYM338), Domagrozumab (PF-06252616), GYM329 (RO7204239), Landogrozumab (LY2495655), Stamulumab (MYO-029), and Trevogrumab (REGN1033). In some embodiments, the GDF8 antibody or the antigen binding fragment thereof comprises 1A2, 21-E5, 8D12, H4H1657N2 (REGN1033), and H4H1669P. See U.S. Pat. No. 8,840,894.

Antibody identifier (HCVR/LCVR): 21-E5 (SEQ ID NO:61/62); 1A2 (SEQ ID NO:65/66); 812-1 (SEQ 1D NO:63/64-); H4H1A1657N2 (SEQ 1D NO:67/68); H4H1669P (SEQ ID NO:69/70).

```
<210> 61
<211> 125
<212> PRT
<213> Artificial Sequence
<220>
<223> Synthetic
<400> 61
Gln Val Gln Leu Val Glu Ser Gly Pro Gly Leu Val Lys Pro Ser Glu
1 5 10 15
Thr Leu Ser Leu Thr Cys Thr Val Tyr Gly Gly Ser Ile Ser Ser Gly
                          20 25 30
Asn Tyr Tyr Trp Gly Trp Ile Arg Gln Pro Pro Gly Lys Gly Leu Glu
                  35 40 45
Trp Ile Gly Thr Ile Tyr Tyr Ser Gly Ser Ala Tyr Tyr Asn Pro Ser
          50 55 60
Leu Lys Ser Arg Val Thr Met Ser Val Asp Thr Ser Lys Asn Gln Phe
65 70 75 80
Ser Leu Lys Leu Ser Ser Val Thr Ala Ala Asp Thr Ala Val Tyr Tyr
                                85 90 95
Cys Val Arg Asp Tyr Tyr Asp Ser Ser Gly His Tyr Tyr Asn Trp Phe
                        100 105 110
Asp Pro Trp Gly Gln Gly Thr Thr Val Thr Val Ser Ser
                115 120 125
<210> 62
<211> 108
<212> PRT
<213> Artificial Sequence
<220>
<223> Synthetic
<400> 62
Ala Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg His Asp
                          20 25 30
Leu Gly Trp Tyr Gln Gln Lys Pro Gly Lys Ala Pro Lys Arg Leu Ile
                  35 40 45
Tyr Ala Ala Ser Ser Leu Gln Ser Gly Val Pro Ser Arg Phe Ser Gly
          50 55 60
Ser Gly Ser Gly Thr Glu Phe Thr Leu Thr Ile Ser Ser Leu Gln Pro
65 70 75 80
Glu Asp Phe Ala Thr Tyr Tyr Cys Leu Gln His Asn Thr Tyr Pro Trp
                                85 90 95
Thr Phe Gly Gln Gly Thr Lys Val Glu Ile Lys Arg
                        100 105
<210> 63
<211> 118
<212> PRT
<213> Artificial Sequence
<220>
<223> Synthetic
<400> 63
Gln Val Gln Leu Val Glu Ser Gly Gly Gly Val Val Gln Pro Gly Arg
1 5 10 15
Ser Leu Arg Leu Ser Cys Ala Ala Ser Gly Phe Thr Phe Ser Arg Tyr
                          20 25 30
Gly Ile His Trp Val Arg Gln Ala Pro Gly Lys Gly Leu Glu Trp Val
                  35 40 45
Ala Val Ile Ser Tyr Asp Gly Ser Asp Glu Tyr Tyr Val Asp Ser Val
          50 55 60
Lys Gly Arg Phe Ser Ile Ser Arg Asp Asn Ser Lys Asn Thr Leu Tyr
65 70 75 80
Leu Gln Met Asn Ser Leu Arg Pro Ala Asp Ser Ala Val Tyr Tyr Cys
                                85 90 95
Val Lys Gly Asp Leu Glu Leu Gly Phe Asp Tyr Trp Gly Gln Gly Thr
                        100 105 110
Leu Val Thr Val Ser Ser
                115
<210> 64
<211> 112
<212> PRT
<213> Artificial Sequence
<220>
<223> Synthetic
<400> 64
Asp Ile Val Met Thr Gln Ala Ala Pro Ser Ile Pro Val Ile Pro Gly
1 5 10 15
Glu Ser Val Ser Met Ser Cys Arg Ser Ser Lys Ser Leu Leu Tyr Ser
                          20 25 30
Asn Gly His Thr Tyr Val Tyr Trp Phe Val Gln Arg Pro Gly Gln Ser
                  35 40 45
Pro Gln Leu Leu Ile Tyr Arg Met Ser Asn Leu Ala Ser Gly Val Pro
          50 55 60
Asp Arg Phe Ser Gly Ser Gly Ser Gly Thr Ala Phe Thr Leu Arg Ile
65 70 75 80
Ser Arg Val Glu Ala Glu Asp Val Gly Val Tyr Tyr Cys Met Gln Asn
                                85 90 95
Leu Glu Phe Pro Leu Thr Phe Gly Ala Gly Thr Lys Leu Glu Leu Lys
                        100 105 110
<210> 65
<211> 122
<212> PRT
<213> Artificial Sequence
<220>
<223> Synthetic
<400> 65
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Ser Ile Thr Tyr
                          20 25 30
Ala Met Ser Trp Val Arg Gln Ala Pro Gly Lys Gly Leu Glu Trp Val
                  35 40 45
Ser Ala Ile Ser Val Ser Gly Thr Asn Thr Tyr Tyr Ala Asp Ser Val
          50 55 60
Lys Gly Arg Phe Thr Ile Ser Arg Asp Lys Ser Lys Asn Met Leu Tyr
65 70 75 80
Leu Gln Met Asn Ser Leu Arg Ala Glu Asp Thr Ala Val Tyr Tyr Cys
                                85 90 95
Ala Lys Asp Leu Leu His Asn Trp Lys Tyr Gly Thr Phe Asp Ile Trp
                        100 105 110
Gly Gln Gly Thr Met Val Thr Val Ser Ser
                  115 120
<210> 66
<211> 107
<212> PRT
<213> Artificial Sequence
<220>
<223> Synthetic
<400> 66
Glu Ile Val Met Thr Gln Ser Pro Ala Thr Leu Ser Val Ser Pro Gly
1 5 10 15
Glu Arg Ala Thr Leu Ser Cys Arg Ala Ser Gln Ser Val Asp Ser Asn
                          20 25 30
Leu Val Trp Tyr Gln Gln Lys Pro Gly Gln Val Pro Arg Leu Leu Ile
                  35 40 45
Tyr Gly Ala Ser Thr Arg Ala Thr Gly Ile Pro Ala Arg Phe Ser Gly
          50 55 60
Ser Gly Ser Gly Thr Glu Phe Thr Leu Thr Ile Ser Ser Leu Gln Ser
65 70 75 80
Glu Asp Phe Ala Val Tyr Tyr Cys Gln Gln Tyr Asn Lys Trp Pro Leu
                                85 90 95
Thr Phe Gly Gly Gly Thr Lys Val Glu Ile Lys
                        100 105
<210> 67
<211> 120
<212> PRT
<213> Artificial Sequence
<220>
<223> Synthetic
<400> 67
Glu Val Gln Val Leu Glu Ser Gly Gly Asp Leu Val Gln Pro Gly Gly
1 5 10 15
Ser Leu Arg Leu Ser Cys Ala Ala Ser Gly Phe Thr Phe Ser Ala Tyr
                          20 25 30
Ala Met Thr Trp Val Arg Gln Ala Pro Gly Lys Gly Leu Glu Trp Val
                  35 40 45
Ser Ala Ile Ser Gly Ser Gly Gly Ser Ala Tyr Tyr Ala Asp Ser Val
          50 55 60
Lys Gly Arg Phe Thr Ile Ser Arg Asp Asn Ser Lys Asn Thr Val Tyr
65 70 75 80
Leu Gln Met Asn Ser Leu Arg Ala Glu Asp Thr Ala Val Tyr Tyr Cys
                                85 90 95
Ala Lys Asp Gly Ala Trp Lys Met Ser Gly Leu Asp Val Trp Gly Gln
                        100 105 110
Gly Thr Thr Val Ile Val Ser Ser
                  115 120
<210> 68
<211> 107
<212> PRT
<213> Artificial Sequence
<220>
<223> Synthetic
<400> 68
Asp Ile Gln Met Thr Gln Ser Pro Ala Ser Leu Ser Ala Ser Val Gly
1 5 10 15
Asp Arg Val Thr Ile Thr Cys Arg Ala Ser Gln Asp Ile Ser Asp Tyr
                          20 25 30
Leu Ala Trp Tyr Gln Gln Lys Pro Gly Lys Ile Pro Arg Leu Leu Ile
                  35 40 45
Tyr Thr Thr Ser Thr Leu Gln Ser Gly Val Pro Ser Arg Phe Arg Gly
          50 55 60
Ser Gly Ser Gly Thr Asp Phe Thr Leu Thr Ile Ser Ser Leu Gln Pro
65 70 75 80
Glu Asp Val Ala Thr Tyr Tyr Cys Gln Lys Tyr Asp Ser Ala Pro Leu
                                85 90 95
Thr Phe Gly Gly Gly Thr Lys Val Glu Ile Lys
                        100 105
<210> 69
<211> 121
<212> PRT
<213> Artificial Sequence
<220>
<223> Synthetic
<400> 69
Gln Val Gln Leu Val Glu Ser Gly Gly Gly Val Val Gln Pro Gly Lys
1 5 10 15
Ser Leu Arg Leu Ser Cys Ala Ala Ser Gly Phe Thr Phe Ser Ser Phe
                          20 25 30
Gly Met His Trp Val Arg Gln Ala Pro Gly Lys Gly Leu Glu Trp Val
                  35 40 45
Ala Val Ile Gly Tyr Asp Gly Gly Asn Glu Tyr Tyr Ala Asp Ser Val
          50 55 60
Lys Gly Arg Phe Thr Ile Ser Arg Asp Asn Ser Lys Asn Thr Leu Asn
65 70 75 80
Leu Gln Met Ser Ser Leu Arg Ala Glu Asp Thr Ala Val Tyr Tyr Cys
                                85 90 95
Ser Thr Ile Ser His Tyr Asp Ile Leu Ser Gly Met Asp Val Trp Gly
                        100 105 110
Arg Gly Thr Thr Val Thr Val Ser Ser
                  115 120
<210> 70
<211> 108
<212> PRT
<213> Artificial Sequence
<220>
<223> Synthetic
<400> 70
Asp Ile Gln Met Thr Gln Ser Pro Ser Ser Val Ser Ala Ser Val Gly
1 5 10 15
Asp Arg Val Thr Ile Thr Cys Arg Ala Ser Gln Gly Ile Ser Asn Trp
                          20 25 30
Leu Ala Trp Tyr Gln Gln Lys Pro Gly Lys Ala Pro Lys Leu Leu Ile
                  35 40 45
Phe Ala Ala Ser Ser Leu Gln Ser Gly Val Pro Ser Arg Phe Ser Gly
          50 55 60
Ser Ala Ser Gly Thr Asp Phe Thr Leu Thr Ile Asn Ser Leu Gln Pro
65 70 75 80
Glu Asp Phe Ala Thr Tyr Tyr Cys Gln Gln Ala Asn Ser Phe Pro Leu
                                85 90 95
Thr Phe Gly Gly Gly Thr Lys Val Glu Ile Lys Arg
                        100 105
```

In one embodiment, the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO: 5.

In another embodiment, the anti-GDF8 antibody or antigen-binding fragment thereof comprises heavy chain complementarity determining regions (HCDR1, HCDR2, and HCDR3) comprising SEQ ID NO: 6, SEQ ID NO: 7, and SEQ ID NO: 8, respectively, and three light chain complementarity determining regions (LCDR1, LCDR2, and LCDR3) comprising SEQ ID NO: 9, SEQ ID NO: 10, and SEQ ID NO: 11, respectively.

### A Linker Moiety

A “linker moiety” as used herein refers to a biologically acceptable peptidyl or non-peptidyl organic group that is covalently bound to an amino acid residue of a toxin peptide analog or other polypeptide chain (e.g., an immunoglobulin HC or LC or immunoglobulin Fc domain) contained in the inventive composition, which linker moiety covalently joins or conjugates the toxin peptide analog or other polypeptide chain to another peptide or polypeptide chain in the composition, or to a half-life extending moiety. In some embodiments of the composition, a half-life extending moiety, as described herein, is conjugated, i.e., covalently bound directly to an amino acid residue of the toxin peptide analog itself, or optionally, to a peptidyl or non-peptidyl linker moiety (including but not limited to aromatic or aryl linkers) that is covalently bound to an amino acid residue of the toxin peptide analog. The presence of any linker moiety is optional. When present, its chemical structure is not critical, since it serves primarily as a spacer to position, join, connect, or optimize presentation or position of one functional moiety in relation to one or more other functional moieties of a molecule of the inventive composition. The presence of a linker moiety can be useful in optimizing pharmacological activity of some embodiments of the inventive composition. The linker, if present, can be made up of amino acids linked together by peptide bonds. The linker moiety, if present, can be independently the same or different from any other linker, or linkers, that may be present in the inventive composition. In some embodiments the linker can be a multivalent linker that facilitates multivalent display of toxin peptide analogs of the present invention; multivalent display of such biologically active compounds can increase binding affinity and/or potency through avidity. The in vivo properties of a therapeutic can be altered (i.e., specific targeting, half-life extension, distribution profile, etc.) through conjugation to a polymer or protein.

In some embodiments, the linker comprise

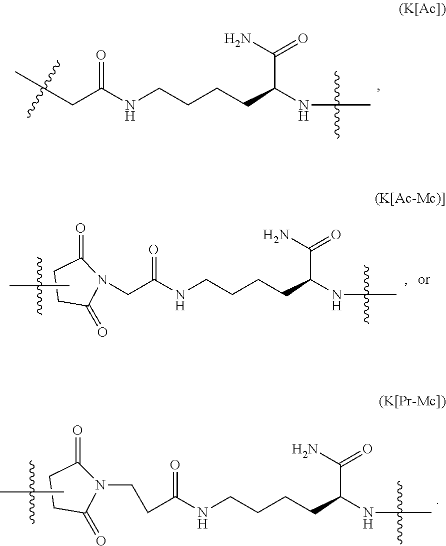

In some embodiments, the linker (linker L) the linker comprises a conjugation reaction product of a reactive moiety from the GLP1R agonist and a reactive moiety from the BA.

In some embodiments, the reactive moiety comprises thiol, maleimido, 2-bromo acetyl, —N₃,

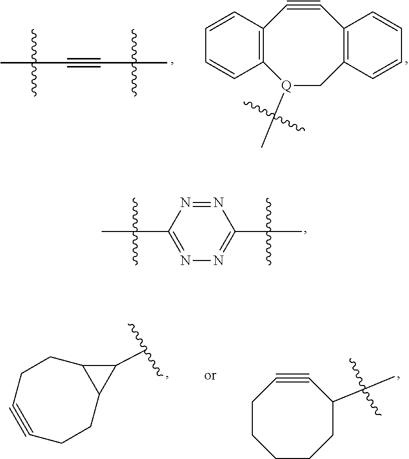

where Q is CH or N.

In some embodiments, the reactive moiety comprises

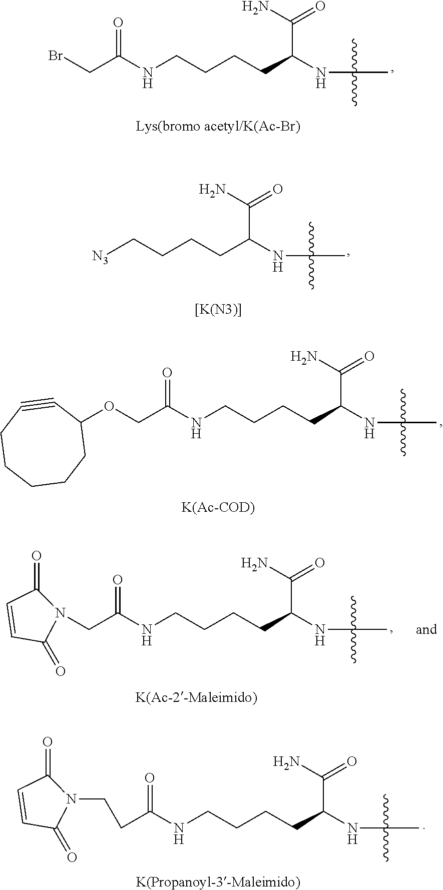

In some embodiments, the reactive moiety of the GDF8 antibody and the reactive moiety of the GLP1R agonist form a linker adduct having a structure of

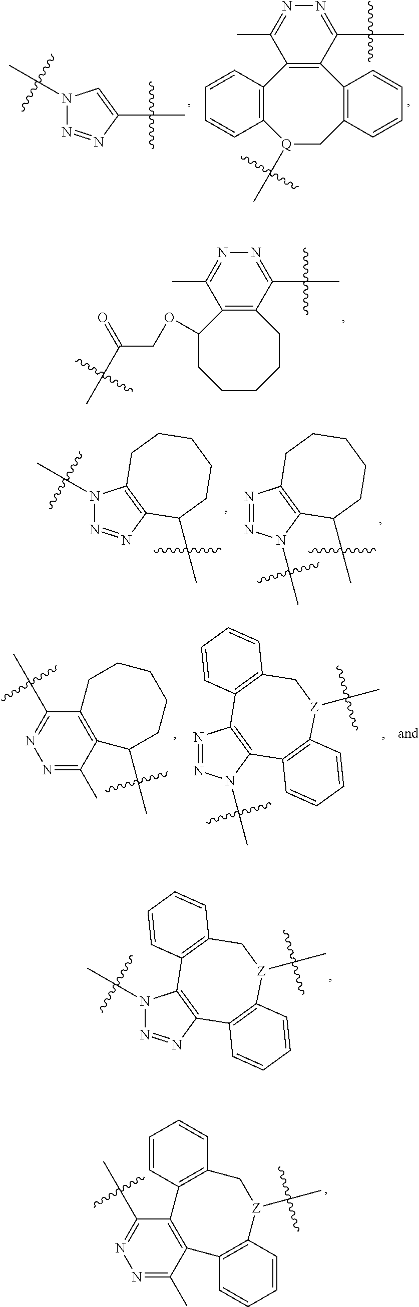

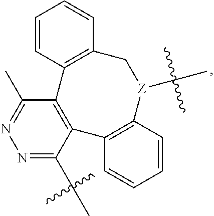

where Z is CH or N.

In some embodiments, the linker (linker L) has a structure of

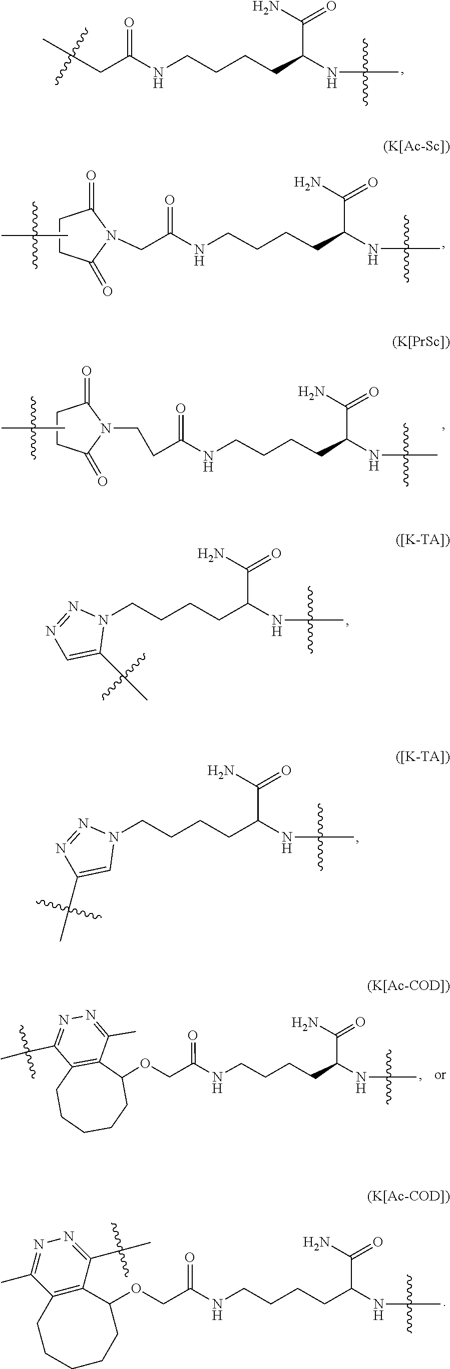

In some embodiments, the linker (linker L) comprises a moiety formed by Click or Diels-Alder reaction (Click or Diels-Alder reaction adduct). In some embodiments, the linker (linker L) comprises a Click or Diels-Alder reaction adduct selected from the group consisting of:

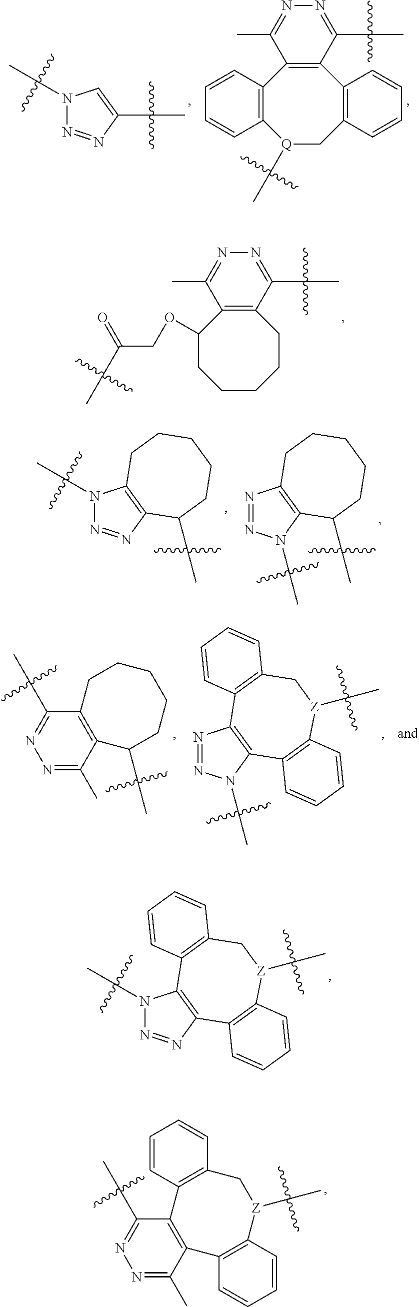

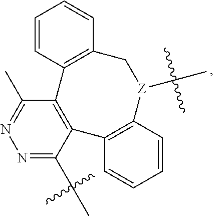

where Z is CH or N.

In some embodiments, the reactive moiety for the Click reaction comprises

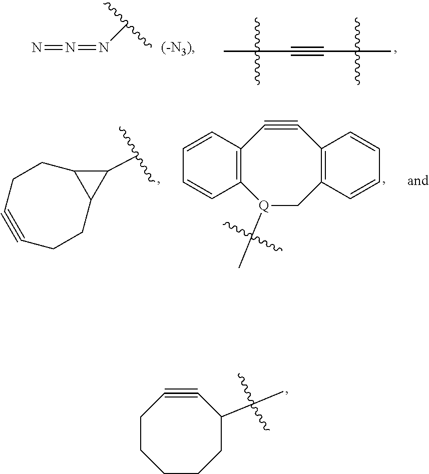

where Q is CH or N.

In some embodiments, the reactive moiety for the Diels-Alder reaction comprises

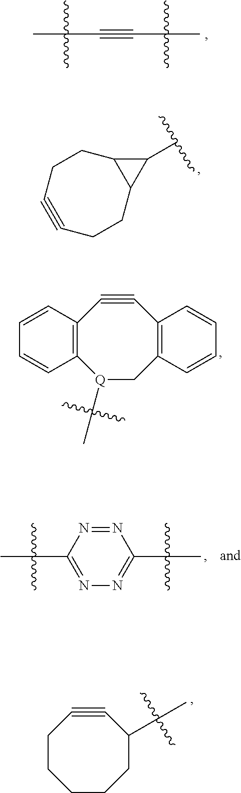

where Q is CH or N.

In some embodiments, the linker comprises

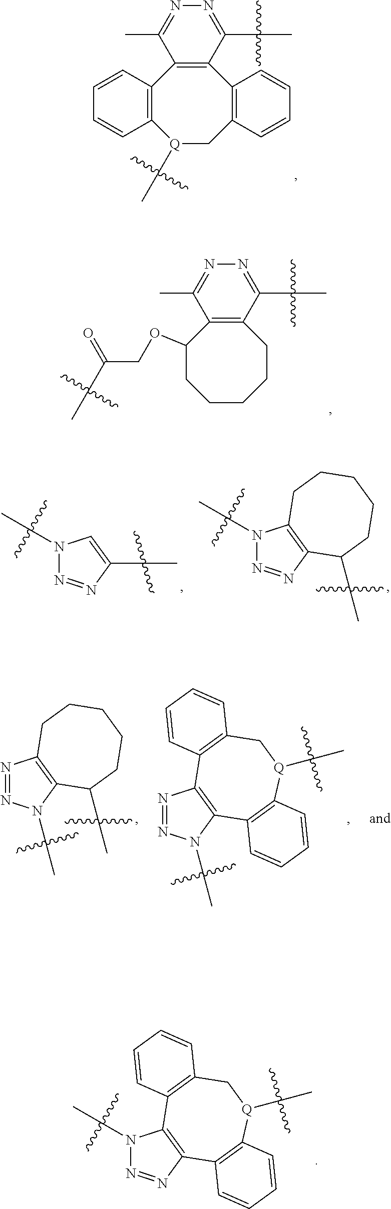

In some embodiments, the linker comprises

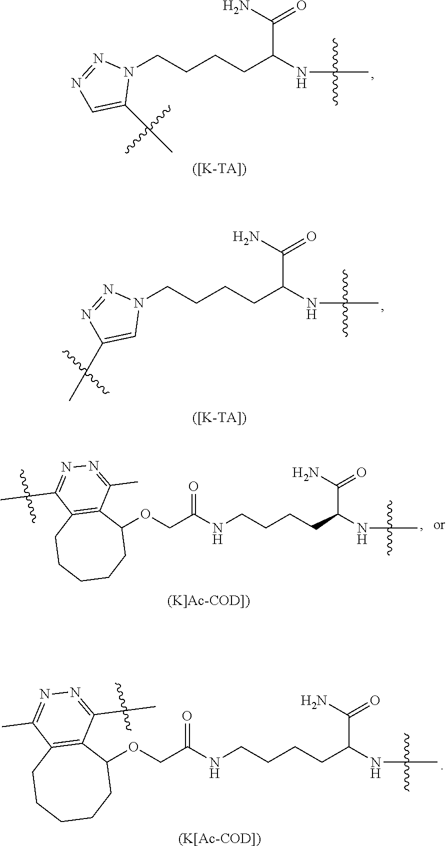

In some embodiments, the linker L comprises a sequence selected from the group consisting of -(Gly₃Ser)ₘ- (SEQ ID NO: 84) and -(Gly₄Ser)ₘ- (SEQ ID NO: 85), where m is an integer from one to six.

In some embodiments, the linker comprises a sequence selected from the group consisting of Gly₃Ser (SEQ ID NO: 72), Gly₄Ser (SEQ ID NO: 73), (Gly₃Ser)₂ (SEQ ID NO: 74), (Gly₄Ser)₂ (SEQ ID NO: 75), (Gly₃Ser)₃ (SEQ ID NO: 76), (Gly₄Ser)₃ (SEQ ID NO: 77), (Gly₃Ser)₄ (SEQ ID NO: 78), (Gly₄Ser)₄ (SEQ ID NO: 79), (Gly₃Ser)₅ (SEQ ID NO: 80), (Gly₄Ser)₅ (SEQ ID NO: 81), (Gly₃Ser)₆ (SEQ ID NO: 82), and (Gly₄Ser)₆ (SEQ ID NO: 83).

In some embodiments, the linker L comprises a sequence selected from the group consisting of -(Gly₃Ser)ₘ-Lys- (SEQ ID NO: 92) and -(Gly₄Ser)ₘ-Lys- (SEQ ID NO: 87), where m is an integer from one to six; and where the side chain of Lys is further functionalized with a reactive moiety for conjugation with the BA.

In some embodiments, the side chain of the Lys can be further functionalized with a reactive moiety (for conjugation with the BA) having a structure of

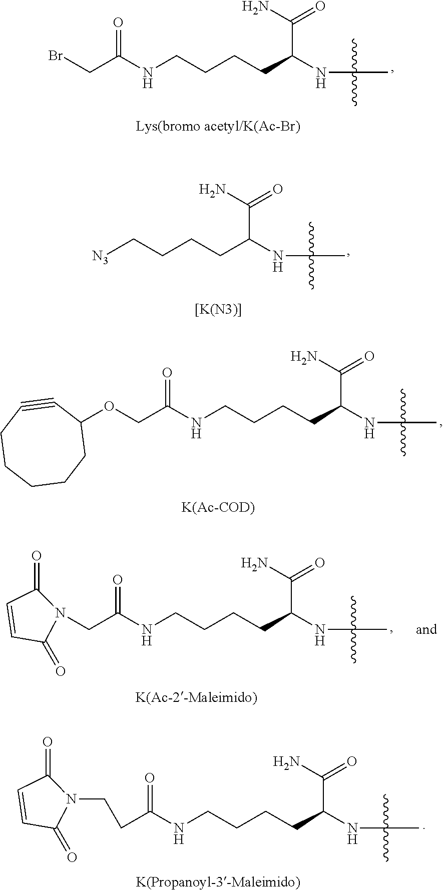

In some embodiments, a handle comprising a reactive moiety for Click or Diels-Alder reaction is first conjugated to the BA via mTG for the following conjugation with a reactive moiety on the side chain of Lys of linker -(Gly₄Ser)ₙ-Lys- (SEQ ID NO: 88). Suitable handle has structure consisting of

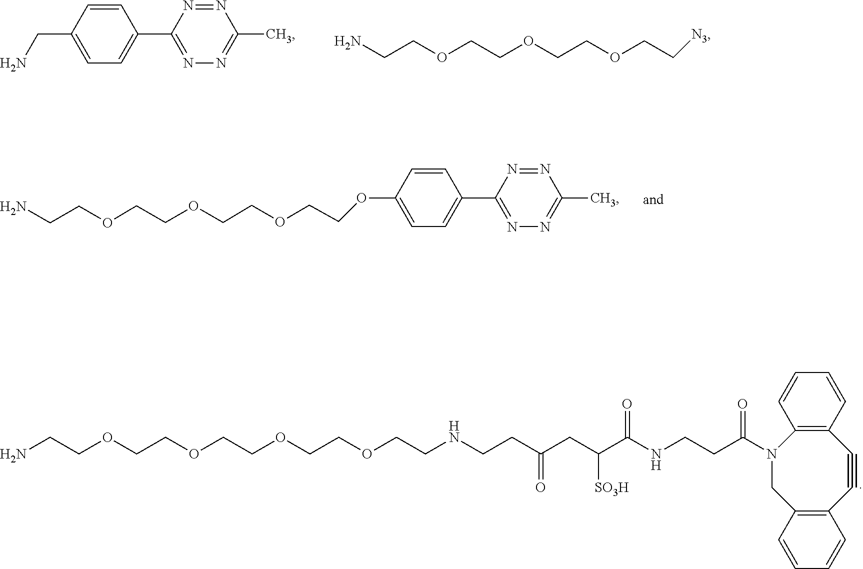 [SMILES: Cc1nnc(-c2ccc(CN)cc2)nn1 ; [N-]=[N+]=NCCOCCOCCOCCN ; Cc1nnc(-c2ccc(OCCOCCOCCOCCN)cc2)nn1 ; NCCOCCOCCOCCOCCNCCC(=O)CC(C(=O)NCCC(=O)N1Cc2ccccc2C#Cc2ccccc21)S(=O)(=O)O]

In some embodiments, the linker further comprises a sequence of -(Gly₃Ser)ₙ-Lys(X)- (SEQ ID NO: 89) or -(Gly₄Ser)ₙ-Lys(X)- (SEQ ID NO: 90), where n is an integer from one to six; and where X is a reactive moiety attached to the side chain of Lys for conjugation with the BP.

### Conjugation of Linker-Payloads to a GDF8 Antibody

The conjugates described herein can be synthesized by coupling the linker-payloads described herein with a binding agent, for example, an antibody under standard conjugation conditions (see, e.g., Doronina et al. Nature Biotechnology 2003, 21, 7, 778, which is incorporated herein by reference in its entirety). When the binding agent is an antibody, the antibody may be coupled to a linker-payload via one or more cysteine or lysine residues of the antibody. Linker-payloads can be coupled to cysteine residues, for example, by subjecting the antibody to a reducing agent, for example, dithiotheritol, to cleave the disulfide bonds of the antibody, purifying the reduced antibody, for example, by gel filtration, and subsequently treating the antibody with a linker-payload containing a suitable reactive moiety, for example, a maleimido group. Suitable solvents include, but are not limited to water, DMA, DMF, and DMSO. Linker-payloads containing a reactive group, for example, an activated ester or acid halide group, can be coupled to lysine residues of the antibody. Suitable solvents include, but are not limited to water, DMA, DMF, and DMSO. Conjugates can be purified using known protein techniques, including, for example, size exclusion chromatography, dialysis, and ultrafiltration/diafiltration.

Binding agents, for example antibodies, can also be conjugated via click chemistry reactions. In some embodiments of the click chemistry reactions, the linker-payload includes a reactive group, for example an alkyne, that is capable of undergoing a 1,3-cycloaddition reaction with an azide. Such suitable reactive groups are described above. The antibody includes one or more azide groups. Such antibodies include antibodies functionalized with, for example, azido-polyethylene glycol groups. In certain embodiments, such functionalized antibody is derived by treating an antibody having at least one glutamine residue, for example, heavy chain Gln295 or light chain Gln55, with a primary amine compound in the presence of the enzyme transglutaminase. In certain embodiments, such functionalized antibody is derived by treating an antibody having at least one glutamine residue, for example, heavy chain Gln297, with a primary amine compound in the presence of the enzyme transglutaminase. Such antibodies include Asn297Gln (N297Q) mutants. In certain embodiments, such functionalized antibody is derived by treating an antibody having at least two glutamine residues, for example, heavy chain Gln295 and heavy chain Gln297, with a primary amine compound in the presence of the enzyme transglutaminase. Such antibodies include Asn297Gln (N297Q) mutants. In certain embodiments, the antibody has two heavy chains as described in this paragraph for a total of two or a total of four glutamine residues.

In certain embodiments, the antibody comprises a glutamine residue at one or more heavy chain positions numbered 295 in the EU numbering system. In the present disclosure, this position is referred to as glutamine 295, or as Gln295, or as Q295. Those of skill will recognize that this is a conserved glutamine residue in the wild type sequence of many antibodies. In other useful embodiments, the antibody can be engineered to comprise a glutamine residue. Techniques for modifying an antibody sequence to include a glutamine residue are within the skill of those in the art (see, e.g., Ausubel et al. Current Protoc. Mol. Biol.).

In certain embodiments, the antibody comprises two glutamine residues, one in each heavy chain. In particular embodiments, the antibody comprises a Q295 residue in each heavy chain. In further embodiments, the antibody comprises one, two, three, four, five, six, seven, eight, or more glutamine residues. These glutamine residues can be in heavy chains, light chains, or in both heavy chains and light chains. Exemplary glutamine residues include Q55. These glutamine residues can be wild-type residues, or engineered residues. The antibodies can be prepared according to standard techniques.

Those of skill will recognize that antibodies are often glycosylated at residue N297, near residue Q295 in a heavy chain sequence. Glycosylation at residue N297 can interfere with a transglutaminase at residue Q295 (Dennler et al., supra) in some cases. Accordingly, in advantageous embodiments, the antibody is not glycosylated. In certain embodiments, the antibody is deglycoslated or aglycosylated. In particular embodiments, an antibody heavy chain has an N297 mutation. Alternatively stated, the antibody is mutated to no longer have an asparagine residue at position 297. In particular embodiments, an antibody heavy chain has an N297Q mutation. Such an antibody can be prepared by site-directed mutagenesis to remove or disable a glycosylation sequence or by site-directed mutagenesis to insert a glutamine residue at a site without resulting in disabled antibody function or binding. In some embodiments, an antibody having a Q295 residue and/or an N297Q mutation contains one or more additional naturally occurring glutamine residues in their variable regions, which can be accessible to transglutaminase and therefore capable of conjugation to a linker or a linker-payload. An exemplary naturally occurring glutamine residue can be found, e.g., at Q55 of the light chain. In such instances, the antibody conjugated via transglutaminase can have a higher than expected DAR value (e.g., a DAR higher than 4) and/or unexpected pharmacological properties and therapeutic advantages. Any such antibodies can be isolated from natural or artificial sources.

The antibody without interfering glycosylation is then reacted with a primary amine compound. In certain embodiments, an aglycosylated antibody is reacted with a primary amine compound to produce a glutaminyl-modified antibody. In certain embodiments, a deglycosylated antibody is reacted with a primary amine compound to produce a glutaminyl-modified antibody.

The primary amine can be any primary amine that is capable of forming a covalent bond with a glutamine residue in the presence of a transglutaminase. Useful primary amines are described below. The transglutaminase can be any transglutaminase deemed suitable by those of skill in the art. In certain embodiments, the transglutaminase is an enzyme that catalyzes the formation of an isopeptide bond between a free amine group on the primary amine compound and the acyl group on the side chain of a glutamine residue. Transglutaminase is also known as protein-glutamine-y-glutamyltransferase. In particular embodiments, the transglutaminase is classified as EC 2.3.2.13. The transglutaminase can be from any source deemed suitable. In certain embodiments, the transglutaminase is microbial. Useful transglutaminases have been isolated from Streptomyces mobaraense, Streptomyces cinnamoneum, Streptomyces griseo-carneum, Streptomyces lavendulae, and Bacillus subtilis. Non-microbial trans glutaminases, including mammalian transglutaminases, can also be used. In certain embodiments, the transglutaminase can be produced by any technique or obtained from any source deemed suitable by the practitioner of skill. In particular embodiments, the transglutaminase is obtained from a commercial source.

In particular embodiments, the primary amine compound comprises a reactive group capable of further reaction after transglutamination. In these embodiments, the glutaminyl-modified antibody can be reacted or treated with a reactive payload compound or a reactive linker-payload compound to form an antibody-payload conjugate. In certain embodiments, the primary amine compound comprises an azide.

In certain embodiments, the glutaminyl-modified antibody is reacted or treated with a reactive linker-payload to form an antibody-payload conjugate. The reaction can proceed under conditions deemed suitable by those of skill in the art. In certain embodiments, the glutaminyl-modified antibody is contacted with the reactive linker-payload compound under conditions suitable for forming a bond between the glutaminyl-modified antibody and the linker-payload compound. Suitable reaction conditions are well known to those in the art.

In certain embodiments, selective transglutamination at a specific glutamine residue of the heavy chain or the light chain over other glutamine residues is preferable and advantageous in terms of pharmacological properties. In certain embodiments, selective transglutamination means conjugation a ligand at the light chain Q55 specifically, but not at Q295. In certain embodiments, selective transglutamination means conjugation a ligand at the light chain Q295 specifically, but not at Q55. And in some other embodiments, selective transglutamination means conjugation means conjugation of a ligand at a specific glutamine residue introduced by recombinant technology. In certain embodiments, a modifiable glutamine residue is introduced by a glutamine tag (Q-tag).

In some instance, selective transglutamination is achieved naturally due to the differentiated accessibility of the glutamine residues, such as selective Q55 or Q-Tag modification/conjugation over Q295 in the presence of glycosylation at N297. In some other instances, selective transglutamination is achievable through a combination of chemical and biochemical process optimization and manipulation. In some instance, a reactive glutamine residue is selectively modified or blocked first, and then the following ligand conjugation is forced to other available glutamine residues of the target antibody to obtain the desired regio-selectivity.

In some embodiments, the reactivity and/or modifiability of certain glutamine residue depends on its spatial environment and the protein sequence of the antibody or antigen binding fragment thereof. For example, antibodies with Q55 do not show the same reactivity toward micro transglutaminase assisted conjugation. Q55 in some antibodies shows a better reactivity than that of others.

The location of conjugation of a ligand/drug to an antibody or antigen binding fragment thereof affects, and to a certain degree, determines the biological activity of the final conjugated product. Modification of a glutamine residue of an antibody or antigen binding fragment thereof with a ligand may lead to a conjugated product with an inactive or much reduced activity toward the target of the ligand. And in some embodiments, conjugation at Q55 affords a more active ADC product than that of conjugation at other glutamine residues. In certain embodiments, an antibody with a Phe46 of the light chain provides a better conjugation efficiency at its Q55 position.

In certain embodiments, those antibodies with a modifiable Q55 comprise anti-GDF8 antibodies, GLP1R antibodies, anti-Activin A/B antibodies, anti-ActRIIA/B antibodies, anti-MSR1 antibodies, anti-CACNG1 antibodies, and antibodies to the following targets CD20, CD226, MERS, HLA-B27, IL-6R, STEAP2, MET×MET, FGFR2b, FLT3, KIT, EGFR, CD1B, PMSA, NECTIN4, FOLR1, EGFRvIII, VPREB1, and LEPR. In certain embodiments, those antibodies with a modifiable Q55 comprise REGN1033 (anti-GDF8 antibody), REGN4320 (anti-MSR1 N297Q), REGN4322 (H1H21231N, anti-MSR1, N297Q), H2aM21339N (anti-HLA-A2/CMV), H4H11283N (anti-HLA-B27), H4sH14137N (anti-CD20), H1H20918P (CD226), H4H20122P (HLA-B27), H4H13767P (HFE2), H4H12587P (IL6R), H4H11281N (HLA-B27), and H1H15208P (MERS).

### Therapeutic Formulation and Administration

The present disclosure provides pharmaceutical compositions comprising the GLP1R agonist-tethered GDF8 antibody conjugates of the present disclosure.

In one aspect, the present disclosure provides compositions comprising a population of GLP1R agonist-tethered GDF8 antibody conjugates according to the present disclosure having a drug-antibody ratio (DAR) of about 0.5 to about 30.0.

In one embodiment, the composition has a DAR of about 1.0 to about 2.5.

In one embodiment, the composition has a DAR of about 2.

In one embodiment, the composition has a DAR of about 3.0 to about 4.5.

In one embodiment, the composition has a DAR of about 4.

In one embodiment, the composition has a DAR of about 6.5 to about 8.5.

In one embodiment, the composition has a DAR of about 8.

The present disclosure provides pharmaceutical dosage form comprising the GLP1R agonist-tethered GDF8 antibody conjugates of the present disclosure or the pharmaceutical compositions of the present disclosure.

The compositions of the disclosure are formulated with suitable carriers, excipients, diluents, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of a GLP1R agonist-tethered GDF8 antibody conjugate administered to a patient may vary depending upon the age and the size of the patient, target disease, conditions, route of administration, and the like. The suitable dose is typically calculated according to body weight or body surface area. When a GLP1R agonist-tethered GDF8 antibody conjugate of the present disclosure is used for therapeutic purposes in an adult patient, it may be advantageous to intravenously administer the GLP1R agonist-tethered GDF8 antibody conjugate of the present disclosure normally at a single dose of about 0.01 to about 20 mg/kg body weight, more preferably about 0.02 to about 7 mg/kg body weight, about 0.03 to about 5 mg/kg body weight, or about 0.05 to about 3 mg/kg body weight. Depending on the severity of the condition, the frequency and the duration of the treatment can be adjusted. Effective dosages and schedules for administering an GLP1R agonist-tethered GDF8 antibody conjugate may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the GLP1R Agonist-Tethered GDF8 Antibody Conjugates

In another aspect, the GLP1R agonist-tethered GDF8 antibody conjugates, disclosed herein are useful, inter alia, for the treatment, prevention and/or amelioration of a disease, disorder, or condition in need of such treatment.

In one aspect, the present disclosure provides a method of treating a condition in a subject in need thereof comprising administering to the subject a therapeutically effective amount of an GLP1R agonist-tethered GDF8 antibody conjugate according to the disclosure, or the composition comprising any compound according to the present disclosure.

In some embodiments, the GLP1R agonist-tethered GDF8 antibody conjugates disclosed herein are useful for treating any disease or disorder in which stimulation, activation, and/or targeting of GLP1R would be beneficial. In particular, the GLP1R agonist-tethered GDF8 antibody conjugate of the present disclosure can be used for the treatment, prevention, and/or amelioration of any disease or disorder associated with or mediated by GLP1R expression or activity.

In some embodiments, the GLP1R agonist-tethered GDF8 antibody conjugates disclosed herein are useful for treating a GLP1R-associated condition. In some embodiments, the GLP1R-associated condition is Type 1 or Type 2 diabetes mellitus. The administered GLP1R agonist-tethered GDF8 antibody conjugate may cause at least one of the following results: induction of insulin secretion, suppression of glucagon release, reduction of blood sugar, improvement of glycemic control, maintenance or increase in lean body mass while reducing fat mass, promotion of islet neogenesis, and delay of gastric emptying or potentiation of glucose resistant islets.

In some embodiments, the GLP1R-associated condition is a neurodegenerative disorder, a cognitive disorder, memory disorder, or learning disorder. The neurodegenerative disorder may be, for example, dementia, senile dementia, mild cognitive impairment, Alzheimer-related dementia, Huntington's chores, tardive dyskinesia, hyperkinesias, mania, Morbus Parkin-son, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis, glaucoma, and Alzheimer's disease.

In some embodiments, the GLP1R-associated condition is a liver disease. The liver disease may be, for example, non-alcoholic fatty liver disease (NAFLD), fatty liver, non-alcoholic steatohepatitis (NASH), and cirrhosis.

In some embodiments, the GLP1R-associated condition is a coronary artery disease. The coronary artery disease may be, for example, cardiomyopathy and myocardial infarction.

In some embodiments, the GLP1R-associated condition is obesity.

In some embodiments, the GLP1R-associated condition is a kidney disease. The kidney disease may be, for example, hypertension or chronic kidney failure.

In some embodiments, the GLP1R-associated condition is an eating disorder. The eating disorder may be, for example, binge eating.

Without wishing to be bound by theory, the compounds (e.g., an GLP1R agonist-tethered GDF8 antibody conjugate) disclosed herein may be employed to attenuate the effects of apoptosis-mediated degenerative diseases of the central nervous system such as Alzheimer's Disease, Creutzfeld-Jakob Disease and bovine spongiform encephalopathy, chronic wasting syndrome and other prion mediated apoptotic neural diseases (see, e.g., Perry and Grieg (2004) Current Drug Targets 6:565-571). Administration of a compound (e.g., an antibody-drug conjugate) disclosed herein may also lead to down-modulation of βAPP and thereby ameliorate Aβ mono- or oligomer-mediated pathologies associated with Alzheimer's Disease (see, e.g., Perry et al. (2003) Journal of Neuroscience Research 72:603-612).

In some embodiments, the GLP1R agonist-tethered GDF8 antibody conjugates disclosed herein may also be used to treat a metabolic disorder. The metabolic disorder may be, for example, obesity, dyslipidemia, metabolic syndrome X, and pathologies emanating from islet insufficiency.

It is also contemplated that the GLP1R agonist-tethered GDF8 antibody conjugates disclosed herein may be used to treat obesity, for example, by reducing body weight while maintaining or increasing lean body mass in a subject.

In some embodiments, the GLP1R agonist-tethered GDF8 antibody conjugates disclosed herein may also be used to treat obesity by reducing fat mass while maintaining or increasing lean body mass in a subject.

In some embodiments, the GLP1R agonist-tethered GDF8 antibody conjugates disclosed herein may also be used to treat Type 2 diabetes by improving glycemic control and maintaining or increasing lean body mass while reducing fat mass in a subject.

In some embodiments, the GLP1R agonist-tethered GDF8 antibody conjugates disclosed herein may also be used to treat obesity and Type 2 diabetes by improving glycemic control and maintaining or increasing lean body mass while reducing fat mass in a subject.

In some embodiments, the GLP1R agonist-tethered GDF8 antibody conjugates disclosed herein may also be used to treat obesity, diabetes, and/or liver diseases associated with increased fat mass in a subject.

In some embodiments, the GLP1R agonist-tethered GDF8 antibody conjugates disclosed herein may also be used to treat a subject of metabolic syndrome by improving glycemic control, maintaining or increasing lean body mass while reducing fat mass in a subject.

Additional diseases that may be treated by an GLP1R agonist-tethered GDF8 antibody conjugate of the present disclosure include autoimmune diseases, in particular, those associated with inflammation, including, but not limited to, autoimmune diabetes, adult onset diabetes, morbid obesity, Metabolic Syndrome X, and dyslipidemia. For example, the anti-GLP1R antibody-drug conjugate can be employed as a growth factor for the promotion of islet growth in persons with autoimmune diabetes. The GLP1R agonist-tethered GDF8 antibody conjugates described herein may also be useful in the treatment of congestive heart failure.

In one aspect, the present disclosure provides a method of selectively targeting an antigen (e.g., GLP1R) on a surface of a cell with a compound. In one embodiment, the method of selectively targeting an antigen (e.g., GLP1R) on a surface of a cell with a compound comprises linking the compound to a targeted antibody. In one embodiment, the compound is a payload as described above. In one embodiment, the cell is a mammalian cell. In one embodiment, the cell is a human cell. In one embodiment, the cell is a pancreatic cell or a brain cell. In another embodiment, the cell is a heart cell, a vascular tissue cell, a kidney cell, an adipose tissue cell, a liver cell, or a muscle cell.

In one aspect, the present disclosure provides a method of enhancing GLP1R activity in an individual in need thereof comprising administering to the individual an effective amount of the compound of any of the embodiments described herein, the composition described herein, or the dosage form described herein.

In certain embodiments, the present disclosure also includes a method of lowering blood glucose levels in an individual in need thereof comprising administering to the individual an effective amount of the compound of any of the embodiments described herein, the composition described herein, or the dosage form described herein.

In certain embodiments, the present disclosure also includes a method of lowering body weight in an individual in need thereof comprising administering to the individual an effective amount of the compound of any of the embodiments described herein, the composition described herein, or the dosage form described herein.

In certain embodiments, the present disclosure also includes the use of an GLP1R agonist-tethered GDF8 antibody conjugate of the present disclosure in the manufacture of a medicament for the treatment of a disease or disorder (e.g., cancer) related to or caused by GLP1R-expressing cells. In one aspect, the present disclosure relates to an GLP1R agonist-tethered GDF8 antibody, as disclosed herein, for use in medicine. In one aspect, the present disclosure relates to a compound comprising an GLP1R agonist-tethered GDF8 antibody conjugate as disclosed herein, for use in medicine.

### Combination Therapies and Formulations

The present disclosure provides methods which comprise administering a pharmaceutical composition comprising any of the exemplary GLP1R agonist-tethered GDF8 antibody conjugates described herein in combination with one or more additional therapeutic agents.

Exemplary additional therapeutic agents that may be combined with or administered in combination with GLP1R agonist-tethered GDF8 antibody conjugates of the present disclosure include, other GLP1R agonists (e.g., an anti-GLP1R antibody or a small molecule agonist of GLP1R or an anti-GLP1R antibody-drug conjugate). Non-limiting examples of GLP1R agonists include exenatide (Byetta, Bydureon), liraglutide (Victoza, Saxenda), lixisenatide (Lyxumia in Europe, Adlyxin in the United States), albiglutide (Tanzeum), dulaglutide (Trulicity), semaglutide (Ozempic), and taspoglutide.

Exemplary additional therapeutic agents may include dual or triple-agonists, including GLP1R/GIPR dual agonists, such as GLP1R/GCGR dual agonists, GLP1R/GIPR/GCGR triple-agonists.

Other agents that may be beneficially administered in combination with the GLP1R agonist-tethered GDF8 antibody conjugates of the disclosure include those that are useful in the treatment of diabetes (e.g., type II diabetes), obesity, and/or other related metabolic diseases.

In some embodiments, the additional therapeutic agent is an antidiabetic agent. Any suitable antidiabetic agents can be used. Non-limiting examples of antidiabetic agents include insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones or TZDs (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, glucokinase activators, squalene synthase inhibitors, other lipid lowering agents and aspirin. In some such embodiments, the antidiabetic agent is an oral antidiabetic agents (OAA) such as metformin, acarbose, or TZDs. In some such embodiments, the antidiabetic agent is metformin.

In some embodiments, the GLP1R agonist and one or more antidiabetic agents may be formulated into the same dosage form, such as a solution or suspension for parenteral administration.

The additional therapeutically active component(s) may be administered just prior to, concurrent with, or shortly after the administration of a compound of the present disclosure; (for purposes of the present disclosure, such administration regimens are considered the administration of an antigen-binding molecule “in combination with” an additional therapeutically active component).

The present disclosure includes pharmaceutical compositions in which GLP1R agonist-tethered GDF8 antibody conjugates of the present disclosure are co-formulated with one or more of the additional therapeutically active component(s) as described elsewhere herein.

### Administration Regimens

According to certain embodiments of the present disclosure, multiple doses of an GLP1R agonist-tethered GDF8 antibody conjugate may be administered to a subject over a defined time course. The methods according to this aspect of the disclosure comprise sequentially administering to a subject multiple doses of a an GLP1R agonist-tethered GDF8 antibody conjugate of the disclosure. As used herein, “sequentially administering” means that each dose of an GLP1R agonist-tethered GDF8 antibody conjugate is administered to the subject at a different point in time, e.g., on different days separated by a predetermined interval (e.g., hours, days, weeks, or months). The present disclosure includes methods which comprise sequentially administering to the patient a single initial dose of an GLP1R agonist-tethered GDF8 antibody conjugate, followed by one or more secondary doses of the GLP1R agonist-tethered GDF8 antibody conjugate, and optionally followed by one or more tertiary doses of the an GLP1R agonist-tethered GDF8 antibody conjugate.

The terms “initial dose,” “secondary doses,” and “tertiary doses,” refer to the temporal sequence of administration of the GLP1R agonist-tethered GDF8 antibody conjugate of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the GLP1R agonist-tethered GDF8 antibody conjugate, but generally may differ from one another in terms of frequency of administration. In certain embodiments, however, the amount of the GLP1R agonist-tethered GDF8 antibody conjugate contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of a GLP1R agonist-tethered GDF8 antibody conjugate which is administered to a patient prior to the administration of the very next dose in the sequence with no intervening doses.

The methods according to this aspect of the disclosure may comprise administering to a patient any number of secondary and/or tertiary doses of a GLP1R agonist-tethered GDF8 antibody conjugate. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### SEQUENCE LISTING

The sequences referred to herein have SEQ ID NOs and sequences as shown in the following informal sequence table:

| | SEQ | |
|---|---|---|
| | ID NO | amino acid sequence |
| GDF8 (mature | 1 | Asp Phe Gly Leu Asp Cys Asp Glu His Ser Thr Glu Ser |
| protein, myostatin) | | Arg Cys Cys Arg Tyr Pro Leu Thr Val Asp Phe Glu Ala |
| | | Phe Gly Trp Asp Trp Ile Ile Ala Pro Lys Arg Tyr Lys Ala |
| | | Asn Tyr Cys Ser Gly Glu Cys Glu Phe Val Phe Leu Gln |
| | | Lys Tyr Pro His Thr His Leu Val His Gln Ala Asn Pro Arg |
| | | Gly Ser Ala Gly Pro Cys Cys Thr Pro Thr Lys Met Ser Pro |
| | | Ile Asn Met Leu Tyr Phe Asn Gly Lys Glu Gln Ile Ile Tyr |
| | | Gly Lys Ile Pro Ala Met Val Val Asp Arg Cys Gly Cys Ser |
| glucagon-like | 2 | HAEGTFTSDVSSYLEGQAAKEFIAWLVKGRG |
| peptide-1(GLP-1) | | His Ala Glu Gly Thr Phe Thr Ser Asp Val Ser Ser Tyr Leu |
| (7-37) | | Glu Gly Gln Ala Ala Lys Glu Phe Ile Ala Trp Leu Val Lys |
| | | Gly Arg Gly |
| GLPIR agonist | 3 | H(Aib)EGTFTSDYSSYLEEQAAKEFIAWLVKGGG |
| (Payload) | | His Aib Glu Gly Thr Phe Thr Ser Asp Tyr Ser Ser Tyr Leu |
| | | Glu Glu Gln Ala Ala Lys Glu Phe Ile Ala Trp Leu Val Lys |
| | | Gly Gly Gly |
| anti-GDF8 ab HCVR | 4 | Glu Val Gln Val Leu Glu Ser Gly Gly Asp Leu Val Gln Pro |
| | | Gly Gly Ser Leu Arg Leu Ser Cys Ala Ala Ser Gly Phe Thr |
| | | Phe Ser Ala Tyr Ala Met Thr Trp Val Arg Gln Ala Pro Gly |
| | | Lys Gly Leu Glu Trp Val Ser Ala Ile Ser Gly Ser Gly Gly |
| | | Ser Ala Tyr Tyr Ala Asp Ser Val Lys Gly Arg Phe Thr Ile |
| | | Ser Arg Asp Asn Ser Lys Asn Thr Val Tyr Leu Gln Met |
| | | Asn Ser Leu Arg Ala Glu Asp Thr Ala Val Tyr Tyr Cys Ala |
| | | Lys Asp Gly Ala Trp Lys Met Ser Gly Leu Asp Val Trp Gly |
| | | Gln Gly Thr Thr Val Ile Val Ser Ser |
| anti-GDF8 ab LCVR | 5 | Asp Ile Gln Met Thr Gln Ser Pro Ala Ser Leu Ser Ala Ser |
| | | Val Gly Asp Arg Val Thr Ile Thr Cys Arg Ala Ser Gln Asp |
| | | Ile Ser Asp Tyr Leu Ala Trp Tyr Gln Gln Lys Pro Gly Lys |
| | | Ile Pro Arg Leu Leu Ile Tyr Thr Thr Ser Thr Leu Gln Ser |
| | | Gly Val Pro Ser Arg Phe Arg Gly Ser Gly Ser Gly Thr Asp |
| | | Phe Thr Leu Thr Ile Ser Ser Leu Gln Pro Glu Asp Val Ala |
| | | Thr Tyr Tyr Cys Gln Lys Tyr Asp Ser Ala Pro Leu Thr Phe |
| | | Gly Gly Gly Thr Lys Val Glu Ile Lys |
| anti-GDF8 ab | 6 | Gly Phe Thr Phe Ser Ala Tyr Ala |
| HCDR1 | | |
| anti-GDF8 ab | 7 | Ile Ser Gly Ser Gly Gly Ser Ala |
| HCDR2 | | |
| anti-GDF8 ab | 8 | Ala Lys Asp Gly Ala Trp Lys Met Ser Gly Leu Asp Val |
| HCDR3 | | |
| anti-GDF8 ab | 9 | Gln Asp Ile Ser Asp Tyr |
| LCDR1 | | |
| anti-GDF8 ab | | Thr Thr Ser |
| LCDR2 | | |
| anti-GDF8 ab | 11 | Gln Lys Tyr Asp Ser Ala Pro Leu Thr |
| LCDR3 | | |
| M6457 | 140 | H[Aib]EGTFTSDYSSYLEEQAAKEFIAWLVKGGG |
| | | (G4S)3-K(Ac-Br)-amide |
| M6562 | 13 | H[Aib]EGTFTSDYSSYLEEQAAKEFIAWLVKGGG |
| | | (G4S)3-K(Ac-COT)-amide |
| M6588 | 14 | H[Aib]EGTFTSDYSSYLEEQAAKEFIAWLVKGGG |
| | | (G4S)2-K(Ac-COT)-amide |
| M6589 | 15 | H[Aib]EGTFTSDYSSYLEEQAAKEFIAWLVKGGG |
| | | (G4S)-K(Ac-COT)-amide |
| M6570 | 16 | H[Aib]EGTFTSDYSSYLEEQAAKEFIAWLVKGGG |
| | | (G4S)3-K(K(N3))-amide |
| M6557 | 17 | H[Aib]EGTFTSDYSSYLEEQAAKEFIAWLVKGGG |
| | | (G4S)3-K(Ac-Mc)-amide |
| M6677 | 18 & | H[Aib]EGTFTSDYSSYLEEQAAKEFIAWLVKGGG |
| | 136 | K[TA-(G4S)3-K(Ac-COT)]-amide |
| M6675 | 19 | H[Aib]EGTFTSDYSSYLEEQAAKEFIAWLVKGGG- |
| | | K(N3)-amide |
| | 20 | H(Aib)EGTFTSDYSSYLEEQAAKEFIAWLVKGGG- |
| | | GGGGSGGGGSGGGGS-K[Propanoyl-Mc] amide |
| | | (Payload -(G₄S)₃-K[Pr-Mc]) |

### EXAMPLES

The following examples illustrate specific aspects of the instant description. The examples should not be construed as limiting, as the examples merely provide specific understanding and practice of the embodiments and their various aspects.

The abbreviations used in the Examples and throughout the specification are shown in Table 1.

TABLE 1
Abbreviations

| | |
|---|---|
| AA | Amino acid |
| ACN | Acetonitrile |
| amAA | α-Methylated amino acid |
| Boc | tert-Butyloxycarbonyl |
| DCM | Dichlormethane |
| Dde | 1-(4,4-diMethyl-2,6-dioxocyclohexylidene)ethyl |
| DIPEA | Diisopropylethylamine |
| DIC | N,N′-Diisopropylcarbodiimide |
| DMF | N,N-Dimethylformamide |
| Fmoc | 9-Fluorenylmethyloxycarbonyl |
| MeOH | Methanol |
| Mc-COOH | 2-maleimido acetic acid |
| HATU | 2-(7-Azabenzotriazol-1-yl)-N,N,N′,N′- |
| | tetramethyluronium hexafluorophosphate |
| HBTU | O-Benzotriazole-N,N,N',N'-tetramethyl-uronium- |
| | hexafluorophosphate |
| HOBT | 1-Hydroxybenzotriazole |
| HPLC | High-performance liquid chromatography |
| MPA | 3-Mercaptopropionicacid |
| MTBE | Methyl tert-Butyl Ether |
| t-Bu | tert-Butyl |
| TEA | Triethylamine |
| TFA | Trifluoroacetic acid |
| TIS | Triisopropylsilane |
| Trt | Trityl |
| Pip | Piperidine |

### Example 1. Materials and Methods

### General Procedures for the Synthesis of Peptides

### Peptide Synthesis

Peptides were synthesized using standard Fmoc chemistry. Most peptides were synthesized with a scale of 0.05 mmol based on the initial loading of the first amino acid.

### Resin Preparation

A solution of Rink Amide MBHA Resin (1.0 eq, Sub 0.33 mmol/g) in DMF (50.0 mL/mmol of Resin) was agitated with N2 at 25° C. for 2 hours. Then 20% piperidine in DMF (50.0 mL mmol of Resin) was added and the reaction mixture was agitated with N2 at 25° C. for 15 min. The resin was washed with DMF (50.0 mL/mmol of Resin for 5 times).

Alternatively, the Rink-amide MBHA resin (initial resin loading 0.33 mmol/g) was swelled with DCM (10 mL/gram pf Resin) briefly and then drained. This was repeated for four times. A solution of 20% piperidine in DMF was then added to the resin. The mixture was agitated twice for 20 min. After the reaction was completed, the resin was washed with DMF (2×5 mL).

### Amide Coupling Reaction

Coupling the 1^{st} AA (Lys as an example): A solution of Oxyma (3.0 eq) and Fmoc-Lys(Dde)-OH (3.0 eq) in DMF (25.0 mL/mmol of Resin) was added to the resin, then DIC (3.0 eq) was added, and the mixture was agitated with N2 at 25° C. for 30 min. The resin was washed with DMF (50.0 mL/mmol of Resin×5).

Coupling of native amino acid: Fmoc-AA-OH (3 eq.)/HBTU (3 eq.)/DIPEA (6 eq.) were dissolved in DMF with a final concentration of 0.2 mM. The mixture was agitated for 60-90 min at room temperature. After the reaction was completed, the resin was washed with DMF (2×5 mL).

Coupling of alpha-methylated AA (amAA): Fmoc-amAA-OH (3 eq.)/HATU (3 eq.)/DIPEA (6 eq.) were dissolved in DMF with a final concentration of 0.2 mM. The mixture was agitated for 16 hours at room temperature. After the reaction was completed, the resin was washed with DMF (2×5 mL).

Coupling of the amino acid after Aib: Fmoc-AA-OH (3 eq.)/HATU (3 eq.)/DIPEA (6 eq.) were dissolved in DMF with a final concentration of 0.2 mM. The mixture was agitated for 2×45 min at room temperature. After the reaction was completed, the resin was washed with DMF (2×5 mL).

Coupling of amino acid after amAA: Fmoc-AA-OH (20 eq.)/HATU (20 eq.)/DIPEA (30 eq.) were dissolved in DMF with a final concentration of 0.5 mM. The mixture was agitated for 16 hours at room temperature. Mini-cleavage LCMS was necessary to monitor the progress of the reaction. After the reaction was completed, the resin was washed with DMF (2×5 mL).

In some cases, after the removal of Fmoc protecting group from N-terminal amino acid, the free amine was protected with Boc-group.

Coupling of Mc-COOH: Mc-COOH (4 eq.)/DIC (4 eq.) were dissolved in DMF with a final concentration of 0.5 mM. The mixture was agitated twice for 40 min at room temperature. Mini-cleavage LCMS was necessary to monitor the progress of the reaction. After the reaction was completed, the resin was washed with DMF (2×5 mL).

### Deprotections

Fmoc deprotection: a solution of 20% piperidine in DMF (100.0 mL) was added to the resin and the resin was agitated with N2 at 25° C. for 15 min. The resin was washed with DMF (100.0 mL×5) and filtered.

Dde deprotection: The resin-bound peptide was treated with hydrazine monohydrate (3% N₂H₄·H₂O/DMF) (2 mmol scale) for 20 min twice and 10% in DMF in 10 mL for 0.05 mmol scale for 30 min. Once the reaction was completed, the resin was drained, and the resin was washed with DMF (100 mL×5 times for large scale) or wash with DMF (10 mL), MeOH (10 mL), DCM (2×10 mL) and DMF (2×10 mL).

### Cleavage

The resin bound peptide was cleaved using TFA/TIS/H₂O/MPA (17:1:1:1) for 1.5-2 hours or TFA/TIS/H₂O/DTT (16:1:1:2) at room temperature for 1.5-2 hours. For peptide containing Atz, 5 vol % of ethanedithiol was added into the cleavage cocktail. Once the reaction was completed, the resin was filtered and washed with small volume of TFA twice.

### Precipitation

The peptide was precipitated with cold isopropyl ether (1.50 L). The mixture was filtered and the filter cake was collected. The filter cake was washed twice with isopropyl ether (1.50 L) and dried under vacuum for 1 hour to afford the crude peptide.

Alternatively, the peptide was precipitated by addition of ten-fold volume of cold MTBE to the TFA solution and mixed thoroughly. The mixture was centrifuged. The supernatant was decanted. The solid containing crude peptide was further washed with MTBE twice, centrifuged, and then dried under reduced pressure.

### Purification

The crude peptide was purified by prep-HPLC (0.01% TFA/CH₃CN/H₂O) to afford the target peptide after lyophilization. Purification conditions are shown in Table 2 and Table 3.

TABLE 2
HPLC Conditions

| | |
|---|---|
| Dissolution condition | DMSO |
| Mobile Phase | A: H₂O |
| | B: CH₃CN |
| Gradient | 10-90-50 min., Retention time: 30 min |
| Column | Spherical, c18, 20-45 um, 100A |
| Flow Rate | 100 mL/Min |
| Wavelength | 220/254 nm |
| Oven Tem. | 30° C. |

TABLE 3
Alternative HPLC Conditions

| | |
|---|---|
| Dissolution condition | Dissolve in 30% ACN-H2O |
| Instrument | Gilson GX-281 |
| Mobile Phase | A: H₂O (0.075% TFA in H₂O) |
| | B: CH₃CN |
| Gradient | 25-45-50 min , Retention time: 28 min |
| Column | luna ,10 um, c18, 100A, 25 mm + Gemin, 5um, |
| | c18, 110A |
| Flow Rate | 20 mL/Min |
| Wavelength | 220/254 nm |
| Oven Tem. | 30° C. |

### Analytical Instruments

Analytical HPLC spectra were recorded with an Agilent 1200 series system. LCMS spectra were recorded with an Agilent 1200 series system coupled with an Agilent 6130 Quadrupole MS detector.

TABLE 4
Sequence Information for Lead Linker-Payload Peptides

| | | C-terminus | SEQ ID |
|---|---|---|---|
| Comp# | Payload (1-31) | linker | NO |
| M6457 | H[Aib]EGTFTSDYSSYLEEQAAKEFIAWLVKGGG | (G4S)3-K(Ac- | 140 |
| | | Br)-amide | |
| M6562 | H[Aib]EGTFTSDYSSYLEEQAAKEFIAWLVKGGG | (G4S)3-K(Ac- | 13 |
| | | COT)-amide | |
| M6588 | H[Aib]EGTFTSDYSSYLEEQAAKEFIAWLVKGGG | (G4S)2-K(Ac- | 14 |
| | | COT)-amide | |
| M6589 | H[ Aib]EGTFTSDYSSYLEEQAAKEFIAWLVKGGG | (G4S)-K(Ac- | 15 |
| | | COT)-amide | |
| M6570 | H[Aib]EGTFTSDYSSYLEEQAAKEFIAWLVKGGG | (G4S)3-K(KN3)- | 16 |
| | | amide | |
| M6557 | H[Aib]EGTFTSDYSSYLEEQAAKEFIAWLVKGGG | (G4S)3-K(Ac- | 17 |
| | | Mc)-amide | |
| M6677 | H[Aib]EGTFTSDYSSYLEEQAAKEFIAWLVKGGGK | [TA-(G4S)3- | 18 & |
| | | K(Ac-COT)]- | 136 |
| | | amide | |
| Payload | H[Aib]EGTFTSDYSSYLEEQAAKEFIAWL | | 3 |
| (1-31) | VKGGG | | |

TABLE 5
Summary of Properties for Lead Peptides

| | | |
|---|---|---|
| M Code | M6457 | M6562 |
| Batch No. | EW54293-1-P1 | EW54293-3-P1 |
| Molecular Formula | C196H290BrN55O69 | C204H301N55O70 |
| Exact Mass | 4597.01 | 4641.17 |
| Molecular Weight | 4600.70 | 4643.97 |
| Counter Ion | TFA | TFA |
| Formula Weight | 5056.78 (+4 TFA) | 5100.05 (+4 TFA) |
| Physical | White Powder | White Powder |
| Appearance | | |
| Storage | -20° C. | -20° C. |
| Weight | 215 mg | 252.1 mg |
| Peptide Sequence | H[Aib]EGTFTSDYSSYLEE | H[Aib]EGTFTSDYSSYLEEQAA |
| | QAAKEFIAWLVKGGGGG | KEFIAWLVKGGGGGGGSGGGG |
| | GGSGGGGSGGGGS-K(Ac- | SGGGGS-K(Ac-COT)-amide (SEQ |
| | Br)-amide (SEQ ID NO: 93) | ID NO: 94) |

TABLE 6
Summary of Properties for Lead Peptides

| M Code | M6588 | M6589 |
|---|---|---|
| Batch No. | EW42729-48-P1 | EW42729-48-P2 |
| Molecular Formula | C193H284N50O64 | C182H267N45O58 |
| Exact Mass | 4326.05 | 4010.93 |
| Molecular Weight | 4328.68 | 4013.40 |
| Counter Ion | TFA | TFA |
| Formula Weight | 4784.76 (+4 TFA) | 4469.48 (+4 TFA) |
| Physical | White Powder | White Powder |
| Appearance | | |
| Storage | -20° C. | -20° C. |
| Weight | 26.4 mg | 36.1 |
| Peptide Sequence | H[Aib]EGTFTSDYSSYLEE | H[Aib]EGTFTSDYSSYLEEQA |
| | QAAKEFIAWLVKGGGGG | AKEFIAWLVKGGGGGGGS- |
| | GGSGGGGS-K(Ac-COT)- | K(Ac-COT)-amide (SEQ ID NO: |
| | amide (SEQ ID NO: 95) | 96) |

TABLE 7
Summary of Properties for Lead Peptides

| | | |
|---|---|---|
| M Code | M6570 | M6557 |
| Batch No. | WSY-0041 | SXD-0043 |
| Molecular Formula | C₂₀₀H2₉₉N₅₉O₆₉ | C₂₀₀H₂₉₂N₅₆O₇₁ |
| Exact Mass | 4631.17 | 4614.10 |
| Molecular Weight | 4633.94 | 4616.86 |
| Counter Ion | TFA | TFA |
| Formula Weight | 5204.04 (+5 TFA) | 5072.94 (+4 TFA) |
| Physical Appearance | White Powder | White Powder |
| Storage | -20° C. | -2° C. |
| Weight | 10.0 mg | 10.0 mg |
| Peptide Sequence | H[Aib]EGTFTSDYSSYLEEQ | H[Aib]EGTFTSDYSSYLEEQ |
| | AAKEFIAWLVKGGG- | AAKEFIAWLVKGGG- |
| | (GGGGS)3-K(KN3)-amide | (GGGGS)3-K(Ac-Mc)-amide |
| | (SEQ ID NO: 97) | (SEQ ID NO: 98) |

TABLE 8
Summary of Properties for Lead Peptides

| M Code | M6677 | M6675 | M6694 |
|---|---|---|---|
| Batch No. | EW54293-1-P1 | WSY-107 | WSY-105 |
| Molecular | C₂₁₅H₃₁₈N₆₀O₇₂ | C₁₆₁H₂₃₆N₄₂O₅₀ | C₅₄H₈₂N₁₈O₂₂ |
| Formula | | | |
| Exact Mass | 4597.01 | 3557.72 | 1334.59 |
| Molecular | 4600.70 | 3559.90 | 1335.35 |
| Weight | | | |
| Counter Ion | TFA | TFA | NA |
| Formula | 5351.34 (+4 TFA) | 4015.98 (+4 TFA) | 1335.35 |
| Weight | | | |
| MS | 1632.3 [M + 3H]³⁺ | 1187.3 [M + 3H]³⁺ | 668.4 [M + 2H]2⁺ |
| Physical | White Powder | White Powder | White Powder |
| Appearance | | | |
| Storage | −20° C. | −20° C. | −20° C. |
| Weight | 65 mg | 215 mg | 460 mg |
| Peptide | H[Aib]EGTFTSDYS | H[Aib]EGTFTSDYS | pent-4-ynoyl |
| Sequence | SYLEEQAAKEFIA | SYLEEQAAKEFIA | GGGSGGGGSGGGG |
| | WLVKGGGK(TA-G | WLVKGGG-K(N₃)- | S-K(Ac-COT)-amide |
| | GGSGGGGSGGGG | amide (SEQ ID NO: | (SEQ ID NO: 101) |
| | S-K(Ac-COT))-amide | 100) | |
| | (SEQ ID NOS 99 & | | |
| | 138) | | |

*K(TA-): TA represents a triazole moiety formed by the alkyne moiety of M6694 and the azide K(N₃) moiety of M6675:

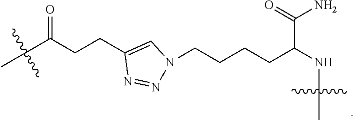 [SMILES: *NC(CCCCn1cc(CCC(*)=O)nn1)C(N)=O]

TABLE 9
Structures of Handles

| Code | MW | Structure |
|---|---|---|
| M404 | 218.14 | 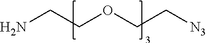 |
| M6092 | 201.23 | 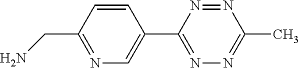 |
| M6093 | 363.42 | 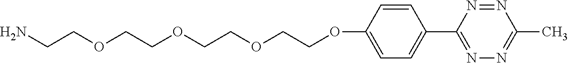 [SMILES: Cc1nnc(-c2ccc(OCCOCCOCCOCCN)cc2)nn1] |
| M2153 | 674.77 | 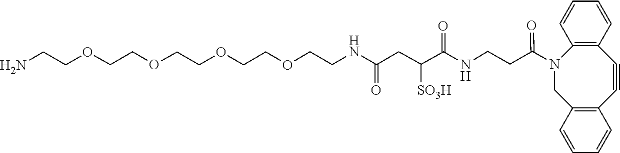 [SMILES: NCCOCCOCCOCCOCCNC(=O)CC(C(=O)NCCC(=O)N1Cc2ccccc2C#Cc2ccccc21)S(=O)(=O)O] |

TABLE 10
Lead GDF8 Antibody Tethered GLP1 Peptide Conjugates

| | | | | Handle | Linker-Payload | | | |
|---|---|---|---|---|---|---|---|---|
| ATL | Ab | Ab | Conj. | M | | M | ATL | |
| NO.* | Target | Name/Seq | Site | Code | LP # | Code | Name/structure | DAR |
| 1* | Anti- | REGN1033 | Cys | n/a | Payload- | M6457 | REGN1033- | 2.3 |
| | GDF8 | | | | (G4S)3-K(Ac-Br) | | M6457 (Cys) | |
| 2 | Anti- | REGN1033 | Q55 | M6092 | payload- | M6562 | REGN1033- | 2.0 |
| | GDF8 | | | | (G4S)3-K(Ac-COT) | | M6092-M6562 | |
| 3 | Anti- | REGN1033 | Q55 | M6092 | payload- | M6588 | REGN1033- | 1.9 |
| | GDF8 | | | | (G4S)2-K(Ac-COT) | | M6092-M6588 | |
| 4 | Anti- | REGN1033 | Q55 | M6092 | payload-(G4S)- | M6589 | REGN1033- | 2.0 |
| | GDF8 | | | | K(Ac-COT) | | M6092-M6589 | |
| 5 | Anti- | REGN1033 | Q55 | M6093 | payload- | M6562 | REGN1033- | 2.1 |
| | GDF8 | | | | (G4S)3-K(Ac-COT) | | M6093-M6562 | |
| 6 | Anti- | REGN1033 | Q55 | M2153 | payload- | M6570 | REGN1033- | 1.7 |
| | GDF8 | | | | (G4S)3-K(KN3) | | M2153-M6570 | |
| 7* | Anti- | REGN1033 | Q55 + | M6092 | payload- | M6562 | REGN1033- | 3.7 |
| | GDF8 | | Q295 | | (G4S)3-K(Ac-COT) | | M6092-M6562 | |
| 8* | Anti- | REGN1033 | Q295 | M6092 | payload- | M6562 | REGN1033- | 1.6 |
| | GDF8 | | | | (G4S)3-K(Ac-Br) | | M6092-M6562 | |
| 9* | Isotype | REGN1945 | Cys | n/a | payload- | M6457 | H4H30045P- | 1.8 |
| | | | | | (G4S)3-K(Ac-COT) | | M6457 | |
| 10 | Isotype | H4H30045P | Q55 | M6092 | payload- | M6562 | H4H30045P- | 2.0 |
| | | | | | (G4S)3-K(Ac-COT) | | M6092-M6562 | |
| 11 | Isotype | H4H30045P | Q55 | M6092 | payload- | M6588 | H4H30045P- | 1.7 |
| | | | | | (G4S)2-K(Ac-COT) | | M6092-M6588 | |
| 12 | Isotype | H4H30045P | Q55 | M6092 | payload-(G4S)- | M6589 | H4H30045P- | 1.7 |
| | | | | | K(Ac-COT) | | M6092-M6589 | |
| 13 | Isotype | H4H30045P | Q55 | M6093 | COMP P- | M6562 | H4H30045P- | 2.2 |
| | | | | | (G4S)3-K(Ac-COT) | | M6093-M6562 | |
| 14 | Isotype | H4H30045P | Q55 | M2153 | COMP P- | M6570 | H4H30045P- | 1.6 |
| | | | | | (G4S)3-K(KN3)* | | M2153-M6570 | |
| 15* | Isotype | H4H30045P | Q55 + | M6092 | COMP P- | M6562 | H4H30045P- | 3.6 |
| | | | Q295 | | (G4S)3-K(Ac-COT) | | M6092-M6562 | |
| 16 | Anti- | REGN1033 | Q55 | M6092 | Payload-K(TA*- | M6677 | REGN1033- | 2.0 |
| | GDF8 | | | | (G4S)3-K(Ac-COT)) | | M6092-M6677 | |
| 17 | isotype | H4H30045P | Q55 | M6092 | Payload-K(TA- | M6677 | H4H30045P- | 2.0 |
| | | | | | (G4S)3-K(Ac-COT)) | | M6092-M6677 | |

*ATLs with a deglycosylated antibody: 1,7, 8, 9, and 15; K(N3) represent an amino acid residue where the e-amino replaced with an azido moiety; *K(TA-): TA represents a triazole moiety formed by Click Reaction between the alkyne moiety of M6694 and the azide K(N₃) moiety of M6675:

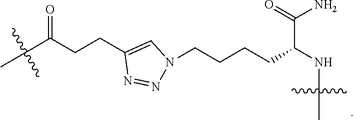 [SMILES: *N[C@H](CCCCn1cc(CCC(*)=O)nn1)C(N)=O]

TABLE 11
Structures of the Prepared Antibody-Tethered Ligands (ATLs, for clarity only one structure is listed for those with multiple regio-isomers)

| ATL | |
|---|---|
| No. | Structures of Lead ATLs |
| 1 | REGN1033-M6457 (conjugation through side chains of Cys residues; antibody is deglycosylated) (SEQ ID NO: 114) |
| | 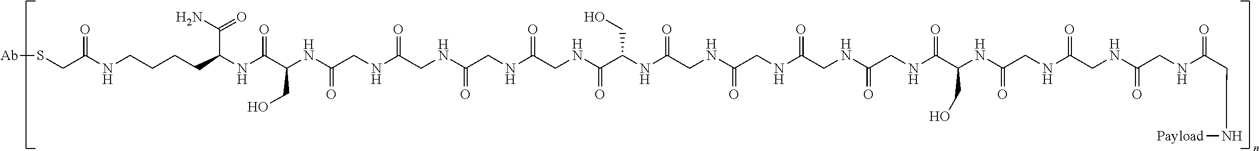 |
| 2 | REGN1033-M6092-M6562 (Q55, n = 2) (SEQ ID NO: 115) |
| | 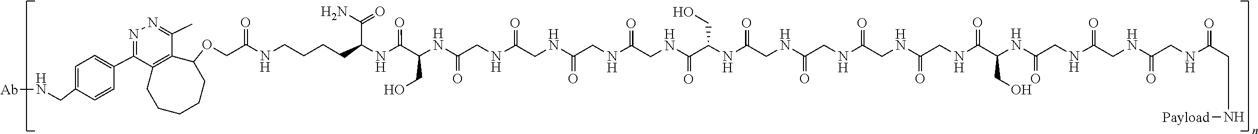 |
| 3 | REGN1033-M6092-M6588 (Q55) (SEQ ID NO: 117) |
| | 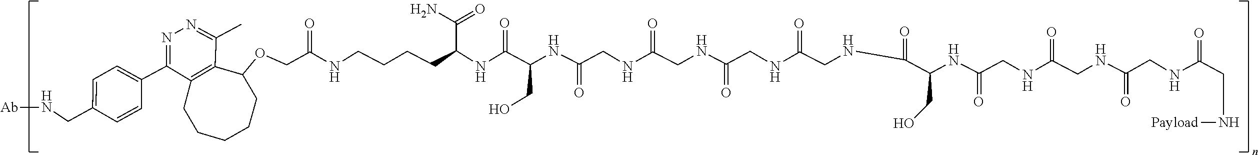 |
| 4 | REGN1033-M6092-M6589 (Q55) (SEQ ID NO: 119) |
| | 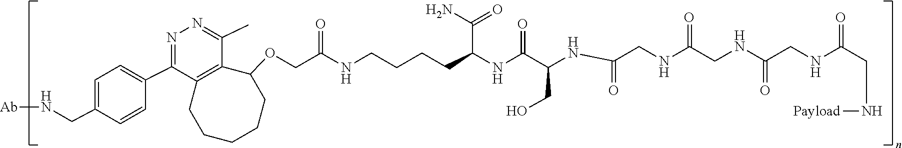 |
| 5 | REGN1033-M6093-M6562 (Q55) (SEQ ID NO: 121) |
| | 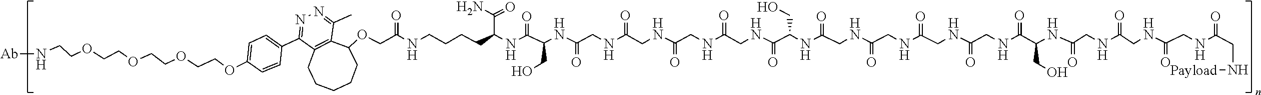 |
| 6 | REGN1033-M2153-M6570 (Q55) (SEQ ID NO: 123) |
| | 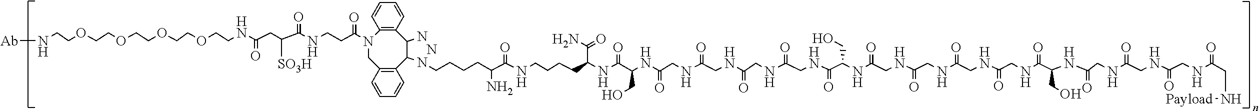 |
| 7 | REGN1033-M6092-M6562 (antibody is deglycosylated, Q55 + Q295, n = 4) (SEQ ID NO: 125) |
| | 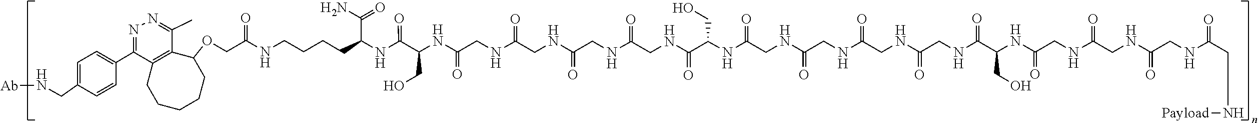 |
| 8 | REGN1033-M6092-M6562 (Q295, antibody is deglycosylated) (SEQ ID NO: 127) |
| | 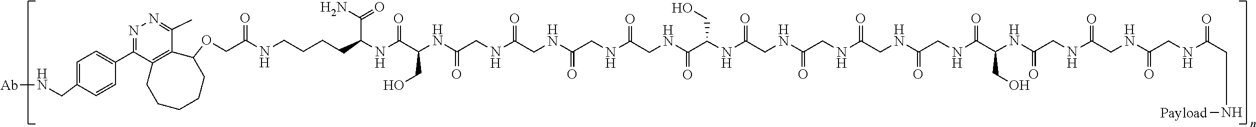 |
| 9 | H4H30045P-M6457 (Cys, antibody is deglycosylated) (SEQ ID NO: 129) |
| | 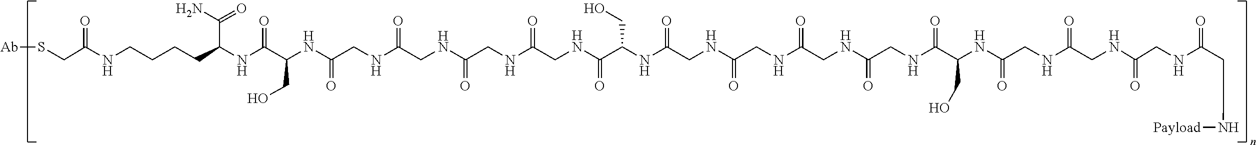 |
| 10 | H4H30045P-M6092-M6562 (Q55) (SEQ ID NO: 130) |
| | 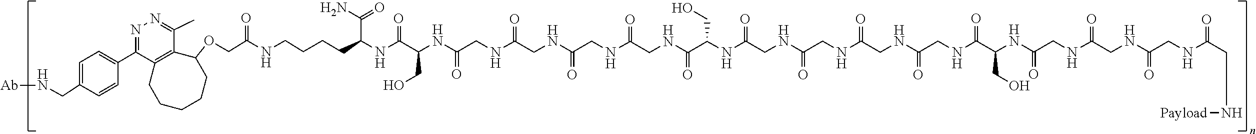 |
| 11 | Isotype antibody conjugate as a control, H4H30045P-M6092-M6588 (Q55) |
| 12 | Isotype antibody conjugate as a control, H4H30045P-M6092-M6589 (Q55) |
| 13 | Isotype antibody conjugate as a control, H4H30045P-M6093-M6562 (Q55) |
| 14 | Isotype antibody conjugate as a control, H4H30045P-M2153-M6570 (Q55) |
| 15 | Isotype antibody conjugate as a control, antibody is deglycosylated, H4H30045P-M6092-M6562 (Q55 + Q295) |
| 16 | REGN1033-M6092-M6677 (Q55) (SEQ ID NO: 132) |
| | 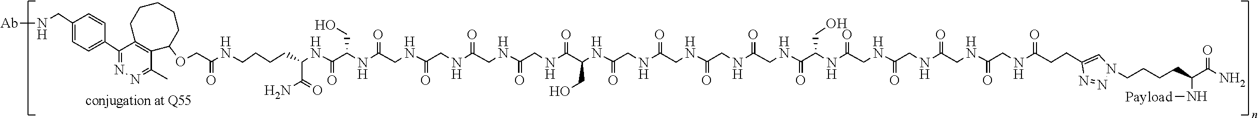 |
| 17 | Isotype antibody conjugate as a control, H4H30045P-M6092-M6677 (Q55) (SEQ ID NO: 134) |
| | Payload: H[Aib]EGTFTSDYSSYLEEQAAKEFIAWLVKGGGGGGGSGGGGSGGGG- (SEQ ID NO: 102) |
| | 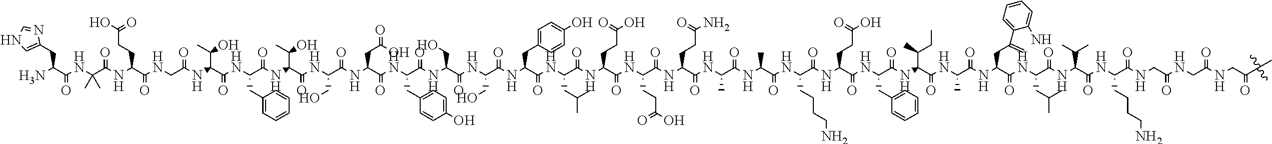 |

### Example 2. Synthesis of M6457

TABLE 12
Properties of M6457

| | |
|---|---|
| M Code | M6457 |
| Batch No. | EW54293-1-P1 |
| Molecular Formula | C196H290BrN55O69 |
| Exact Mass | 4597.01 |
| Molecular Weight | 4600.70 |
| Counter Ion | TFA |
| Formula Weight | 5056.78 (+4 TFA) |
| Physical | White Powder |
| Appearance | |
| Storage | -20° C. |
| Weight | 215 mg |
| Peptide Sequence | H[Aib]EGTFTSDYSSYLEEQAAKEFIA |
| | WLVKGGGGGGGSGGGGSGGGGS-K(Ac- |
| | Br)-CONH2 (SEQ ID NO: 103) |

TABLE 13
Reagents and Reaction Conditions

| | Materials: AA with Oxyma (3.0 eq), | Reaction |
|---|---|---|
| Step # | DIC (3.0 eq) | time |
| 1 | Fmoc-Lys(Dde)-OH (3.0 eq) | 80 min |
| 2 | Fmoc-Gly-Ser(PSI(Me,Me)Pro)-OH | 60 min |
| | (3.0 eq) | |
| 3 | Fmoc-Gly-Gly-Gly-OH (3.0 eq) | 80 min |
| 4 | Fmoc-Gly-Ser(PSI(Me,Me)Pro)-OH | 16 hours* |
| | (3.0 eq) | |
| 5 | Fmoc-Gly-Gly-Gly-OH (3.0 eq) | 90 min |
| 6 | Fmoc-Gly-Ser(PSI(Me,Me)Pro)-OH | 80 min |
| | (3.0 eq) | |
| 7 | Fmoc-Gly-Gly-Gly-OH (3.0 eq) | 60 min |
| 8 | Fmoc-DmbGly-OH (3.0 eq) | 16 hours |
| 9 | Fmoc-Gly-Gly-OH (3.0 eq) | 60 min |
| 10 | Fmoc-Lys(Boc)-OH (3.0 eq) | 60 min |
| 11 | Fmoc-Val-OH (3.0 eq) | 80 min |
| 12 | Fmoc-Leu-OH (3.0 eq) | 16 hours |
| 13 | Fmoc-Trp(Boc)-OH (3.0 eq) | 90 min |
| 14 | Fmoc-Ala-OH (3.0 eq) | 60 min |
| 15 | Fmoc-Ile-OH (3.0 eq) | 80 min |
| 16 | Fmoc-Phe-OH (3.0 eq) | 16 hours |
| 17 | Fmoc-Glu(OtBu)-OH (3.0 eq) | 80 min |
| 18 | Fmoc-Lys(Boc)-OH (3.0 eq) | 90 min |
| 19 | Fmoc-Ala-OH (3.0 eq) | 16 hours |
| 20 | Fmoc-Ala-OH (3.0 eq) | 120 min |
| 21 | Fmoc-Gln(Trt)-OH (3.0 eq) | 16 hours |
| 22 | Fmoc-Glu(OtBu)-OH (3.0 eq) | 100 min |
| 23 | Fmoc-Glu(OtBu)-OH (3.0 eq) | 16 hours |
| 24 | Fmoc-Leu-OH (3.0 eq) | 90 min |
| 25 | Fmoc-Tyr(tBu)-OH (3.0 eq) | 100 min |
| 26 | Fmoc-Ser(tBu)-OH (3.0 eq) | 16 hours |
| 27 | Fmoc-Ser(tBu)-OH (3.0 eq) | 100 min |
| 28 | Fmoc-Tyr(tBu)-OH (3.0 eq) | 120 min |
| 29 | Fmoc-Asp(OtBu)-OH (3.0 eq) | 16 hours |
| 30 | Fmoc-Ser(tBu)-OH (3.0 eq) | 100 min |
| 31 | Fmoc-Thr(tBu)-OH (3.0 eq) | 100 min |
| 32 | Fmoc-Phe-OH (3.0 eq) | 16 hours |
| 33 | Fmoc-Thr(tBu)-OH (3.0 eq) | 120 min |
| 34 | Fmoc-Gly-OH (3.0 eq) | 90 min |
| 35 | Fmoc-Giu(OtBu)-OH (3.0 eq) | 16 hours |
| 36 | Fmoc-Aib-OH (3.0 eq) | 90 min |
| 37 | Fmoc-His(Trt)-OH (3.0 eq) | 120 min |
| 38 | De-Fmoc: 20% Piperidine in DMF | 20 min |
| 39 | Boc20 (3.0 eq), DIEA (3.0 eq) | 60 min |
| 41 | Deprotection of Dde: 3% hydrazine | 20 min |
| | hydrate | |
| 42 | 2-Bromo acetic acid (AcBr, 3.0 eq)/ | 90 min |
| | 3 eq. DIC | |
| 43 | Cleavage: 90.0% TFA/ 2.5% H₂O/5.0% | |
| | DTT/2.5% TIS | |
| | Crude amount: 5 g | |
| | Isolated amount: 215 mg, 95.91% | |
| | Yield: 2.19% Yield | |

*Note:
the reaction was carried out overnight (for 16 hours), may not require overnight reaction.

M6457 (Table 12) was synthesized using standard solid phase peptide synthesis (SPPS) followed by de-protection (removal of Dde group) on resin and reaction with BrAc (Bromo-acetyl) on resin (Table 13).

Resin preparation: a solution of Rink Amide MBHA Resin (2.0 mmol, 1.0 eq, Sub 0.33 mmol/g) in DMF (100.0 mL) was agitated with N₂ at 25° C. for 2 hours. Then 20% piperidine in DMF (100.0 mL) was added in the resin and agitated with N₂ at 25° C. for 15 min. The resin was washed with DMF (100.0 mL×5).

AA Coupling: a solution of Oxyma (3.0 eq) and Fmoc-Lys(Dde)-OH (3.0 eq) in DMF (50.0 mL) was added to the resin, then the DIC(3.0 eq) was added, the mixture was agitated with N₂ at 25° C. for 30 min. The resin was washed with DMF (100.0 mL×5).

Fmoc Deprotection: 20% piperidine in DMF (100.0 mL) was added to the resin and the reaction mixture was agitated with N₂ at 25° C. for 15 min. The resin was filtered and washed with DMF (100.0 mL×5).

AA elongation: amino acids (2-37) (Table 13) were coupled using the above-noted procedures for AA Coupling and Fmoc Deprotection.

A solution of DIEA (3.00 eq) and Boc₂O (3.00 eq) in DMF (50 mL) was added to the resin and the reaction mixture was agitated with N₂ for 1 hour at 25° C. Then the resin was washed with DMF (100 mL*5).

Deprotection of Dde: 3% N₂H₄·H₂O/DMF was added to the resin and the reaction was carried out for 20 min. This reaction was repeated for one more time. Then the resin was drained and washed with DMF (100 mL×5).

2-Bromo acetic acid (AcBr) coupling: a solution of AcBr (5.0 eq) and DIC (6.0 eq) in DMF (50 mL) was added to the resin and the reaction mixture was agitated with N₂ for 1 hour at 25° C. The resin was then washed with DMF (100 mL×5).

Peptide Cleavage and Purification: a cleavage solution (150 mL, 90.0% TFA/2.50% TIS/2.50% H₂O/5.0% DTT) was added to the flask containing resin at room temperature and the reaction mixture was stirred for 2 hours. The resin was filtered and filtrate was collected. The peptide was precipitated with cold isopropyl ether (1.50 L). The precipitate was filtered and the filter cake was collected and washed with isopropyl ether (1.50 L×2). The crude peptide was dried under vacuum for 1 hour to obtain the crude peptide (5.0 g). The formation of the crude peptide was confirmed via LCMS (EW54293-1-P1A1, Rₜ=11.048 min). The crude peptide was purified by prep-HPLC (TFA condition: A: 0.075% TFA in H₂O, B: ACN) to give the final product (215 mg, 43.74 mol, 2.19% yield, 95.91% purity, TFA) as a white solid.

LCMS (TOF) M6457: Rₜ=1.093 min, MS cal.: 4600.70, MS observed: [M+3H]³⁺=1354.3470; [M+H]³⁺=4600.0331. HPLC M6457: Rₜ=11.489 min, purity: 95.91%.

### Example 3. Synthesis of M6562

TABLE 14
Properties of M6562

| | |
|---|---|
| M Code | M6562 |
| Batch No. | EW54293-3-P1 |
| Molecular Formula | C204H301N55O70 |
| Exact Mass | 4641.17 |
| Molecular Weight | 4643.97 |
| Counter Ion | TFA |
| Formula Weight | 5100.05 (+4 TFA) |
| Physical Appearance | White Powder |
| Storage | -20° C. |
| Weight | 252.1 mg |
| Peptide Sequence | H[Aib]EGTFTSDYSSYLEEQAAKEFIAWLVKGGGGGGG |
| | SGGGGSGGGGS-K(Ac-COT)-CONH2 (SEQ ID NO: 104) |

TABLE 15
Reagents and Reaction Conditions

| | | Reacting |
|---|---|---|
| Step # | Materials/Oxyma(3.0 eq), DIC(3.0 eq) | time |
| 1 | Fmoc-Lys(Boc)-OH (3.0 eq) | 80 min |
| 2 | Fmoc-Gly-Ser(PSI(Me,Me)Pro)-OH | 60 min |
| | (3.0 eq) | |
| 3 | Fmoc-Gly-Gly-Gly-OH (3.0 eq) | 80 min |
| 4 | Fmoc-Gly-Ser(PSI(Me,Me)Pro)-OH | 16 hours* |
| | (3.0 eq) | |
| 5 | Fmoc-Gly-Gly-Gly-OH (3.0 eq) | 90 min |
| 6 | Fmoc-Gly-Ser(PSI(Me,Me)Pro)-OH | 80 min |
| | (3.0 eq) | |
| 7 | Fmoc-Gly-Gly-Gly-OH (3.0 eq) | 60 min |
| 8 | Fmoc-DmbGly-OH (3.0 eq) | 16 hours |
| 9 | Fmoc-Gly-Gly-OH (3.0 eq) | 60 min |
| 10 | Fmoc-Lys(Dde)-OH (3.0 eq) | 60 min |
| 11 | Fmoc-Val-OH (3.0 eq) | 80 min |
| 12 | Fmoc-Leu-OH (3.0 eq) | 16 hours |
| 13 | Fmoc-Trp(Boc)-OH (3.0 eq) | 90 min |
| 14 | Fmoc-Ala-OH (3.0 eq) | 60 min |
| 15 | Fmoc-Ile-OH (3.0 eq) | 80 min |
| 16 | Fmoc-Phe-OH (3.0 eq) | 16 hours |
| 17 | Fmoc-Glu(OtBu)-OH (3.0 eq) | 80 min |
| 18 | Fmoc-Lys(Dde)-OH (3.0 eq) | 90 min |
| 19 | Fmoc-Ala-OH (3.0 eq) | 16 hours |
| 20 | Fmoc-Ala-OH (3.0 eq) | 120 min |
| 21 | Fmoc-Gln(Trt)-OH (3.0 eq) | 16 hours |
| 22 | Fmoc-Glu(OtBu)-OH (3.0 eq) | 100 min |
| 23 | Fmoc-Glu(OtBu)-OH (3.0 eq) | 16 hours |
| 24 | Fmoc-Leu-OH (3.0 eq) | 90 min |
| 25 | Fmoc-Tyr(tBu)-OH (3.0 eq) | 100 min |
| 26 | Fmoc-Ser(tBu)-OH (3.0 eq) | 16 hours |
| 27 | Fmoc-Ser(tBu)-OH (3.0 eq) | 100 min |
| 28 | Fmoc-Tyr(tBu)-OH (3.0 eq) | 120 min |
| 29 | Fmoc-Asp(OtBu)-OH (3.0 eq) | 16 hours |
| 30 | Fmoc-Ser(tBu)-OH (3.0 eq) | 100 min |
| 31 | Fmoc-Thr(tBu)-OH (3.0 eq) | 100 min |
| 32 | Fmoc-Phe-OH (3.0 eq) | 16 hours |
| 33 | Fmoc-Thr(tBu)-OH (3.0 eq) | 120 min |
| 34 | Fmoc-Gly-OH (3.0 eq) | 90 min |
| 35 | Fmoc-Giu(OtBu)-OH (3.0 eq) | 16 hours |
| 36 | Fmoc-Aib-OH (3.0 eq) | 90 min |
| 37 | Fmoc-His(Trt)-OH (3.0 eq) | 120 min |
| | De-Dde and De-Fmoc: 3% hydrazine | 20 min |
| | hydrate | |
| 38 | Fmoc-Cl (6.0 eq), DIEA (12.0 eq)/ Fmoc- | 30 min |
| | OSu(12.0 eq), DIEA (24.0 eq) | |
| 38 | Cleavage: 90.0% TFA/ 2.5% H2O/5.0% | 90-120 min |
| | DTT/2.5% TIS | |
| 39 | COT-Osu (1.2 eq) / DIEA (3eq) | 30 min |
| 40 | De-Fmoc/20% Piperidine in DMF | 20 min |

*Note:
the reaction was carried out overnight (for 16 hours), may not require overnight reaction.

### Synthesis of Intermediate 1 (M6562-Int 1)

Resin preparation: a solution of Rink Amide MBHA Resin (6 g, 2.0 mmol, 1.0 eq, Sub 0.33 mmol/g) in DMF (100.0 mL) was agitated with N₂ at 25° C. for 2 hours. Then 20% piperidine in DMF (100.0 mL) was added to the resin and the mixture was agitated with N₂ at 25° C. for 15 min. The resin was washed with DMF (100.0 mL×5).

AA Coupling: a solution of Oxyma (3.0 eq) and Fmoc-Lys(Boc)-OH (3.0 eq) in DMF (50.0 mL) was added to the resin, then the DIC (3.0 eq) was added, the mixture was agitated with N₂ at 25° C. for 30 min. The resin was washed with DMF (100.0 mL×5).

Fmoc Deprotection: 20% piperidine in DMF (100.0 mL) was added to the resin and the reaction mixture was agitated with N₂ at 25° C. for 15 min. The resin was filtered and washed with DMF (100.0 mL×5).

AA elongation: Amino acids (2-38) (Table 15) were coupled using the above-noted procedure for AA Coupling and Fmoc Deprotection.

After coupling, the resin was washed 5 times with DMF (100.0 mL). After last step, the resin was washed with MeOH (100.0 mL) for 3 times and dried under vacuum.

Deprotection of Dde: 3% N₂H₄·H₂O/DMF (10.0 mL) was added to a solution of the resin (0.10 mmol) and the reaction was carried out for 20 min. This reaction was then repeated one more time. The resin was drained and washed with DMF (10.0 mL×5).

Fmoc Coupling: a solution of Fmoc-Cl (6.0 eq) and DIEA (12.0 eq) in DMF (5.0 mL) was added to the resin and the reaction mixture was agitated with N₂ for 1 hour at 25° C. The resin was then washed with DMF (10.0 mL*5).

Alternative methods for Deprotection of Dde and Fmoc Coupling are described below.

To a solution of the resin (1.9 mmol) was added 3% N₂H₄·H₂O/DMF (150.0 mL) and the reaction was carried out for 20 min. The resin was drained and washed with DMF (150.0 mL*5).

Coupling: a solution of Fmoc-Osu (12.0 eq) and DIEA (24.0 eq) in DMF (75.0 mL) was added to the resin and the reaction mixture was agitated with N₂ for 1 hour at 25° C. The resin was then washed 5 times with DMF (150.0 mL).

Peptide Cleavage and Purification: a cleavage solution (200.0 mL, 90.0% TFA/2.50% TIS/2.50% H₂O/5.0% DTT) was added to the flask containing resin at room temperature and the reaction mixture was stirred for 2 hours. The resin was filtered and filtrate was collected. The peptide was precipitated with cold isopropyl ether (2.0 L). The precipitate was filtered and the filter cake was collected and washed twice with isopropyl ether (2.0 L). The crude peptide was dried under vacuum 1 hour to afford the crude peptide (5.0 g). The formation of the crude peptide was confirmed via Crude LCMS (Rₜ=12.736 min). The crude peptide was purified by prep-HPLC (TFA condition: A: 0.075% TFA in H₂O, B: ACN) to give the product M6562-Int 1 (700 mg, about 70% purity, TFA) as a white solid. Rₜ=12.736 min, MS cal.: 5136.50, MS observed: [M+3H]³⁺=1716.9.

### Synthesis of M6562

To a solution of M6562-Int 1 (680.0 mg, 1.0 eq, TFA) in DMSO (20.0 mL) was added DIEA (3.0 eq) and COT-Osu (1.20 eq). The mixture became turbid, and the reaction mixture was stirred at 20° C. for 30 min. LCMS showed that one main peak (Rₜ=2.08 min) with the desired mass. A solution of 10% diethylamine in DMSO (20.0 mL) was added dropwise to the mixture at 25° C. The mixture became clear and was stirred at 25° C. for 20 min. LCMS showed that one main peak (Rₜ=1.748 min) with the desired mass. The crude product was purified by reversed-phase HPLC (A: H₂O, B: ACN) eluting with A:0.075% TFA in H₂O, B: ACN to give the final product (252.7 mg, 98.28% purity, TFA) as a white solid. LCMS (M6562): Rₜ=1.095 min, MS cal.: 4643.97, MS observed: [M+3H]³⁺=1548.7483. HPLC (M6562) Rₜ=10.726 min, purity: 98.28%.

### Example 4. Synthesis of M6588

TABLE 16
Properties of M6588

| | |
|---|---|
| M Code | M6588 |
| Batch No. | EW42729-48-P1 |
| Molecular Formula | C193H284N50O64 |
| Exact Mass | 4326.05 |
| Molecular Weight | 4328.68 |
| Counter Ion | TFA |
| Formula Weight | 4784.76 (+4 TFA) |
| Physical Appearance | White Powder |
| Storage | -20° C. |
| Weight | 26.4 mg |
| Peptide Sequence | H[Aib]EGTFTSDYSSYLEEQAAKEFIAWLVK |
| | GGGGGGGSGGGGS-K(Ac-COT)-CONH2 |
| | (SEQ ID NO: 105) |

TABLE 17
Reagents and Reaction Conditions (Resin used is the Rink
Amide MBHA (loading, 0.33 mmol/g, 6.0 g))

| | Materials: AA with Oxyma (3.0 eq), | |
|---|---|---|
| Step # | DIC (3.0 eq) | Reacting time |
| 1 | Fmoc-Lys(Boc)-OH (3.0 eq) | 80 min |
| 2 | Fmoc-Gly-Ser(PSI(Me,Me)Pro)-OH | 60 min |
| | (3.0 eq) | |
| 3 | Fmoc-Gly-Gly-Gly-OH (3.0 eq) | 80 min |
| 4 | Fmoc-Gly-Ser(PSI(Me,Me)Pro)-OH | 16 hours* |
| | (3.0 eq) | |
| 5 | Fmoc-Gly-Gly-Gly-OH (3.0 eq) | 90 min |
| 6 | Fmoc-DmbGly-OH (3.0 eq) | 80 min |
| 7 | Fmoc-Gly-Gly-OH (3.0 eq) | 60 min |
| 8 | Fmoc-Lys(Dde)-OH (3.0 eq) | 16 hours |
| 9 | Fmoc-Val-OH (3.0 eq) | 60 min |
| 10 | Fmoc-Leu-OH (3.0 eq) | 60 min |
| 11 | Fmoc-Trp(Boc)-OH (3.0 eq) | 80 min |
| 12 | Fmoc-Ala-OH (3.0 eq) | 16 hours |
| 13 | Fmoc-Ile-OH (3.0 eq) | 90 min |
| 14 | Fmoc-Phe-OH (3.0 eq) | 60 min |
| 15 | Fmoc-Glu(OtBu)-OH (3.0 eq) | 80 min |
| 16 | Fmoc-Lys(Dde)-OH (3.0 eq) | 16 hours |
| 17 | Fmoc-Ala-OH (3.0 eq) | 80 min |
| 18 | Fmoc-Ala-OH (3.0 eq) | 90 min |
| 19 | Fmoc-Gln(Trt)-OH (3.0 eq) | 16 hours |
| 20 | Fmoc-Glu(OtBu)-OH (3.0 eq) | 120 min |
| 21 | Fmoc-Glu(OtBu)-OH (3.0 eq) | 16 hours |
| 22 | Fmoc-Leu-OH (3.0 eq) | 100 min |
| 23 | Fmoc-Tyr(tBu)-OH (3.0 eq) | 16 hours |
| 24 | Fmoc-Ser(tBu)-OH (3.0 eq) | 90 min |
| 25 | Fmoc-Ser(tBu)-OH (3.0 eq) | 100 min |
| 26 | Fmoc-Tyr(tBu)-OH (3.0 eq) | 16 hours |
| 27 | Fmoc-Asp(OtBu)-OH (3.0 eq) | 100 min |
| 28 | Fmoc-Ser(tBu)-OH (3.0 eq) | 120 min |
| 29 | Fmoc-Thr(tBu)-OH (3.0 eq) | 16 hours |
| 30 | Fmoc-Phe-OH (3.0 eq) | 100 min |
| 31 | Fmoc-Thr(tBu)-OH (3.0 eq) | 100 min |
| 32 | Fmoc-Gly-OH (3.0 eq) | 16 hours |
| 33 | Fmoc-Giu(OtBu)-OH (3.0 eq) | 120 min |
| 34 | Fmoc-Aib-OH (3.0 eq) | 90 min |
| 35 | Fmoc-His(Trt)-OH (3.0 eq) | 16 hours |
| 36 | De Dde & De-Fmoc: 3% hydrazine | 20 min |
| | hydrate | |
| 37 | Fmoc-COT-Osu (12.0 eq) /DIEA | 30 min |
| | (24.0 eq) | |
| 38 | Cleavage: 90.0% TFA/ 2.5% H2O/ | 90-120 min |
| | 5.0% DTT/2.5% TIS | |
| 39 | COT-Osu (1.2 eq) /DIEA (3eq) | 30 min |
| 40 | De-Fmoc: 20% Piperidine in DMF | 20 min |

*Note:
the reaction was carried out overnight (for 16 hours), may not require overnight reaction.

### Synthesis of Intermediate 1 (M6588-Int 1)

Resin preparation: A solution of Rink Amide MBHA Resin (0.30 mmol, 1.0 eq, Sub 0.33 mmol/g) in DMF (20.0 mL) was agitated with N₂ at 25° C. for 2 hours. Then 20% piperidine in DMF (20.0 mL) was added to the resin and the reaction mixture was agitated with N₂ at 25° C. for 15 min. The resin was washed with DMF (20.0 mL×5).

Coupling: a solution of Oxyma (3.0 eq) and Fmoc-Lys(Boc)-OH (3.0 eq) in DMF (10.0 mL) was added to the resin, then the DIC(3.0 eq) was added, and the mixture was agitated with N₂ at 25° C. for 30 min. The resin was washed with DMF (20.0 mL×5).

Fmoc Deprotection: 20% piperidine in DMF (20.00 mL) was added to the resin and the reaction mixture was agitated with N₂ at 25° C. for 15 min. The resin was filtered and washed 5 times DMF (20.0 mL).

AA elongation: Amino acids (2-36) (Table 17) were coupled using the above-noted procedure for Coupling and Fmoc Deprotection.

After coupling, the resin was washed 5 times with DMF (20.0 mL). After last step, the resin was washed 3 times with MeOH (20.0 mL) and dried under vacuum.

Dde deprotection: 3% N₂H₄·H₂O/DMF (20.0 mL) was added to the resin and the reaction was carried out for 20 min. This reaction was repeated 3 times. The resin was drained and washed 5 times with DMF (20.0 mL).

Fmoc-Coupling: a solution of Fmoc-Osu (12.0 eq) and DIEA (24.0 eq) in DMF (10.0 mL) was added to the resin and the reaction mixture was agitated with N₂ for 1 hour at 25° C. The resin was then washed 5 times with DMF (20.0 mL).

Peptide Cleavage and Purification: a cleavage solution (30.0 mL, 90.0% TFA/2.50% TIS/2.50% H₂O/5.0% DTT) was added to the flask containing resin at room temperature and the reaction mixture was stirred for 2 hours. The resin was filtered and filtrate was collected. The peptide was precipitated with cold isopropyl ether (300 mL). The precipitate was filtered and the filter cake was collected and washed 2 times with isopropyl ether (300 mL). The crude peptide was dried under vacuum 1 hour to obtain the crude peptide (1.20 g). The crude peptide was purified by prep-HPLC (TFA condition: A: 0.075% TFA in H₂O, B: ACN) to give the product M6588-Int 1 (200 mg, about 70% purity, TFA) as a white solid.

### Synthesis of M6588

To a solution of M6588-Int 1 (200.0 mg, 1.0 eq, about 70% purity, TFA) in DMSO (10.0 mL) was added DIEA (3.0 eq) and COT-Osu (1.20 eq). The mixture became turbid and was stirred at 20° C. for 30 min. LCMS showed that one main peak (Rt=2.286 min) with desired mass was detected. Then 10% diethylamine in DMSO (10.0 mL) was added dropwise at 25° C. The mixture became clear and was stirred at 25° C. for 20 min. LCMS showed that one main peak (Rₜ=9.494 min) with desired mass was detected. The crude product was purified by reversed-phase column (A: H₂O, B: ACN) and prep-HPLC (TFA condition, A:0.075% TFA in H₂O, B: ACN) to give the final product M6588 (26.4 mg, 98.96% purity, TFA) as a white solid, which was confirmed by LCMS (Rt=1.102 min) and HPLC. LCMS (M6588-Int 1): Rₜ=2.286 min, MS cal.: 4995.41, MS observed: [M+3H]³⁺=1666.2. LCMS (M6588): Rₜ=9.494 min, MS cal.: 4328.68, MS observed: [M+3H]³⁺=1443.8.

### Example 5. Synthesis of M6589

M6589 was prepared by using a similar procedures as described above for the synthesis of M6588.

Analytical characterization of M6589: LCMS Rₜ=1.124 min, MS cal.: 4010.93, MS observed: [M+3H]³⁺=1338.6521. HPLC: Rₜ=12.028 min, 97.36% purity.

TABLE 18
Properties of M6589

| | |
|---|---|
| M Code | M6589 |
| Batch No. | EW42729-48-P2 |
| Molecular Formula | C182H267N45O58 |
| Exact Mass | 4010.93 |
| Molecular Weight | 4013.40 |
| Counter Ion | TFA |
| Formula Weight | 4469.48 (+4 TFA) |
| Physical Appearance | White Powder |
| Storage | -20° C. |
| Weight | 36.1 |
| Peptide Sequence | H[Aib]EGTFTSDYSSYLEEQAAKEFIAWLVKGGGGG |
| | GGS-K(Ac-COT)-CONH2 (SEQ ID NO: 106) |

### Example 6. Synthesis of M6570

TABLE 19
Properties of M6570

| | |
|---|---|
| M Code | M6570 |
| Batch No. | WSY-0041 |
| Molecular Formula | C₂₀₀H₂₉₉N₅₉O₆₉ |
| Exact Mass | 4631.17 |
| Molecular Weight | 4633.94 |
| Counter Ion | TFA |
| Formula Weight | 5204.04 (+5 TFA) |
| Physical Appearance | White Powder |
| Storage | -20° C. |
| Weight | 10.0 mg |
| Peptide Sequence | H[Aib]EGTFTSDYSSYLEEQAAKEFIAWLVKGGG- |
| | (GGGGS)3-K(K(N3))-CONH2 (SEQ ID NO: 107) |

The Rink Amide MBHA (loading, 0.30 mmol/g, 0.30 g) was used. For each coupling, 4 eq. amino acid (AA) together with 4 eq. HBTU and 8 eq. DIPEA were used. The coupling reactions were carried out for a period according to Table 20.

TABLE 20
Reagents and Reaction Conditions

| Step # | Materials | Reacting time |
|---|---|---|
| 1 | Fmoc-Lys(Dde)-OH | 40 min |
| 2 | Fmoc-Gly-Ser(ψMe,Me pro)-OH | 40 min |
| 3 | Fmoc-Gly3-OH | 40 min |
| 4 | Fmoc-Gly-Ser(ψMe,Me pro)-OH | 40 min |
| 5 | Fmoc-Gly3-OH | 40 min |
| 6 | Fmoc-Gly-Ser(ψMe,Me pro)-OH | 40 min x 2 |
| 7 | Fmoc-Gly3-OH | 40 min |
| 8 | Fmoc-Gly3-OH | 40 min x 2 |
| 9 | Fmoc-Lys(Boc)-OH | 40 min x 2 |
| 10 | Fmoc-Val-OH | 40 min x 2 |
| 11 | Fmoc-Leu-OH | 40 min x 2 |
| 12 | Fmoc-Trp(Boc)-OH | 40 min x 2 |
| 13 | Fmoc-Ala-OH | 40 min x 2 |
| 14 | Fmoc-Ile-OH | 40 min x 2 |
| 15 | Fmoc-Phe-OH | 40 min x 2 |
| 16 | Fmoc-Glu(OtBu)-OH | 40 min x 2 |
| 17 | Fmoc-Lys(Boc)-OH | 40 min x 2 |
| 18 | Fmoc-Ala-OH | 40 min x 2 |
| 19 | Fmoc-Ala-OH | 40 min x 2 |
| 20 | Fmoc-Gln(Trt)-OH | 40 min x 2 |
| 21 | Fmoc-Glu(OtBu)-OH | 40 min x 2 |
| 23 | Fmoc-Glu(OtBu)-OH | 40 min x 2 |
| 24 | Fmoc-Leu-OH | 40 min x 2 |
| 25 | Fmoc-Tyr(tBu)-OH | 40 min x 2 |
| 26 | Fmoc-Ser(tBu)-OH | 40 min x 2 |
| 27 | Fmoc-Ser(tBu)-OH | 40 min x 2 |
| 28 | Fmoc-Tyr(tBu)-OH | 40 min x 2 |
| 29 | Fmoc-Asp(OtBu)-OH | 40 min x 2 |
| 30 | Fmoc-Ser(tBu)-OH | 40 min x 2 |
| 31 | Fmoc-Thr(tBu)-OH | 40 min x 2 |
| 32 | Fmoc-Phe-OH | 40 min x 2 |
| 33 | Fmoc-Thr(tBu)-OH | 40 min x 2 |
| 34 | Fmoc-Gly-OH | 40 min x 2 |
| 35 | Fmoc-Glu(OtBu)-OH | 40 min x 2 |
| 36 | Fmoc-Aib-OH | 40 min x 2 |
| 37 | Boc-His(Trt)-OH | 40 min x 2 |
| 38 | Dde removal: 10% hydrazine | 30 min |
| | monohydrate in DMF | |
| 39 | Fmoc-Lys(N3)-OH | 40 min x 2 |
| 41 | De-Fmoc: 20% Piperidine in | 2 x 10 min |
| | DMF | |
| 41 | Cleavage: 90.0% TFA/ 2.5% | 90 min |
| | H2O/5.0% 3-Mpa/2.5% TIS | |
| 42 | Crude amount 150 mg | |
| 43 | Isolated amount 10 mg | |

### Analytical Characterization Results:

Purity by HPLC: 96.12% (214 nm), RT=9.20 min
Mobile Phase: A: 0.05% TFA in water; B: 0.05% TFA in ACN
Gradient: 2000 B for 1 min; 20-80% B within 20 min
Flow Rate: 1.0 mL/min
Column Temperature: 40° C.
Column: XBridge Peptide BEH C18, 4.6 Å~150 mm, 3.5 μm
LCMS: (ESI) m/z: 1545.3 [M+3H]/³⁺, 1159.2 [M+4H]/⁴⁺, 927.5 [M+5H]/⁵⁺.

### Example 7. Synthesis of M6557

TABLE 21
Properties of M6557

| | |
|---|---|
| M Code | M6557 |
| Batch No. | SXD-0043 |
| Molecular Formula | C₂₀₀H₂₉₂N₅₆O₇₁ |
| Exact Mass | 4614.10 |
| Molecular Weight | 4616.86 |
| Counter Ion | TFA |
| Formula Weight | 5072.94 (+4 TFA) |
| Physical Appearance | White Powder |
| Storage | -20° C. |
| Weight | 10.0 mg |
| Peptide Sequence | H[AibJEGTFTSDYSSYLEEQ |
| | AAKEFIAWLVKGGG-(GGGGS)3-K(Ac-Mc)-CONH₂ |
| | (SEQ ID NO: 108) |

TABLE 22
Reagents and Reaction Conditions: Resin: Rink Amide
MBHA (loading, 0.30 mmol/g, 0.30 g)

| | Materials: AA(4eq) HBTU(4eq) | |
|---|---|---|
| Step # | DIPEA(8eq) | Reacting time |
| 1 | Fmoc-Lys(Dde)-OH | 40 min |
| 2 | Fmoc-Gly-Ser(4 Me,Me pro)-OH | 40 min |
| 3 | Fmoc-Gly3-OH | 40 min |
| 4 | Fmoc-Gly-Ser(4 Me,Me pro)-OH | 40 min |
| 5 | Fmoc-Gly3-OH | 40 min |
| 6 | Fmoc-Gly-Ser(4 Me,Me pro)-OH | 40 min x 2 |
| 7 | Fmoc-Gly3-OH | 40 min |
| 8 | Fmoc-Gly3-OH | 40 min x 2 |
| 9 | Fmoc-Lys(Boc)-OH | 40 min x 2 |
| 10 | Fmoc-Val-OH | 40 min x 2 |
| 11 | Fmoc-Leu-OH | 40 min x 2 |
| 12 | Fmoc-Trp(Boc)-OH | 40 min x 2 |
| 13 | Fmoc-Ala-OH | 40 min x 2 |
| 14 | Fmoc-Ile-OH | 40 min x 2 |
| 15 | Fmoc-Phe-OH | 40 min x 2 |
| 16 | Fmoc-Glu(OtBu)-OH | 40 min x 2 |
| 17 | Fmoc-Lys(Boc)-OH | 40 min x 2 |
| 18 | Fmoc-Ala-OH | 40 min x 2 |
| 19 | Fmoc-Ala-OH | 40 min x 2 |
| 20 | Fmoc-Gln(Trt)-OH | 40 min x 2 |
| 21 | Fmoc-Glu(OtBu)-OH | 40 min x 2 |
| 23 | Fmoc-Glu(OtBu)-OH | 40 min x 2 |
| 24 | Fmoc-Leu-OH | 40 min x 2 |
| 25 | Fmoc-Tyr(tBu)-OH | 40 min x 2 |
| 26 | Fmoc-Ser(tBu)-OH | 40 min x 2 |
| 27 | Fmoc-Ser(tBu)-OH | 40 min x 2 |
| 28 | Fmoc-Tyr(tBu)-OH | 40 min x 2 |
| 29 | Fmoc-Asp(OtBu)-OH | 40 min x 2 |
| 30 | Fmoc-Ser(tBu)-OH | 40 min x 2 |
| 31 | Fmoc-Thr(tBu)-OH | 40 min x 2 |
| 32 | Fmoc-Phe-OH | 40 min x 2 |
| 33 | Fmoc-Thr(tBu)-OH | 40 min x 2 |
| 34 | Fmoc-Gly-OH | 40 min x 2 |
| 35 | Fmoc-Glu(OtBu)-OH | 40 min x 2 |
| 36 | Fmoc-Aib-OH | 40 min x 2 |
| 37 | Boc-His(Trt)-OH | 40 min x 2 |
| 38 | Dde removal: 10% | 30 min |
| | hydrazine in DMF | |
| 39 | Mc-COOH/ HBTU(4eq) | 40 min x 2 |
| | DIPEA(8eq) | |
| 40 | De-Fmoc: 20% Piperidine in DMF | 2 x 10 min |
| 41 | Cleavage: 90.0% TFA/ 2.5% | 2 h |
| | H2O/5.0% 3-Mpa/2.5% TIS | |
| 42 | Crude product 150 mg | |
| 43 | Isolated amount 10 mg | |

### Analytical Characterization Results:

HPLC purity: 94.01% (214 nm), RT=9.33 min
Mobile Phase: A: 0.05% TFA in water; B: 0.05% TFA in ACN
Gradient: 20% B for 1 min; 20-80% B within 20 min
Flow Rate: 1.0 mL/min
Column Temperature: 40° C.
Column: XBridge Peptide BEH C18, 4.6 Å~150 mm, 3.5 μm
LCMS: (ESI) m/z: 1539.8 [M+3H]/³⁺, 1155.0 [M+4H]/⁴⁺, 924.2 [M+5H]/⁵⁺, 770.3 [M+6H]/⁶⁺.

### Example 8. Synthesis of M6677

M6677 was synthesized from a Cu(I)-catalyzed azide-alkyne 1,3-dipolar cycloaddition (CuAAC) between M6675 (peptide azide) and M6694 (alkyne linker) in solution phase reaction.

TABLE 23
Peptide Sequence and Summary Properties

| M Code | M6677 | M6675 | M6694 |
|---|---|---|---|
| Molecular | C₂₁₅H₃₁₈N₆₀O₇₂ | C₁₆₁H₂₃₆N₄₂O₅₀ | C₅₄H₈₂N₁₈O₂₂ |
| Formula | | | |
| Exact Mass | 4597.01 | 3557.72 | 1334.59 |
| Molecular | 4600.70 | 3559.90 | 1335.35 |
| Weight | | | |
| Counter Ion | TFA | TFA | NA |
| Formula | 5351.34 (+4 TFA) | 4015.98 (+4 TFA) | 1335.35 |
| Weight | | | |
| MS | 1632.3 [M + 3H]³⁺ | 1187.3 [M + 3H]³⁺ | 668.4 [M + 2H]²⁺ |
| Physical | White Powder | White Powder | White Powder |
| Appearance | | | |
| Storage | −20° C. | −20° C. | −20° C. |
| Weight | 65 mg | 215 mg | 460 mg |
| Peptide | H[Aib]EGTFTSDYS | H[Aib]EGTFTSDYS | pent-4-ynoyl |
| Sequence | SYLEEQAAKEFIA | SYLEEQAAKEFIA | GGGSGGGGSGGGG |
| | WLVKGGGK(TA-G | WLVKGGGK(N3) | S-K(Ac-COT)-CONH2 |
| | GGSGGGGSGGGG | (SEQ ID NO: 110) | (SEQ ID NO: 111) |
| | S-K(Ac-COT))- | | |
| | CONH2 (SEQ ID | | |
| | NOS 109 & 139) | | |

*K(TA-): TA represents a triazole moiety formed by Click Reaction between the alkyne moiety of M6694 and the azide K(N₃) moiety of M6675:

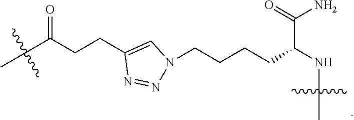 [SMILES: *N[C@H](CCCCn1cc(CCC(*)=O)nn1)C(N)=O]

### Synthesis of M6675 (Payload)

The Rink Amide MBHA (loading: 0.33 mmol/g) (1.51 g, 0.5 mmol) was used. Each coupling is carried out with 2 eq. of AA, 2 eq. of HBTU, and 4 eq. of DIPEA in DMF (15 mL) (Table 24). The reactions were carried out at 25° C.

TABLE 24
Reagents and Reaction Conditions for the Synthesis
of M6675 (Payload)

| Step | AA material | Time |
|---|---|---|
| 1 | Fmoc-K(N₃)-OH (2.0 eq.) | 60 min |
| 2 | Fmoc-Gly3-OH (2.0 eq.) | 60 min |
| 3 | Fmoc-Lys(Boc)-OH (2.0 eq.) | 60 min |
| 4 | Fmoc-Val-OH (2.0 eq.) | 60 min |
| 5 | Fmoc-Leu-OH (2.0 eq.) | 60 min |
| 6 | Fmoc-Trp(Trt)-OH (2.0 eq.) | 60 min |
| 7 | Fmoc-Ala-OH (2.0 eq.) | 60 min |
| 8 | Fmoc-Ile-OH (2.0 eq.) | 60 min |
| 9 | Fmoc-Phe-OH (2.0 eq.) | 60 min |
| 10 | Fmoc-Glu(OtBu)-OH (2.0 eq.) | 60 min |
| 11 | Fmoc-Lys(Boc)-OH (2.0 eq.) | 60 min |
| 12 | Fmoc-Ala-OH (2.0 eq.) | 60 min |
| 13 | Fmoc-Ala-OH (2.0 eq.) | 60 min |
| 14 | Fmoc-Gln(Trt)OH (2.0 eq.) | 60 min |
| 15 | Fmoc-Glu(OtBu)-OH (2.0 eq.) | 60 min |
| 16 | Fmoc-Glu(OtBu)-OH (2.0 eq.) | 60 min |
| 17 | Fmoc-Leu-OH (2.0 eq.) | 60 min |
| 18 | Fmoc-Tyr(tBu)-OH (2.0 eq.) | 60 min |
| 19 | Fmoc-Ser(tBu)-OH (2.0 eq.) | 60 min |
| 20 | Fmoc-Ser(tBu)-OH (2.0 eq.) | 60 min |
| 21 | Fmoc-Tyr(tBu)-OH (2.0 eq.) | 60 min |
| 22 | Fmoc-Asp(OtBu)-OH (2.0 eq.) | 60 min |
| 23 | Fmoc-Ser(tBu)-OH (2.0 eq.) | 60 min |
| 24 | Fmoc-Thr(tBu)-OH (2.0 eq.) | 60 min |
| 25 | Fmoc-Phe-OH (2.0 eq.) | 60 min |
| 26 | Fmoc-Thr(tBu)-OH (2.0 eq.) | 60 min |
| 27 | Fmoc-Gly-OH (2.0 eq.) | 60 min |
| 28 | Fmoc-Glu(OtBu)-OH (2.0 eq.) | 60 min |
| 29 | Fmoc-Aib-OH (2.0 eq.) | 60 min |
| 30 | Boc-His(Trt)-OH (2.0 eq.) | 90 min |
| | Cleavage: 90.0% TFA/ 2.5% H2O/ | 90 min |
| | 5.0% 3-Mpa*/2.5% TIS, 20 mL | |
| | (12 mL/g resin) | |
| | Crude 800 mg | |
| | Isolated 160 mg, 94.78% | |
| | Yield 9.2% | |

*3-Mpa = 3-Mercaptopropionic acid

The peptide was synthesized by standard solid phase peptide synthesis (SPPS) strategy, including resin preparation and loading; amide coupling and de-Fmoc reaction; cleavage and purification.

### Swelling of Resin

A solution of Rink Amide MBHA Resin (1.51 g, 0.50 mmol, 1.0 eq., Sub 0.33 mmol/g) in DMF (15 mL) was agitated with N₂ at 25° C. for 2 hours. Then 20% piperidine in DMF (15 mL) was added and the mixture was agitated with N₂ at 25° C. for 15 min. This procedure was repeated twice. The resin was washed with DMF (15 mL×6).

### 1^{st} Amino-Acid Loading to Resin

The Resin loading rate was 0.33 mmol/g resin; therefore, the first AA (0.5 mmol) was loaded to 1.51 g resin. A solution of HBTU (379 mg, 1.0 mmol, 2.0 eq.), DIPEA (341 μL, 2.0 mmol, 4.0 eq.) and Fmoc- Lys(N₃)—OH (394 mg, 1.0 mmol, 2.0 eq.) in DMF (15 mL) was added to the resin (1.51 g, 0.50 mmol) and the mixture was agitated with N₂ at 25° C. for 60 min. The resin was washed with DMF (15 mL×6).

### Coupling of Native Amino Acid

Fmoc-AA-OH (2.0 eq.)/HBTU (2.0 eq.)/DIPEA (4.0 eq.) was dissolved in DMF (15 mL). The mixture was agitated for 60-90 min at room temperature. After the reaction was completed, the resin was washed with DMF (10 mL/g of Resin×6).

Coupling of the amino acid after Aib (step 30): Boc-His(Trt)-OH (2.0 eq.)/HATU (2.0 eq.)/DIPEA (4.0 eq.) was dissolved in DMF (15 mL). The mixture was agitated for 90 min at room temperature. After the reaction was completed, the resin was washed with DMF (10 mL/g of Resin×6).

Fmoc Deprotection (after Each Coupling Reaction of Fmoc-Amino Acid)

20% piperidine in DMF (15 mL) was added to the resin, which was agitated with N₂ at 25° C. for 15 min; this procedure was repeated twice. The residue was washed with DMF (15 mL×6) and filtered to get the resin-peptide with amine at N-terminal to be used for next coupling.

### Cleavage

The resin bound peptide was cleaved using TFA/H₂O/3-Mpa/TIS (v/v/v/v=90.0/2.5/5.0/2.5, 18 mL) for 90 min. The mixture was filtered and the solution was collected. The residual resin was washed with TFA (5 mL×2). The combined solutions were poured into MTBE (pre-cooled to −10° C., 150 mL) and shaken to precipitate the crude peptide M6675. The suspension was centrifuged, and the supernatant was decanted. The residual solid was further washed with MTBE (100 mL×2) and centrifuged. After decanting the supernatant, the residual solid was then dried under reduced pressure overnight to give crude peptide M6675 as an off-white solid.

### Purification

The crude peptide (~800 mg) was dissolved in a mixed solvent of acetonitrile and water (v/v=1:1, 100 mL), purified by prep-HPLC (Column: Gemini C18 21.2×250 mm, 10 μm, 110 Å; Mobile Phase: A: water (containing 0.05% TFA), B: ACN; Gradient: 30-40% B in A within 30 min) and lyophilized to afford M6675 (165 mg, yield: 9.2%) as a white powder. ESI m/z: 1187.3 (M/3+H)⁺, 1780.3 (M/2+H)⁺.

### Analytical Characterization of M6675

HPLC purity of M6675: 94.78% (214 nm), RT=10.73 min. HPLC condition: Mobile Phase: A: 0.05% TFA in water; B: 0.05% TFA in CAN. Gradient: 20% B for 1 min; 20-80% B within 20 min. Flow Rate: 1.0 mL/min. Column Temperature: 40° C. Column: XBridge Peptide BEH C18, 4.6×150 mm, 3.5 μm.

LCMS M6675: (ESI) m/z: 1780.3 [M+2H]/2+, 1187.3 [M+3H]/3+, 890.8 [M+4H]/⁴⁺.

### Synthesis of M6694 (Linker)

### Synthesis of M6694-1

Peptide chain assembly was carried out on a resin support using standard Fmoc chemistry for peptide synthesis. Rink Amide MBHA (loading, 0.35 mmol/g, 5.71 g, 2 mmol) was used. The reactions were carried out at 25° C. Each coupling was carried out 4 eq. of Fmoc-protected building block (Table 25), 4 eq. HBTU, 8 eq. DIPEA in DMF (60 mL) for a period according to the Table 25.

TABLE 25
Reagents and Reaction Conditions for the Synthesis of M6694-1

| Step | | | Time |
|---|---|---|---|
| # | Materials | solvent | (min) |
| 1 | Fmoc-Lys(Boc)-OH, (4 eq.) | DMF (60 mL) | 60 |
| 2 | Fmoc-Gly-Ser(psi(Me,Me)pro)-OH (4 eq.) | DMF (60 mL) | 60 |
| 3 | Fmoc-Gly3-OH, (4 eq.) | DMF (60 mL) | 60 |
| 4 | Fmoc-Gly-Ser(psi(Me,Me)pro)-OH (4 eq.) | DMF (60 mL) | 60 |
| 5 | Fmoc-Gly3-OH, (4 eq.) | DMF (60 mL) | 60 |
| 6 | Fmoc-Gly-Ser(psi(Me,Me)pro)-OH (4 eq.) | DMF (60 mL) | 60 |
| 7 | Fmoc-Gly3-OH, (4 eq.) | DMF (60 mL) | 60 |
| 8 | PYA-OH*, (4 eq.) | DMF (60 mL) | 60 |

*PYA-OH: 4-Pentynoic acid

M6694-1 was synthesized by standard solid phase peptide synthesis strategy, including resin preparation and loading; amide coupling and de-Fmoc reaction; cleavage and purification.

### Resin Preparation

A solution of Rink Amide MBHA (5.71 g, 2.0 mmol, 1.0 eq., Sub 0.35 mmol/g) in DMF (60 mL) was agitated with N₂ at 25° C. for 0.5 hour. Then 20% piperidine in DMF (60 mL) was added and the mixture was agitated with N₂ at 25° C. for 15 min. The procedure was repeated twice. The resin was washed with DMF (60 mL×6).

### 1^{st} Amino-Acid Loading to Resin

A solution of HBTU (3.03 g, 8.0 mmol, 4.0 eq.), DIPEA (2.65 mL, 16.0 mmol, 8.0 eq.) and Fmoc-Lys(Boc)-OH (3.15 g, 8.0 mmol, 4.0 eq.) in DMF (60 mL) was added to the resin (5.71 g, 2.0 mmol), and the mixture was agitated with N₂ at 25° C. for 60 min. The resin was washed with DMF (60 mL×6).

### Amide Coupling Reaction (Coupling of Native Amino Acid or 4-Pentynoic Acid)

Fmoc-AA-OH or 4-pentynoic acid (4.0 eq.)/HBTU (4.0 eq.)/DIPEA (8.0 eq.) was dissolved in DMF (60 mL). The mixture was agitated for 60 min at room temperature. After the reaction was completed, the resin was washed with DMF (10 mL/g of Resin×6).

Deprotections (after Each Coupling Reaction of Native Fmoc-Amino Acid)

20% piperidine in DMF (50 mL) was added and agitated the resin with N₂ at 25° C. for 45 min. The resin was washed with DMF (50 mL×5) and filtered to get the resin-peptide with amine at N-terminal, which was used directly for next coupling.

### Cleavage

The resin bound peptide was cleaved using TFA/H₂O/3-Mpa/TIS (v/v/v/v=90.0/2.5/5.0/2.5, 180 mL) for 90 min. The mixture was filtered and the solution was collected. The residual resin was washed with TFA (20 mL×2). The combined solutions were poured into MTBE (pre-cooled to −10° C., 1500 mL) and shaken to precipitate the crude peptide M6694-1. The suspension was centrifuged and the supernatant was decanted. The residual solid was further washed with MTBE (1000 mL×2) and centrifuged. After decanting the supernatant, the residual solid was then dried under reduced pressure overnight to give crude peptide M6694-1 as an off-white solid.

### Purification

The crude peptide (~1500 mg) was dissolved in a mixed solvent of acetonitrile and water (v/v=1/1, 150 mL), purified by prep-HPLC (Column: XBridge OBD C18 19 mm×250 mm, 10 μm, 130 Å; Mobile Phase: A: water (containing 0.05% TFA), B: ACN; Gradient: 2-32% B in A within 30 min) and lyophilized to afford M6694-1 (450 mg, yield: 20%) as a white powder. ESI m/z: 1173.9 [M+H]⁺, 586.3 [M/2+H]⁺.

### Analytical Characterization for M6694-1

HPLC of M6694-1 after purification (>95%, RT=4.32 min). LCMS of M6694-1 after purification: (ESI) m/z: 1173.9 [M+H]/⁺, 586.3 [M+2H]/²⁺.

Purity by HPLC: purity >95% (214 nm), RT=4.32 min. Mobile Phase: A: 0.05% TFA in water; B: 0.05% TFA in CAN. Gradient: 5% B for 1 min, 5-65% B within 20 min. Flow Rate: 1.0 mL/min, Column Temperature: 40° C. Column: XBridge Peptide BEH C18, 4.6×150 mm, 3.5 μm

### Synthesis of M6694 (Linker)

The bifunctional linker M6694 was synthesized by amide coupling from M6694-1 and activated COT-OSu ester (M6694-2, CAS: 1425803-45-7) in liquid phase.

To a stirred solution of peptide M6694-1 (0.45 g, 0.38 mmol) in a mixed solvent of acetonitrile and water (v/v=1:1, 30 mL) were added DIPEA (98 mg, 0.76 mmol) and M6694-2 (0.11 g, 0.76 mmol) subsequently at room temperature, and the reaction mixture was stirred at room temperature for 3 hours, which was monitored by LCMS until the materials were consumed. A white viscous oil was then precipitated from the solution. The resulting mixture was centrifuged, and the supernatant was decanted. To the residue was added water (30 mL), and the mixture was stirred at room temperature for 10 minutes. The resulting suspension was centrifuged, and the supernatant was decanted. The residual semi-solid was slurried in ethyl acetate (30 mL) and then centrifuged to decant the supernatant. The residual semi-solid was then slurried in MTBE (30 mL) and then centrifuged to decant the supernatant. The residue was lyophilized to provide M6694 (0.42 g, 81% yield) as a white solid. ESI m/z: 1335.4 [M+H]⁺, 668.4 [M/2+H]⁺. HPLC of M6694 after purification (96.22% (214 nM), RT=17.53 min).

### Synthesis of M6677

TABLE 26
Synthesis of M6677 (Linker-Payload)

| | M6677 | M6675 | M6694 |
|---|---|---|---|
| Chemical | C215H318N60O72 | C161H236N42O50 | C54H82N18O22 |
| Form | | | |
| Exact Mass | 4892.31 | 3557.72 | 1335.35 |

The linker-peptide M6677 was synthesized by CuAAC (Copper (I)-catalyzed Azide-Alkyne Cycloaddition) between the azido group of the peptide M6675 and the terminal alkyne of the linker M6694, while the COT group didn't take part in the reaction under this specific reaction condition. Several experiments at various scales were conducted. The amounts of various reaction components used in the preparation were shown in Table 27 below.

TABLE 27
One Example of Reagents For Preparation
of M6677 at a Small Scale

| Compound | MF | MW | Amount | | μmol | equiv. | Note |
|---|---|---|---|---|---|---|---|
| M6675 | 4016 | 3560 | 20 | mg | 5 | 1.0 | |
| M6694 | 1335 | 1335 | 13 | mg | 10 | 2.0 | |
| CuSO₄ | 160 | 160 | 80 | uL | 50 | 10 | 100 mg/mL |
| | | | | | | | in H₂O |
| Ascorbic | 176 | 176 | 80 | uL | 50 | 10 | 100 mg/mL |
| Acid | | | | | | | in H₂O |
| tBuOH/H₂O | 1:1 | | 5 | mL | | | |

Synthesis of M6677 at a relatively larger scale: to a solution of the linker M6694 (0.10 g, 80 μmol, 2.0 eq.) in tert-butanol (1 mL) were added aq. ascorbic acid (0.72 mL, 100 mg/mL, 10 eq.) and aq. copper sulfate (0.64 mL, 100 mg/mL, 10 eq.). The mixture was added into a solution of the peptide M6675 (0.16 g, 40 μmol) in a mixed solvent tert-butanol and water (v/v=1:1, 20 mL). The resulting mixture was shaken at room temperature for 10 minutes, which was monitored by LCMS until the peptide M6675 was totally consumed. The resulting mixture was directly purified by prep-HPLC (Column: XBridge OBD C18 19 mm×250 mm, 10 μm, 130 Å; Mobile Phase: A: water (containing 0.05% TFA), B: ACN; Gradient: 20-80% B in A within 30 min) to provide M6677 (65 mg, 30% yield) as a white solid after lyophilization.

ESI m/z: 1632.3 [M/3+H]⁺, 1224.4 [M/4+H]⁺. HPLC of M6677 after purification (95.33% (214 nM), RT=9.78 min).

### Example 9. Antibody-Ligand Conjugations

Unique Reactivity of the Side Chain of Q55 of the Anti-GDF8 Antibody Toward a Primary Amine Via Microbial Transglutaminase (mTG)

### Site Specific Conjugation to Q295 Using Deglycosylated Anti-GDF8 Human IgG4

Initially site-specific conjugation at Q295 of heavy chain (HC) of the anti-GDF8 human IgG4 antibody was explored, which would afford a conjugate with a Drug-to-Antibody Ratio (DAR) of 2, theoretically, since there is one Q295 on each HC. A solution of 10 mg of anti-GDF8 human IgG4 antibody (REGN1033) in a buffer containing 10 mM Histidine and 50% w/v Sucrose at pH 6.3 was deglycosylated by incubating overnight at 37° C. with 0.2 units of PNGase F per mg of antibody. Subsequently, a solution of 2 mg of the deglycosylated anti-GDF8 human IgG4 was mixed with 100 molar equivalents of the conjugation handle for Click reaction, M404, followed by addition of wild-type microbial transglutaminase (mTG) from Zedira at a concentration of 1 unit per mg of antibody. The reaction mixture was incubated at 37° C. for 5 hours with gentle shaking for the transglutamination of M404 with the side chain of glutamine residues of the GDF8 antibody REGN1033. Samples were taken at 2-hour and 5-hour time points using desalting columns and submitted for ESI-MS analysis. Surprisingly, the results showed a Drug-to-Antibody Ratio (DAR) of 3.7 and 3.8 for the 2-hour and 5-hour time points, respectively, instead of DAR 2 as was originally envisioned on the two Q295 residues of the antibody HCs. Further detailed LC-MS analysis with partial digestion of the antibody-handle conjugate revealed that, the side chains of the two Q55 residues of the antibody were modified with the handle, in addition to the conjugation at the side chains of the two Q295 residues of the anti-GDF8 antibody. Those results demanded further investigations of the complications of those different conjugation sites assisted by mTG.

Conjugation of Handles M404 & M6092 to the Anti-GDF8 Antibody without Deglycosylation

The conjugation results above of a DAR 3.8 using a degylcosylated anti-GDF8 antibody prompted the investigation of the conjugation using the antibody without prior deglycosylation. The experiments revealed that the side chains of Q55 residues were more reactive than that of Q295 residues of the HC mediated by mTG. Under comparable conjugation conditions, conjugation of the handle occurred selectively and predominately at the side chains of Q55, not Q295, using the anti-GDF8 antibody directly without prior deglycosylation.

It was discovered that the anti-GDF8 antibody-handle intermediate, M404 vs. M6092, showed quite different stability. To verify the LC (Light Chain) loading efficiency with REGN1033, conjugation of a handle to the antibody without prior deglycosylation was conducted under the same conditions using two different handles, M404 and M6092, for Click reaction. The results indicated that mTG assisted conjugation of the M6092 handle completed more rapidly and the resulting product anti-GDF8 antibody-handle conjugate exhibited a greater stability than that of M404 handle. For ESI-MS analysis, two samples from each conjugate were taken: one was analyzed for DAR on the same day, and the other was analyzed the following day. For the conjugate of the M404 handle, a significant decrease in loading was observed in the sample analyzed the following day, whereas no change in DAR was noted for the M6092 handle (Table 28). M404 seems to be more prone to mTG mediated deconjugation compared to M6092 likely due to structural difference.

TABLE 28
Q55 Loading for REGN1033(GDF8)-M404
and REGN1033(GDF8)-M6092

| | Q55 loading | Q55 loading |
|---|---|---|
| | of handle | of handle |
| Handle | on same day | on next day |
| REGN1033(GDF8)-M404 | 70% | 14% |
| REGN1033(GDF8)-M6092 | 100% | 100% |

Antibody-Tethered Ligand (ATL) Conjugate Via Microbial Transglutaminase (mTG) Assisted Site-Specific Conjugation

### Attachment of a Handle for Conjugation to the Antibody by mTG

Anti-GDF8 human IgG4 antibody (REGN1033) or isotype control antibody containing a Q-tag on light chain and a Q-tag on heavy chain (H4H30045P) was mixed with 30-150 molar equivalents of the conjugation handle. The resulting solution was mixed with microbial transglutaminase (mTG; 0.5-5 units per mg of antibody, wild-type mTG from Zedira; mutated mTG from Merck kGaA) resulting in a final concentration of the antibody at 5-20 mg/mL. The reaction mixture was incubated at 25-37° C. for 0.5-8 hours with gentle shaking. The reaction was monitored by ESI-MS, and upon completion excess handle linker-payload, protein aggregates and MTG were removed by size exclusion chromatography (SEC) or Protein A affinity chromatography (ProA), which afforded the antibody-handle intermediate for conjugation with the linker-payload.

### Click Reaction Between the Linker-Payload and the Handle of the Antibody-Handle Intermediate to Afford the Final Product of the Antibody-Tethered Ligand Conjugate

The purified antibody-handle intermediate was mixed with 5-15 molar equivalent of a linker payload. The reaction mixture was incubated at 25-37° C. for 2-48 hours with gentle shaking. The reaction was monitored by ESI-MS and upon completion, the excess linker-payload, protein aggregates, and residual mTG were removed by size exclusion chromatography (SEC) or Protein A affinity chromatography (ProA), buffer exchanged into the formulation buffer and sterile filtered. The protein concentration was measured by a UV-vis spectrophotometer. Overall protein recovery was 50-80% and the antibody-tethered ligand conjugate (ATL) monomer purity (>90%) was determined by analytical SEC. ATLs were further characterized by liquid chromatography electrospray ionization mass spectrometry (LC-ESI-MS) to calculate the drug-to-antibody ratio (DAR>1.7).

### Conjugation Method for Generating Antibody-Tethered Ligand (ATL) on Light Chain Q-Tag of an Anti-GDF8 Antibody Using the Wild-Type mTG

### Attachment of a Handle to the Antibody

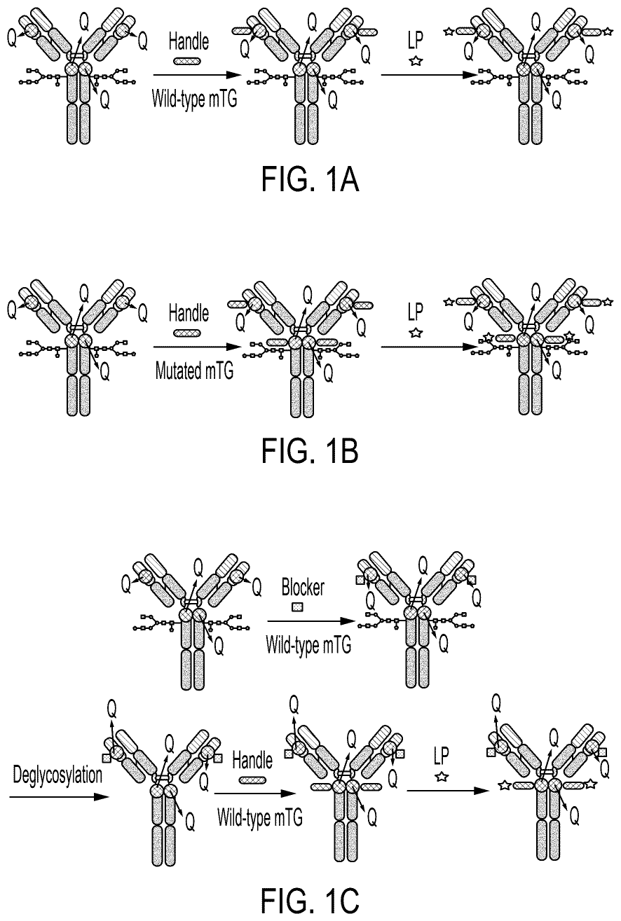

A specific example for the site-specific conjugation scheme shown in FIG. 1A. Anti-GDF8 antibody (49.3 mg) was mixed with 30 molar equivalents of conjugation handle (M6092), followed by the addition of wild-type microbial transglutaminase (Zedira, 0.5 unit mTG per mg of antibody), resulting in a final antibody concentration of 19 mg/mL. The reaction mixture was incubated at 37° C. for 30 minutes with gentle shaking. The reaction was monitored by ESI-MS and upon completion excess handle and MTG were removed by size exclusion chromatography (SEC) (AKTA avant, Superdex 200 pg).

### Conjugation of the Linker-Payload to the Antibody-Handle Intermediate to Prepare the Product of the Antibody-Tethered Ligand Conjugate Via Click Reaction

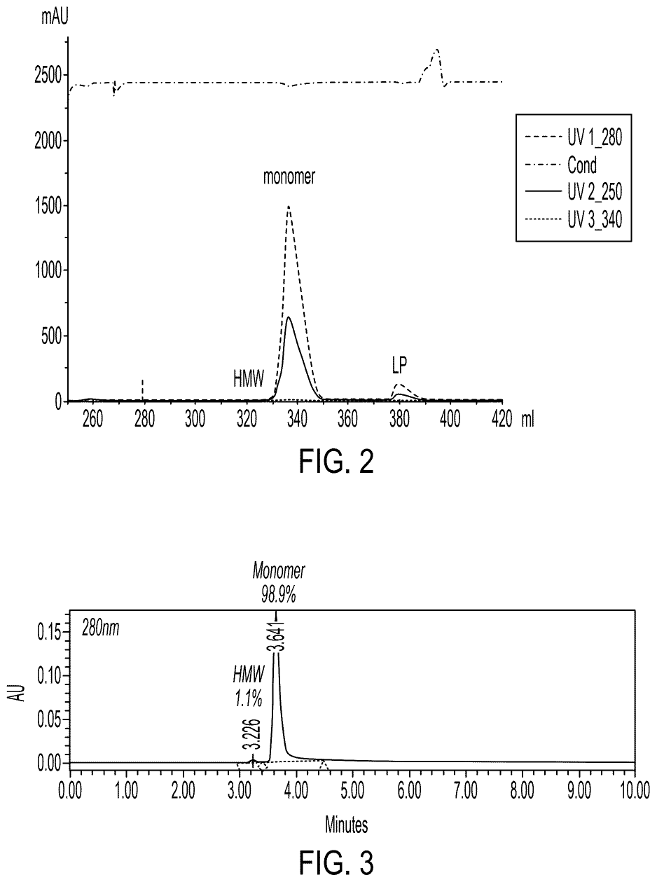

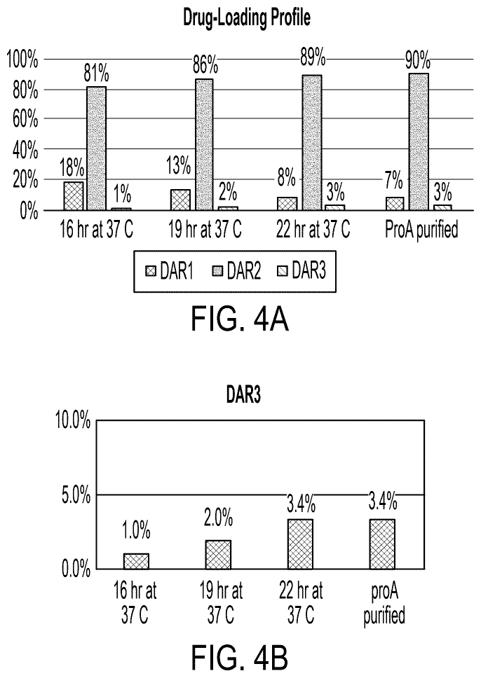

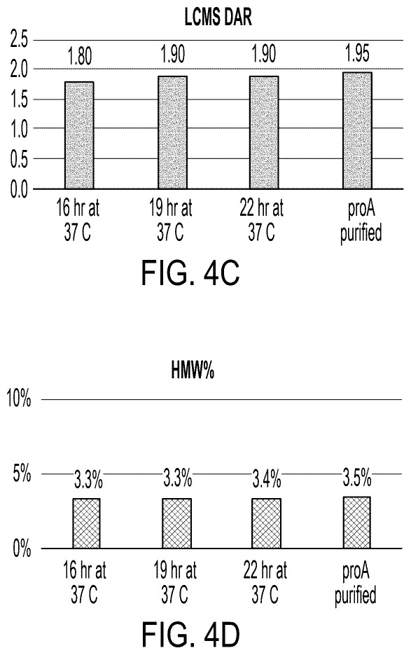

A solution of 38 mg of the purified antibody-handle conjugation intermediate was mixed with 5 molar equivalents of linker payload (M6562). The reaction mixture was incubated at 37° C. for 19 hours with gentle shaking for the Click reaction to proceed to complete the conjugation. The progress of the Click reaction was monitored by ESI-MS and upon completion aggregate and excess amount of linker payload were removed by size exclusion chromatography (SEC) (AKTA avant, Superdex 200 pg) (FIG. 2). The purified antibody-tethered ligand (ATL) conjugate was formulated into PBS buffer with 5% glycerol and sterile filtered. The protein concentration was measured by a UV-vis spectrophotometer. Overall protein recovery was 89% and ATL monomer purity was determined by analytical SEC (FIG. 3). ATLs were further characterized by liquid chromatography electrospray ionization mass spectrometry (LC-ESI-MS) to calculate the drug-to-antibody ratio (FIGS. 4A-4D).

### Conjugation Method for Generating Antibody-Tethered Ligand (ATL) on Light Chain and Heavy Chain Q-Tag of an Anti-GDF8 Antibody Using a Mutated mTG

### Attachment of the Handle to the Antibody to Afford the Antibody-Handle Conjugation Intermediate

A specific example for the site-specific conjugation scheme shown in FIG. 1B. Anti-GDF8 antibody (50 mg) was mixed with 80 molar equivalents of conjugation handle (M6092), followed by the addition of mutated microbial transglutaminase (Merck kGA, 2.0 unit mTG per mg of antibody), resulting in a final antibody concentration of 18.1 mg/mL. The reaction mixture was incubated at 37° C. for 2 hours with gentle shaking for the attachment of the handle to the antibody to afford the antibody-handle conjugation intermediate. The mTG assisted reaction was monitored by ESI-MS and upon completion excess handle and mTG were removed by size exclusion chromatography (SEC) (AKTA avant, Superdex 200 pg).

### Conjugation of the Linker Payload to the Antibody-Handle Conjugation Intermediate to Afford the Product of Antibody-Tethered Ligand (ATL) Conjugate

A solution of 5 mg of the purified antibody-handle conjugation intermediate was then mixed with 10 molar equivalents of linker payload (M6562). The reaction mixture was incubated at 37° C. for 22 hours with gentle shaking for the Click reaction to proceed to complete the conjugation of the linker payload. The reaction was monitored by ESI-MS and upon completion aggregate and excess linker payload were removed by size exclusion chromatography (SEC) (AKTA avant, Superdex 200 increase). The purified conjugate was formulated into PBS buffer with 5% glycerol and sterile filtered. The protein concentration was measured by a UV-vis spectrophotometer. ATL monomer purity was determined by analytical SEC. ATLs were further characterized by liquid chromatography electrospray ionization mass spectrometry (LC-ESI-MS) to calculate the drug-to-antibody ratio.

### Conjugation Method for Generating Antibody-Tethered Ligand (ATL) on Heavy Chain Q-Tag of an Anti-GDF8 Antibody

### Blocking the Side Chain of Q55

Another example for the site-specific conjugation at Q295 is shown in FIG. 1C. A solution of 48.3 mg anti-GDF8 antibody was mixed with 150 molar equivalents of Q55 blocker (M6549), followed by the addition of wild-type microbial transglutaminase (Zedira, 0.5 unit mTG per mg of antibody), resulting in a final antibody concentration of 14.8 mg/mL. The reaction mixture was incubated at 37° C. for 2 hours with gentle shaking. The reaction was monitored by ESI-MS and upon completion excess blocker and MTG were removed by size exclusion chromatography (SEC) (AKTA avant, Superdex 200 pg) to afford Q55 blocked anti-GDF8 antibody.

### Attachment of a Handle for Click Reaction to the Q55-Blocked Anti-GDF8 Antibody

Deglycosylation of the Q55 blocked anti-GDF8 antibody was carried out with 0.2 Unit PNGase F per mg of antibody, and incubated at 37° C. overnight. The deglycosylated Q55 blocked anti-GDF8 antibody was purified. A solution of 45.2 mg of the deglycosylated Q55 blocked antibody was mixed with 30 molar equivalents of conjugation handle (M6092), followed by the addition of wild-type microbial transglutaminase (mTG, Zedira, 0.5 unit mTG per mg of antibody). The reaction mixture was incubated at 37° C. for 3.5 hours with gentle shaking for the conjugation of M6092 to the side chain of Q295 to proceed. The reaction was monitored by ESI-MS. Upon completion, excess amount of handle M6092 and mTG were removed by size exclusion chromatography (SEC) (AKTA avant, Superdex 200 pg), which affords the Q55-blocked, deglycosylated antibody-handle intermediate. Since Q55 is blocked with M6092, only the side chains of Q295 are open for mTG assisted conjugation.

### Conjugation of the Linker Payload M6562 Via Click Reaction

A solution of 5 mg of the purified Q55 blocked, deglycosylated antibody-handle conjugation intermediate was then mixed with 5 molar equivalents of linker payload (M6562). The reaction mixture was incubated at 37° C. for 2 hours with gentle shaking for the Click reaction to proceed to afford the final product antibody-tethered ligand (ATL) conjugate. The reaction was monitored by ESI-MS and upon completion aggregate and excess amount of linker payload were removed by size exclusion chromatography (SEC) (AKTA avant, Superdex 200 increase). The purified conjugate was formulated into PBS buffer with 5% glycerol and sterile filtered. The protein concentration was measured by a UV-vis spectrophotometer. The monomer component of the final ATL product was determined by analytical SEC. ATLs were further characterized by liquid chromatography electrospray ionization mass spectrometry (LC-ESI-MS) to calculate the drug-to-antibody ratio (DAR).

### Analytical Methods for Characterizing ATLs

Analytical size exclusion chromatography (SEC) was performed to determine ATL monomer purity. Sample was run on an ACQUITY Protein BEH SEC column (200A, 1.7 um, 4.6 mm×150 mm) installed on an ACQUITY UPLC instrument (Waters) using 10 mM phosphate, 1.0 M sodium perchlorate, 5% v/v isopropanol as mobile phase at a flow rate of 0.3 mL/min and monitored UV-vis absorbance at 280 nm using an eλ PDA detector (Waters). The analytical SEC result (FIG. 3) indicated 99.6% monomer purity.

Liquid chromatography electrospray ionization mass spectrometry (LC-ESI-MS) analysis was performed to determine the drug distribution profile and to calculate the average drug-to-antibody ratio (DAR). Each sample (20 μL at 1 mg/mL) was digested by IdeS (an immunoglobulin-degrading enzyme; 30 minutes at 37° C.) then loaded onto an ACQUITY UPLC Protein BEH C4 column (10K psi, 300A, 1.7 um, 75 um×100 mm). Mass spectrum was acquired on a Waters Synapt G2-Si mass spectrometer (Tables 29-31). The major peak is DAR 2 species in the LC-ESI-MS spectrum of an IdeS digested anti-GDF8 ATL sample. The calculated average DAR value was 1.96.

TABLE 29
Mass Species (m/z) of IdeS Digested Ab and Site-Specific
Antibody Conjugates (i.e., (Fab)₂ species; ATLs)

| | | | Ab- | |
|---|---|---|---|---|
| ATL | | Ab- | Handle- | |
| No. | Ab | Handle | LP | ATL name/Seq |
| 2 | 96282 | 96643 | 105879 | REGN1033-M6092-M6562 (Q55) |
| 3 | 96282 | 96648 | 105251 | REGN1033-M6092-M6588 (Q55) |
| 4 | 96282 | 96648 | 104620 | REGN1033-M6092-M6589 (Q55) |
| 5 | 96282 | 96972 | 106206 | REGN1033-M6093-M6562 (Q55) |
| 6 | 96282 | 97598 | 106863 | REGN1033-M2153-M6570 (Q55) |
| 10 | 97143 | 97513 | 106741 | H4H30045P-M6092-M6562 (Q55) |
| 11 | 97143 | 97513 | 106113 | H4H30045P-M6092-M6588 (Q55) |
| 12 | 97143 | 97513 | 105482 | H4H30045P-M6092-M6589 (Q55) |
| 13 | 97143 | 97831 | 107068 | H4H30045P-M6093-M6562 (Q55) |
| 14 | 97143 | 98457 | 107726 | H4H30045P-M2153-M6570 (Q55) |

TABLE 30
Mass Species (m/z) of Deglycosylated Ab
and Site-Specific Conjugates

| ATL | | | Ab- | |
|---|---|---|---|---|
| No. | Ab | Ab-Handle | Handle-LP | ATL name |
| 7 | 143796 | 144528 | 162995 | REGN1033-M6092-M6562 |
| | | | | (Q55+Q295) |
| 15 | 144664 | 145385 | 163862 | H4H30045P-M6092-M6562 |
| | | | | (Q55+Q295) |

TABLE 31
Mass Species (m/z) of Reduced and Deglycosylated Ab and Interchain Cysteine Conjugates

| ATL | Mass Spec (m/z) | | | | | | |
|---|---|---|---|---|---|---|---|
| No. | LC | LC1 | HC | HC1 | HC2 | HC3 | ATL name |
| 1 | 23364 | 27884 | 48534 | 53054 | 57573 | 62094 | REGN1033-M6457 (Cys) |
| 8 | 23366 | N/A | 48833 | 48719 | N/A | N/A | REGN1033-M6092-M6562 (Q295) |
| 9 | 23498 | 28018 | 49087 | 53576 | 58096 | 62616 | H4H30045P-M6457 (Cys) |

TABLE 32
Summary of Prepared Antibody-Tethered Ligands (ATLs)

| | | | | Handle | Linker-Payload | | | |
|---|---|---|---|---|---|---|---|---|
| ATL | Ab | Ab | Conj. | M | | M | ATL | |
| NO .* | Target | Name/Seq | Site | Code | LP # | Code | Name/structure | DAR |
| 1* | Anti- | REGN1033 | Cys | n/a | Payload-(G4S)3- | M6457 | REGN1033- | 2.3 |
| | GDF8 | | | | K(Ac-Br) | | M6457 (Cys) | |
| 2 | Anti- | REGN1033 | Q55 | M6092 | payload-(G4S)3- | M6562 | REGN1033- | 2.0 |
| | GDF8 | | | | K(Ac-COT) | | M6092-M6562 | |
| 3 | Anti- | REGN1033 | Q55 | M6092 | payload-(G4S)2- | M6588 | REGN1033- | 1.9 |
| | GDF8 | | | | K(Ac-COT) | | M6092-M6588 | |
| 4 | Anti- | REGN1033 | Q55 | M6092 | payload-(G4S)- | M6589 | REGN1033- | 2.0 |
| | GDF8 | | | | K(Ac-COT) | | M6092-M6589 | |
| 5 | Anti- | REGN1033 | Q55 | M6093 | payload-(G4S)3- | M6562 | REGN1033- | 2.1 |
| | GDF8 | | | | K(Ac-COT) | | M6093-M6562 | |
| 6 | Anti- | REGN1033 | Q55 | M2153 | payload-(G4S)3- | M6570 | REGN1033- | 1.7 |
| | GDF8 | | | | K(KN3) | | M2153-M6570 | |
| 7* | Anti- | REGN1033 | Q55 + | M6092 | payload-(G4S)3- | M6562 | REGN1033- | 3.7 |
| | GDF8 | | Q295 | | K(Ac-COT) | | M6092-M6562 | |
| 8* | Anti- | REGN1033 | Q295 | M6092 | payload-(G4S)3- | M6562 | REGN1033- | 1.6 |
| | GDF8 | | | | K(Ac-Br) | | M6092-M6562 | |
| 9* | Isotype | REGN1945 | Cys | n/a | payload-(G4S)3- | M6457 | H4H30045P- | 1.8 |
| | | | | | K(Ac-COT) | | M6457 | |
| 10 | Isotype | H4H30045P | Q55 | M6092 | payload-(G4S)3- | M6562 | H4H30045P- | 2.0 |
| | | | | | K(Ac-COT) | | M6092-M6562 | |
| 11 | Isotype | H4H30045P | Q55 | M6092 | payload-(G4S)2- | M6588 | H4H30045P- | 1.7 |
| | | | | | K(Ac-COT) | | M6092-M6588 | |
| 12 | Isotype | H4H30045P | Q55 | M6092 | payload-(G4S)- | M6589 | H4H30045P- | 1.7 |
| | | | | | K(Ac-COT) | | M6092-M6589 | |
| 13 | Isotype | H4H30045P | Q55 | M6093 | COMP P-(G4S)3- | M6562 | H4H30045P- | 2.2 |
| | | | | | K(Ac-COT) | | M6093-M6562 | |
| 14 | Isotype | H4H30045P | Q55 | M2153 | COMP P-(G4S)3- | M6570 | H4H30045P- | 1.6 |
| | | | | | K(KN3)* | | M2153-M6570 | |
| 15* | Isotype | H4H30045P | Q55+ | M6092 | COMP P-(G4S)3- | M6562 | H4H30045P- | 3.6 |
| | | | Q295 | | K(Ac-COT) | | M6092-M6562 | |
| 16 | Anti- | REGN1033 | Q55 | M6092 | Payload-K(TA*- | M6677 | REGN1033- | 2.0 |
| | GDF8 | | | | (G4S)3-K(Ac-COT)) | | M6092-M6677 | |
| 17 | isotype | H4H30045P | Q55 | M6092 | Payload-K(TA- | M6677 | H4H30045P- | 2.0 |
| | | | | | (G4S)3-K(Ac-COT)) | | M6092-M6677 | |

*ATLs with a deglycosylated antibody: 1,7, 8, 9, and 15; K(N3) represent an amino acid residue where the E-amino replaced with an azido moiety; *K(TA-): TA represents a triazole moiety formed by Click Reaction between the alkyne moiety of M6694 and the azide K(N₃) moiety of M6675:

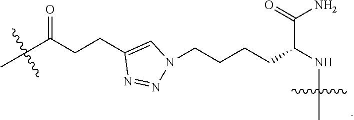 [SMILES: *N[C@H](CCCCn1cc(CCC(*)=O)nn1)C(N)=O]

Process Optimization for Preparation of REGN1033-M6092-M6562 Conjugate that Further Reduced the Components of High Molecular Weight (HMW) Aggregates and Higher DAR Species (Mainly DAR 3)

TABLE 33
Process Optimization for Production
of REGN1033-M6092-M6562 Conjugate

| | | Conc. | | | Endo- | | | Free |
|---|---|---|---|---|---|---|---|---|
| | | (mg/ | | HMW | toxin | Product | Yield | drug |
| Batch | Lot # | mL) | DAR | (%) | (EU/mg) | (mg) | (%) | % |
| 1 | L100 | 18.85 | 1.95 | 3.5% | <0.03 | 190 | 86% | n/a* |
| 2 | L96 | 3.96 | 2.0 | 6.8% | <0.1 | 190 | n/a | n/a |
| 3 | L83 | 3.41 | 2.0 | 1.2% | <0.15 | 38 | 91% | n/a |
| 4 | L81 | 1.61 | 1.6 | 2.1% | n/a | 5 | 80% | n/a |

*n/a: not available.

### Step-1: Attachment of a Handle M6092 to Antibody REGN1033 Assisted by mTG to Give REGN1033-M6092 Intermediate—Conjugation and Purification

In a 50 mL Falcon tube, 190 mg of anti-GDF8 antibody REGN1033-L32 (1.3 umol, 950 uL, 200 mg/mL in 10 mM Histidine, 5% Sucrose, pH 6.3) was diluted with 8.5 mL PBS to 20 mg/mL; 30 eq. methyltetrazine-amine (M6092, MW 201 Da, 235 μL of 40 mg/mL solution in MQ water, pH 7.0) was added; followed by Zedira MTG (0.5 U/mg Ab, 353 U/mL in HEPES buffer). Reaction mixture was incubated at 25° C. for 2 hours with gentle stirring, purified by Protein A chromatography, neutralized to pH 7.0, sterile filtered and stored at 4° C.

Crude M6092 conjugate was loaded on to a ProA column (MabSelect SuRe, 9.7 mL CV, flowrate 2-5 mL/min). The column was washed with 20 mM NaPi pH 7.3 (2.72 mS/cm) until 280 nm and 250 nm UV absorbance returned to baseline. The conjugate was eluted in 40 mM acetic acid pH 3.0; 1 M Tris-HCl pH 8 was added to raise pH to 7.0, followed by sterile filtration. 180 mg of ProA purified REGN1033-M6092 (23.5 mg/mL) was stored at 4° C.

### Step-2: Conjugation of Linker-Payload M6562 Via Click Reaction to the Antibody-Handle REGN1033-M6092 to Prepare REGN1033-M6092-M6562 Conjugate Product

In a 50 mL Falcon tube, 180 mg of REGN1033-M6092 (1.25 μmol, 7.66 mL, 23.5 mg/mL in tris/acetate buffer pH 7.0) was mixed with 5 eq of linker payload M6562 (MW 4644 Da., 25.5 mg/mL, 5 mM in DMSO). Reaction mixture was incubated 22 hours at 37° C. with gentle mixing, purified by ProA chromatography, neutralized to pH 7.0, formulated in PBS with 5% glycerol, sterile filtered and stored at −80° C.

Crude ATL was loaded on to a ProA column (MabSelect SuRe, 9.7 mL CV, flowrate 2-5 mL/min). The column was washed with 20 mM NaPi pH 7.3 (2.72 mS/cm) until 280 nm and 250 nm UV absorbance returned to baseline. The conjugate was eluted in 40 mM acetic acid pH 3.0; 1 M Tris-HCl pH 8 was added to raise pH to 7.0, 10×PBS and glycerol was added to formulate ATL in PBS with 5% glycerol, followed by sterile filtration. 163 mg of REGN1033-M6092-M6562 (18.85 mg/mL) was stored at −80° C.

TABLE 34
Yield of Each Step and Overall Process Yield

| | Process Yield |
|---|---|
| M6092 conjugation | 95% |
| M6562 conjugation | 91% |
| Overall | 87% |

It is important to mention that mTG must be completely removed or deactivated from the conjugation intermediate before the second step of conjugation.

When incubated at 37° C. overnight, even the residual amount of mTG can cause LP and ATL crosslinking, leading to ATL aggregation and excessive DAR3 species as measured LC-MS. The impact of residue mTG was confirmed by pretreating the conjugation intermediate with an mTG quencher, iodoacetamine (IAA). Unfortunately, neither SEC nor ProA could completely remove mTG from the conjugation intermediate (i.e., REGN1033-M6092), and iodoacetamide treatment is not manufacturing friendly. It was discovered that the residue mTG can be mostly deactivated by DMSO, which is a more practical approach. DMSO can be added either by preparing the linker payload stock solution in DMSO or by adding DMSO into the reaction mixture. While 10-15% DMSO can minimize the formation of aggregates and DAR3 species, a trend of slowly increasing these impurities with extended reaction time was observed (FIGS. 4A-4D). Therefore, it is desirable to develop a method that can effectively deactivate residue mTG before the linker payload conjugation. One approach is that after loading REGN1033-M6092 onto the ProA column, deactivate and wash off mTG with extensive column volumes of DMSO/buffer.

### Generation of Antibody-Tethered Ligand (ATL) Via Interchain Cysteine Conjugation

Interchain cysteine conjugates of anti-GDF8 human IgG4 antibody REGN1033 and isotype control antibody were prepared via partial reduction of the antibody with tris(2-carboxyethyl)phosphine (TCEP) followed by reaction of the reduced cysteine residues with bromoacetyl functionalized linker-payload. Specifically, antibodies were partially reduced by adding 2-3-fold molar excess of TCEP for 1-5 hours at 37° C. Linker-payloads were added at linker-payload/antibody molar ratio of 5-10 and reacted for an additional 2-20 hours at 25° C. The crude conjugates were purified via size exclusion chromatography (SEC), formulated in PBS with 5% glycerol then stored at −80° C. The protein concentration was measured by a UV-vis spectrophotometer. ADC monomer purity (>90%) was determined by analytical SEC. The ADCs were further characterized via liquid chromatography electrospray ionization mass spectrometry (LC-ESI MS) to calculate the drug-antibody ratio (DAR).

### Conjugation Method for Generating Antibody-Tethered Ligand (ATL) Through Partial Reduction of Interchain Disulfide Bonds of an IgG4 Antibody

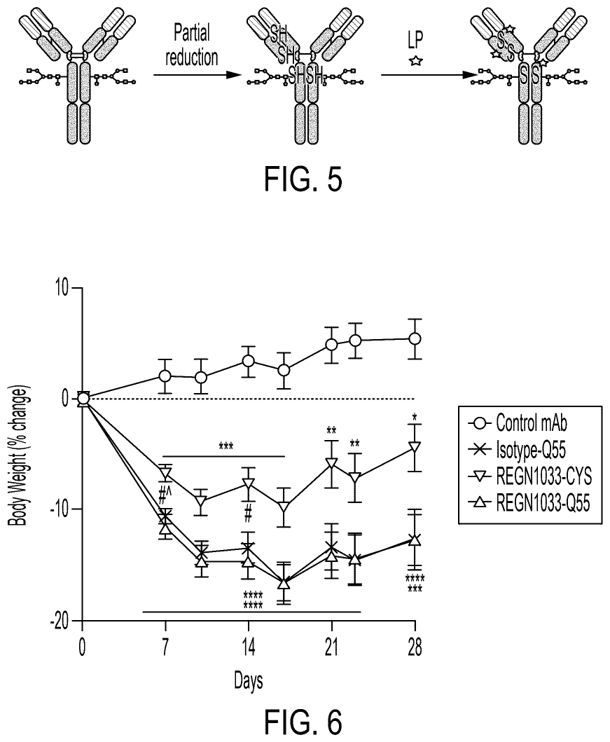

A specific example for the interchain cysteine conjugation scheme shown in FIG. 5. Anti-GDF8 human IgG4 antibody REGN1033 (70 mg) (20 mg/mL in PBS) was partially reduced by adding a 2.5-fold molar excess of tris(2-carboxyethyl)phosphine (TCEP) for 3.5 hours at 37° C. A solution of linker-payload (M6457, 40 mg/mL in MQ water) was added at a linker-payload/antibody molar ratio of 5 and reacted for an additional 22 hours at 25° C. The crude conjugate was purified via size exclusion chromatography (SEC) (AKTA avant, Superdex 200 PG), formulated in PBS with 5% Glycerol, then stored at −80° C. The protein concentration was measured by UV-vis spectrophotometer. ADC monomer purity was 99.7% by analytical SEC. The ADC was further characterized by liquid chromatography electrospray ionization mass spectrometry (LC-ESI MS) to calculate the drug-antibody ratio (LCMS DAR=2.3).

### Example 10. In Vitro Evaluations of GLP1R Agonist-Tethered GDF8 Antibody Conjugates Toward GLP1R Activation and the Blocking of GDF8 Activities Using CRE-Luc Assay and CAGA-Luc Assay

Cell lines and Growth Media:
HEK293/FSC11/pCDNA3.1+GLP1R+1 nM GLP1 (ACL6822)
   Growth media: DMEM+10% FBS+1× Penicillin-Streptomycin+500 μg/ml G418
   Referred to as HEK293/CRE-Luc/hGLP1R
A204/CAGAx12 Luc Cl. 1B3 (ACL14907)
   Growth media: McCoy's 5A Medium+10% FBS+1× Penicillin-Streptomycin+250 ug/ml G418
   Referred to as A204/CAGA-Luc

The following reagents, materials, and instruments were used: Dulbecco's Modified Eagle's Medium (DMEM) (Gibco, #11995065); McCoy's 5A (Modified) Medium (Gibco, #16600082); Fetal Bovine Serum (FBS) (Seradigm, #1500-500); Penicillin-Streptomycin (100×) (Gibco, #15140122), Geneticin Selective Antibiotic (G418) (Gibco, #11811098); Dulbecco's phosphate-buffered saline (PBS), no calcium, no magnesium (Gibco, #14190144); T75 cell culture treated flask (Corning, #430641U); 0.025% trypsin and 0.75 mM EDTA (1×) (Sigma-Aldrich, #SM-2004-C), Opti-MEM I Reduced Serum Medium (Gibco, #31985070); Masterblock, 384 Well, PP, Deep Well, V-Bottom, Natural, Sterile (Greiner Bio-One #781271); Luminescence assay plates, 384-Well, Cell Culture-Treated, Flat-Bottom Microplate (Falcon #353988); VIAFLO 384 (Integra); TopSeal-A PLUS (Revvity, #6050185); ONE-Glo Luciferase Assay System (Promega, #E6130); and EnVision Plate Reader (Revvity). Additional reagents are shown in Table 35.

TABLE 35
Reagents

| Reagent | Lot | Description | Source | Catalog # |
|---|---|---|---|---|
| GLP-1 | 436292 | GLP1R | Phoenix | 028-11 |
| (7-36) | | endogenous | Pharmaceuticals | |
| amide | | ligand | | |
| GDF-8/ | EZG7523072 | GDF8 | R&D Systems | 788-G8 |
| Myostatin | | | | |
| Protein | | | | |
| M6457 | M6457-L8 | Linker-Payload | Regeneron | NA |
| M6562 | M6562-L4 | Linker-Payload | Regeneron | NA |
| M6677 | M6677-L1 | Linker-Payload | Regeneron | NA |

TABLE 36
Antibodies and Antibody-Tethered Ligands (ATLs)

| mAb/ATL | Lot | Linker-Payload |
|---|---|---|
| REGN1033 | REGN1033-L70 | None |
| REGN1033-M6457 | REGN1033-L94 | M6457 |
| REGN1945-M6457 | REGN1945-L171 | M6457 |
| REGN1033-M6562 (Q55) | REGN1033-L83 | M6562 |
| REGN1033-M6562 (Q55) | REGN1033-L100 | M6562 |
| H4H30045P-M6562 (Q55) | H4H30045P-L7 | M6562 |
| REGN1033-M6562 (Q295) | REGN1033-L82 | M6562 |
| REGN1033-M6677 (Q55) | REGN1033-L102 | M6677 |

Glucagon-like peptide 1 receptor, GLP1R, is a member of the secretin family (Class B) of G protein-coupled receptors (GPCRs). Upon binding of its ligand, GLP-1, GLP1R initiates a downstream signaling cascade through Gαs G-proteins that raises intracellular cyclic AMP (cAMP) levels, which leads to the transcriptional regulation of target genes (Donnelly D., “The Structure and Function of the Glucagon-Like Peptide-1 Receptor and its Ligands,” British Journal of Pharmacology 166:27-41 (2012), which is hereby incorporated by reference in its entirety).

To test the GLP1R agonist activity of linker-payloads (LPs) and antibody conjugates of the invention, a cell-based cAMP responsive luciferase reporter assay was developed. To generate the assay cell line, the firefly luciferase gene was placed under the control of four copies of a cAMP response element (4×CRE) located upstream of a minimal promoter and transfected into HEK293 HZ cells (a highly transfectable subclone that was authenticated via STR profiling to be 81% identical to HEK293) and referred to herein as HEK293/CRE-Luc cells. HEK293/CRE-Luc cells were then engineered to express full-length human GLP1R (GenBank: AAI12127.1) (HEK293/CRE-Luc/hGLP1R).

Myostatin or growth differentiation factor 8 (GDF8) is a secreted ligand and member of the transforming growth factor beta (TGF-b) family that negatively regulates muscle growth. Myostatin signals through heteromeric complexes of type I (activin receptor-like kinase 4 and 5) and type II receptors (activin receptor type IIA and IIB) to activate different signaling pathways, including Smad2/3-mediated TGF-b signaling, which regulates transcription of target genes (Baig et al., “Myostatin and its Regulation: A Comprehensive Review of Myostatin Inhibiting Strategies,” Front Physiol. 13:876078 (2022), which is hereby incorporated by reference in its entirety).

To assess the ability of anti-GDF8 antibody/Antibody conjugates to block GDF8-induced signaling, a luciferase reporter assay using the cell line A204 (a human rhabdomyosarcoma cell line that endogenously expresses GDF8 receptors) was developed. The A204 cell line (ATCC, #HTB-82) was stably transfected with a Smad2/3-luciferase reporter plasmid (CAGAx12-Luc), and a clonal cell line that displayed robust GDF8-mediated luminescent signal was isolated (referred to herein as A204/CAGA-Luc cells).

For luminescence assays, cells were seeded into 384-well plates (Falcon #353988) at 6,000 cells/well in assay media (Opti-MEM, 1% FBS, 1× Penicillin-Streptomycin) and incubated overnight at 37° C. Serial dilutions of test articles and dilution of GDF8 were performed in assay media. Test articles were added to cells using VIAFLO 384 (Integra) with the last well in each serial dilution series serving as a blank control containing only assay media. Constant concentration of GDF8 was added to cells using VIAFLO 384 for assays performed in the presence of GDF8. After a 5-hours incubation at 37° C., luciferase activity was determined by addition of ONE-Glo reagent (Promega, #E6130) followed by measurement of relative light units (RLUs) on an EnVision Plate Reader (Revvity). For CRE-Luc assay, the antibody conjugate concentration was adjusted based on the drug-to-antibody ratio (DAR) of each test article by multiplying antibody concentration by DAR. For GDF8 blocking assay (CAGA-Luc assay), the antibody conjugate concentration was calculated based on the antibody concentration.

EC/IC₅₀ values for luminescence assays were determined using a four-parameter logistic equation over a 12-point dose response curve (GraphPad Prism). The blank was plotted as a continuation of the serial dilution.

The maximum signal relative to GLP-1 (Eₘₐₓ (% GLP-1)) in CRE-Luc assay was calculated using the following equation:

$${E}_{max} ( \% GLP - 1 ) = ( {RLU}_{max test article} - {RLU}_{blank} ) / ( {RLU}_{max GLP - 1} - {RLU}_{blank} ) \times 100$$

The maximum blocking percentage (Max blocking (%)) in CAGA-Luc assay was calculated using the following equation:

$$Max blocking ( \% ) = ( {RLU}_{max test article} - {RLU}_{min test article} ) / ( {RLU}_{max test article} - {RLU}_{blank} ) \times 100$$

### Results

Anti-GDF8 monoclonal antibody (mAb) REGN1033 was conjugated to GLP1R agonist LPs through different antibody conjugation methods/sites to generate anti-GDF8 Antibody conjugates: conjugation to LP M6457 via native disulfide bond bridging (REGN1033-M6457); conjugation to LP M6562 via light chain glutamine at position 55 (Q55) (REGN1033-M6562 (Q55)) or via heavy chain glutamine at position 295 (Q295) (REGN1033-M6562 (Q295)); and conjugation to LP M6677 via light chain Q55 (REGN1033-M6677 (Q55)). Isotype control Antibody conjugates were generated by conjugation of non-GDF8 binding antibodies via native disulfide bond bridging (REGN1945-M6457) and via light chain Q55 (H4H30045P-M6562 (Q55)).

As shown in Table 37, the endogenous GLP1R ligand, GLP-1 (7-36) amide (Phoenix Pharmaceuticals, #028-11) (referred to as GLP-1), increased CRE-dependent luciferase reporter activity in HEK293/CRE-Luc/hGLP1R cells with EC₅₀ values ranging from 93.2 to 101 pM whereas GLP1R agonist LPs M6457, M6562, and M6677 induced CRE-dependent luciferase activity with EC₅₀ values of 10.3, 11.2, and 31.2 pM, respectively. Anti-GDF8 mAb REGN1033 did not induce CRE-dependent luciferase reporter activity in HEK293/CRE-Luc/hGLP1R cells.

Anti-GDF8 Antibody conjugates increased CRE-dependent luciferase reporter activity in HEK293/CRE-Luc/hGLP1R cells, in the absence of GDF8, with different EC₅₀ values: 200 pM for REGN1033-M6457, 57.5 pM for REGN1033-M6562 (Q55), 2.39 nM for REGN1033-M6562 (Q295), and 131 pM for REGN1033-M6677 (Q55). GLP1R agonist activity of anti-GDF8 Antibody conjugates was affected, at different degrees, by the presence of 100 nM GDF8 in the CRE-Luc assay: EC₅₀ value of 1.83 nM for REGN1033-M6457, 5.00 nM for REGN1033-M6562 (Q55), 5.05 nM for REGN1033-M6562 (Q295), and 1.40 nM for REGN1033-M6677 (Q55). Isotype control Antibody conjugates induced CRE-dependent luciferase activity in HEK293/CRE-Luc/hGLP1R cells, in the absence of GDF8, with EC₅₀ values of 212 pM for REGN1945-M6457 and 63.5 pM for H4H30045P-M6562 (Q55). The presence of 100 nM GDF8 in the CRE-Luc assay had minor influence in the potency of isotype antibody conjugate H4H30045P-M6562 (Q55) (EC₅₀ value of 107 pM) (Table 37).

LPs and Antibody conjugates increased CRE-dependent luciferase reporter activity in HEK293/CRE-Luc/hGLP1R cells in the presence or absence of 100 nM GDF8 with Eₘₐₓ relative to GLP-1 ranging from 76.5% to 89.0% (Table 37).

TABLE 37
CRE-Dependent Reporter Activity Induced by GLP1R Agonists
and mAb/Antibody Conjugates in HEK293/CRE-Luc/hGLP1R Cells
in the Absence of GDF8 or in the Presence of 100 nM GDF8

| | | HEK293/CRE-Luc/hGLP1R | | | | |
|---|---|---|---|---|---|---|
| | | Without GDF8 | | With 100 nM GDF8 | | |
| | | | Eₘₐₓ | | Eₘₐₓ | |
| | | EC₅₀ | (% | EC₅₀ | (% | |
| Test article | LP | (M) | GLP-1) | (M) | GLP-1) | Experiment |
| GLP-1 (7-36) amide | | 9.32E−11 | 100.0 | NT | NT | A |
| | | 9.45E−11 | 100.0 | | | B * |
| | | 1.01E−10 | 100.0 | | | C * |
| M6457 | | 1.03E−11 | 82.1 | NT | NT | A |
| M6562 | | 1.12E−11 | 82.8 | NT | NT | A |
| M6677 | | 3.12E−11 | 78.6 | NT | NT | B |
| REGN1033 | | >5.00E−07 | 0.8 | NT | NT | C |
| (anti-GDF8 mAb) | | | | | | |
| REGN1033-M6457 | M6457 | 2.00E−10 | 83.1 | 1.83E−09 | 81.1 | A |
| (anti-GDF8 AC) | | | | | | |
| REGN1945-M6457 | M6457 | 2.12E−10 | 89.0 | NT | NT | A |
| (isotype AC) | | | | | | |
| REGN1033-M6562 | M6562 | 5.75E−11 | 85.8 | 5.00E−09 | 88.2 | A |
| (Q55) (anti-GDF8 AC) | | | | | | |
| H4H30045P-M6562 | M6562 | 6.35E−11 | 85.6 | 1.07E−10 | 76.5 | A |
| (Q55) (isotype AC) | | | | | | |
| REGN1033-M6562 | M6562 | 2.39E−09 | 86.1 | 5.05E−09 | 88.1 | A |
| (Q295) (anti-GDF8 AC) | | | | | | |
| REGN1033-M6677 | M6677 | 1.31E−10 | 82.4 | 1.40E−09 | 86.3 | B |
| (Q55) (anti-GDF8 AC) | | | | | | |

>=EC₅₀ values could not be determined with accuracy because luminescence signal did not reach saturation within the tested concentration range. EC₅₀ is reported as greater than the highest tested concentration.
NT, not tested.
* GLP-1 (7-36) amide included in independent experiments A, B, and C with similar results.

As shown in Table 38, anti-GDF8 mAb REGN1033 and anti-GDF8 Antibody conjugates (REGN1033-M6457, REGN1033-M6562 (Q55), REGN1033-M6562 (Q295), and REGN1033-M6677 (Q55)) blocked GDF8-dependent reporter activity in A204/CAGA-Luc cells, with IC₅₀ values ranging from 593 pM to 4.72 nM and maximum blocking percentage ranging from 98.1 to 100.1%. Isotype control antibody conjugates (REGN1945-M6457 and H4H30045P-M6562 (Q55)) did not promote significant blocking of GDF8-dependent signaling in A204/CAGA-Luc cells.

TABLE 38
Blocking of GDF8-Dependent Reporter Activity in
A204/CAGA-Luc Cells by mAb/Antibody Conjugates (AC)

| | | A204/CAGA-Luc | | |
|---|---|---|---|---|
| | | With 1 nM GDF8 | | |
| | | | Max | |
| Test article | LP | IC₅₀ (M) | blocking (%) | Experiment |
| REGN1033 | | 8.70E−10 | 99.4 | A |
| (anti-GDF8 mAb) | | 3.30E−09 | 98.1 | B * |
| REGN1033-M6457 | M6457 | 1.41E−09 | 99.7 | A |
| (anti-GDF8 AC) | | | | |
| REGN1945-M6457 | M6457 | >1.00E−07 | 17.7 | A |
| (isotype AC) | | | | |
| REGN1033-M6562 | M6562 | 5.93E−10 | 100.1 | A |
| (Q55) | | | | |
| H4H30045P-M6562 | M6562 | >1.00E−07 | 13.7 | A |
| (Q55) (isotype) | | | | |
| REGN1033-M6562 | M6562 | 4.72E−09 | 99.0 | B |
| (Q295) | | | | |
| REGN1033-M6677 | M6677 | 6.58E−10 | 99.1 | A |
| (Q55) | | | | |

> = IC₅₀ values could not be determined with accuracy because luminescence signal did not reach the lower plateau within the tested concentration range. IC₅₀ is reported as greater than the highest tested concentration.
* REGN1033 included in independent experiments A and B with similar results.

These results show that anti-GDF8 Antibody conjugates with GLP1R agonists as LPs can activate GLP1R in cell-based bioassay while maintaining the GDF8 blocking ability of the unconjugated REGN1033 mAb.

### Example 11. Biacore Kinetics of Anti-GDF8 Antibody Conjugates Binding to Dimeric Human GDF8 in an Antibody Capture Format at 25° C.

Equilibrium dissociation constants (K_{D} values) of anti-GDF8 antibody conjugates binding to dimeric human GDF8 were determined using real-time surface plasmon resonance biosensor technology on a Biacore T-200 instrument. Dimeric human GDF8 was expressed without a tag (REGN30: NENSEQKENVEKEGLCNACTWRQNTKSSRIEAIKIQILSKLRLETAPNISKDVIRQLLPKA PPLRELIDQYDVQRDDSSDGSLEDDDYHATTETIITMPTESDFLMQVDGKPKCCFFKFSS KIQYNKVVKAQLWIYLRPVETPTTVFVQILRLIKPMKDGTRYTGIRSLKLDMNPGTGIW QSIDVKTVLQNWLKQPESNLGIEIKALDENGHDLAVTFPGPGEDGLNPFLEVKVTDTPK RRRRDFGLDCDEHSTESRCCRYPLTVDFEAFGWDWIIAPKRYKANYCSGECEFVFLQKY PHTHLVHQANPRGSAGPCCTPTKMSPINMLYFNGKEQIIYGKIPAMVVDRCGCS, SEQ ID NO: 21). Briefly, the CM5 Biacore sensor surface was derivatized by amine coupling a monoclonal mouse anti-human Fc monoclonal antibody. All Biacore binding studies were performed in a buffer composed of 10 mM HEPES pH 7.4, 150 mM NaCl, 0.05% v/v Surfactant P20 (HBS-EP running buffer). Anti-GDF8 antibody conjugates were captured onto the anti-hFc surface, and different concentrations of dimeric human GDF8, ranging from 0.6 nM to 48.5 nM in 3-fold serial dilutions, were injected at a flow rate of 30 μL/minute. Antibody-ligand association was monitored for 5 minutes, and dissociation was monitored for 10 minutes. At the end of each cycle, the hIgG capture surface was regenerated using a 10 second injection of 20 mM phosphoric acid. All binding kinetics experiments were performed at 25° C.

The specific SPR-Biacore sensorgrams were obtained by a double referencing procedure. The double referencing was performed by first subtracting the signal of each injection over a reference surface (anti-hFc) from the signal over the experimental surface (anti-hFc-captured anti-GDF8-antibody conjugates) thereby removing contributions from refractive index changes. In addition, running buffer injections were performed to allow subtraction of the signal changes resulting from the dissociation of captured antibodies from the coupled anti-hFc surface. Kinetic association (kₐ) and dissociation (k_{d}) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = \frac{kd}{ka} , and {t}_{1 / 2} ( min ) = \frac{ln ( 2 )}{60 * kd}$$

Human GDF8 binding kinetics results to anti-GDF8-antibody conjugates obtained at 25° C. are summarized in Table 39. The unconjugated anti-GDF8 mAb had similar or weaker binding affinity to dimeric human GDF8 as the anti-GDF8- antibody conjugates.

TABLE 39
Kinetic and Equilibrium Binding Parameters of Human GDF8
binding to Surface-Captured Anti-GDF8-Antibody Conjugates

| | | | hGDF8 | | | | |
|---|---|---|---|---|---|---|---|
| | | | (REGN30) | | | | |
| | | mAb | Bound at | | | | |
| Antibody | Linker | Capture | 48.5 nM | kₐ | k_{d} | K_{D} | t_{1/2} |
| REGN # | Payload | (RU) | (RU) | (1/Ms) | (1/s) | (m) | (min) |
| REGN1033 | none | 185.2 ± 1.5 | 49.3 | 4.40E+06 | 8.03E−05 | 1.83E−11 | 143.8 |
| (L70) | | | | | | | |
| REGN1033 | M6457 | 191.1 ± 1.9 | 44.6 | 5.41E+06 | 3.89E−05 | 7.18E−12 | 297.4 |
| (L80) | | | | | | | |
| REGN1033 | M6562 | 209.0 ± 3.7 | 49.7 | 4.60E+06 | 6.72E−05 | 1.46E−11 | 171.9 |
| (L81) | | | | | | | |
| REGN1033 | M6562 | 206.2 ± 2.0 | 52.4 | 4.90E+06 | 8.42E−05 | 1.72E−11 | 137.3 |
| (L82) | | | | | | | |
| REGN1033 | M6562 | 222.9 ± 2.6 | 55.8 | 1.43E+07 | 4.27E−05 | 2.98E−12 | 270.5 |
| (L83) | | | | | | | |
| REGN1033 | M6677 | | | | | | |
| (L83) | | | | | | | |

### Example 12. First In Vivo Study: In Vivo Comparison of Cys-Conjugated GLP-1 Anti-Myostatin Antibody (REGN1033) to Q55-Conjugated Antibody for Inducing Fat Mass Loss while Preserving Lean Mass

TABLE 40
mAb Clone IDs

| | | |
|---|---|---|
| H4H300045P | Lot# H4H30045P-L2 | Stock 51.5 |
| (Control mAb) | | mg/ml |
| H4H300045P^{Q55--GLP-1} | Lot# H4H30045P-L7 | Stock 2.96 |
| (Isotype-M6562-Q55) | | mg/ml |
| REGN1033^{CYS-GLP-1} | Lot# REGN1033-L83 | Stock 3.41 |
| (REGN1033-M6457-CYS) | | mg/ml |
| REGN1033^{Q55-GLP-1} | Lot# REGN1033-L80 | Stock is 5.97 |
| (REGN1033-M6562-Q55) | | mg/ml |

A GLP1R agonist was conjugated to REGN1033 (anti-myostatin antibody through using a native cysteine (REGN1033-M6457) as well as an isotype control antibody (Isotype Control-M6457 is the antibody conjugate). It was found that while both Isotype Control-M6457 and REGN1033-M6457 lowered body weight and blood glucose, Isotype Control-M6457 also reduced lean mass which was completely prevented with myostatin blockade. Thus REGN1033-M6457 was capable of reducing body weight and preserving lean mass in a single molecule.

Subsequently a GLP1R agonist was conjugated to REGN1033 using a native glutamine in the light chain (REGN1033-M6562) as an alternate conjugation method. The same GLP1R agonist was also conjugated to a control antibody which also had a conjugatable glutamine in its light chain (Isotype-M6562), which binds human FGFR3 and does not cross with mouse FGFR3 (Control mAb is the unconjugated antibody alone). This study was conducted to test how REGN1033-M6562 compares to REGN1033-M6457 to lower body weight and increase muscle mass in diet-induced obese (DIO) male mice. For the study, 24 to 26-week-old male mice, who were on a 60% high-fat diet for 17-19 weeks, were separated into four groups of 6-10 mice each based on their baseline fat mass and body weight. The four treatment groups were as follows: 1) Control mAb: single treatment of isotype control at 10 mg/kg; 2) Isotype control-M6562: single treatment at 10 mg/kg; 3) REGN1033-M6457: single treatment at 10 mg/kg; or 4) REGN1033-M6562: single treatment at 10 mg/kg. Each mouse was given 4 treatment doses over a four-week period (dosing schedule and groups are listed in Table 41).

TABLE 41
Study Groups and Dosing

| Group | (N) | Treatment (dose) | Group | Frequency | Route |
|---|---|---|---|---|---|
| 1 | 10 | Isotype Control | Control mAb | Weekly | S.C. |
| | | H4H300045P | | | |
| | | (10 mg/kg) | | | |
| 2 | 9 | H4H300045PQ^{55--GLP-1} | Isotype-M6562 | Weekly | S.C. |
| | | (10 mg/kg) | | | |
| 3 | 6 | REGN1033^{CYS-GLP-1} | REGN1033- | Weekly | S.C. |
| | | (10 mg/kg) | M6457 | | |
| 4 | 9 | REGN1033^{Q55-GLP-1} | REGN1033- | Weekly | S.C. |
| | | (10 mg/kg) | M6562 | | |

Body weights were taken throughout the study and body composition was measured by EchoMRI at baseline and on study days 14 and 23. Fed glucose was measured by glucometer from the tail prior to dosing every week. On day 28, the mice were euthanized in a staggered fashion and the following organ weights were measured: tibialis anterior (TA) muscle, quadriceps muscle (Quad), gastrocnemius muscle (GA; dissection also included the soleus), subcutaneous white adipose (scWAT), gonadal white adipose tissue (gWAT), mesenteric adipose tissue (mWAT), pancreas, and liver.

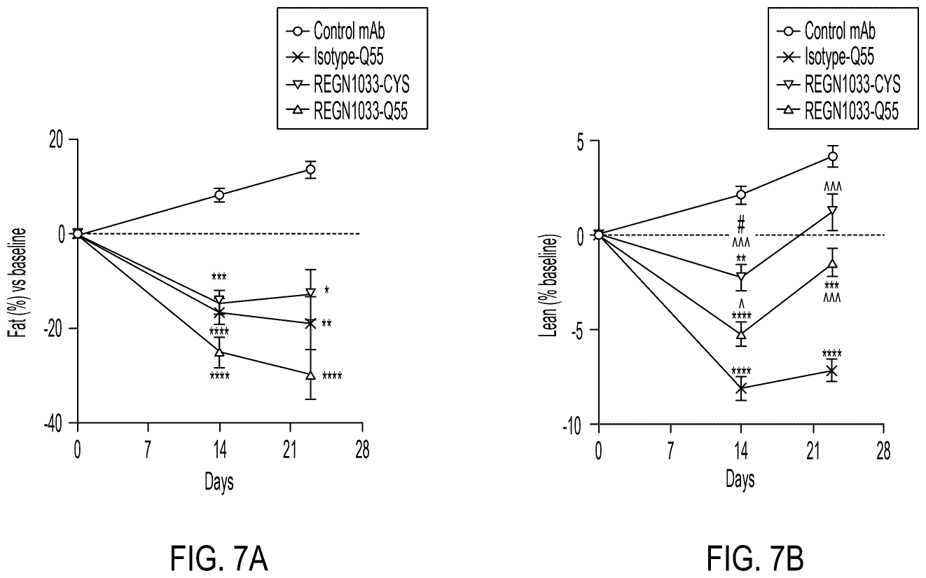

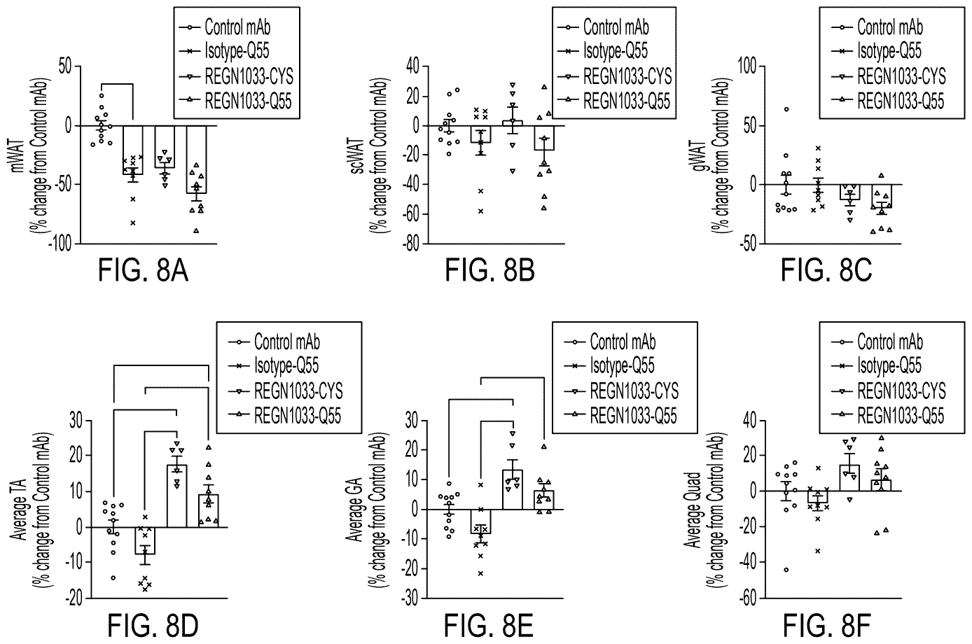

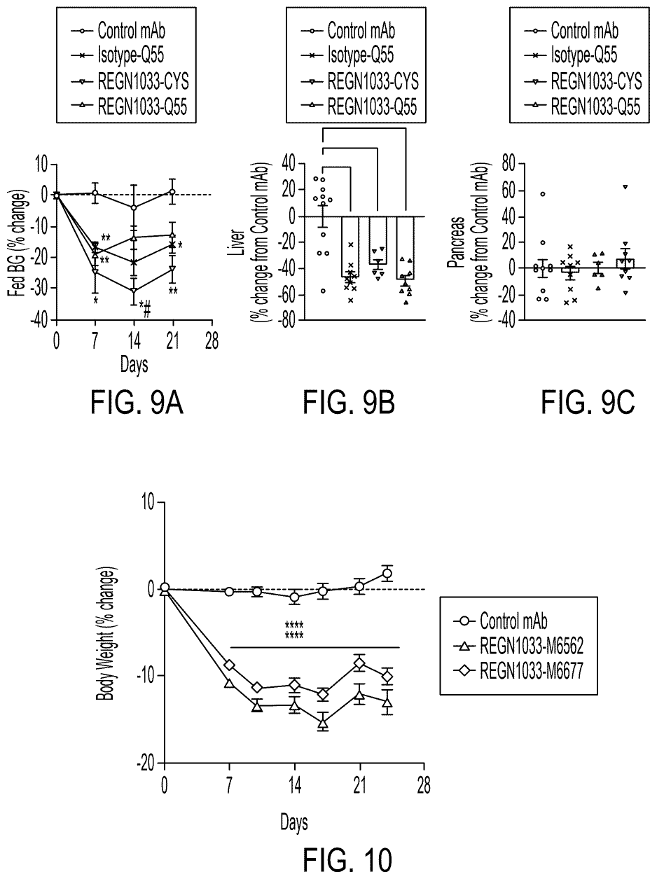

Change in body weight, total body fat, and total lean mass were calculated as the percent difference from each time point compared to each baseline reading. Body weights are shown in FIG. 6 and body composition is shown in FIGS. 7A-7B. Terminal muscle and fat weights are shown in FIGS. 8A-8F, while terminal pancreas and liver weights, as well as fed glucose are shown in FIGS. 9A-9C. Changes over time were assessed by two-way ANOVA while the final results were compared by one-way ANOVA.

### Body Weight

Percent change in body weight is shown in FIG. 6. All the groups which received a GLP1R agonist conjugated to either REGN1033 or the Isotype Control lost 4-13% of their body weight over the four-week study. The control group gained 5% body weight since these mice were non-weight stable, so GLP1R agonism was able to not only reduce body weight from baseline, but also prevent additional weight gain from the high-fat diet.

### Body Composition

Body composition was assessed at baseline, D14 of the study (following the first two doses) and at D23 (following all four doses). Change from baseline is plotted in FIGS. 7A-7B for % change in fat mass (FIG. 7A) and % change in lean mass (FIG. 7B) from baseline.

### Fat Mass

The isotype control mAb group gained 14% fat mass over the four-week study. All groups which received GLP-1 lost fat mass with the Isotype-M6562control group losing 19% and the REGN1033-M6562 group losing nearly 30%, both greater than the REGN1033-M6457 group which lost 13%. Thus the GLP1R agonist conjugated to REGN1033 at Q55 reduces fat mass to a greater extent than when conjugated to native cysteines.

### Lean Mass

The isotype control mAb group gained 4% lean mass over the four-week study. In contrast, the Isotype control-M6562 control group lost 7% lean mass which was mitigated or prevented with myostatin blockade. REGN1033-M6457 had a 1% increase in lean mass at the end of the study while REGN1033-M6562 had a 1% decrease, both significantly more than the Isotype-M6562 group. Thus the GLP1R agonist conjugated to an anti-myostatin antibody at either position is capable of reducing fat mass and body weight while maintaining lean mass using a single molecule.

The terminal fat and muscle weights showed similar effects (FIGS. 8A-8F). GLP1R agonist reduced mesenteric fat to a greater extent than either subcutaneous or visceral fat (FIGS. 8A-8C) while REGN1033 was able to reverse GLP1R agonist induced skeletal muscle loss in all three muscles samples: tibialis anterior, gastrocnemius, and quadriceps (FIGS. 8D-8F) by either Q55 or CYS conjugation.

### Blood Glucose, Liver, and Pancreas Weight

Percent change in fed glucose, liver weight, and pancreas weight is shown in FIGS. 9A-9C. All the groups which received GLP1R agonist conjugated to either REGN1033 or Control mAb reduced blood glucose and liver weight over the four-week study (FIGS. 9A-9B). The reduction in blood glucose was maintained over the duration of the study and no reduction was seen in the Control mAb group. No change in pancreas weight was seen in any group.

REGN1033-M6562reduced fat mass and body weight to a greater extent than the REGN1033-M6457 conjugate while preserving lean mass to a similar extent. This suggests the GLP1R agonist conjugated through Q55 may be more effective at lowering body weight than native cysteine conjugation with similar effects on lean mass preservation.

### Example 13. Second In Vivo Study: In Vivo Comparison of Two Antibody Conjugates REGN1033-M6562 and REGN1033-M6677 for Inducing Fat Mass Loss while Preserving Lean Mass

As previously described a GLP-1R agonist was conjugated to REGN1033 (anti-myostatin antibody) through a native glutamine in the light chain (REGN1033-M6562) and was compared to another REGN1033 (anti-myostatin antibody) conjugated through a native glutamine in the light chain where the linker payload was generated through a different synthetic route (REGN1033-M6677). This study was conducted to test how REGN1033-M6562 compares to REGN1033-M6677 to lower body weight and increase muscle mass in diet-induced obese (DIO) male mice. For the study, 26-week-old male mice, who were on a 60% high-fat diet for 17-19 weeks, were separated into three groups of 10 mice each based on their baseline fat mass and body weight. The three treatment groups were as follows: 1) Control mAb: isotype control H4H300045P at 10 mg/kg; 2) REGN1033-M6562 at 10 mg/kg; 3) REGN1033-M6677 at 10 mg/kg. Each mouse was given 4 treatment doses over a four-week period (dosing schedule and groups are listed in Table 42).

TABLE 42
Study Groups and Dosing

| Group | (N) | Treatment (dose) | Group | Frequency | Route |
|---|---|---|---|---|---|
| 1 | 10 | Isotype Control | Control mAb | Weekly | S.C. |
| | | H4H300045P | | | |
| | | (10 mg/kg) | | | |
| 2 | 10 | REGN1033^{Q55-GLP-1} | REGN1033- | Weekly | S.C. |
| | | (M6562) (10 mg/kg) | M6562 | | |
| 3 | 10 | REGN1033^{Q55-GLP-1} | REGN1033- | Weekly | S.C. |
| | | (M6677) (10 mg/kg) | M6677 | | |

Body weights were taken throughout the study and body composition was measured by EchoMRI at baseline and on study days 14 and 27. Fed glucose was measured by glucometer from the tail prior to dosing every week. On day 28, the mice were euthanized in a staggered fashion and the following organ weights were measured: tibialis anterior (TA) muscle, quadriceps muscle (Quad), gastrocnemius muscle (GA; dissection also included the soleus), subcutaneous white adipose (scWAT), gonadal white adipose tissue (gWAT), mesenteric adipose tissue (mWAT), pancreas, and liver.

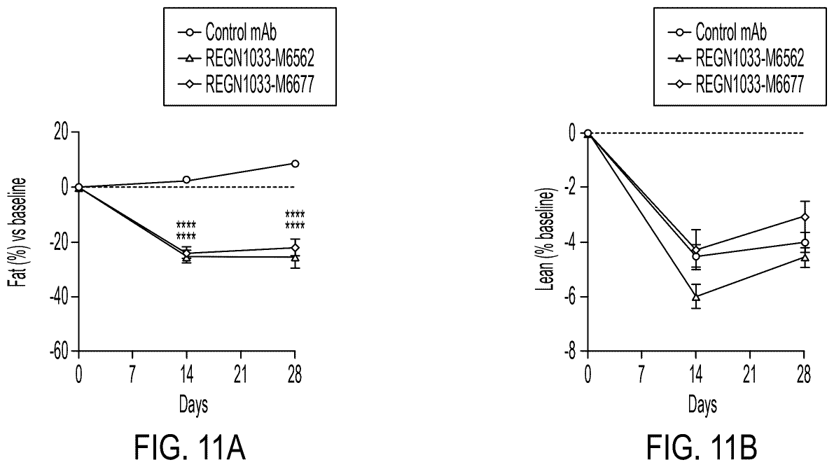

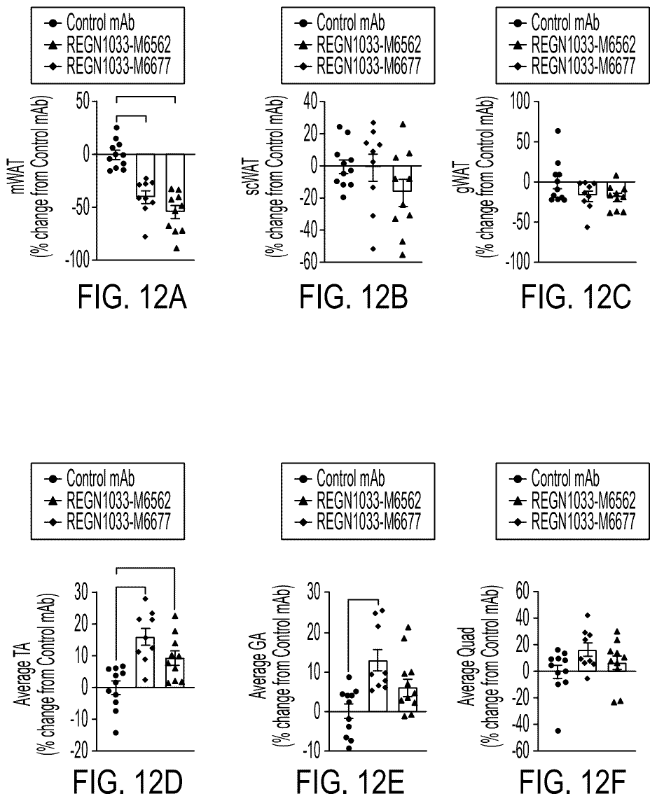

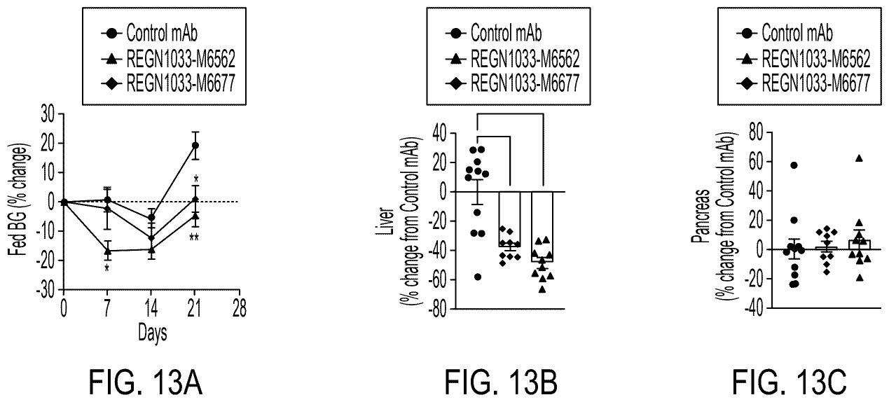

Change in body weight, total body fat, and total lean mass were calculated as the percent difference from each time point compared to each baseline reading. Body weights are shown in FIG. 10 and body composition is shown in FIGS. 11A-11B. Terminal muscle and fat weights are shown in FIGS. 12A-12F, while terminal pancreas and liver weights, as well as fed glucose are shown in FIGS. 13A-13C. Changes over time were assessed by two-way ANOVA while the final results were compared by one-way ANOVA.

### Body Weight

Percent change in body weight is shown in FIG. 10. All the groups which received a GLP1R agonist conjugated to REGN1033 lost 10-13% of their body weight over the four-week study.

### Body Composition

Body composition was assessed at baseline, D14 of the study (following the first two doses) and at D23 (following all four doses). Change from baseline is plotted in FIGS. 11A-11B for % change in fat mass (FIG. 11A) and % change in lean mass (FIG. 11B) from baseline.

### Fat Mass

All groups which received a GLP1R agonist peptidomimetic conjugate lost fat mass with the REGN1033-M6562 group losing nearly 26% and the REGN-M6677 group losing nearly 22%.

### Lean Mass

REGN1033-M6562 had a −4.5% change in lean mass while the REGN1033-M6677 had a −3% change in lean mass at the end of the study vs. −4.0% for the control.

The terminal fat and muscle weights showed similar effects for the fat (FIGS. 12A-12F). GLP1R agonist reduced mesenteric fat to a greater extent than either subcutaneous or visceral fat (FIGS. 12A-12C) while REGN1033 was able to reverse GLP1R agonist induced skeletal muscle loss in the tibialis anterior (TA) and gastrocnemius (GA) and slightly increased the quadriceps (FIGS. 12D-12F).

### Blood Glucose, Liver and Pancreas Weight

Percent change in fed glucose, liver weight, and pancreas weight is shown in FIGS. 13A-13C. All the groups which received a GLP1R agonist conjugated to REGN1033 reduced blood glucose and liver weight over the four-week study (FIGS. 13A-13B). The reduction in blood glucose was maintained over the duration of the study. No change in pancreas weight was seen in any group.

REGN1033-M6562 and REGN1033-M6677 reduced fat mass and body weight similarly, while preserving lean mass to a similar extent.

### Example 14. Site Specific Antibody Conjugation at Q55 of the Light Chain

The surprising conjugation selectivity over Q55 of the light chain REGN1033 and the discovery that the conjugate products showed excellent in vivo and in vitro activities promoted further explorations of these site specific conjugations in other antibodies, including GLP1R antibodies, anti-Activin A/B antibodies, anti-ActRIIA/B antibodies, anti-MSR1 antibodies, anti-CACNG1 antibodies, and antibodies to the following targets CD20, CD226, MERS, HLA-B27, IL-6R, STEAP2, MET×MET, FGFR2b, FLT3, KIT, EGFR, CD1B, PMSA, NECTIN4, FOLR1, EGFRvIII, VPREB1, and LEPR. Specific examples include REGN4320 (anti-MSR1 N297Q), REGN4322 (H1H21231N, anti-MSR1, N297Q), H2aM21339N (anti-HLA-A2/CMV), H4H11283N (anti-HLA-B27), and H4sH14137N (anti-CD20), H1H20918P (CD226), H4H20122P (HLA-B27), H4H13767P (HFE2), H4H12587P (IL6R), H4H11281N (HLA-B27), and H1H15208P (MERS).

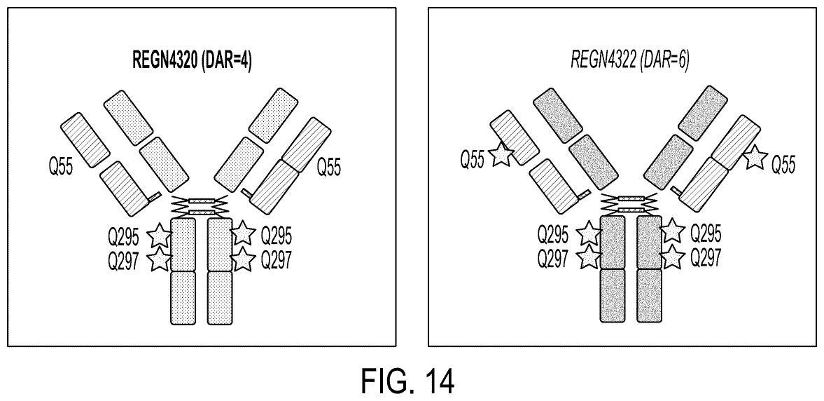

As demonstrated in Table 43 from several conjugation experiments, it appears that L46F of the light chain (LC) from ULC VK V1-39-J5 mAbs is beneficial toward Q55 reactivity for transglutaminase-based site-specific conjugation (FIG. 14). mAbs with a sequence of FLIYAASSLQSGVPSR (SEQ ID NO: 112) on the variable region of LC were able to conjugate with linker-payload via MTG based site-specific conjugations.

TABLE 43
Comparison of Conjugation Efficiency
at Q55 Among Several Antibodies

| | AA46 on | Q55 on | | |
|---|---|---|---|---|
| | ULC VK1- | ULC VK1- | Q for TG | |
| Antibody | 39-J5 | 39-J5 | reaction | DAR |
| REGN4320 | L | Q | Q295 | 4 |
| (anti-MSR1 N297Q) | | | Q297 | |
| REGN4322 | F | Q | Q295 | 6 |
| (anti-MSR1 N297Q) | | | Q297 | |
| | | | Q55 | |
| H2aM21339N | L | Q | none | 0 |
| (anti-HLA-A2/CMV) | | | | |
| H4H11283N | F | Q | Q55 | 1.8 |
| (anti-HLA-B27) | | | | |

All three a-MSR1-LXR ADCs induced similar LXR-Luc activity, although each had different conjugation yield (Table 44). Detailed methods are reported in Han, et al., US 2022/0112158 A1, Apr. 14, 2022.

TABLE 44
In vitro Activities of Antibody Conjugates

| | | | | | | ADC Activity |
|---|---|---|---|---|---|---|
| a-MSR1- | | Sequence | Linker- | | ADC | (DAR adjusted) |
| LXR ADC | mAb | features | pay-load | DAR | purification | post 40 h |
| REGN4320- | REGN4320 | Q55 at VK | M301 | 4 | Low yield | Full activation, |
| M301 | (H1H21227N- | not used | | | (<10%) | EC50 = 1.2 nM |
| | N297Q) | for TG | | | Low | |
| | | reaction | | | concentration | |
| | | | | | (<0.1 mg/ml) | |
| REGN4322- | REGN4322 | Q55 at VK | M301 | 6 | difficult to | Full activation, |
| M301 | (H1H21231N- | used for TG | | | purify due to | EC50 = 1.7 nM |
| | N297Q) | reaction | | | 6 of | |
| | | | | | hydrophobic | |
| | | | | | drug load | |
| REGN4323- | REGN4323 | | M301 | 4 | Good yield | Full activation, |
| M301 | (H1H21234N- | | | | (>50%) | EC50 = 1.9 nM |
| | N297Q) | | | | | |

### Sequences of Related Antibodies:

| REGN4320_Sequence | |
|---|---|
| REGN4320_HC: | |
| (SEQ ID NO: 22) | |
| QVQLVQSGAEVKKPGASVKVSCKVSGYTLTELSIHWVRQVPGKGLEWM | |
| GGFDPEEGETIFAQEFRDRVTLTEDTSPDTAYMELSSLKSEDAAVYYCTTPRYCNNGICY | |
| DYWGQGTLVTVSSASTKGPSVFPLAPSSKSTSGGTAALGCLVKDYFPEPVTVSWNSGAL | |
| TSGVHTFPAVLQSSGLYSLSSVVTVPSSSLGTQTYICNVNHKPSNTKVDKKVEPKSCDKT | |
| HTCPPCPAPELLGGPSVFLFPPKPKDTLMISRTPEVTCVVVDVSHEDPEVKFNWYVDGV | |
| EVHNAKTKPREEQ₂₉₅YQSTYRVVSVLTVLHQDWLNGKEYKCKVSNKALPAPIEKTISKA | |
| KGQPREPQVYTLPPSRDELTKNQVSLTCLVKGFYPSDIAVEWESNGQPENNYKTTPPVL | |
| DSDGSFFLYSKLTVDKSRWQQGNVFSCSVMHEALHNHYTQKSLSLSPGK* | |
| REGN4320_LC: | |
| (SEQ ID NO: 23) | |
| DIQMTQSPSSLSASVGDRVTITCRASQSISNYLNWYQQKPGKAPKLLIYTA | |
| SSLQ₅₅SGVPSRFSGSGSGTDFTLTISSLQTEDFATYYCQQSYSNFPITFGQGTRLEIKRTVA | |
| APSVFIFPPSDEQLKSGTASVVCLLNNFYPREAKVQWKVDNALQSGNSQESVTEQDSKD | |
| STYSLSSTLTLSKADYEKHKVYACEVTHQGLSSPVTKSFNRGEC* | |
| REGN4322_Sequence | |
| HC: 49226.50 Da | |
| (SEQ ID NO: 24) | |
| EVQLVESGGGLVQPGGSLRLSCAASGFTFSDHYMDWVRQAPGKGLEWV | |
| GRTRNKANSHTTEYTASVTGRFTISRDDSRNSLYLQMNSLKTEDTAVYYCVRAGIIGTLF | |
| DYWGQGTLVTVSSASTKGPSVFPLAPSSKSTSGGTAALGCLVKDYFPEPVTVSWNSGAL | |
| TSGVHTFPAVLQSSGLYSLSSVVTVPSSSLGTQTYICNVNHKPSNTKVDKKVEPKSCDKT | |
| HTCPPCPAPELLGGPSVFLFPPKPKDTLMISRTPEVTCVVVDVSHEDPEVKFNWYVDGV | |
| EVHNAKTKPREEQ₂₉₅YQSTYRVVSVLTVLHQDWLNGKEYKCKVSNKALPAPIEKTISKA | |
| KGQPREPQVYTLPPSRDELTKNQVSLTCLVKGFYPSDIAVEWESNGQPENNYKTTPPVL | |
| DSDGSFFLYSKLTVDKSRWQQGNVFSCSVMHEALHNHYTQKSLSLSPGK* | |
| LC: 23466.13Da | |
| (SEQ ID NO: 25) | |
| DIQMTQFPSSLSASVGDRVTITCRASQSIS_SFLNWFQQKPGKAPKFLIYAA | |
| SSLQ₅₅SGVPS | |
| RFSGSGSGTDFTLTISSLQPEDFATYYCQQSYSSPPITFGQGTRLEIKRTVAAPSVFIFPPSD | |
| EQLKSGTASVVCLLNNFYPREAKVQWKVDNALQSGNSQESVTEQDSKDSTYSLSSTLTL | |
| SKADYEKHKVYACEVTHQGLSSPVTKSFNRGEC* | |
| REGN4323_Sequence | |
| HC: 49246.70 Da | |
| (SEQ ID NO: 26) | |
| QVQLQESGPGLVKPSETLSLTCTVTGGSISRNYWSWIRQPPGKGLEWIGYI | |
| YYSGSIDYNPSLKSRVTISVDTSKNQFSLKLSSMTAADTAVYYCARDRWNWKYGMDV | |
| WGQGTTVIVSSASTKGPSVFPLAPSSKSTSGGTAALGCLVKDYFPEPVTVSWNSGALTS | |
| GVHTFPAVLQSSGLYSLSSVVTVPSSSLGTQTYICNVNHKPSNTKVDKKVEPKSCDKTH | |
| TCPPCPAPELLGGPSVFLFPPKPKDTLMISRTPEVTCVVVDVSHEDPEVKFNWYVDGVE | |
| VHNAKTKPREEQYQSTYRVVSVLTVLHQDWLNGKEYKCKVSNKALPAPIEKTISKAKG | |
| QPREPQVYTLPPSRDELTKNQVSLTCLVKGFYPSDIAVEWESNGQPENNYKTTPPVLDS | |
| DGSFFLYSKLTVDKSRWQQGNVFSCSVMHEALHNHYTQKSLSLSPGK* | |
| LC: 23669.34 Da | |
| (SEQ ID NO: 27) | |
| EIVLTQSPGTLSLSPGERATLSCRASQTVRNNYLAWYHQKPGQAPRLLIY | |
| GASSRATGIPDRESGSGSGTDFTLTISRLEPEDFTVYYCHQYGNSPWTFGQGTKMEIKRT | |
| VAAPSVFIFPPSDEQLKSGTASVVCLLNNFYPREAKVQWKVDNALQSGNSQESVTEQDS | |
| KDSTYSLSSTLTLSKADYEKHKVYACEVTHQGLSSPVTKSFNRGEC*. | |
| Additional protein sequences of cited antibodies: | |
| Trevogrumab, REGN1033 heavy chain sequence: | |
| (SEQ ID NO: 28) | |
| EVQVLESGGDLVQPGGSLRLSCAASGFTFSAYAMTWVRQAPGKGLEWVSAISGSGGSA | |
| YYADSVKGRFTISRDNSKNTVYLQMNSLRAEDTAVYYCAKDGAWKMSGLDVWGQGTT | |
| VIVSSASTKGPSVFPLAPCSRSTSESTAALGCLVKDYFPEPVTVSWNSGALTSGVHTFPAV | |
| LQSSGLYSLSSVVTVPSSSLGTKTYTCNVDHKPSNTKVDKRVESKYGPPCPPCPAPEFLG | |
| GPSVFLFPPKPKDTLMISRTPEVTCVVVDVSQEDPEVQFNWYVDGVEVHNAKTKPREEQ | |
| FNSTYRVVSVLTVLHQDWLNGKEYKCKVSNKGLPSSIEKTISKAKGQPREPQVYTLPPSQ | |
| EEMTKNQVSLTCLVKGFYPSDIAVEWESNGQPENNYKTTPPVLDSDGSFFLYSRLTVDKS | |
| RWQEGNVFSCSVMHEALHNHYTQKSLSLSLGK | |
| Trevogrumab, REGN1033 light chain sequence: | |
| DIQMTQSPASLSASVGDRVTITCRASQDISDYLAWYQQKPGKIPRLLIYTTSTLQ*SGVPS | |
| RFRGSGSGTDFTLTISSLQPEDVATYYCQKYDSAPLTFGGGTKVEIKRTVAAPSVFIFPPSD | |
| EQLKSGTASVVCLLNNFYPREAKVQWKVDNALQSGNSQESVTEQDSKDSTYSLSSTLTL | |
| SKADYEKHKVYACEVTHQGLSSPVTKSFNRGEC (SEQ ID NO: 29; *Q55 represents the | |
| conjugation site of the GLP1 agonist) | |
| Apitegromab heavy chain | |
| (SEQ ID NO: 30) | |
| QVQLVESGGGVVQPGRSLRLSCAASGFTFSSYGMHWVRQAPGKGLEWVAVISYDGSN | 100 |
| KYYADSVKGRFTISRDNSKNTLYLQMNSLRAEDTAVYYCARDL | |
| LVRFLEWSHYYGMDVWGQGTTVTVSSASTKGPSVFPLAPCSRSTSESTAALGCLVKDY | 200 |
| FPEPVTVSWNSGALTSGVHTFPAVLQSSGLYSLSSVVTVPSS | |
| SLGTKTYTCNVDHKPSNTKVDKRVESKYGPPCPPCPAPEFLGGPSVFLFPPKPKDTLMIS | 300 |
| RTPEVTCVVVDVSQEDPEVQFNWYVDGVEVHNAKTKPREE | |
| QFNSTYRVVSVLTVLHQDWLNGKEYKCKVSNKGLPSSIEKTISKAKGQPREPQVYTLPP | 400 |
| SQEEMTKNQVSLTCLVKGFYPSDIAVEWESNGQPENNYKTT | |
| PPVLDSDGSFFLYSRLTVDKSRWQEGNVFSCSVMHEALHNHYTQKSLSLSLG | |
| Apitegromab light chain | |
| (SEQ ID NO: 31) | |
| QSVLTQPPSASGTPGQRVTISCSGSSSNIGSNTVHWYQQLPGTAPKLLIYSDNQRPSGVPD | 100 |
| RFSGSKSGTSASLAISGLQSEDEADYYCAAWDDSLNGVF | |
| GGGTKLTVLGQPKAAPSVTLFPPSSEELQANKATLVCLISDFYPGAVTVAWKADSSPVK | 200 |
| AGVETTTPSKQSNNKYAASSYLSLTPEQWKSHRSYSCQVTH | |
| EGSTVEKTVAPTECS | 215 |
| Bimagrumab heavy chain Sequence | |
| (SEQ ID NO: 32) | |
| QVQLVQSGAEVKKPGASVKVSCKASGYTFTSSYINWVRQAPGQGLEWMGTINPVSGST | 100 |
| SYAQKFQGRVTMTRDTSISTAYMELSRLRSDDTAVYYCARGG | |
| WFDYWGQGTLVTVSSASTKGPSVFPLAPSSKSTSGGTAALGCLVKDYFPEPVTVSWNSG | 200 |
| ALTSGVHTFPAVLQSSGLYSLSSVVTVPSSSLGTQTYICNV | |
| NHKPSNTKVDKRVEPKSCDKTHTCPPCPAPEAAGGPSVFLFPPKPKDTLMISRTPEVTCV | 300 |
| VVDVSHEDPEVKFNWYVDGVEVHNAKTKPREEQYNSTYRV | |
| VSVLTVLHQDWLNGKEYKCKVSNKALPAPIEKTISKAKGQPREPQVYTLPPSREEMTKN | 400 |
| QVSLTCLVKGFYPSDIAVEWESNGQPENNYKTTPPVLDSDG | |
| SFFLYSKLTVDKSRWQQGNVFSCSVMHEALHNHYTQKSLSLSPGK | |
| Bimagrumab Light chain sequence | |
| (SEQ ID NO: 33) | |
| QSALTQPASVSGSPGQSITISCTGTSSDVGSYNYVNWYQQHPGKAPKLMIYGVSKRPSGV | 100 |
| SNRFSGSKSGNTASLTISGLQAEDEADYYCGTFAGGSYYG | |
| VFGGGTKLTVLGQPKAAPSVTLFPPSSEELQANKATLVCLISDFYPGAVTVAWKADSSPV | 200 |
| KAGVETTTPSKQSNNKYAASSYLSLTPEQWKSHRSYSCQV | |
| THEGSTVEKTVAPTECS | |
| Domagrozumab heavy chain | |
| (SEQ ID NO: 34) | |
| EVQLLESGGGLVQPGGSLRLSCAASGFTFSSYAMSWVRQAPGKGLEWVSTISSGGSYTS | 100 |
| YPDSVKGRFTISRDNSKNTLYLQMNSLRAEDTAVYYCAKQD | |
| YAMNYWGQGTLVTVSSASTKGPSVFPLAPSSKSTSGGTAALGCLVKDYFPEPVTVSWN | 200 |
| SGALTSGVHTFPAVLQSSGLYSLSSVVTVPSSSLGTQTYICN | |
| VNHKPSNTKVDKKVEPKSCDKTHTCPPCPAPEAAGAPSVFLFPPKPKDTLMISRTPEVTC | 300 |
| VVVDVSHEDPEVKFNWYVDGVEVHNAKTKPREEQYNSTYR | |
| VVSVLTVLHQDWLNGKEYKCKVSNKALPAPIEKTISKAKGQPREPQVYTLPPSREEMTK | 400 |
| NQVSLTCLVKGFYPSDIAVEWESNGQPENNYKTTPPVLDSD | |
| GSFFLYSKLTVDKSRWQQGNVFSCSVMHEALHNHYTQKSLSLSPG | |
| Domagrozumab light chain | |
| (SEQ ID NO: 35) | |
| DIQMTQSPSSLSASVGDRVTITCKASQDVSTAVAWYQQKPGKAPKLLIYSASYRYTGVP | 100 |
| SRFSGSGSGTDFTLTISSLQPEDFATYYCQQHYSTPWTFGG | |
| GTKVEIKRTVAAPSVFIFPPSDEQLKSGTASVVCLLNNFYPREAKVQWKVDNALQSGNS | 200 |
| QESVTEQDSKDSTYSLSSTLTLSKADYEKHKVYACEVTHQG | |
| LSSPVTKSFNRGEC | |
| Landogrozumab heavy chain | |
| (SEQ ID NO: 36) | |
| EVOLVESGGGLVQPGGSLRLSCAASGLTFSRYPMSWVRQAPGKGLVWVSAITSSGGSTY | 100 |
| YSDTVKGRFTISRDNAKNTLYLQMNSLRAEDTAVYYCARLP | |
| DYWGQGTLVTVSSASTKGPSVFPLAPCSRSTSESTAALGCLVKDYFPEPVTVSWNSGALT | 200 |
| SGVHTFPAVLQSSGLYSLSSVVTVPSSSLGTKTYTCNVDH | |
| KPSNTKVDKRVESKYGPPCPPCPAPEFLGGPSVFLFPPKPKDTLMISRTPEVTCVVVDVSQ | 300 |
| EDPEVQFNWYVDGVEVHNAKTKPREEQFNSTYRVVSVLT | |
| VLHQDWLNGKEYKCKVSNKGLPSSIEKTISKAKGQPREPQVYTLPPSQEEMTKNQVSLT | 400 |
| CLVKGFYPSDIAVEWESNGQPENNYKTTPPVLDSDGSFFLY | |
| SRLTVDKSRWQEGNVFSCSVMHEALHNHYTQKSLSLSLG | |
| Landogrozumab light chain | |
| (SEQ ID NO: 37) | |
| EIVLTQSPGTLSLSPGERATLSCRASSSVSSSYLHWYQQKPGQAPRLLIYSTSNLVAGIPDR | 100 |
| FSGSGSGTDFTLTISRLEPEDFAVYYCQHHSGYHFTFG | |
| GGTKVEIKRTVAAPSVFIFPPSDEQLKSGTASVVCLLNNFYPREAKVQWKVDNALQSGN | 200 |
| SQESVTEQDSKDSTYSLSSTLTLSKADYEKHKVYACEVTHQ | |
| GLSSPVTKSFNRGEC. | 191 |

### Example 15. Characterization of Stereo Isomers Generated from Click Reaction During Conjugation with Selective Enzyme Digestion and High-Resolution LC-MS

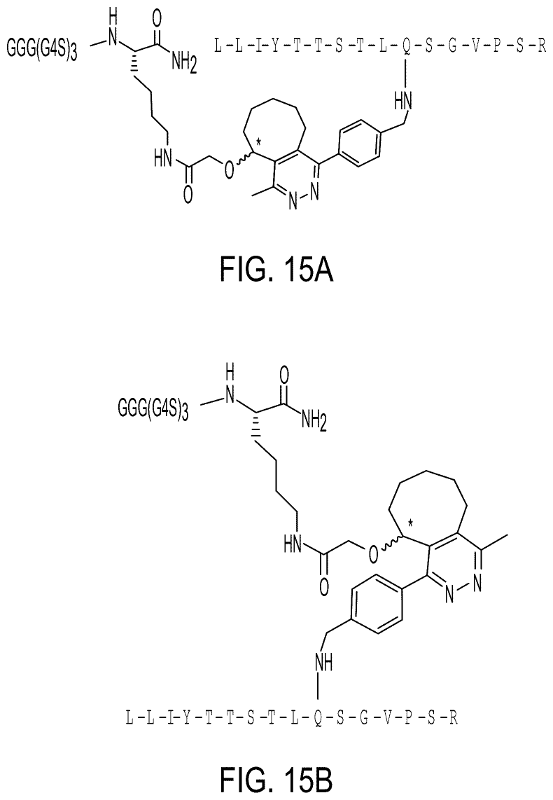

Due to the unsymmetric nature of the reactive moieties participated in the Click reaction (e.g. also called inverse electron demand Diels-Alder (IEDDA) reaction) during the conjugation process, cis- and trans- stereo isomers were generated. Together with the chiral center of the (cyclooct-2-yn-1-yloxy) acetyl moiety, there are four potential stereoisomers, e.g. cis-R, cis-S, trans-R, and trans-S. As shown in FIG. 15, those four isomers were identified and confirmed using selective trypsin digestion followed by characterization with high-resolution LCMS detailed in the section below.

### Sample Preparation and Analysis for Peptide Mapping Experiment

REGN1033-M6092-M6562 was added into 0.15% RapiGest in 0.1 M Tris-HCl solution (pH 7.5) to reach final concentration at 0.5 μg/μL, followed by the addition of 0.1 M tris (2-carboxyethyl)phosphine hydrochloride (TCEP) to 5 mM. The sample was vortexed and then incubated at 65° C. for 15 min. After the incubation, 0.1 M iodoacetamide was added to reach final concentration of 5 mM before incubated at 37° C. in dark for 30 min. The sample was then digested by MS grade trypsin (Promega) with 1:20 (w/w) enzyme to substrate ratio overnight at 37° C. The resulting sample pH was adjusted to <2.0 by 10% TFA and incubated for additional 30 min at 37° C. The mixture was centrifuged, and the supernatant was collected for LC-MS/MS analysis on Orbitrap Exploris 480 Mass Spectrometer coupled with Vanquish UHPLC.

The separation was carried on Waters ACQUITY Peptide BEH C18 column (130 Å 1.7 μm 2.1 mm×150 mm) with mobile phase A: 0.1% formic acid in water, mobile phase B: 0.1% formic acid in acetonitrile. LC gradient was from 0.5% mobile phase B to 45% mobile phase B over 85 min at a flow rate of 0.25 ml/min with column temperature of 45° C. All eluted peptides were monitored first by photodioarray detector (PDA) and then by Orbitrap MS analyzer equipped with heated-ESI probe operating in positive mode at 3.5 kV spray voltage. MS analyzer was set at the following parameter: MS1 resolution: 60,000; MS1 scan range: 200-2000 m/z, MS1 AGC target: 100%; MS2 resolution: 15,000; MS2 AGC target: standard; normalized collision energy=30%, and RF lens at 50%. Raw data was analyzed by BiopharmFinder 4.1 (Thermo Scientific) to identify the peptides based on MS1 and MS2 spectra with mass accuracy setting <15 ppm.

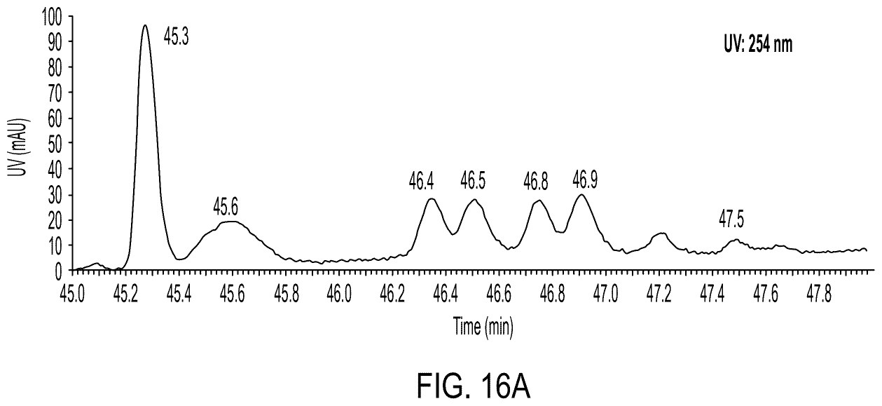

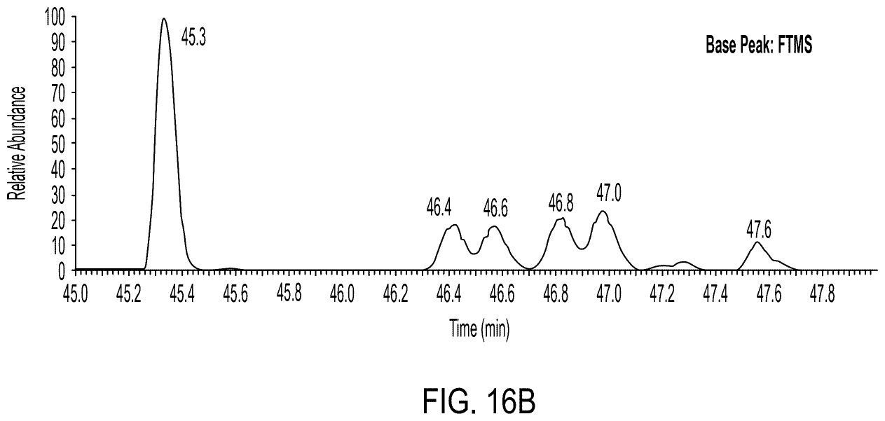

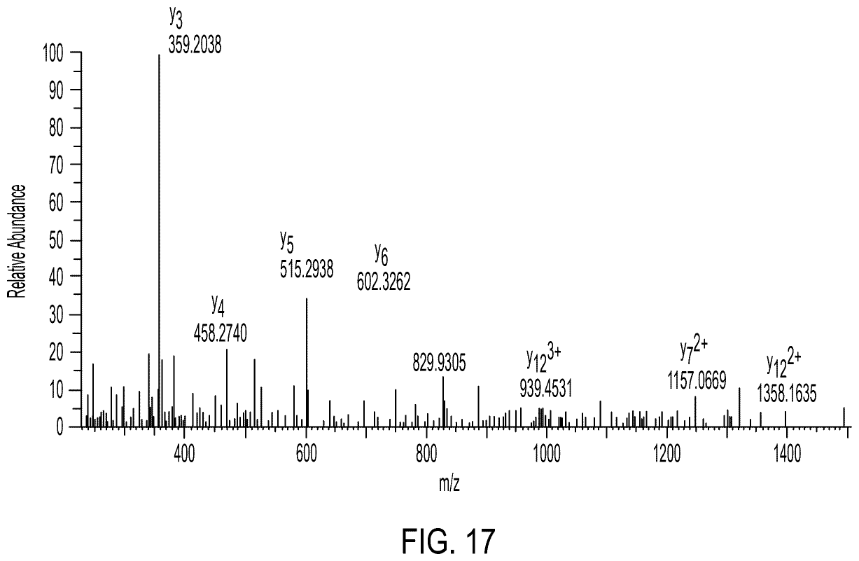

Digested peptides bearing a portion of M6092-M6562 (MS1 accuracy <3 ppm) were observed in MS and UV chromatograms at four different retention time by peptide mapping using MS/MS technology. Four peaks showing identical MS/MS spectra are identified (FIG. 16A, UV spectrum monitored at 254 nm; FIG. 16B, FTMS signals). Given the existence of chiral center in M6562 and the regio-chemistry of the conjugation coupling reaction, four major peaks at different retention times correspond to isomeric combination of R/S and cis/trans configuration (FIGS. 16A-16B). It is interesting to observe that those four stereo isomers were roughly in an equal amount based on the intensity of the MS signals. As shown in FIG. 17, the labelled MS signals were fragmented ion matched to theoretical fragment from the peptide, identified by the software. Data process software (Biopharmfinder in this case) makes theoretical calculation based on peptide sequence and potential modifications on amino acids to predict the fragmented ion, then match them with experimental MS/MS spectra (usually within 10 ppm mass difference) to confirm the sequence/modifications and locate the modification sites. y-ion is from C-term, b-ion is from N-term of the cleavage of peptide bond (see below), e.g. y2(2+) mean y2 ion from C-term of peptide but bearing two positive charges. The structural information of those interested molecular fragments from the trypsin digestion and their corresponding theoretical and calculated masses are summarized in the Table 45 and Table 46.

TABLE 45
Identified Fragmented Ion y and b Ion From MS/MS
Spectrum of Tryptic Peptide Conjugated With M6092-M6562

| | b ion | | y ion | |
|---|---|---|---|---|
| AA | Theoretical | ΔMass | Theoretical | ΔMass |
| Sequence | m/z | (ppm) | (m/z) | (ppm) |
| L | N/A | N/A | N/A | N/A |
| L | N/A | N/A | N/A | N/A |
| I | 340.2595 | −0.39 | N/A | N/A |
| Y | N/A | N/A | N/A | N/A |
| T | N/A | N/A | N/A | N/A |
| T | N/A | N/A | N/A | N/A |
| S | N/A | N/A | N/A | N/A |
| T | 893.4979 | −9.81 | N/A | N/A |
| L | N/A | N/A | N/A | N/A |
| Q | N/A | N/A | N/A | N/A |
| S | N/A | N/A | 602.3257 | +0.68 |
| G | N/A | N/A | 515.2936 | −0.63 |
| V | N/A | N/A | 458.2722 | +4.63 |
| P | N/A | N/A | 359.2037 | −0.16 |
| S | N/A | N/A | 262.1510 | −1.28 |
| R | N/A | N/A | N/A | N/A |

Peptide fragment-LLIYTTSTLQ*SGVPSR-(SEQ ID NO: 113) was part of the REGN1033 antibody generated during trypsin digestion (see also FIGS. 15 and 17).

TABLE 46
Identified m/z Value of Four Peaks of the Stereo Isomers

| Retention | Observed | Theoretical | |
|---|---|---|---|
| time | monoisotopic | monoisotopic | Δ mass |
| (min) | mass (Da) | mass (Da) | (ppm) |
| 46.41 | 3316.6506 | 3316.6436 | 2.12 |
| 46.57 | 3316.6501 | 3316.6436 | 1.97 |
| 46.82 | 3316.6497 | 3316.6436 | 1.83 |
| 46.97 | 3316.6504 | 3316.6436 | 2.05 |

### Example 16. Additional Examples of Linker-Payload Peptides

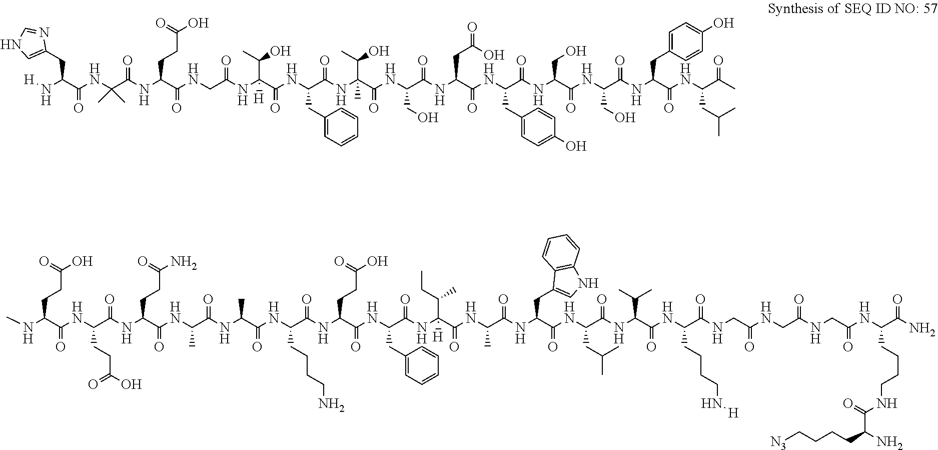

Peptide Sequence:
(SEQ ID NO: 57)
H[Aib]EGTFTSDYSSYLEEQAAKEFIAWLVKGGGK[K(N3)]-
CONH2.

Molecular Formula: C167H248N44O51. Molecular Weight: 3688.08. Counter Ion: TFA. Formula Weight: 4258.18 (+5 TFA). Physical Appearance: White Powder. Storage: −20° C. Weight: 10.0 mg.

Coupling was carried out using Syroll Synthesizer (Table 47).

TABLE 47
Synthesis of SEQ ID NO: 57
Resin: Rink Amide MBHA (loading, 0.30 mmol/g, 0.15 g)

| | | Coupling | Reacting | | | Coupling | Reacting |
|---|---|---|---|---|---|---|---|
| Step # | Materials | reagents | time | Step # | Materials | reagents | time |
| 1 | Fmoc- | AA(4 eq) | 40 min × 2 | 22 | Fmoc- | AA(4 eq) | 40 min × 2 |
| | Lys(Dde)-OH | HBTU(4 eq) | | | Tyr(tBu)- | HBTU(4 eq) | |
| | | DIPEA(8 eq) | | | OH | DIPEA(8 eq) | |
| 2 | Fmoc-Gly-OH | AA(4 eq) | 40 min | 23 | Fmoc- | AA(4 eq) | 40 min × 2 |
| | | HBTU(4 eq) | | | Asp(OtBu)- | HBTU(4 eq) | |
| | | DIPEA(8 eq) | | | OH | DIPEA(8 eq) | |
| 3 | Fmoc-Gly- | AA(4 eq) | 40 min | 24 | Fmoc- | AA(4 eq) | 40 min × 2 |
| | Gly-OH | HBTU(4 eq) | | | Ser(tBu)- | HBTU(4 eq) | |
| | | DIPEA(8 eq) | | | OH | DIPEA(8 eq) | |
| 4 | Fmoc- | AA(4 eq) | 40 min | 25 | Fmoc- | AA(4 eq) | 40 min × 2 |
| | Lys(Boc)-OH | HBTU(4 eq) | | | Thr(tBu)- | HBTU(4 eq) | |
| | | DIPEA(8 eq) | | | OH | DIPEA(8 eq) | |
| 5 | Fmoc-Val-OH | AA(4 eq) | 40 min | 26 | Fmoc-Phe- | AA(4 eq) | 40 min × 2 |
| | | HBTU(4 eq) | | | OH | HBTU(4 eq) | |
| | | DIPEA(8 eq) | | | | DIPEA(8 eq) | |
| 6 | Fmoc-Leu- | AA(4 eq) | 40 min | 27 | Fmoc- | AA(4 eq) | 40 min × 2 |
| | OH | HBTU(4 eq) | | | Thr(tBu)- | HBTU(4 eq) | |
| | | DIPEA(8 eq) | | | OH | DIPEA(8 eq) | |
| 7 | Fmoc- | AA(4 eq) | 40 min × 2 | 28 | Fmoc-Gly- | AA(4 eq) | 40 min × 2 |
| | Trp(Boc)-OH | HBTU(4 eq) | | | OH | HBTU(4 eq) | |
| | | DIPEA(8 eq) | | | | DIPEA(8 eq) | |
| 8 | Fmoc-Ala-OH | AA(4 eq) | 40 min × 2 | 29 | Fmoc- | AA(4 eq) | 40 min × 2 |
| | | HBTU(4 eq) | | | Glu(OtBu)- | HBTU(4 eq) | |
| | | DIPEA(8 eq) | | | OH | DIPEA(8 eq) | |
| 9 | Fmoc-Ile-OH | AA(4 eq) | 40 min × 2 | 30 | Fmoc-Aib- | AA(4 eq) | 40 min × 2 |
| | | HBTU(4 eq) | | | OH | HBTU(4 eq) | |
| | | DIPEA(8 eq) | | | | DIPEA(8 eq) | |
| 10 | Fmoc-Phe- | AA(4 eq) | 40 min × 2 | 31 | Boc- | AA(4 eq) | 40 min × 2 |
| | OH | HBTU(4 eq) | | | His(Trt)- | HBTU(4 eq) | |
| | | DIPEA(8 eq) | | | OH | DIPEA(8 eq) | |
| 11 | Fmoc- | AA(4 eq) | 40 min × 2 | 32 | Dde | 10% hydrazine | 30 min |
| | Glu(OtBu)- | HBTU(4 eq) | | | removal | in DMF | |
| | OH | DIPEA(8 eq) | | | | | |
| 12 | Fmoc- | AA(4 eq) | 40 min × 2 | 33 | Fmoc- | AA(4 eq) | 40 min × 2 |
| | Lys(Boc)-OH | HBTU(4 eq) | | | Lys(N3)- | HBTU(4 eq) | |
| | | DIPEA(8 eq) | | | OH | DIPEA(8 eq) | |
| 13 | Fmoc-Ala-OH | AA(4 eq) | 40 min × 2 | 34 | | | |
| | | HBTU(4 eq) | | | | | |
| | | DIPEA(8 eq) | | | | | |
| 14 | Fmoc-Ala-OH | AA(4 eq) | 40 min × 2 | 35 | | | |
| | | HBTU(4 eq) | | | | | |
| | | DIPEA(8 eq) | | | | | |
| 15 | Fmoc- | AA(4 eq) | 40 min × 2 | 36 | | | |
| | Gln(Trt)-OH | HBTU(4 eq) | | | | | |
| | | DIPEA(8 eq) | | | | | |
| 16 | Fmoc- | AA(4 eq) | 40 min × 2 | 37 | | | |
| | Glu(OtBu)- | HBTU(4 eq) | | | | | |
| | OH | DIPEA(8 eq) | | | | | |
| 17 | Fmoc- | AA(4 eq) | 40 min × 2 | 38 | | | |
| | Glu(OtBu)- | HBTU(4 eq) | | | | | |
| | OH | DIPEA(8 eq) | | | | | |
| 18 | Fmoc-Leu- | AA(4 eq) | 40 min × 2 | 39 | | | |
| | OH | HBTU(4 eq) | | | | | |
| | | DIPEA(8 eq) | | | | | |
| 19 | Fmoc- | AA(4 eq) | 40 min × 2 | 40 | | | |
| | Tyr(tBu)-OH | HBTU(4 eq) | | | | | |
| | | DIPEA(8 eq) | | | | | |
| 20 | Fmoc- | AA(4 eq) | 40 min × 2 | 41 | | | |
| | Ser(tBu)-OH | HBTU(4 eq) | | | | | |
| | | DIPEA(8 eq) | | | | | |
| 21 | Fmoc- | AA(4 eq) | 40 min × 2 | 42 | | | |
| | Ser(tBu)-OH | HBTU(4 eq) | | | | | |
| | | DIPEA(8 eq) | | | | | |

| | |
|---|---|
| De-Fmoc | 20% Piperidine in DMF 2 × 10 min |
| Cleavage | 90.0% TFA/ 2.5% H2O/5.0% 3-Mpa/2.5% TIS |
| Crude | 150 mg |
| amount: | |
| Isolated | 10 g |
| amount | |

### Procedure

Step 1: the MBHA resin swelled in DMF (10 ml) for 1 hour and filtered.

Step 2: 20% piperidine/DMF was added into the reaction column. The mixture was bubbled with N₂ for 10 min and filtered. The resin was washed with DMF twice. Another 20% piperidine/DMF was added into the reaction column. The mixture was bubbled with N₂ for 10 min and filtered. The resin was washed with DMF (10 ml*6).

Step 3: a Kaiser test was taken. If the test was positive, moved to the next step. If the test was negative, Step 2 was repeated again until the Kaiser test result was positive.

Step 4: Fmoc-AA-OH (4 eq, 0.8 mmol), HBTU (4 eq, 0.8 mmol), HOBT (4 eq, 0.8 mmol), DIEA (8 eq, 1.6 mmol), and DMF (5 ml) were added into the reaction column. Then the reaction mixture was bubbled with nitrogen gas for 1 hour. The resin was filtered and washed with DMF (10 mL*6).

Step 5: 20% piperidine/DMF was added into the reaction column. The mixture was bubbled with N₂ for 10 min and filtered. The resin was washed with DMF twice. Another 20% piperidine/DMF was added into the reaction column. The mixture was bubbled with N₂ for 10 min and filtered. The resin was washed with DMF (10 ml*6).

Step 6: A Kaiser test for the resin was performed. If the test was positive, the next step was performed. Is the test was negative, Step 5 was performed again until the result of the Kaiser test was positive.

Step 7: AC₂O (3 eq, 0.6 mmol), DIEA (6 eq, 1.2 mmol), and DMF (5 ml) were added into the reaction column. The mixture was bubbled with N₂ for 10 min and filtered. The resin was washed with DMF (10 ml*6).

Step 8: A Kaiser test for the resin was performed. If the test was negative, the next step was performed. Is the test was positive, Step 7 was performed again.

Step 9: the peptide resin was shrank with MTBE (10 ml) for 2 min, twice.

Cleavage: dry resin (700 mg) was placed in a flask and 7 mL of cleavage solution (TFA:Tis:EDT:H2O=90:2.5:5:2.5, v/v/v/v) was added. The reaction was allowed to proceed for 3 hours. The resin was removed by filtration under pressure. The TFA was dried with nitrogen gas. The crude peptide (150 mg) was obtained as a white solid. The peptide was purified by prep-HPLC (0.05% TFA/CH₃CN/H₂O) to afford the target peptide (10 mg) as a white solid.

Data and Result: HPLC purity 93.65%, rt. 9.79 min; MS m/z (100%) highest m/z peak: 1230.7 [M+3H]/³⁺, 922.8 [M+4H]/⁴⁺.

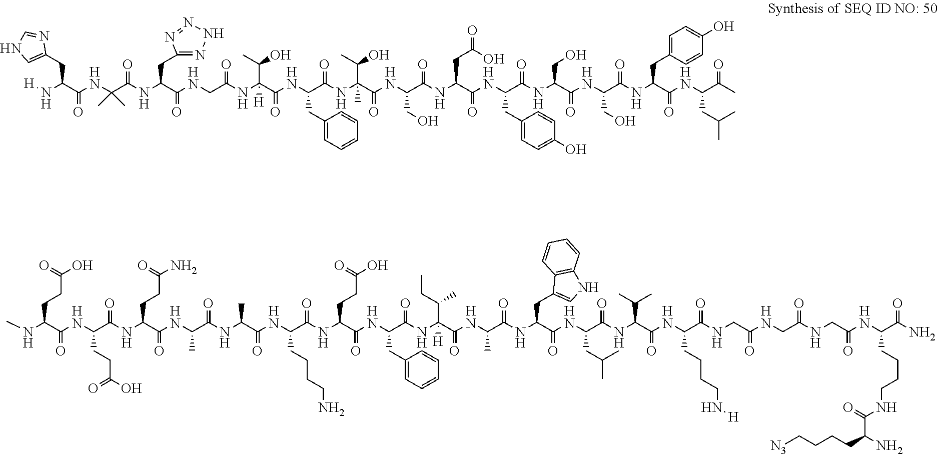

Peptide Sequence:
(SEQ ID NO: 50)
H[Aib][Atz]GTFTSDYSSYLEEQAAKEFIAWLVKGGGK[K(N3)]-
CONH2.

Molecular Formula: C166H246N48O49. Molecular Weight: 3698.08. Counter Ion: TFA. Formula Weight: 4382.20 (+6 TFA). Physical Appearance: White Powder. Storage: -20° C. Weight: 10.0 mg.

Coupling was carried out using the Syroll Synthesizer (Table 48).

TABLE 48
Synthesis of SEQ ID NO: 50
Resin: Rink Amide MBHA (loading, 0.30 mmol/g, 0.15 g)

| | | Coupling | Reacting | | | Coupling | Reacting |
|---|---|---|---|---|---|---|---|
| Step # | Materials | reagents | time | Step # | Materials | reagents | time |
| 1 | Fmoc- | AA(4 eq) | 40 min × 2 | 22 | Fmoc- | AA(4 eq) | 40 min × 2 |
| | Lys(Dde)- | HBTU(4 eq) | | | Tyr(tBu)- | HBTU(4 eq) | |
| | OH | DIPEA(8 eq) | | | OH | DIPEA(8 eq) | |
| 2 | Fmoc-Gly- | AA(4 eq) | 40 min | 23 | Fmoc- | HBTU(4 eq) | 40 min × 2 |
| | OH | HBTU(4 eq) | | | Asp(OtBu)- | AA(4 eq) | |
| | | DIPEA(8 eq) | | | OH | DIPEA(8 eq) | |
| 3 | Fmoc-Gly- | AA(4 eq) | 40 min | 24 | Fmoc- | AA(4 eq) | 40 min × 2 |
| | Gly-OH | HBTU(4 eq) | | | Ser(tBu)- | HBTU(4 eq) | |
| | | DIPEA(8 eq) | | | OH | DIPEA(8 eq) | |
| 4 | Fmoc- | AA(4 eq) | 40 min | 25 | Fmoc- | AA(4 eq) | 40 min × 2 |
| | Lys(Boc)- | HBTU(4 eq) | | | Thr(tBu)- | HBTU(4 eq) | |
| | OH | DIPEA(8 eq) | | | OH | DIPEA(8 eq) | |
| 5 | Fmoc-Val- | AA(4 eq) | 40 min | 26 | Fmoc-Phe- | AA(4 eq) | 40 min × 2 |
| | OH | HBTU(4 eq) | | | OH | HBTU(4 eq) | |
| | | DIPEA(8 eq) | | | | DIPEA(8 eq) | |
| 6 | Fmoc-Leu- | AA(4 eq) | 40 min | 27 | Fmoc- | AA(4 eq) | 40 min × 2 |
| | OH | HBTU(4 eq) | | | Thr(tBu)- | HBTU(4 eq) | |
| | | DIPEA(8 eq) | | | OH | DIPEA(8 eq) | |
| 7 | Fmoc- | AA(4 eq) | 40 min × 2 | 28 | Fmoc-Gly- | AA(4 eq) | 40 min × 2 |
| | Trp(Boc)- | HBTU(4 eq) | | | OH | HBTU(4 eq) | |
| | OH | DIPEA(8 eq) | | | | DIPEA(8 eq) | |
| 8 | Fmoc-Ala- | AA(4 eq) | 40 min × 2 | 29 | Fmoc-Atz- | AA(4 eq) | 40 min × 2 |
| | OH | HBTU(4 eq) | | | OH | HBTU(4 eq) | |
| | | DIPEA(8 eq) | | | | DIPEA(8 eq) | |
| 9 | Fmoc-Ile- | AA(4 eq) | 40 min × 2 | 30 | Fmoc-Aib- | AA(4 eq) | 40 min × 2 |
| | OH | HBTU(4 eq) | | | OH | HBTU(4 eq) | |
| | | DIPEA(8 eq) | | | | DIPEA(8 eq) | |
| 10 | Fmoc-Phe- | AA(4 eq) | 40 min × 2 | 31 | Boc- | AA(4 eq) | 40 min × 2 |
| | OH | HBTU(4 eq) | | | His(Trt)- | HBTU(4 eq) | |
| | | DIPEA(8 eq) | | | OH | DIPEA(8 eq) | |
| 11 | Fmoc- | HBTU(4 eq) | 40 min × 2 | 32 | Dde | 10% | 30 min |
| | Glu(OtBu)- | AA(4 eq) | | | removal | hydrazine in | |
| | OH | DIPEA(8 eq) | | | | DMF | |
| 12 | Fmoc- | AA(4 eq) | 40 min × 2 | 33 | Fmoc- | AA(4 eq) | 40 min × 2 |
| | Lys(Boc)- | HBTU(4 eq) | | | Lys(N3)- | HBTU(4 eq) | |
| | OH | DIPEA(8 eq) | | | OH | DIPEA(8 eq) | |
| 13 | Fmoc-Ala- | AA(4 eq) | 40 min × 2 | 34 | | | |
| | OH | HBTU(4 eq) | | | | | |
| | | DIPEA(8 eq) | | | | | |
| 14 | Fmoc-Ala- | AA(4 eq) | 40 min × 2 | 35 | | | |
| | OH | HBTU(4 eq) | | | | | |
| | | DIPEA(8 eq) | | | | | |
| 15 | Fmoc- | AA(4 eq) | 40 min × 2 | 36 | | | |
| | Gln(Trt)- | HBTU(4 eq) | | | | | |
| | OH | DIPEA(8 eq) | | | | | |
| 16 | Fmoc- | HBTU(4 eq) | 40 min × 2 | 37 | | | |
| | Glu(OtBu)- | AA(4 eq) | | | | | |
| | OH | DIPEA(8 eq) | | | | | |
| 17 | Fmoc- | HBTU(4 eq) | 40 min × 2 | 38 | | | |
| | Glu(OtBu)- | AA(4 eq) | | | | | |
| | OH | DIPEA(8 eq) | | | | | |
| 18 | Fmoc-Leu- | AA(4 eq) | 40 min × 2 | 39 | | | |
| | OH | HBTU(4 eq) | | | | | |
| | | DIPEA(8 eq) | | | | | |
| 19 | Fmoc- | AA(4 eq) | 40 min × 2 | 40 | | | |
| | Tyr(tBu)- | HBTU(4 eq) | | | | | |
| | OH | DIPEA(8 eq) | | | | | |
| 20 | Fmoc- | AA(4 eq) | 40 min × 2 | 41 | | | |
| | Ser(tBu)- | HBTU(4 eq) | | | | | |
| | OH | DIPEA(8 eq) | | | | | |
| 21 | Fmoc- | AA(4 eq) | 40 min × 2 | 42 | | | |
| | Ser(tBu)- | HBTU(4 eq) | | | | | |
| | OH | DIPEA(8 eq) | | | | | |

| | |
|---|---|
| De-Fmoc | 20% Piperidine in DMF 2 × 10 min |
| Cleavage | 90.0% TFA/ 2.5% H2O/5.0% 3-Mpa/2.5% TIS |
| Crude | 150 mg |
| amount: | |
| Isolated | 10 mg |
| amount | |

### Procedure

Step 1: the MBHA resin was swelled in DMF (10 ml) for 1 hour and filtered.

Step 2: 20% piperidine/DMF was added into the reaction column. The mixture was bubbled with N₂ for 10 min and filtered. The resin was washed with DMF twice. Another 20% piperidine/DMF was added into the reaction column. The mixture was bubbled with N₂ for 10 min and filtered. The resin was washed with DMF (10 ml*6).

Step 3: A Kaiser test for the resin was performed. If the test was positive, the next step was carried our. If the test was negative, Step 2 was performed again until the Kaiser test result was positive.

Step 4: Fmoc-AA-OH (4 eq, 0.8 mmol), HBTU (4 eq, 0.8 mmol), HOBT (4 eq, 0.8 mmol), DIEA (8 eq, 1.6 mmol), and DMF(5 ml) were added into the reaction column. Then the mixture was bubbled with N₂ for 1 hour and filtered. The resin was washed with DMF (10 ml*6).

Step 5: 20% piperidine/DMF was added into the reaction column. The mixture was bubbled with N₂ for 10 min and filtered. The resin was washed with DMF twice. Another 20% piperidine/DMF was added into the reaction column. The mixture was bubbled with N₂ for 10 min and filtered. The resin was washed with DMF (10 ml*6).

Step 6: A Kaiser test for the resin was performed. If the test was positive, the next step was performed. If the test was negative, Step 5 was performed again until the result of the Kaiser test was positive.

Step 7: AC₂O (3 eq, 0.6 mmol), DIEA (6 eq, 1.2 mmol), and DMF (5 ml) were added into the reaction column. Then the mixture was bubbled with N₂ for 1 hours and filtered. The resin was washed with DMF (10 ml*6).

Step 8: A Kaiser test was performed. If the test was negative, the next step was performed. If the test was positive, Step 7 was repeated.

Step 9: The resin was shrunk with MTBE (10 ml) for 2 min twice.

Cleavage: the dry resin (700 mg) was placed in a flask and 7 mL of cleavage solution (TFA:Tis:EDT:H2O=90:2.5:5:2.5, v/v/v/v) was added. The reaction was carried out for 3 hours. The resin was removed by filtration under pressure. The TFA was dried with nitrogen gas. Crude peptide (150 mg) was obtained as a white solid. The peptide was purified by prep-HPLC (0.05% TFA/CH₃CN/H₂O) to afford the target peptide (10 mg) as a white solid.

Data and Result: LC-MS/HPLC (after purification) RT 9.92 min, purity 93.24%; 1233.4 [M+3H]/³⁺.

### Synthesis of M6571

Peptide Sequence:
(SEQ ID NO: 55)
H[Aib]EGT[amF(2F)]TSDYSSYLEEQAAKEFIAWLVKGGGGGGGSG
GGGGGGGSK[K(N3)]-CONH2

Molecular Formula: C₂₀₁H₃₀₀FN₅₉O₆₉. Exact Mass: 4663.18. Molecular Weight: 4665.95. Counter Ion: TFA. Formula Weight: 5236.05 (+5 TFA). Physical Appearance: White Powder. Storage: −20° C. Weight: 1.9 mg.

The peptide was synthesized using standard Fmoc chemistry. The below synthesis was described with a scale of 0.05 mmol based on the initial loading of the first amino acid.

Swelling of resin: 0.15 g the Rink-amide MBHA resin (initial resin loading 0.3 mmol/g) was swelled using DCM (15 mL×4 times). 20% piperidine in DMF solution was added to the resin. The mixture was agitated for 2×20 min. After the reaction was completed, the resin was washed with DMF (2×5 mL).

Coupling of native amino acid and Side chain (Cas No. 1188328-37-1): Fmoc-AA-OH or 1188328-37-1 (3 eq.)/HBTU (3 eq.)/DIPEA (6 eq.) was dissolved in DMF with a final concentration of 0.2 mM. The mixture was agitated for 60-90 min at room temperature. After the reaction was completed, the resin was washed with DMF (2×5 mL). N-terminal amino acid was coupled with Boc-protected amino acid. Lys with side chain was coupled as Fmoc-Lys(Dde)-OH.

Coupling of alpha-methylated AA (amAA): Fmoc-amAA-OH (3 eq.), HATU (3 eq.), and DIPEA (6 eq.) were dissolved in DMF with a final concentration of 0.2 mM. The mixture was agitated for 16 hours at room temperature. After the reaction was completed, the resin was washed with DMF (2×5 mL).

Coupling of the amino acid after Aib: Fmoc-AA-OH (3 eq.), HATU (3 eq.), and DIPEA (6 eq.) were dissolved in DMF with a final concentration of 0.2 mM. The mixture was agitated for 2×45 min at room temperature. After the reaction was completed, the resin was washed with DMF (2×5 mL).

Coupling of amino acid after amAA: Fmoc-AA-OH (20 eq.), HATU (20 eqv.), and DIPEA (30 eq.) were dissolved in DMF with a final concentration of 0.5 mM. The mixture was agitated for 16 hours at room temperature. Mini cleavage LCMS was necessary to monitor the progress of the reaction. After the reaction was completed, the resin was washed with DMF (2×5 mL).

Dde deprotection: The resin-bound peptide was treated with hydrazine monohydrate (10% in DMF) 10 mL and reacted for 30 min. Once the reaction was completed, the mixture was washed with DMF (10 mL), MeOH (10 mL), DCM (2×10 mL), and DMF (2×10 mL).

Cleavage: The resin bound peptide was cleaved using TFA/TIS/H₂O/MPA (17:1:1:1) for 2 hours. For peptide containing Atz, 5 vol % of EDT was added into the cleavage cocktail. Once completed, the resin was filtered and washed with small volume of TFA twice. The peptide was precipitated by addition of tenfold volume of cold MTBE to the TFA solution and mixed thoroughly. The mixture was centrifuged. The supernatant was decanted. The solid containing crude peptide was further washed with MTBE twice, centrifuged, and then dried under reduced pressure.

Purification: The crude peptide was purified by prep-HPLC (0.01% TFA/CH₃CN/H₂O) to afford the target peptide after lyophilization.

Analytical Data: (ESI) m/z: 1555.9 [M+3H]³⁺, 1167.2 [M+4H]⁴⁺, 934.0 [M+5H]⁵⁺. 96.55% (214 nm), RT=16.82 min, HPLC purity 96.55%.

### Synthesis of M6560

Molecular Formula: C₁₉₉H₂₉₇N₆₃O₆₇
Exact Mass: 4641.18
Molecular Weight: 4643.94
Counter Ion: TFA
Formula Weight: 5214.04 (+5 TFA)
Physical Appearance: White Powder
Storage: −20° C.
Weight: 5.0 mg

Peptide Sequence:
(SEQ ID NO: 56)
H[Aib][Atz]GTFTSDYSSYLEEQAAKEFIAWLV
KGGGGGGGSGGGGSGGGGSK[K(N3)]-CONH₂

The peptide was synthesized using standard Fmoc chemistry. The below synthesis was described with a scale of 0.05 mmol based on the initial loading of the first amino acid.

Swelling of resin: 0.15 g the Rink-amide MBHA resin (initial resin loading 0.3 mmol/g) was swelled using DCM (15 mL×4 times). 20% piperidine in DMF solution was added into the resin. The mixture was agitated for 2×20 min. After the reaction was completed, the resin was washed with DMF (2×5 mL).

Coupling of native amino acid and Side chain (Cas No. 1188328-37-1): Fmoc-AA-OH or 1188328-37-1 (3 eq.), HBTU (3 eq.), and DIPEA (6 eq.) were dissolved in DMF with a final concentration of 0.2 mM. The mixture was agitated for 60-90 min at room temperature. After the reaction was completed, the resin was washed with DMF (2×5 mL). N-terminal amino acid was coupled with Boc-protected amino acid. Lys with side chain was coupled as Fmoc-Lys(Dde)-OH.

Coupling of alpha-methylated AA (amAA): Fmoc-amAA-OH (3 eq.), HATU (3 eq.), and DIPEA (6 eq.) were dissolved in DMF with a final concentration of 0.2 mM. The mixture was agitated for 16 hours at room temperature. After the reaction was completed, the resin was washed with DMF (2×5 mL).

Coupling of the amino acid after Aib: Fmoc-AA-OH (3 eq.), HATU (3 eq.), and DIPEA (6 eq.) were dissolved in DMF with a final concentration of 0.2 mM. The mixture was agitated for 2×45 min at room temperature. After the reaction was completed, the resin was washed with DMF (2×5 mL).

Coupling of amino acid after amAA: Fmoc-AA-OH (20 eq.), HATU (20 eqv.), and DIPEA (30 eqv.) were dissolved in DMF with a final concentration of 0.5 mM. The mixture was agitated for 16 hours at room temperature. Mini cleavage LCMS was necessary to monitor the progress of the reaction. After the reaction was completed, the resin was washed with DMF (2×5 mL).

Dde deprotection: The resin-bound peptide was treated with hydrazine monohydrate (10% in DMF) 10 mL and reacted for 30 min. Once the reaction was completed, the mixture was washed with DMF (10 mL), MeOH (10 mL), DCM (2×10 mL), and DMF (2×10 mL).

Cleavage: The resin bound peptide was cleaved using TFA/TIS/H₂O/MPA (17:1:1:1) for 2 hours. For peptide containing Atz, 5 vol % of EDT was added into the cleavage cocktail. Once completed, the resin was filtered, washed with small volume of TFA twice. The peptide was precipitated by adding tenfold volume of cold MTBE to the TFA solution and mixed thoroughly. The mixture was centrifuged. The supernatant was decanted. The solid containing crude peptide was further washed with MTBE twice, centrifuged, and then dried under reduced pressure.

Purification: The crude peptide was purified by prep-HPLC (0.01% TFA/CH₃CN/H₂O) to afford the target peptide after lyophilization. HPLC purity: 91.25% (214 nm), RT=9.13 min; LCMS: (ESI) m/z: 1548.4 [M+3H]³⁺, 1161.8 [M+4H]⁴⁺, 929.6 [M+5H]⁵⁺.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A composition comprising an GLP1R agonist-tethered GDF8 antibody conjugate, or a pharmaceutically acceptable salt thereof, wherein the conjugate comprises
a) an antibody, or an antigen-binding fragment thereof, that specifically binds to and/or blocks the biological activity of Growth and Differentiation Factor-8 (GDF8, SEQ ID NO: 1);
b) at least one Glucagon-like peptide-1 (GLP-1) receptor (GLP1R) agonist; and
c) at least one linker that covalently connects the at least one GLP1R agonist to the GDF8 antibody, or the antigen-binding fragment thereof.

2.-24. (canceled)

25. A GLP1R agonist-tethered GDF8 antibody conjugate, or a pharmaceutically acceptable salt thereof, having a Formula (I): 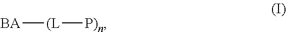
wherein
BA is an antibody, or an antigen-binding fragment thereof, that specifically binds to and/or blocks the biological activity of human Growth and Differentiation Factor-8 (GDF8, SEQ ID NO: 1);
P is a Glucagon-like peptide-1 (GLP-1) receptor (GLP1R) agonist;
L is a linker that covalently links the P to the BA; and
n ranges from about 1 to about 16.

26.-56. (canceled)

57. A compound, or a pharmaceutically acceptable salt thereof, having the structure of Formula (IV): 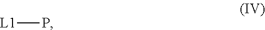
wherein
L1 is a linker comprising a sequence selected from the group consisting of -(Gly₃Ser)ₙ-Lys(X)- (SEQ ID NO: 89) and -(Gly₄Ser)ₙ-Lys(X)- (SEQ ID NO: 90), wherein n is an integer from one to six; and wherein the side chain of Lys is functionalized with a reactive moiety X for conjugation with a target antibody or antigen binding fragment thereof, and
P is a Glucagon-like peptide-1 (GLP-1) receptor (GLP1R) agonist.

58.-64. (canceled)

65. A method for treating obesity by reducing body weight while maintaining or increasing lean body mass in a subject, comprising administering a therapeutically effective amount of a GLP-1 agonist-tethered GDF8 antibody conjugate of claim 26 to the subject in need thereof.

66. A method for treating obesity by reducing fat mass while maintaining or increasing lean body mass in a subject, comprising administering a therapeutically effective amount of a GLP-1 agonist-tethered GDF8 antibody conjugate of claim 26 to the subject in need thereof.

67. A method for treating Type 2 diabetes by improving glycemic control and maintaining or increasing lean body mass while reducing fat mass in a subject, comprising administering a therapeutically effective amount of a GLP-1 agonist-tethered GDF8 antibody conjugate of claim 26 to the subject in need thereof.

68. A method for treating obesity and Type 2 diabetes by improving glycemic control and maintaining or increasing lean body mass while reducing fat mass in a subject, comprising administering a therapeutically effective amount of a GLP-1 agonist-tethered GDF8 antibody conjugate of claim 26 to the subject in need thereof.

69. A method for treating obesity, diabetes, and/or liver diseases associated with increased fat mass in a subject, comprising administering a therapeutically effective amount of a GLP-1 agonist-tethered GDF8 antibody conjugate of claim 26 to the subject in need thereof.

70. A method for treating obesity, diabetes, and/or liver diseases associated with increased fat mass in a subject, comprising administering a therapeutically effective amount of a GLP-1 agonist-tethered GDF8 antibody conjugate of claim 26, together with one or more other therapeutic agents, to the subject in need thereof.

71. A method for treating a subject of metabolic syndrome by improving glycemic control, maintaining or increasing lean body mass while reducing fat mass in a subject, comprising administering a therapeutically effective amount of a GLP-1 agonist-tethered GDF8 antibody conjugate of claim 26 to the subject in need thereof.

72. A method for treating a subject of metabolic syndrome by improving glycemic control, maintaining or increasing lean body mass while reducing fat mass in a subject, comprising administering a therapeutically effective amount of a GLP-1 agonist-tethered GDF8 antibody conjugate of claim 26, together with one or more other therapeutic agents, to the subject in need thereof.

73.-80. (canceled)

81. A process for manufacturing a conjugate of GLP1R agonist tethered GDF8 antibody or antigen binding fragment thereof comprising a) covalently attaching a handle comprising a first reactive moiety for Click or Diels-Alder reaction, in the presence of microbial transglutaminase; b) exposing a GLP1R agonist comprising a second reactive moiety for Click or Diels-Alder reaction, wherein the first and the second reactive moieties are complimentary to each other and form a stable conjugate; and c) isolating or purifying the conjugate of GLP1R agonist tethered GDF8 antibody or antigen binding fragment thereof.

82.-86. (canceled)

87. A conjugate, or a pharmaceutically acceptable salt thereof, comprising an antigen-binding protein (BA) and a GLP1R agonist, wherein the BA specifically binds to and/or blocks the activity of GDF8 (SEQ ID NO: 1), and the GLP1R agonist is conjugated to the BA through a linker.

88.-100. (canceled)

101. A process for conjugating a drug, a ligand, or a handle, site-specifically to an isolated antibody or antigen binding fragment thereof, wherein the site of conjugation is at Gln/Q55 of the light chain of the antibody, and wherein the site-specific conjugation is assisted by microbial transglutaminase (mTG).

102.-113. (canceled)

114. A compound of Formula (V): H-Aib-AA₁-G-T-AA₂-T-S-D-AA₃-AA₄-S-Y-L-E-E-Q-A-A-AA₅-E-AA₆-I-A-W-L-V-AA₇-G-G-G (SEQ ID NO: 71) (V),
wherein
H is His;
Aib is 2-Aminoisobutyric acid;
AA₁ is E or 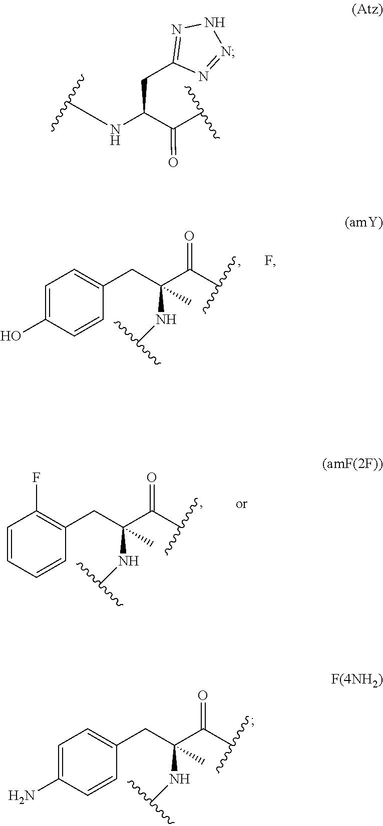 [SMILES: *N[C@@H](Cc1nn[nH]n1)C(*)=O ; *N[C@@](C)(Cc1ccc(O)cc1)C(*)=O ; *N[C@@](C)(Cc1ccccc1F)C(*)=O ; *N[C@@](C)(Cc1ccc(N)cc1)C(*)=O]
AA₃ is 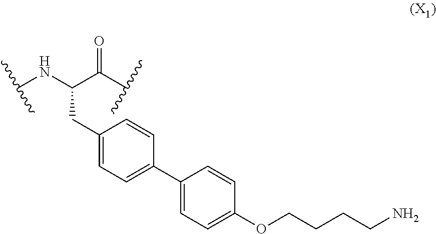 [SMILES: *N[C@@H](Cc1ccc(-c2ccc(OCCCCN)cc2)cc1)C(*)=O]
or Y;
AA₄ is S or 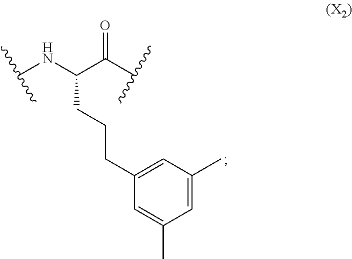 [SMILES: *N[C@@H](CCCc1cc(C)cc(C)c1)C(*)=O]
AA₅ is K or 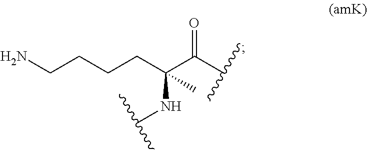 [SMILES: *N[C@@](C)(CCCCN)C(*)=O]
AA₆ is F or 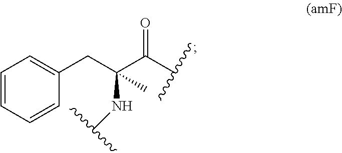 [SMILES: *N[C@@](C)(Cc1ccccc1)C(*)=O]
and
AA₇ is K or amK.

115. The compound of claim 114, wherein AA₁ is E.

116. The compound of claim 114, wherein AA₂ is F.

117. The compound of claim 114, wherein AA₂ is amY.

118. The compound of claim 114, wherein AA₃ is Y.

119. The compound of claim 114, wherein AA₄ is S.

120. The compound of claim 114, wherein AA₅ is K.

121. The compound of claim 114, wherein AA₆ is F.

122. The compound of claim 114, wherein AA₇ is K.

123. The compound of claim 114, wherein the compound is selected form the group consisting of:
(SEQ ID NO: 3)
H(Aib)EGTFTSDYSSYLEEQAAKEFIAWLVKGGG,
(SEQ ID NO: 38)
H[Aib]EGT-amY-TSD-X1-SSYLEEQAAKEFIAWLVKGGG,
(SEQ ID NO: 39)
H[Aib]EGT-amY-TSD-X1-SSYLEEQAA-amK-E-amF-IAWLV-
amK-GGG,
(SEQ ID NO: 40)
H[Aib]EGT-amF(2F)-TSDYSSYLEEQAAKEFIAWLVKGGG,
(SEQ ID NO: 41)
H[Aib]Atz-GTFTSDYSSYLEEQAAKEFIAWLVKGGG,
(SEQ ID NO: 43)
H[Aib]EGT-amY-TSD-X1-SSYLEEQAA-amK-E-amF-IAWLV-
amK-GGG,
(SEQ ID NO: 44)
H[Aib]EGT-amF(2F)-TSDYSSYLEEQAAKEFIAWLVKGGG,
(SEQ ID NO: 45)
H[Aib]EGT-F(4NH2)-TSDYSSYLEEQAAKEFIAWLVKGGG,
(SEQ ID NO: 46)
H[Aib]EGTFTSDY-X2-SYLEEQAAKEFIAWLVKGGG,
(SEQ ID NO: 42)
H[Aib]EGT-amF(2F)-TSDYSSYLEEQAAKEFIWLVKGGG,
and
(SEQ ID NO: 12)
H[Aib]Atz-GTFTSDYSSYLEEQAAKEFIAWLVKGGG.

124.-139. (canceled)

140. A GLP1R agonist-tethered antibody conjugate, or a pharmaceutically acceptable salt thereof, wherein the conjugate comprises
a) an antibody, or an antigen-binding fragment thereof;
b) at least one Glucagon-like peptide-1 (GLP-1) receptor (GLP1R) agonist according to claim 114; and
c) one linker or a bond that covalently connects the at least one said GLP1R agonist to the antibody, or the antigen-binding fragment thereof.

141.-147. (canceled)
